(19) United States
(12) Patent Application Publication
(10) Pub. No.: US 2021/0115127 A1
(43) Pub. Date: Apr. 22, 2021

(54) ANTI-IL-27 ANTIBODIES AND USES THEREOF

(71) Applicant: Surface Oncology, Inc., Cambridge, MA (US)

(72) Inventors: Jamie STRAND, Arlington, MA (US); Jonathan HILL, Salem, MA (US); Devan MOODLEY, West Roxbury, MA (US)

(73) Assignee: Surface Oncology, Inc., Cambridge, MA (US)

(21) Appl. No.: 17/033,469

(22) Filed: Sep. 25, 2020

Related U.S. Application Data
(60) Provisional application No. 62/906,008, filed on Sep. 25, 2019.
(60) Provisional application No. 63/081,705, filed on Sep. 22, 2020.

(51) Int. Cl. C07K 16/24; A61P 35/00; C07K 16/28

(52) U.S. Cl. CPC C07K 16/244; A61P 35/00; C07K 16/2818; A61K 2039/505; C07K 2317/76; C07K 2317/92; C07K 2317/565

(57) ABSTRACT

The present disclosure relates to anti-IL-27 antibodies, and antigen-binding portions thereof. The disclosure also relates to methods for treating or ameliorating one or more symptoms of a disease, such as cancer, by administering the antibodies or antigen-binding portion thereof. The disclosure also relates to methods for detecting IL-27 in, for example, a subject or a sample.

### CROSS REFERENCE TO RELATED APPLICATIONS

This PCT application claims the priority benefit of U.S. Provisional Application Nos. 62/906,008 filed Sep. 25, 2019; and 63/081,705 filed Sep. 22, 2020; each of which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4416_0090003_Seqlisting_ST25.txt; Size: 156,863 bytes; and Date of Creation: Sep. 25, 2020), filed with the application, is incorporated herein by reference in its entirety.

### FIELD

The present disclosure relates generally to compositions and methods for modulating IL-27 signaling. More particularly, the present disclosure relates to immunogenic compositions (e.g., antibodies, antibody fragments, and the like) that bind to IL-27 and modulate IL-27 signaling.

### BACKGROUND

In recent years, an increasing body of evidence suggests that the immune system operates as a significant barrier to tumor formation and progression. The principle that naturally occurring T cells with anti-tumor potential or activity exist in a patient with cancer has rationalized the development of immunotherapeutic approaches in oncology. Immune cells, such as T cells, macrophages, and natural killer cells, can exhibit anti-tumor activity and effectively control the occurrence and growth of malignant tumors. Tumor-specific or -associated antigens can induce immune cells to recognize and eliminate malignancies (Chen & Mellman, (2013) Immunity 39(1):1-10). In spite of the existence of tumor-specific immune responses, malignant tumors often evade or avoid immune attack through a variety of immunomodulatory mechanisms resulting in the failure to control tumor occurrence and progression (Motz & Coukos, (2013) Immunity 39(1):61-730). Indeed, an emerging hallmark of cancer is the exploitation of these immunomodulatory mechanisms and the disablement of anti-tumor immune responses, resulting in tumor evasion and escape from immunological killing (Hanahan and Weinberg (2011) Cell 144(5):646-674).

IL-27 is a heterodimeric cytokine, composed of two subunits (EBI3 and IL-27p28). IL-27 is structurally related to both the IL-12 and IL-6 cytokine families. IL-27 binds to and mediates signaling through a heterodimer receptor consisting of IL-27Rα (WSX1) and gp130 chains, which mediate signaling predominantly through STAT1 and STAT3. Initial reports characterized IL-27 as an immune-enhancing cytokine that supports CD4+ T cell proliferation, T helper (Th)1 cell differentiation, and IFN-γ production, often acting in concert with IL-12. Subsequent studies have shown that IL-27 displays complex immunomodulatory functions, resulting in either proinflammatory or anti-inflammatory effects depending on the biological context and experimental models being used. IL-27 may drive the expression of different immune-regulatory molecules in human cancer cells, which may support local derangement of the immune response in vivo (Fabbi et al., (2017) Mediators Inflamm 3958069. Published online 2017 Feb. 1. doi:10.1155/2017/3958069, and references contained therein).

Despite the significant advances being made in cancer treatment and management, there is still an ongoing need for new and effective therapies for treating and managing cancer.

### SUMMARY OF THE DISCLOSURE

Disclosed herein are antibodies, or antigen binding portions thereof, that antagonize IL-27 and specifically bind to an epitope comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii). In some aspects, the antibody, or antigen binding portion thereof, specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, or Glu164 of SEQ ID NO: 2 (IL-27p28).

In some aspects, the antibody, or antigen binding portion thereof, of the present disclosure specifically binds to an epitope comprising Asp146, Arg149, and/or Phe153 of SEQ ID NO: 2 (IL-27p28). In some aspects, the epitope further comprises His150 and/or Leu156 of SEQ ID NO: 2 (IL-27p28). In some aspects, the epitope further comprises Gln37, Leu38, Glu42, Leu142, and/or Glu164 of SEQ ID NO: 2 (IL-27p28). In some aspects, the epitope further comprises Glu46, Va149, Ser50, and/or Leu162 of SEQ ID NO: 2 (IL-27p28). In some aspects, the epitope further comprises one or more amino acids of Leu53, Lys56, Asp143, Arg145, Leu147, Arg152, Ala157, Gly159, Phe160, Asn161, or Pro163 of SEQ ID NO: 2 (IL-27p28).

In some aspects, the antibody, or antigen binding portion thereof, of the present disclosure specifically binds to an epitope consisting or consisting essentially of Gln37, Leu38, Glu42, Glu46, Va149, Ser50, Leu142, Asp146, Arg149, His150, Phe153, Leu156, Leu162, and Glu164 of SEQ ID NO: 2 (IL-27p28).

In some aspects, the antibody, or antigen binding portion thereof, of the present disclosure specifically binds to an epitope consisting or consisting essentially of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28).

In other aspects, the antibody, or antigen binding portion thereof, that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) does not comprise heavy and light chain CDRs selected from the group consisting of: (i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs:, 18 and 19, respectively; (ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively; (iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively; (iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively; (v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or (vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In other aspects, the antibody, or antigen binding portion thereof, that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) does not comprise heavy and light chain CDRs selected from the group consisting of: (i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively; (ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively; (iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively; (iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 87 and 88, respectively; (v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or (vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the heavy chain CDR1 of the antibody, or antigen binding portion thereof, does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146). In some aspects, the heavy chain CDR1 of the antibody, or antigen binding portion thereof, does not consist of N-FTF[S/A/R][S/R][T/Y][G/S]MN-C (SEQ ID NO: 148) and/or the heavy chain CDR2 does not consist of N-[G/S]ISSS[S/G][S/A]YI[L/Y]YADSVKG-C (SEQ ID NO: 149).

In other aspects, the antibody, or antigen binding portion thereof, that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) does not comprise: (i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or (ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the antibody, or antigen binding portion thereof, that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP[X]ₙ₌₀₋₁T-C (SEQ ID NO: 156), respectively.

Disclosed herein are antibodies, or antigen binding portions thereof, that exhibit at least one or more of the following properties: (i) binds to human IL-27 with an equilibrium dissociation constant (K_{D}) of 15 nM or less; (ii) blocks binding of IL-27 to IL-27 receptor; (iii) inhibits or reduces STAT1 and/or STAT3 phosphorylation in a cell; (iv) inhibits or reduces IL-27-mediated inhibition of CD161 expression in a cell; (v) inhibits or reduces IL-27-mediated PD-L 1 and/or TIM-3 expression in a cell; and (vi) induces or enhances PD-1-mediated secretion of one or more cytokines from a cell.

In some aspects, the isolated antibody, or antigen binding portion thereof, binds to human IL-27 with an equilibrium dissociation constant (K_{D}) of 15 nM or less.

In other aspects, the isolated antibody, or antigen binding portion thereof, inhibits or reduces STAT1 and/or STAT3 phosphorylation in a cell. In some aspects the isolated antibody, or antigen binding portion thereof, reduces STAT1 and/or STAT3 phosphorylation in an immune cell or a cancer cell.

In some aspects, the isolated antibody, or antigen binding portion thereof, inhibits or reduces inhibition of CD161 expression in a cell. In some aspects, the isolated antibody, or antigen binding portion thereof, inhibits or reduces inhibition of CD161 expression in an immune cell.

In other aspects, the isolated antibody, or antigen binding portion thereof, inhibits or reduces PD-L1 and/or TIM-3 expression in a cell. In some aspects, the isolated antibody, or antigen binding portion thereof, inhibits or reduces PD-L1 and/or TIM-3 expression in an immune cell or a cancer cell. In some aspects, the isolated antibody, or antigen binding portion thereof, inhibits or reduces PD-L1 expression in a cancer cell.

In some aspects, the isolated antibody, or antigen binding portion thereof, induces or enhances the PD1-mediated secretion of one or more cytokines from a cell. In some aspects, the one or more cytokines is IFNg (IFNγ), IL-17, TNFa (TNFα), or IL-6. In some aspects, the antibody, or antigen binding portion thereof, is selected from the group consisting of an IgG1, an IgG2, an IgG3, an IgG4, an IgM, an IgA1 an IgA2, an IgD, and an IgE antibody. In other aspects, the antibody, or antigen binding portion thereof, is an IgG1 antibody or an IgG4 antibody. In some aspects, the antibody, or antigen binding portion thereof, comprises an Fc domain comprising at least one mutation. Also disclosed herein are pharmaceutical compositions comprising any one of the described isolated antibodies, or antigen binding portions thereof, and a pharmaceutically acceptable carrier. Also disclosed are nucleic acids comprising a nucleotide sequence encoding the light chain, heavy chain, or both light and heavy chains of the isolated antibody, or antigen binding portion thereof. Disclosed herein is an expression vector comprising the nucleic acid. Further disclosed is a cell transformed with the expression vector.

The present disclosure also provides methods for producing an antibody that specifically binds human IL-27, or an antigen binding portion thereof, comprising maintaining a cell transformed with the expression vector under conditions permitting expression of the antibody or antigen binding portion thereof. In some aspects, the method further comprises obtaining the antibody, or antigen binding portion thereof.

Disclosed herein is a method to inhibit or reduce STAT1 and/or STAT3 phosphorylation in a cell comprising contacting the cell with the antibody, or antigen binding portion thereof, wherein the antibody, or antigen binding portion thereof, inhibits or reduces STAT1 and/or STAT3 phosphorylation in a cell.

Further disclosed is a method to inhibit or reduce inhibition of CD161 expression in a cell, comprising contacting the cell with the antibody, or antigen binding portion thereof, wherein the antibody, or antigen binding portion thereof, inhibits or reduces inhibition of CD161 expression in a cell.

Also disclosed is a method to inhibit or reduce PD-L1 and/or TIM-3 expression in a cell, comprising contacting the cell with the antibody, or antigen binding portion thereof, wherein the antibody, or antigen binding portion thereof, inhibits PD-L1 and/or TIM-3 expression in a cell.

Also disclosed is a method to induce or enhance secretion of one or more cytokines from a cell, comprising contacting the cell with the antibody, or antigen binding portion thereof, wherein the antibody, or antigen binding portion thereof, induces or enhances PD-1 mediated secretion of one or more cytokines from a cell.

Further disclosed is a method of stimulating an immune response in a subject, comprising administering to the subject an effective amount of a disclosed isolated antibody, or antigen binding fragment or a disclosed pharmaceutical composition.

Further disclosed is a method of treating cancer in a subject, comprising administering to the subject an effective amount of a disclosed isolated antibody, or antigen binding fragment or a disclosed pharmaceutical composition.

Disclosed herein is a method of stimulating an immune response, or treating a cancer in a subject. The method comprises administering to the subject an effective amount of a disclosed isolated antibody, or antigen binding portion thereof or a disclosed pharmaceutical composition, wherein the antibody, antigen binding portion thereof, or the pharmaceutical composition inhibits or reduces STAT1 and/or STAT3 phosphorylation in a cell, thereby stimulating immune response, or treating the cancer.

Further disclosed is a method of stimulating an immune response, or treating a cancer in a subject. The method comprises administering to the subject an effective amount of a disclosed isolated antibody, or antigen binding portion thereof or a disclosed pharmaceutical composition, wherein the antibody, antigen binding portion thereof, or the pharmaceutical composition inhibits or reduces inhibition of CD161 expression in a cell, thereby stimulating the immune response, or treating the cancer.

Further disclosed is a method of stimulating an immune response, or treating a cancer in a subject. The method comprises administering to the subject an effective amount of a disclosed isolated antibody, or antigen binding portion thereof or a disclosed pharmaceutical composition, wherein the antibody, antigen binding portion thereof, or the pharmaceutical composition inhibits or reduces PD-L1 and/or TIM-3 expression on a cell, thereby stimulating the immune response, or treating the cancer.

Further disclosed is a method of stimulating an immune response, or treating a cancer in a subject. The method comprises administering to the subject an effective amount of a disclosed isolated antibody, or antigen binding portion thereof or a disclosed pharmaceutical composition, wherein the antibody, antigen binding portion thereof, or the pharmaceutical composition induces or enhances PD-1-mediated secretion of one or more cytokines from a cell, thereby stimulating the immune response, or treating the cancer.

In some aspects, the cancer treated by the method is chosen from lung cancer (e.g., non-small cell lung cancer), sarcoma, testicular cancer, ovarian cancer, pancreas cancer, breast cancer (e.g., triple-negative breast cancer), melanoma, head and neck cancer (e.g., squamous head and neck cancer), colorectal cancer, bladder cancer, endometrial cancer, prostate cancer, thyroid cancer, hepatocellular carcinoma, gastric cancer, brain cancer, lymphoma (e.g., DL-BCL), leukemia (e.g., AML) or renal cancer (e.g., renal cell carcinoma, e.g., renal clear cell carcinoma).

Disclosed herein is a method of enhancing one or more activities of an anti-PD-1 antibody (e.g., enhances PD-1-mediated cytokine secretion; enhances anti-PD-1 mediated TNFα secretion; enhances anti-PD-1 mediated IL-6 secretion from a cell exposed to anti-PD-1 antibodies). The method comprises exposing a cell to a disclosed antibody, or antigen binding portion thereof, concurrently with or sequentially to an anti-PD-1 antibody, thereby to enhance one or more activities of anti-PD1 antibodies.

Further disclosed is a pharmaceutical composition comprising an anti-PD-1 antibody, a disclosed antibody, or antigen binding portion thereof, and a pharmaceutically acceptable carrier.

Also disclosed is a kit comprising an anti-PD-1 antibody, and a disclosed antibody, or antigen binding portion thereof, for concurrent or sequential administration, and instructions for its use.

Disclosed herein are any one of the disclosed methods of stimulating an immune response or treating a cancer wherein the disclosed isolated antibody, or antigen binding portion thereof, is administered in combination with one or more additional therapeutic agents or procedure. The second therapeutic agent or procedure is selected from the group consisting of: a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, or a combination thereof. In some aspects, the one or more additional therapeutic agents is a PD-1 antagonist, a PD-L1 inhibitor, a TIM-3 inhibitor, a LAG-3 inhibitor, a TIGIT inhibitor, a CD112R inhibitor, a TAM inhibitor, a STING agonist, a 4-1BB agonist, a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof. In other aspects, the one or more additional therapeutic agents is a PD-1 antagonist. In some aspects, the PD-1 antagonist is selected from the group consisting of: PDR001, nivolumab, pembrolizumab, pidilizumab, MEDI0680, REGN2810, TSR-042, PF-06801591, and AMP-224. In some aspects, the PD-L1 inhibitor is selected from the group consisting of: FAZ053, Atezolizumab, Avelumab, Durvalumab, and BMS-936559. In other aspects, wherein the one or more additional therapeutic agents is selected from the group consisting of Sunitinib (SUTENT®), Cabozantinib (CABOMETYX®), Axitinib (INLYTA®), Lenvatinib (LENVIMA®), Everolimus (AFINITOR®), Bevacizumab (AVASTIN®), epacadostat, NKTR-214 (CD-122-biased agonist), Tivozanib (FOTIVDA®), abexinostat, Ipilimumab (YERVOY®), tremelimumab, Pazopanib (VOTRIENT®), Sorafenib (NEXAVAR®), Temsirolimus (TORISEL®), Ramucirumab (CYRAMZA®), niraparib, savolitinib, vorolanib (X-82), Regorafenib (STIVARGO®), Donafenib (multikinase inhibitor), Camrelizumab (SHR-1210), pexastimogene devacirepvec (JX-594), Ramucirumab (Cyramza®), apatinib (YN968D1), encapsulated doxorubicin (THERMODOX®), Tivantinib (ARQ197), ADI-PEG 20, binimetinib, apatinib mesylate, nintedanib, lirilumab, Nivolumab (OPDIVO®), Pembrolizumab (KEYTRUDA®), Atezolizumab (TECENTRIQ®), Avelumab (BAVENCIO®), Durvalumab (IMFIMZI®), Cemiplimab-rwlc (LIBTAYO®), tislelizumab, and spartalizumab. In some aspects, the one or more additional therapeutic agents is a TIM-3 inhibitor, optionally wherein the TIM-3 inhibitor is MGB453 or TSR-022. In some aspects, the one or more additional therapeutic agents is a LAG-3 inhibitor, optionally wherein the LAG-3 inhibitor is selected from the group consisting of LAG525, BMS-986016, and TSR-033. In some aspects, the one or more additional therapeutic agents is a TIGIT inhibitor. In other aspects, the one or more additional therapeutic agents is a CD112R inhibitor. In some aspects, the one or more additional therapeutic agents is a TAM (Axl, Mer, Tyro) inhibitor. In some aspects, wherein the one or more additional therapeutic agents is a 4-1BB agonist. In other aspects, the one or more additional therapeutic agents is a Tyrosine Kinase Inhibitor (TKI). In some aspects the TKI is selected from imatinib, dasatinib, nilotinib, bosutinib, or ponatinib. In some aspects, the one or more additional agents is a an agent targeting the adenosine axis. In some aspects the agent targeting the adenosine axis is selected from a CD39 antagonist, a CD73 antagonist, a A2AR antagnoist, A2BR antagonist or a dual A2AR/A2BR antagonist. In some aspects, the one or more additional therapeutic agents is a CD39 antagonist. Examples of CD39 antagnoists include those described in US2019/0284295 (Surface Oncology, Inc.), which is herein incorporated by reference. In some aspects, the one or more additional therapeutic agents is a CD73 antagonist. Examples of CD73 antagonists include small molecule CD73 inhibitors such as AB421 (Arcus), a CD73 antibody, or antigen binding portion thereof, that binds to CD73 such as MEDI9447 (Medimmune), BMS-986179 (Bristol Meyers Squibb), or such as described in US2018/0009899 (Corvus), which is incorporated herein by reference in its entirety. In some aspects, the one or more additional therapeutic agents is a A2AR antagnoist, A2BR antagonist or a dual A2AR/A2BR antagonist. Examples of A2AR, A2BR and dual A2AR/A2BR antagonists include Preladenant/SCH 420814 (Merck/Schering, CAS Registry Number: 377727-87-2), which is described in Hodgson et al., (2009) J Pharmacol Exp Ther 330(1):294-303 and incorporated herein by reference in its entirety; ST-4206 (Leadiant Biosciences), which is described in U.S. Pat. No. 9,133,197 and incorporated herein by reference in its entirety; KW-6356 (Kyowa Hakko Kogyo), Tozadenant/SYN-115 (Acorda), Istradefylline/KW-6002 (Kyowa Hakko Kogyo, CAS Registry Number: 155270-99-8), which is described in LeWitt et al., (2008) Ann Neurol 63(3):295-302 and is incorporated herein by reference in its entirety; theophylline (CAS Registry Number: 58-55-9), NIR178 (Novartis); AB928 (Arcus Biosciences), GBV-2034 (Globavir), Vipadenant (Redox/Juno), AZD4635/HTL-1071 (AstraZeneca/Heptares), which is described in WO2011/095625 and is incorporated herein by reference in its entirety; CPI-444/V81444 (Corvus/Genentech), which is described in WO 2009/156737 and is incorporated herein by reference in its entirety; PBF509 (Palobiofarma/Novartis), which is described in U.S. Pat. No. 8,796,284 and WO 2017/025918 and are incorporated herein by reference in their entirety; A2AR antagonists described in U.S. Pat. Nos. 8,114,845, 9,029,393, US20170015758, or US20160129108, all of which are incorporated herein by reference in their entirety; and ATL-444, MSX-3, SCH-58261, SCH-412,348, SCH-442,416, VER-6623, VER-6947, VER-7835, CGS-15943, or ZM-241,385. In some aspects, the one or more additional therapeutic agents is a CCR8 antagonist. In some aspects the CCR8 antagnoist is selected from a small molecule and an antibody. In some aspects, the one or more additional therapeutic agents is a CTLA4 antagonist. In some aspects, the CTLA4 antagonist is selected from the group consisting of: Yervoy® (ipilimumab or antibody 10D1, described in PCT Publication WO 01/14424), tremelimumab (formerly ticilimumab, CP-675,206), monoclonal or an anti-CTLA-4 antibody described in any of the following publications: WO 98/42752; WO 00/37504; U.S. Pat. No. 6,207,156; Hurwitz et al. (1998) Pro. Natl. Acad. Sci. USA 95(17): 10067-10071; Camacho et al. (2004) J. Clin. Oncology 22(145): antibodies tract No. 2505 (antibody CP-675206); and Mokyr et al. (1998) Cancer Res. 58:5301-5304. Any of the anti-CTLA-4 antibodies disclosed in WO2013/173223 can also be used. In some aspects, the one or more additional therapeutic agents is a VEG-F inhibitor. In some aspects the VEG-F inhibitor is selected from cabozantinib, paopanib, bevacizumab, sunitinib, axitinib, lenvantinib, sorafenib, regorafenib, ponatinib, cabozantinib, vandetanib, ramucirumab, or bevacizumab.

Disclosed herein is a use of the disclosed antibody, or antigen binding portion thereof, or the disclosed pharmaceutical composition for stimulating an immune response in a subject, or for treating cancer in a subject, optionally for use in in combination with one or more additional therapeutic agents or procedure.

Further disclosed is a kit comprising the disclosed antibody, or antigen binding portion thereof, or the disclosed pharmaceutical composition, and instructions for use in stimulating an immune response in a subject, or treating cancer in a subject, optionally with instructions for use in combination with one or more additional therapeutic agents or procedure.

Also disclosed is a kit comprising the disclosed antibody, or antigen binding portion thereof, and instructions for use in detecting IL-27 in a sample from a subject, optionally with instructions for use to detect an IL-27-associated cancer in a subject.

### Definitions

Terms used in the claims and specification are defined as set forth below unless otherwise specified.

It must be noted that, as used in the specification and the appended claims, the singular forms “a,” “an” and “the” include plural references unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “agonist” refers to any molecule that partially or fully promotes, induces, increases, and/or activates a biological activity of a native polypeptide disclosed herein. Suitable agonist molecules specifically include agonist antibodies or antibody fragments, fragments or amino acid sequence variants of native polypeptides, peptides or proteins. In some aspects, activation in the presence of the agonist is observed in a dose-dependent manner. In some aspects, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the signal measured with a negative control under comparable conditions. Also disclosed herein, are methods of identifying agonists suitable for use in the methods of the disclosure. For example, these methods include, but are not limited to, binding assays such as enzyme-linked immuno-absorbent assay (ELISA), FORTE BIO® systems, and radioimmunoassay (MA). These assays determine the ability of an agonist to bind the polypeptide of interest (e.g., a receptor or ligand) and therefore indicate the ability of the agonist to promote, increase or activate the activity of the polypeptide. Efficacy of an agonist can also be determined using functional assays, such as the ability of an agonist to activate or promote the function of the polypeptide. For example, a functional assay may comprise contacting a polypeptide with a candidate agonist molecule and measuring a detectable change in one or more biological activities normally associated with the polypeptide. The potency of an agonist is usually defined by its EC₅₀ value (concentration required to activate 50% of the agonist response). The lower the EC₅₀ value the greater the potency of the agonist and the lower the concentration that is required to activate the maximum biological response.

As used herein, the term “alanine scanning” refers to a technique used to determine the contribution of a specific wild-type residue to the stability or function(s) (e.g., binding affinity) of given protein or polypeptide. The technique involves the substitution of an alanine residue for a wild-type residue in a polypeptide, followed by an assessment of the stability or function(s) (e.g., binding affinity) of the alanine-substituted derivative or mutant polypeptide and comparison to the wild-type polypeptide. Techniques to substitute alanine for a wild-type residue in a polypeptide are known in the art.

The term “ameliorating” refers to any therapeutically beneficial result in the treatment of a disease state, e.g., cancer, including prophylaxis, lessening in the severity or progression, remission, or cure thereof.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “amount” or “level” is used in the broadest sense and refers to a quantity, concentration or abundance of a substance (e.g., a metabolite, a small molecule, a protein, an mRNA, a marker). When referring to a metabolite or small molecule (e.g. a drug), the terms “amount”, “level” and “concentration” are generally used interchangeably and generally refer to a detectable amount in a biological sample. “Elevated levels” or “increased levels” refers to an increase in the quantity, concentration or abundance of a substance within a sample relative to a control sample, such as from an individual or individuals who are not suffering from the disease or disorder (e.g., cancer) or an internal control. In some aspects, the elevated level of a substance (e.g., a drug) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., HPLC). “Reduced levels” refers to a decrease in the quantity, concentration or abundance of a substance (e.g., a drug) in an individual relative to a control, such as from an individual or individuals who are not suffering from the disease or disorder (e.g., cancer) or an internal control. In some aspects, a reduced level is little or no detectable quantity, concentration or abundance. In some aspects, the reduced level of a substance (e.g., a drug) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, HPLC).

When referring to a protein, mRNA or a marker, such as those described herein, the terms “level of expression” or “expression level” in general are used interchangeably and generally refer to a detectable amount of a protein, mRNA, or marker in a biological sample. In some aspects, a detectable amount or detectable level of a protein, mRNA or a marker is associated with a likelihood of a response to an agent, such as those described herein. “Expression” generally refers to the process by which information contained within a gene is converted into the structures (e.g., a protein marker, such as PD-L1) present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). “Elevated expression,” “elevated expression levels,” or “elevated levels” refers to an increased expression or increased levels of a substance within a sample relative to a control sample, such as an individual or individuals who are not suffering from the disease or disorder (e.g., cancer) or an internal control. In some aspects, the elevated expression of a substance (e.g., a protein marker, such as PD-L1) in a sample refers to an increase in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g., FACS). “Reduced expression,” “reduced expression levels,” or “reduced levels” refers to a decrease expression or decreased levels of a substance (e.g., a protein marker) in an individual relative to a control, such as an individual or individuals who are not suffering from the disease or disorder (e.g., cancer) or an internal control. In some aspects, reduced expression is little or no expression. In some aspects, the reduced expression of a substance (e.g., a protein marker) in a sample refers to a decrease in the amount of the substance of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to the amount of the substance in a control sample, as determined by techniques known in the art (e.g, FACS).

As used herein, the term “angiogenesis” or “neovascularization” refers to the process by which new blood vessels develop from pre-existing vessels (Varner et al., (1999) Angiogen. 3:53-60; Mousa et al., (2000) Angiogen. Slim. Inhib. 35:4244; Kim et al., (2000) Amer. J. Path. 156:1345-1362; Kim et al., (2000) J. Biol. Chem. 275:33920-33928; Kumar et al. (2000) Angiogenesis: From Molecular to Integrative Pharm, 169-180). Endothelial cells from pre-existing blood vessels or from circulating endothelial stern cells (Takahashi et al., (1995) Nat. Med. 5:434-438; Isner et al. (1999) J. Clin. Invest. 103:U31-1236) become activated to migrate, proliferate, and differentiate into structures with lumens, forming new blood vessels, in response to growth factor or hormonal cues, or hypoxic or ischemic conditions. During ischemic, such as occurs in cancer, the need to increase oxygenation and delivery of nutrients apparently induces the secretion of angiogenic factors by the affected tissue; these factors stimulate new blood vessel formation Several additional terms are related to angiogenesis.

The term “antagonist,” as used herein, refers to an inhibitor of a target molecule and may be used synonymously herein with the term “inhibitor.” As used herein, the term “antagonist” refers to any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides or proteins. In some aspects, inhibition in the presence of the antagonist is observed in a dose-dependent manner. In some aspects, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions. Also disclosed herein, are methods of identifying antagonists suitable for use in the methods of the disclosure. For example, these methods include, but are not limited to, binding assays such as enzyme-linked immuno-absorbent assay (ELISA), ForteBio®systems, radioimmunoassay (MA), Meso Scale Discovery assay (e.g., Meso Scale Discovery Electrochemiluminescence (MSD-ECL), and bead-based Luminex® assay. These assays determine the ability of an antagonist to bind the polypeptide of interest (e.g., a receptor or ligand) and therefore indicate the ability of the antagonist to inhibit, neutralize or block the activity of the polypeptide. Efficacy of an antagonist can also be determined using functional assays, such as the ability of an antagonist to inhibit the function of the polypeptide or an agonist. For example, a functional assay may comprise contacting a polypeptide with a candidate antagonist molecule and measuring a detectable change in one or more biological activities normally associated with the polypeptide. The potency of an antagonist is usually defined by its IC₅₀ value (concentration required to inhibit 50% of the agonist response). The lower the IC₅₀ value the greater the potency of the antagonist and the lower the concentration that is required to inhibit the maximum biological response.

As used herein, the phrase “antibody that antagonizes human IL-27, or an antigen binding portion thereof” refers to an antibody that antagonizes at least one art-recognized activity of human IL-27 (e.g., IL-27 biological activity and/or downstream pathway(s) mediated by IL-27 signaling or other IL-27-mediated function), for example, relating to a decrease (or reduction) in human IL-27 activity that is at least 5%, 10%, 20%, 30%, 40%, 50%, 60%, 70%, 80%, 90%, or more. Additional examples of IL-27 biological activities and/or downstream pathway(s) mediated by IL-27 signaling or other IL-27-mediated function are described in additional detail below and elsewhere herein.

As used herein, the term “anti-IL-27 antagonist antibody” (interchangeably termed “anti-IL-27 antibody”) refers to an antibody that specifically binds to IL-27 and inhibits IL-27 biological activity and/or downstream pathway(s) mediated by IL-27 signaling or other IL-27-mediated function. An anti-IL-27 antagonist antibody encompasses antibodies that block, antagonize, suppress, inhibit or reduce an IL-27 biological activity (e.g., ligand binding, enzymatic activity), including downstream pathways mediated by IL-27 signaling or function, such as receptor binding and/or elicitation of a cellular response to IL-27 or its metabolites. In some aspects, an anti-IL-27 antagonist antibody provided by the disclosure binds to human IL-27 and prevents, blocks, or inhibits binding of human IL-27 to its cognate or normal receptor (e.g., IL-27 receptor), or one or more receptor subunits (e.g., gp130 and/or IL-27Rα (also known as WSX1/TCCR)). In some aspects, the anti-IL-27 antagonist antibody prevents, blocks, or inhibits the binding of human IL-27 to the gp130. In some aspects, the anti-IL-27 antagonist antibody prevents, blocks, or inhibits the binding of human IL-27 to the IL-27Ra. In some aspects, the anti-IL-27 antagonist antibody prevents, blocks, or inhibits the dimerization of IL-27 monomers. In some aspects, the anti-IL-27 antibody does not specifically bind to the EBI3 monomer. In some aspects, the anti-IL-27 antibody specifically binds to the IL-27p28 monomer. In some embodiments, the anti-IL-27 antibody specifically binds to a non-contiguous epitope comprising P28, but does not bind to the EBI3 monomer. In some aspects, the anti-IL-27 antibody inhibits or reduces STAT1 and/or STAT3 phosphorylation in a cell. In some aspects, the anti-IL-27 antibody inhibits or reduces inhibition of CD161 expression in a cell (e.g., ameliorates or relieves IL-27 mediated inhibition of CD161 expression in a cell). In some aspects, the anti-IL-27 antibody inhibits or reduces PD-L1 and/or TIM-3 expression in a cell. In some aspects, the anti-IL-27 induces or enhances PD-1-mediated secretion of one or more cytokines from a cell. In some aspects, an anti-IL-27 antagonist antibody binds to human IL-27 and stimulates or enhances an anti-tumor response. In some aspects, the anti-IL-27 antagonist antibody binds to human IL-27 with an affinity of 15 nM or less. In some aspects, the anti-IL-27 antagonist antibody binds to human IL-27 and comprises a wild type or mutant IgG1 heavy chain constant region or a wild type or mutant IgG4 heavy chain constant region. Examples of anti-IL-27 antagonist antibodies are provided herein.

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. As used herein, the term “antibody fragment,” “antigen-binding fragment,” or similar terms refer to a fragment of an antibody that retains the ability to bind to a target antigen (e.g., IL-27) and inhibit the activity of the target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al., (2001) J. Immunol. Methods 248(1):47-66; Hudson and Kortt, (1999) J. Immunol. Methods 231(1):177-189; Poljak, (1994) Structure 2(12):1121-1123; Rondon and Marasco, (1997) Annu. Rev. Microbiol. 51:257-283, the disclosures of each of which are incorporated herein by reference in their entirety.

As used herein, the term “antibody fragment” also includes, e.g., single domain antibodies such as camelized single domain antibodies. See, e.g., Muyldermans et al., (2001) Trends Biochem. Sci. 26:230-235; Nuttall et al., (2000) Curr. Pharm. Biotech. 1:253-263; Reichmann et al., (1999) J. Immunol. Meth. 231:25-38; PCT application publication nos. WO 94/04678 and WO 94/25591; and U.S. Pat. No. 6,005,079, all of which are incorporated herein by reference in their entireties. In some aspects, the disclosure provides single domain antibodies comprising two VH domains with modifications such that single domain antibodies are formed.

In some aspects, an antigen-binding fragment includes the variable region of a heavy chain polypeptide and the variable region of a light chain polypeptide. In some aspects, an antigen-binding fragment described herein comprises the CDRs of the light chain and heavy chain polypeptide of an antibody.

The term “antigen presenting cell” or “APC” is a cell that displays foreign antigen complexed with MHC on its surface. T cells recognize this complex using T cell receptor (TCR). Examples of APCs include, but are not limited to, B cells, dendritic cells (DCs), peripheral blood mononuclear cells (PBMC), monocytes (such as THP-1), B lymphoblastoid cells (such as C1R.A2, 1518 B-LCL) and monocyte-derived dendritic cells (DCs). Some APCs internalize antigens either by phagocytosis or by receptor-mediated endocytosis.

The term “antigen presentation” refers to the process by which APCs capture antigens and enables their recognition by T cells, e.g., as a component of an MHC-I and/or MHC-II conjugate.

As used herein, the term “apoptosis” refers to the process of programmed cell death that occurs in multicellular organisms (e.g. humans). The highly regulated biochemical and molecular events that result in apoptosis can lead to observable and characteristic morphological changes to a cell, including membrane blebbing, cell volume shrinkage, chromosomal DNA condensation and fragmentation, and mRNA decay. A common method to identify cells, including T cells, undergoing apoptosis is to expose cells to a fluorophore-conjugated protein (Annexin V). Annexin V is commonly used to detect apoptotic cells by its ability to bind to phosphatidylserine on the outer leaflet of the plasma membrane, which is an early indicator that the cell is undergoing the process of apoptosis.

As used herein, the term “B cell” (alternatively “B lymphocyte”) refers to a type of white blood cell of the lymphocyte subtype. B cells function in the humoral immunity component of the adaptive immune system by secreting antibodies. B cells also present antigen and secrete cytokines. B cells, unlike the other two classes of lymphocytes, T cells and natural killer cells, express B cell receptors (BCRs) on their cell membrane. BCRs allow the B cell to bind to a specific antigen, against which it will initiate an antibody response.

As used herein, the term “binds to immobilized IL-27,” refers to the ability of an antibody of the disclosure to bind to IL-27, for example, expressed on the surface of a cell or which is attached to a solid support.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

Traditionally, the recombinant production of bispecific antibodies is based on the co-expression of two immunoglobulin heavy-chain/light-chain pairs, where the two heavy chain/light chain pairs have different specificities (Milstein and Cuello, (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. For further details of illustrative currently known methods for generating bispecific antibodies see, e.g., Suresh et al., (1986) Methods Enzymol. 121:210; PCT Publication No. WO 96/27011; Brennan et al., (1985) Science 229:81; Shalaby et al., J. Exp. Med. (1992) 175:217-225; Kostelny et al., (1992) J. Immunol. 148(5):1547-1553; Hollinger et al., (1993) Proc. Natl. Acad. Sci. USA 90:6444-6448; Gruber et al., (1994) J. Immunol. 152:5368; and Tutt et al., (1991) J. Immunol. 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., tri specific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some aspects, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

As used herein, “cancer antigen” or “tumor antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, the term “cancer-specific immune response” refers to the immune response induced by the presence of tumors, cancer cells, or cancer antigens. In certain aspects, the response includes the proliferation of cancer antigen specific lymphocytes. In certain aspects, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In certain aspects, the cancer-specific immune response is a T cell response.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The anti-IL-27 antibodies described herein can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

As used herein, the term “CD112R” refers to a member of poliovirus receptor-like proteins and is a co-inhibitory receptor for human T cells. CD112R is an inhibitory receptor primarily expressed by T cells and NK cells and competes for CD112 binding with the activating receptor CD226. The interaction of CD112 with CD112R is of higher affinity than with CD226 and thereby effectively regulates CD226 mediated cell activation. Anti-CD112R antagonists that block the interaction with CD112 limit inhibitory signaling directly downstream of CD112R while simultaneously promoting greater immune cell activation by increasing CD226 interactions with CD112. As used herein the term “CD112R inhibitor” refers to an agent that disrupts, blocks or inhibits the biological function or activity of CD112R.

As used herein, the term “CD137” (alternatively “4-1BB”) refers to a member of the tumor necrosis factor (TNF) receptor superfamily. 4-1BB is a co-stimulatory immune checkpoint molecule, primarily for activated T cells. Crosslinking of CD137 enhances T cell proliferation, IL-2 secretion, survival and cytolytic activity. As used herein, the term “4-1BB agonist” refers to an agent that stimulates, induces or increases one or more function of 4-1BB. An exemplary 4-1BB agonist is Utomilumab (PF-05082566), a fully human IgG2 monoclonal antibody that targets this 4-1BB to stimulate T cells.

As used herein, the term “CD161” (alternatively known as Killer cell lectin-like receptor subfamily B, member 1 (KLRB1); NK1.1, or NKR-P1A) refers to a member of the C-type lectin superfamily. CD161 is a marker of T cells and CD161 expression has been associated with T cell infiltration into the tumor microenvironment for a number of different cancer types. CD161 is further described in Fergusson et al., (2014) Cell Reports 9(3):1075-1088, which is incorporated herein by reference it its entirety.

As used herein, the term “IL-27” or “interleukin 27” refers to the IL-27 cytokine. IL-27 is related to the IL-6/IL-12 cytokine families, and is a heterodimeric cytokine that comprises a first subunit known as Epstein-Barr Virus Induced Gene 3 (EBI3; also known as IL-27 subunit β and IL-27B) and a second subunit known as IL-27p28 (also known as IL30, IL-27 subunit α and IL-27A). IL-27 is predominantly synthesized by activated antigen-presenting cells including monocytes, endothelial cells and dendritic cells (Jankowski et al. (2010) Arch Immunol. Ther. Exp. 58:417-425, Diakowski et al. (2013) Adv. Clin. Exp. Med. (2013) 22(5): 683-691). Although IL-27 can have proinflammatory effects, many studies suggest an important role of IL-27 as an immunosuppressive agent (Shimizu et al. (2006) J. Immunol. 176:7317-7324, Hisada et al. (2004) Cancer Res. 64:1152-1156, Diakowski (2013) supra). Although it was initially described as a factor promoting the initiation of Th1 responses, IL-27 was later found to play a major T-cell suppressive function by limiting Th1 responses, inhibiting Th2 and Th17 cell differentiation, and regulating the development of Tr1 and other T regulatory cell populations (Dietrich et al. (2014) J. Immunol. 192:5382-5389). In addition to its role as an immunoregulator, IL-27 also regulates angiogenesis, hematopoiesis, and osteocalstogenesis (Id.).

IL-27 signals through a heterodimeric type I cytokine receptor (the IL-27 receptor or IL-27R) that comprises a first subunit known as WSX1 (also known as IL-27 receptor subunit a, IL-27RA, T-Cell Cytokine Receptor Type 1 (TCCR), and Cytokine Receptor-Like 1 (CRL1)) and a second subunit known as gp130 (also known as Interleukin-6 Signal Transducer (IL6ST), Interleukin-6 Receptor Subunit β (IL-6RB), and Oncostatin M Receptor). gp130 is also a receptor subunit for the IL-6 family cytokines (Liu et al. (2008) Scan. J. Immunol. 68:22-299, Diakowski (2013) supra). IL-27 signaling through IL-27R activates multiple signaling cascades, including the JAK-STAT and p38 MAPK pathways.

EBI3 is also believed to have biological functions independent of p28 or the IL-27 heterodimer. For example, EBI3 also interacts with p35 to form the heterodimeric cytokine IL-35 (Yoshida et al. (2015) Annu. Rev Immunol. 33:417-43) and has been shown to be selectively overexpressed in certain cell types without a corresponding increase in p28 or IL-27 (Larousserie et al. (2005) Am. J. Pathol. 166(4):1217-28).

An amino acid sequence of an exemplary human EBI3 protein is provided in SEQ ID NO: 1 (NCBI Reference Sequence: NP_005746.2; N-mtpqlllalvlwascppcsgrkgppaaltlprvqcrasrypiavdcswtlppapnstspvsfiatyrlgmaarghswpclqqtptstsctitdvq lfsmapyvinvtavhpwgssssfvpfitehiikpdppegvrlsplaerqlqvqweppgswpfpeifslkywirykrqgaarfhrvgpieatsf ilravrpraryyvqvaaqdltdygelsdwslpatatmslgk-C). An amino acid sequence of an exemplary human p28 protein is provided in SEQ ID NO: 2 (NCBI Reference Sequence: NP_663634.2; N-mgqtagdlgwrlsllllplllvgagvwgfprppgrpqlslqelrreftvslhlarkllsevrgqahrfaeshlpgvnlyllplgeqlpdvsltfqaw rrlsdperlcfisttlqpfhallgglgtqgrwtnmermqlwamrldlrdlqrhlrfqvlaagfnlpeeeeeeeeeeeeerkgllpgalgsalqgpa qvswpqllstyrllhslelvlsravrellllskaghsvwplgfptlspqp-C). An amino acid sequence of an exemplary human WSX1 protein is provided in SEQ ID NO: 3 (NCBI Reference Sequence: NP_004834.1; N-mrggrgapfwlwplpklallpllwvlfqrtrpqgsagplqcygvgplgdlncsweplgdlgapselhlqsqkyrsnktqtvavaagrswvai preqltmsdkllvwgtkagqplwppvfvnletqmkpnaprlgpdvdfseddpleatvhwapptwpshkvlicqfhyrrcqeaawtllep elktipltpveiqdlelatgykvygrcrmekeedlwgewspilsfqtppsapkdvwvsgnlcgtpggeeplllwkapgpcvqvsykvwf wvggrelspegitcccslipsgaewarysavnatswepltnlsivadsasaprsvavssiagstellvtwqpgpgeplehvvdwardgdple klnwvrlppgnlsallpgnftvgvpyritvtavsasglasassvwgfreelaplvgptlwrlqdappgtpaiawgevprhqlrghlthytlcaq sgtspsvcmnvsgntqsvtlpdlpwgpcelwvtastiagqgppgpilrlhlpdntlrwkvlpgilflwglfllgcglslatsgrcyhlrhkvlpr wvwekvpdpansssgqphmeqvpeaqplgdlpileveemepppvmessqpaqatapldsgyekhflptpeelgllgpprpqvla-C). An amino acid sequence of an exemplary human gp130 protein is provided in SEQ ID NO: 4 (NCBI Reference Sequence: NP_002175.2; N-mltlqtwlvqalfiflttestgelldpcgyispespvvqlhsnftavcvlkekcmdyfhvnanyivwktnhftipkeqytiinrtassvtftdiasl niqltcniltfgqleqnvygitiisglppekpknlscivnegkkmrcewdggrethletnftlksewathkfadckakrdtptsctvdystvyfv nievwveaenalgkvtsdhinfdpvykvkpnpphnlsvinseelssilkltwtnpsiksviilkyniqyrtkdastwsqippedtastrssftvq dlkpfteyvfrircmkedgkgywsdwseeasgityedrpskapsfwykidpshtqgyrtvqlvwktlppfeangkildyevtltrwkshlq nytvnatkltvnitndrylatltvrnlvgksdaavltipacdfqathpvmdlkafpkdnmlwvewttpresvkkyilewcvlsdkapcitdw qqedgtvhrtylrgnlaeskcylitvtpvyadgpgspesikaylkqappskgptvrtkkvgkneavlewdqlpvdvqngfirnytifyrtiig netavnvdsshteytlssltsdtlymvrmaaytdeggkdgpeftfttpkfaqgeieaivvpvclafllttllgvlfcfnkrdlikkhiwpnvpdps kshiaqwsphtpprhnfnskdqmysdgnftdvsvveieandkkpfpedlksldlfkkekinteghssgiggsscmsssrpsisssdeness qntsstvqystvvhsgyrhqvpsvqvfsrsestqpildseerpedlqlvdhydggdgilprqqyfkqncsqhesspdishferskqvssvnee dfvrlkqqisdhisqscgsgqmkmfqevsaadafgpgtegqverfetvgmeaatdegmpksylpqtvrqggympq-C).

As used herein the term “compete”, when used in the context of antigen-binding proteins (e.g., immunoglobulins, antibodies, or antigen-binding fragments thereof) that compete for binding to the same epitope, refers to a interaction between antigen-binding proteins as determined by an assay (e.g., a competitive binding assay; a cross-blocking assay), wherein a test antigen-binding protein (e.g., a test antibody) inhibits (e.g., reduces or blocks) specific binding of a reference antigen-binding protein (e.g., a reference antibody) to a common antigen (e.g., IL-27 or a fragment thereof).

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In certain aspects, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain aspects, the antibodies of the disclosure are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

It will also be understood by one of ordinary skill in the art that the antibodies suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The antibodies suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain aspects, a string of amino acids can be replaced with a structurally similar string that differs in order and/or composition of side chain family members. Alternatively, in certain aspects, mutations may be introduced randomly along all or part of a coding sequence, such as by saturation mutagenesis, and the resultant mutants can be incorporated into binding polypeptides of the invention and screened for their ability to bind to the desired target.

As used herein, the term antigen “cross-presentation” refers to presentation of exogenous protein antigens to T cells via MEW class I and class II molecules on APCs.

As used herein, the term “cross-reacts” refers to the ability of an antibody of the disclosure to bind to IL-27 from a different species. For example, an antibody of the present disclosure which binds human IL-27 may also bind another species of IL-27. As used herein, cross-reactivity is measured by detecting a specific reactivity with purified antigen in binding assays (e.g., SPR, ELISA) or binding to, or otherwise functionally interacting with, cells physiologically expressing IL-27. Methods for determining cross-reactivity include standard binding assays as described herein, for example, by Biacore™ surface plasmon resonance (SPR) analysis using a Biacore 2000 SPR instrument (Biacore AB, Uppsala, Sweden), or flow cytometric techniques.

As used herein, the term “cytotoxic T lymphocyte (CTL) response” refers to an immune response induced by cytotoxic T cells. CTL responses are mediated primarily by CD8⁺ T cells.

As used herein, the term “dendritic cell” or “DC” refers to type of antigen-presenting cells that are bone marrow (BM)-derived leukocytes and are the most potent type of antigen-presenting cells. DCs are capture and process antigens, converting proteins to peptides that are presented on major histocompatibility complex (MHC) molecules recognized by T cells. DCs are heterogeneous, e.g. myeloid and plasmacytoid DCs; although all DCs are capable of antigen uptake, processing and presentation to naive T cells, the DC subtypes have distinct markers and differ in location, migratory pathways, detailed immunological function and dependence on infections or inflammatory stimuli for their generation. During the development of an adaptive immune response, the phenotype and function of DCs play a role in initiating tolerance, memory, and polarized T-helper 1 (Th1), Th2 and Th17 differentiation.

As used herein, the term “dendritic cell activation” refers to the transition from immature to mature dendritic cell; and the activated dendritic cells encompass mature dendritic cells and dendritic cells in the process of the transition, wherein the expression of CD80 and CD86 that induce costimulatory signals are elevated by the activating stimuli. Mature human dendritic cells are cells that are positive for the expression of CD40, CD80, CD86, and HLA-class II (e.g., HLA-DR). An immature dendritic cell can be distinguished from a mature dendritic cell, for example, based on markers selected from the group consisting of CD80 and CD86. An immature dendritic cell is weakly positive and preferably negative for these markers, while a mature dendritic cell is positive. Discrimination of mature dendritic cells is routinely performed by those skilled in the art, and the respective markers described above and methods for measuring their expression are also well known to those skilled in the art.

As used herein, the term “EC₅₀” refers to the concentration of an antibody or an antigen-binding portion thereof, which induces a response, either in an in vitro or an in vivo assay, which is 50% of the maximal response, i.e., halfway between the maximal response and the baseline.

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “epitope” or “antigenic determinant” refers to a site on an antigen to which an immunoglobulin or antibody specifically binds. The term “epitope mapping” refers to a process or method of identifying the binding site, or epitope, of an antibody, or antigen binding fragment thereof, on its target protein antigen. Epitope mapping methods and techniques are provided herein. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from IL-27 are tested for reactivity with the given anti-IL-27 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present disclosure are antibodies that bind to an epitope on IL-27 which comprises all or a portion of an epitope recognized by the particular antibodies described herein (e.g., the same or an overlapping region or a region between or spanning the region).

Also encompassed by the present disclosure are antibodies that bind the same epitope and/or antibodies that compete for binding to human IL-27 with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as IL-27. Numerous types of competitive binding assays are known, for example: solid phase direct or indirect radioimmunoassay (MA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label MA using I-125 label (see Morel et al., Mol. Immunol. 25(1):7 (1988)); solid phase direct biotin-avidin EIA (Cheung et al., Virology 176:546 (1990)); and direct labeled MA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope and mass spectrometry combined with hydrogen/deuterium (H/D) exchange which studies the conformation and dynamics of antigen:antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the term “Fc-mediated effector functions” or “Fc effector functions” refer to the biological activities of an antibody other than the antibody's primary function and purpose. For example, the effector functions of a therapeutic agnostic antibody are the biological activities other than the activation of the target protein or pathway. Examples of antibody effect functions include C1q binding and complement dependent cytotoxicity; Fc receptor binding; antibody-dependent cell-mediated cytotoxicity (ADCC); phagocytosis; down regulation of cell surface receptors (e.g., B cell receptor); lack of activation of platelets that express Fc receptor; and B cell activation. Many effector functions begin with Fc binding to an Fcγ receptor. In some aspects, the tumor antigen-targeting antibody has effector function, e.g., ADCC activity. In some aspects, a tumor antigen-targeting antibody described herein comprises a variant constant region having increased effector function (e.g. increased ability to mediate ADCC) relative to the unmodified form of the constant region.

As used herein, the term “Fc receptor” refers to a polypeptide found on the surface of immune effector cells, which is bound by the Fc region of an antibody. In some aspects, the Fc receptor is an Fcγ receptor. There are three subclasses of Fcγ receptors, FcγRI (CD64), FcγRII (CD32) and FycRIII (CD16). All four IgG isotypes (IgG1, IgG2, IgG3 and IgG4) bind and activate Fc receptors FcγRI, FcγRIIA and FcγRIIIA FcγRIIB is an inhibitory receptor, and therefore antibody binding to this receptor does not activate complement and cellular responses. FcγRI is a high affinity receptor that binds to IgG in monomeric form, whereas FcγRIIA and FcγRIIA are low affinity receptors that bind IgG only in multimeric form and have slightly lower affinity. The binding of an antibody to an Fc receptor and/or C1q is governed by specific residues or domains within the Fc regions. Binding also depends on residues located within the hinge region and within the CH2 portion of the antibody. In some aspects, the agonistic and/or therapeutic activity of the antibodies described herein is dependent on binding of the Fc region to the Fc receptor (e.g., FcγR). In some aspects, the agonistic and/or therapeutic activity of the antibodies described herein is enhanced by binding of the Fc region to the Fc receptor (e.g., FcγR).

A list of certain Fc receptor sequences employed in the instant disclosure is set forth as Table 13 below.

As used herein, the term “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

As used herein, the term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (See, e.g., Lonberg et al., (1994) Nature 368(6474): 856-859); Lonberg, (1994) Handbook of Experimental Pharmacology 113:49-101; Lonberg & Huszar, (1995) Intern. Rev. Immunol. 13:65-93, and Harding & Lonberg, (1995) Ann. N.Y. Acad. Sci. 764:536-546). However, the term “human antibody” does not include antibodies in which CDR sequences derived from the germline of another mammalian species, such as a mouse, have been grafted onto human framework sequences (i.e. humanized antibodies).

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The terms “inducing an immune response” and “enhancing an immune response” are used interchangeably and refer to the stimulation of an immune response (i.e., either passive or adaptive) to a particular antigen. The terms “induce” as used with respect to inducing CDC or ADCC refer to the stimulation of particular direct cell killing mechanisms.

As used herein, the term “immunogenic cell death” (alternatively known as “immunogenic apoptosis” refers to a cell death modality associated with the activation of one or more signaling pathways that induces the pre-mortem expression and emission of damaged-associated molecular pattern (DAMPs) molecules (e.g., adenosine triphosphate, ATP) from the tumor cell, resulting in the increase of immunogenicity of the tumor cell and the death of the tumor cell in an immunogenic manner (e.g., by phagocytosis). As used herein, the term “immunogenic cell death-inducing agent” refers to a chemical, biological, or pharmacological agent that induces an immunogenic cell death process, pathway, or modality.

As used herein, the terms “inhibits”, “reduces” or “blocks” (e.g., referring to inhibition or reduction of human IL-27-mediated phosphorylation of STAT1 and/or STAT3 in a cell) are used interchangeably and encompass both partial and complete inhibition/blocking. The inhibition/blocking of IL-27 reduces or alters the normal level or type of activity that occurs without inhibition or blocking. Inhibition and blocking are also intended to include any measurable decrease in the binding affinity of IL-27 when in contact with an anti-IL-27 antibody as compared to IL-27 not in contact with an anti-IL-27 antibody, e.g., inhibits binding of IL-27 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the terms “inhibits angiogenesis,” “diminishes angiogenesis,” and “reduces angiogenesis” refer to reducing the level of angiogenesis in a tissue to a quantity which is at least 10%, LS %, 20%, 25%. 30%, 35%, 40%, 45%, 50%, 55%, 60%, 65%, 70%, 75%. 80%, 85%, 90%, 95%, 99% or less than the quantity in a corresponding control tissue, and most preferably is at the same level which is observed in a control tissue.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an anti-IL-27 antibody).

The term “in vivo” refers to processes that occur in a living organism.

As used herein, the term “isolated antibody” is intended to refer to an antibody which is substantially free of other antibodies having different antigenic specificities (e.g., an isolated antibody that specifically binds to human IL-27 is substantially free of antibodies that specifically bind antigens other than IL-27). An isolated antibody that specifically binds to an epitope may, however, have cross-reactivity to other IL-27 proteins from different species. However, the antibody continues to display specific binding to human IL-27 in a specific binding assay as described herein. In addition, an isolated antibody is typically substantially free of other cellular material and/or chemicals. In some aspects, a combination of “isolated” antibodies having different IL-27 specificities is combined in a well-defined composition.

As used herein, the term “isolated nucleic acid molecule” refers to nucleic acids encoding antibodies or antibody portions (e.g., VH, VL, CDR3) that bind to IL-27, is intended to refer to a nucleic acid molecule in which the nucleotide sequences encoding the antibody or antibody portion are free of other nucleotide sequences encoding antibodies or antibody portions that bind antigens other than IL-27, which other sequences may naturally flank the nucleic acid in human genomic DNA. For example, a sequence selected from a sequence set forth in Table 12 corresponds to the nucleotide sequences comprising the heavy chain (VH) and light chain (VL) variable regions of anti-IL-27 antibody monoclonal antibodies described herein.

As used herein, “isotype” refers to the antibody class (e.g., IgM or IgG1) that is encoded by heavy chain constant region genes. In some aspects, a human monoclonal antibody of the disclosure is of the IgG1 isotype. In some aspects, a human monoclonal antibody of the disclosure is of the IgG2 isotype. In some aspects, a human monoclonal antibody of the disclosure is of the IgG3 isotype. In some aspects, a human monoclonal antibody of the disclosure is of the IgG4 isotype. As is apparent to a skilled artisan, identification of antibody isotypes (e.g., IgG1, IgG2, IgG3, IgG4, IgM, IgA1 IgA2, IgD, and IgE) is routine in the art and commonly involves a combination of sequence alignments with known antibodies, published Fc variant sequences and conserved sequences.

As used herein, the term “isotype switching” refers to the phenomenon by which the class, or isotype, of an antibody changes from one Ig class to one of the other Ig classes.

As used herein the term “KD” or “K_{D}” refers to the equilibrium dissociation constant of a binding reaction between an antibody and an antigen. The value of K_{D} is a numeric representation of the ratio of the antibody off-rate constant (kd) to the antibody on-rate constant (ka). The value of K_{D} is inversely related to the binding affinity of an antibody to an antigen. The smaller the K_{D} value the greater the affinity of the antibody for its antigen. Affinity is the strength of binding of a single molecule to its ligand and is typically measured and reported by the equilibrium dissociation constant (K_{D}), which is used to evaluate and rank order strengths of bimolecular interactions.

As used herein, the term “kd” or “k_{d}” (alternatively “koff” or “k_{off}”) is intended to refer to the off-rate constant for the dissociation of an antibody from an antibody/antigen complex. The value of k_{d} is a numeric representation of the fraction of complexes that decay or dissociate per second, and is expressed in units sec⁻¹.

As used herein, the term “ka” or “kₐ” (alternatively “kon” or “kₒₙ”) is intended to refer to the on-rate constant for the association of an antibody with an antigen. The value of ka is a numeric representation of the number of antibody/antigen complexes formed per second in a 1 molar (1M) solution of antibody and antigen, and is expressed in units M⁻¹sec⁻¹.

As used herein, the term “leukocyte” refers to a type of white blood cell involved in defending the body against infective organisms and foreign substances. Leukocytes are produced in the bone marrow. There are 5 main types of white blood cells, subdivided between 2 main groups: polymorphonuclear leukocytes (neutrophils, eosinophils, basophils) and mononuclear leukocytes (monocytes and lymphocytes).

As used herein, the term “lymphocytes” refers to a type of leukocyte or white blood cell that is involved in the immune defenses of the body. There are two main types of lymphocytes: B-cells and T-cells.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

As used herein, “MHC molecules” refers to two types of molecules, MHC class I and MHC class II. MHC class I molecules present antigen to specific CD8+ T cells and MHC class II molecules present antigen to specific CD4+ T cells. Antigens delivered exogenously to APCs are processed primarily for association with MHC class II. In contrast, antigens delivered endogenously to APCs are processed primarily for association with MHC class I.

As used herein, the term “monoclonal antibody” refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “monocyte” refers to a type of leukocyte and can differentiate into macrophages and dendritic cells to effect an immune response.

As used herein, the term “natural killer (NK) cell” refers to a type of cytotoxic lymphocyte. These are large, usually granular, non-T, non-B lymphocytes, which kill certain tumor cells and play an important role in innate immunity to viruses and other intracellular pathogens, as well as in antibody-dependent cell-mediated cytotoxicity (ADCC).

As used herein, the term “naturally occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

As used herein, the term “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “patient” includes human and other mammalian subjects that receive either prophylactic or therapeutic treatment.

As used herein, the term “PD-1 antagonist” refers to any chemical compound or biological molecule that inhibits the PD-1 signaling pathway or that otherwise inhibits PD-1 function in a cell (e.g. an immune cell). In some aspects, a PD-1 antagonist blocks binding of PD-L1 to PD-1 and/or PD-L2 to PD-1. In some aspects, the PD-1 antagonist specifically binds PD-1. In some aspects, the PD-1 antagonist specifically binds PD-L1.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition which reduces the frequency of, or delays the onset of, symptoms of a medical condition in a subject relative to a subject which does not receive the composition.

As used herein, the term “purified” or “isolated” as applied to any of the proteins (antibodies or fragments) described herein refers to a polypeptide that has been separated or purified from components (e.g., proteins or other naturally occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the term “Programmed Cell Death Protein 1” or “PD-1” refers to the Programmed Cell Death Protein 1 polypeptide, an immune-inhibitory receptor belonging to the CD28 family and is encoded by the PDCD1 gene in humans. Alternative names or synonyms for PD-1 include: PDCD1, PD1, CD279 and SLEB2. PD-1 is expressed predominantly on previously activated T cells, B cells, and myeloid cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773.

As used herein, the term “Programmed Death Ligand-1” or “PD-L1” is one of two cell surface glycoprotein ligands for PD-1 (the other being PD-L2) that downregulates T cell activation and cytokine secretion upon binding to PD-1. Alternative names and synonyms for PD-L1 include: PDCD1L1, PDL1, B7H1, B7-4, CD274 and B7-H. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

PD-1 is known as an immune-inhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to a decrease in T-cell receptor mediated proliferation (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

For several cancers, tumor survival and proliferation is sustained by tumor-mediated immune checkpoint modulation. This modulation can result in the disruption of anti-cancer immune system functions. For example, recent studies have indicated that the expression of immune checkpoint receptors ligands, such as PD-L1 or PD-L2, by tumor cells can downregulate immune system activity in the tumor microenvironment and promote cancer immune evasion. particularly by suppressing T cells. PD-L1 is abundantly expressed by a variety of human cancers (Dong et al., (2002) Nat Med 8:787-789). The receptor for PD-L1, PD-1, is expressed on lymphocytes (e.g., activated T cells) and is normally involved in down-regulating the immune system and promoting self-tolerance, particularly by suppressing T cells. However, when PD-1 receptors expressed on T cells bind to cognate PD-L1 ligands on tumor cells, the resulting T cell suppression contributes to an impaired immune response against the tumor (e.g., a decrease in tumor infiltrating lymphocytes or the establishment of immune evasion by cancer cells).

In large sample sets of e.g. ovarian, renal, colorectal, pancreatic, liver cancers and melanoma, it was shown that PD-L1 expression correlated with poor prognosis and reduced overall survival irrespective of subsequent treatment (see e.g., Dong et al., (2002) Nat Med 8(8):793-800; Yang et al., (2008) Invest Ophthalmol Vis Sci 49(6):2518-2525; Ghebeh et al., (2006) Neoplasia 8:190-198; Hamanishi et al., (2007) Proc Nat Acad Sci USA 104:3360-3365; Thompson et al., (2006) Clin Genitourin Cancer 5:206-211; Nomi et al., (2005) Clin Cancer Res 11:2947-2953; Inman et al., (2007) Cancer 109:1499-1505; Shimauchi et al., (2007) Int J Cancer 121:2585-2590; Gao et al., (2009) Clin Cancer Res 15:971-979; Nakanishi et al., (2007) Cancer Immunol Immunother 56:1173-1182; Hino et al., (2010) Cancer 116(7):1757-1766). Similarly, PD-1 expression on tumor lymphocytes was found to mark dysfunctional T cells in breast cancer (Kitano et al., (2017) ESMO Open 2(2):e000150) and melanoma (Kleffel et al., (2015) Cell 162(6):1242-1256). PD-1 antagonists, such as those that affect the function of the PD-1/PD-L1/PD-L2 signaling axis and/or disrupt the interaction between PD-1 and PD-L1 and/or PD-L2, for example, have been developed and represent a novel class of anti-tumor inhibitors that function via modulation of immune cell-tumor cell interaction.

As used herein, the term “rearranged” refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin gene locus can be identified by comparison to germline DNA; a rearranged locus will have at least one recombined heptamer/nonamer homology element.

As used herein, the term “recombinant host cell” (or simply “host cell”) is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

As used herein, the term “reference antibody” (used interchangeably with “reference mAb”) or “reference antigen-binding protein” refers to an antibody, or an antigen-binding fragment thereof, that binds to a specific epitope on IL-27 and is used to establish a relationship between itself and one or more distinct antibodies, wherein the relationship is the binding of the reference antibody and the one or more distinct antibodies to the same epitope on IL-27. As used herein, the term connotes an anti-IL-27 antibody that is useful in a test or assay, such as those described herein, (e.g., a competitive binding assay), as a competitor, wherein the assay is useful for the discovery, identification or development, of one or more distinct antibodies that bind to the same epitope.

As used herein, the terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁶ M, such as approximately less than 10⁻⁷, 10⁻⁸ M, 10⁻⁹ M or 10⁻¹⁰ M or even lower when determined by surface plasmon resonance (SPR) technology in a BIACORE 2000 instrument using recombinant human IL-27 as the analyte and the antibody as the ligand and binds to the predetermined antigen with an affinity that is at least two-fold greater than its affinity for binding to a non-specific antigen (e.g., BSA, casein) other than the predetermined antigen or a closely-related antigen. In certain aspects, an antibody that specifically binds to IL-27 binds with an equilibrium dissociation constant (K_{D}) of approximately less than 100 nM (10⁻⁷ M), optionally approximately less than 50 nM (5×10⁻⁸ M), optionally approximately less than 15 nM (1.5×10⁻⁸ M), optionally approximately less than 10 nM (10⁻⁸ M), optionally approximately less than 5 nM (5×10⁻⁹ M), optionally approximately less than 1 nM (10⁻⁹ M), optionally approximately less than 0.1 nM (10⁻¹⁰ M), optionally approximately less than 0.01 nM (10⁻¹¹ M), or even lower, when determined by surface plasmon resonance (SPR) technology in a BIACORE 2000 instrument using recombinant human IL-27 as the analyte and the antibody as the ligand, where binding to the predetermined antigen occurs with an affinity that is at least two-fold greater than the antibody's affinity for binding to a non-specific antigen (e.g., BSA, casein) other than the predetermined antigen or a closely-related antigen The phrases “an antibody recognizing an antigen” and “an antibody specific for an antigen” are used interchangeably herein with the term “an antibody which binds specifically to an antigen.”

As used herein, the term “STAT1 phosphorylation” refers to the phosphorylation of the Signal Transducer and Activator of Transcription 1 (STAT1) polypeptide, a transcription factor encoded by the STAT1 gene in humans. STAT molecules are phosphorylated by receptor associated kinases, that cause activation and dimerization by forming homo- or heterodimers which translocate to the nucleus to work as transcription factors. STAT1 can be activated (i.e., phosphorylated) in response to signaling via several ligands, including IL-27. IL-27 signaling through the IL-27R results in phosphorylation of STAT1 (pSTAT1). STAT1 has a key role in gene expression involved in survival of the cell, viability or pathogen response. Methods to determine STAT1 phosphorylation as a result of IL-27 signaling include, but are not limited to, flow cytometric analysis of cells labeled with antibodies that specifically recognize phosphorylated STAT1 (see e.g., Tochizawa et al., (2006) J Immunol Methods 313 (1-2): 29-37).

As used herein, the term “STAT3 phosphorylation” refers to the phosphorylation of the Signal Transducer and Activator of Transcription 3 (STAT3) polypeptide, a transcription factor encoded by the STAT3 gene in humans. STAT3 mediates the expression of a variety of genes in response to cell stimuli, and thus plays a key role in many cellular processes such as cell growth and apoptosis. Methods to determine STAT3 phosphorylation as a result of IL-27 signaling include, but are not limited to, analysis of cells or cell extracts labeled with antibodies that specifically recognize phosphorylated STAT3 (see e.g., Fursov et al., (2011) Assay Drug Dev Technol 9(4):420-429).

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (I Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the) (BLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsC1 banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present disclosure, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

As used herein, the term “STING” (alternatively TMEM173) refers to the Stimulator of Interferon Genes, a protein that functions both as a direct cytosolic DNA sensor and as an adaptor protein. In humans, STING is encoded by the TMEM173 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it and nearby cells. An exemplary amino acid sequence for STING is provided by the NCBI Genbank database under the accession number NP_001288667.

The term “T cell” refers to a type of white blood cell that can be distinguished from other white blood cells by the presence of a T cell receptor on the cell surface. There are several subsets of T cells, including, but not limited to, T helper cells (a.k.a. Tx cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺ FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

As used herein, the terms “therapeutically effective amount” or “therapeutically effective dose,” or similar terms used herein are intended to mean an amount of an agent (e.g., an anti-IL-27 antibody or an antigen-binding fragment thereof) that will elicit the desired biological or medical response (e.g., an improvement in one or more symptoms of a cancer).

As used herein, the term “TAM receptor” refers to the TAM receptor protein tyrosine kinases (TYRO3, AXL and MER). TAM receptors are involved in the regulation of immune system homeostasis. In a cancer setting, TAM receptors have a dual regulatory role, controlling the initiation and progression of tumor development and, at the same time, the associated anti-tumor responses of diverse immune cells. Further description of TAM receptors is found in Paolino and Penninger (2016) Cancers 8(97): doi:10.3390/cancers8100097). As used herein, the term “TAM receptor inhibitor” or “TAM inhibitor” refers to an agent that inhibits, blocks or reduces the function or activity of a TAM receptor.

As used herein, the term “TIGIT” or “T-cell immunoreceptor with Ig and ITIM domains” refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term also encompasses naturally occurring variants of TIGIT, e.g., splice variants or allelic variants. The amino acid sequence of an exemplary human TIGIT may be found under UniProt Accession Number Q495A1.

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “tumor microenvironment” (alternatively “cancer microenvironment”; abbreviated TME) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

As used herein, the term “unrearranged” or “germline configuration” refers to the configuration wherein the V segment is not recombined so as to be immediately adjacent to a D or J segment.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

### BRIEF DESCRIPTION OF THE DRAWINGS

FIG. 1 is a table that provides affinity data for anti-IL-27 antibodies that are capable of binding to an epitope comprising one or more amino acids of Gln37, Leu38, Glu42, Glu46, Va149, Ser50, Leu142, Asp146, Arg149, His150, Phe153, Leu156, Leu162, and Glu164 of SEQ ID NO: 2 (IL-27p28). Affinity measurements were performed using ForteBio and Meso Scale Discovery methods.

FIG. 2A is a graph depicting the inhibition of IL-27-mediated phosphorylation of STAT1 in human PBMCs by anti-IL-27 antibodies, as indicated, as measured by flow cytometry. FIG. 2B is a graph depicting the inhibition of IL-27-mediated phosphorylation of STAT1 in U937 cells by anti-IL-27 antibodies, as indicated, as measured by flow cytometry. FIG. 2C is a graph depicting the inhibition of IL-27-mediated phosphorylation of STAT1 in HUT-78 cells by anti-IL-27 antibodies, as indicated, as measured by flow cytometry.

FIG. 3 is graph showing that an anti-IL-27 antibody of the present disclosure (“anti-IL-27 Ab1”) inhibits IL-27-mediated pSTAT1 in human whole blood T cells.

FIG. 4 is a graph depicting the reversal of IL-27-mediated inhibition of CD161 expression in T cells by a range of concentrations of anti-IL-27 antibodies, as indicated. CD161 expression was determined using flow cytometry.

FIG. 5A is a graph depicting the extent of anti-IL-27 antibodies to enhance the PD-1-mediated secretion of TNFα in human PBMCs as measured by ELISA. FIG. 5B is a graph depicting the extent of anti-IL-27 antibodies to enhance the PD-1-mediated secretion of IL-6 in human PBMCs as measured by ELISA. FIGS. 5C-5D show that IL-27 inhibits cytokine production (IL-17A, FIG. 5C; and IFNγ, FIG. 5D) following PD-1 blockade and is restored in combination with anti-IL-27 Ab1 (Abbreviations: Ctrl=control, ns=not significant, PBMCs=peripheral blood mononuclear cells, rhIL-27=recombinant human IL-27). FIGS. 5E-511 summarize observed cytokine induction of TNFα (FIG. 5E), IFNγ (FIG. 5F), IL-6 (FIG. 5G), and IL-17A (FIG. 5H) in activated PBMC cultures from several individual donors including healthy control, and patients with RCC, HCC and ovarian cancer, when such cells were contacted with anti-IL-27 Ab1 antibody, αPD-1 antibody, or a combination of anti-IL-27 Ab1 and αPD-1 antibodies.

FIG. 6A is a graph depicting the inhibition of IL-27-mediated expression of PD-L1 by treatment of human monocytes with anti-IL-27 antibody as determined by flow cytometry. FIG. 6B is a graph depicting the inhibition of IL-27-mediated expression of TIM3 by treatment of human monocytes with anti-IL-27 antibody, as determined by flow cytometry. FIG. 6C is a graph depicting the inhibition of IL-27-mediated expression of PD-L1 by treatment of resting human T cells with anti-IL-27 antibody, as determined by flow cytometry.

FIG. 7A is a dotplot depicting the number of surface lung B16F10 metastatic nodules (pulmonary nodules) from B16F10 tumor-bearing mice treated with anti-IL27 antibody (anti-IL-27 Ab1), isotype control antibody, αWSX-1 antibody or combined αPD-1 and αCTLA-4 antibodies, as indicated, as determined by visual counting of nodules from lungs isolated from mice. FIG. 7B provides a graph depicting the growth kinetics of bioluminescent B16-Luc tumors in mice treated with anti-IL-27 antibody (anti-IL-27 Ab1) or isotype control antibody, as determined by bioluminescent imaging analysis. FIGS. 7C-7F show a series of images of fixed, sectioned lung tissue stained with hematoxylin and eosin isolated from B16F10 tumor-bearing mice treated with anti-IL27 antibody (anti-IL-27 Ab1) (FIG. 7D), isotype control antibody (FIG. 7C), αWSX-1 antibody (FIG. 7E) or combined αPD-1 and αCTLA-4 antibodies (FIG. 7F), as indicated. FIG. 7G is a dotplot depicting the total tumor area as a percentage of total tissue area of fixed, sectioned lung tissue B16F10 tumor tissue stained with hematoxylin and eosin isolated from B16F10 tumor-bearing mice treated with anti-IL27 antibody (anti-IL-27 Ab1), isotype control antibody, αWSX-1 antibody or combined αPD-1 and αCTLA-4 antibodies, as indicated, as determined by image analysis software. A similar reduction in surface lung metastasis number and total tumor area was observed with IL-27RA (WSX-1) mediated antibody blockade and with anti-PD-1+anti-CTLA-4 combination therapy.

FIG. 8A provides a volcano plot depicting microarray data of genes with an expression change >1.0 log₂ fold change (black dots) in splenocytes isolated from mice overexpressing IL-27 following treatment with IL-27 minicircles. The x axis shows log 2 fold change of gene expression (IL-27 minicircle treated vs control). The y axis is a t test p value showing probability of fold change for each gene. FIG. 8B provides a graph depicting the expression level of select immunomodulatory genes, as indicated, in splenocytes as in FIG. 8A. FIGS. 8C-8F show ectopic expression of human IL-27 induces inhibitory receptor expression (by flow cytometry analysis) on murine T cells in vivo and that anti-IL-27 Ab1 reduces inhibitory receptor expression on T cells in vivo after IL-27 minicircle treatment. Six-week-old female Balb/c mice were injected with empty vector (control) or hIL-27 minicircle (FIGS. 8C and 8D). PBMCs and (FIGS. 8E and 8F) total splenocytes were collected 5 days after transfection and cells were stained and analyzed by flow cytometry. Expression of the indicated markers were analyzed on CD4+ T cells (FIGS. 8C and 8E) and CD8+ T cells (FIGS. 8D and 8F). Analysis was performed using FlowJo software. FIG. 8G shows that anti-IL-27 Ab1 inhibits detection of minicircle-derived human IL-27 in murine plasma.

FIG. 9 is a crystal ribbon structure of an IL-27-anti-IL-27 Ab1 complex determined using the molecular replacement software Phaser (McCoy et al., (2007) J. Appl. Cyrst. 40: 658-74) and Molrep (Vagin et al., (1997) J. Appl. Cyrst. 30: 1022-25). Heavy chain, light chain, p28, and EBI-3 are colored in yellow, red, grey, and green respectively. FIG. 9 shows that anti-IL-27 Ab1 is bound to the p28 molecule of IL-27.

FIGS. 10A-10B are graphs showing human IL-27 heterodimer binding affinity to WSX-1 (FIG. 10A) and gp130 (FIG. 10B) in the presence (dark grey line) or absence (light grey line) of anti-IL-27 Ab1, as measured by surface plasmon resonance.

FIG. 11 is a ribbon diagram of p28, showing the residues where anti-IL-27 Ab1 binds p28. LC=light chain of anti-IL-27 Ab1; HC=heavy chain of anti-IL-27 Ab1

FIG. 12 is a ribbon diagram of the structural alignment of IL-27/anti-IL-27 Ab1 Fab with IL-23/IL-23R (PDB ID: SMZV). Superimposition of complexes using p28 and p19 were aligned in 3-dimensional space.

FIG. 13 is a ribbon diagram of the structural alignment of IL-27/anti-IL-27 Ab1 Fab with IL-6/IL-6Ra/gp130. Superimposition of complexes using p28 and IL-6 were aligned in 3-dimensional space.

FIGS. 14A-14B are ribbon diagrams of the binding interface of p28 and EBI3, with FIG. 14B showing an enlargement of FIG. 14A to illustrate the location of salt bridge interactions and aromatic/hydrophobic interactions between p28 and EBI3.

FIGS. 15A-15B are images of sequence alignments of p28 (FIG. 15A) and EBI3 (FIG. 15B) across several animal species. Arrows point to conserved salt bridging amino acids and conserved hydrophobic amino acids, as indicated.

FIG. 16A is a ribbon diagram illustrating the structural alignment of IL-27 heterodimer with IL-6/IL-6Ra. FIGS. 16B-16C are sequence alignments of IL-27 and IL-6/IL-6Ra. Arrows point to conserved salt bridging amino acids and conserved hydrophobic amino acids. FIG. 16D is a ribbon diagram illustrating several p28 interactions with EBI3 that are conserved with IL-6Ra.

FIG. 17 is a table presenting binding affinity data for human IL-27 and gp130, WSX-1, and an anti-p28 antibody.

FIG. 18A is a sequence alignment of the mouse and human p28 amino acid sequences.

FIG. 18B is a ribbon diagram, focused in at residue 162 (Leu in the human sequence, and Cys in the mouse sequence).

FIG. 19A shows the electrostatic surface potential of human IL-27. FIG. 19B shows the primary sequence of human IL-27, showing the αA, αB, αC, αD helices, and unresolved CD loop with poly-Glu sequence.

FIG. 20A is graphical representation illustrating differential expression of EBI3, IL-27p28, and IL-27RA in RCC tumor (1) and normal kidney tissue (2). FIGS. 20B-20D are Kaplan-Meier curves (percent death-free survival in days) for RCC patients stratified by high (1) or low (2) expression of EBI3 (FIG. 20B), IL-27p28 (FIG. 20C), and IL-27RA (FIG. 20D). Data were generated using TCGA as previously described (see, e.g., Li et al., Cancer Research. 2017; 77(21):e108-e110; Li et al., Genome Biology 2016; 17(1):174).

FIGS. 21A-21B show IL-27 induced gene expression signatures in activated human CD4⁺ T cells. FIG. 21A is a fold change scatter plot of IL-27-treated CD4⁺ T cells as compared to untreated controls for two separate donors. FIG. 21B shows the top 31 genes in the IL-27 signature in CD4⁺ T cells. Fifteen of the 31 genes (marked with a star) were associated with poor outcome. Data were generated using TCGA.

FIGS. 22A-22B are graphical representations of the genome-wide hazard ratios associated with the expression of IL-27 signature genes in RCC (FIG. 22A) and BRCA (FIG. 22B) tumor samples. Data were generated using TCGA.

FIG. 23A is graphical representations of EBI3 plasma levels in patients with RCC, as compared to healthy donor serum and serum from a pregnant female (positive control), as measured using an EBI3-specific antibody pair. FIG. 23B shows EBI3 levels in a separate cohort of patients with RCC grouped by tumor stage. FIG. 23C shows overall survival and FIG. 23D shows disease-free survival in patients with RCC, stratified by serum EBI3 levels.

FIGS. 24A-24B are graphical representations of the effect of anti-IL-27 Ab1 on tumor growth and lung metastases in an orthotopic Renca model. FIGS. 24A and 24B show the net primary tumor weight (kidney) and the number of lung metastases in control and anti-IL-27 Ab1-treated Renca mice. (*P<0.05; unpaired t-test)

FIGS. 25A-25B show the effect of anti-IL-27 Ab1 as a single agent on mean orthotopic Hepa1-6 tumor flux overtime as compared to isotype control (FIG. 25B) in the orthotopic Hepa1-6-luc tumor model (FIG. 25A). Error bars indicate standard error.

FIGS. 26A-26F show dose-dependent inhibition of orthotopic Hepa1-6 tumor growth following serial administration of anti-IL-27 Ab1 (FIG. 26A). FIG. 26B shows mean bioluminescence imaging (“BLI”, photons/second) at 5, 8, 13, and 16 days post implant for control and anti-IL-27 Ab1 (5 mg/kg, 25 mg/kg, and 50 mg/kg) dosing. FIGS. 26C-26F show BLI (photons/second) at 5, 8, 13, and 16 days post implant for individual animals in control (FIG. 26C) and anti-IL-27 Ab1 5 mg/kg (FIG. 26D), 25 mg/kg (FIG. 26E), and 50 mg/kg groups (FIG. 26F).

FIGS. 27A-27C show modulation of gene expression in Hepa1-6 livers following administration of anti-IL-27 Ab1 (FIGS. 27A and 27B). FIG. 27C is a volcano plot of genes modulated by anti-IL-27 Ab1 administration. Tables 11A-11B, below, provide lists of upregulated and downregulated genes represented in FIG. 27B.

FIGS. 28A-28E are graphical representations illustrating the expression of various IL-27 component genes (FIG. 28A); CD274, TIGIT, LAG3, HAVCR2, and PDCD1 (FIG. 28B); TGFA and TGFB1 (FIG. 28C); AFP (FIG. 28D); and TNFRSF10B, TNFRSF1A, and PDGFA (FIG. 28E) following anti-IL-27 Ab1 administration.

FIGS. 29A-29B are graphical representations of the relative expression of various macrophage and NK transcript marker genes in the tumor microenvironment (TME) following anti-IL-27 Ab1 administration.

FIG. 30 is a graphical representation of the expression of NK-associated receptors following administration of either anti-IL-27 Ab1 or an isotype control.

FIG. 31 shows the relative expression of various cell surface markers following administration of anti-IL-27 Ab1 as compared to an IgG isotype control. Ratios were obtained by normalizing target marker transcript level to PTPRC level. Directionality is expressed as difference between anti-IL-27 Ab1 ratio and IgG ratio.

FIGS. 32A-32D are bar graphs showing the expression of IL17A (FIG. 32A), IFNg (IFNγ) (FIG. 32B), TNFa (TNFα) (FIG. 32C), and IL-10 (FIG. 32D) in cultured PBMCs stimulated with anti-CD3 (0.25 μg/mL) for 3 days in the presence or absence of IL-27 (100 ng/mL).

FIGS. 33A-33D are scatter plots showing the expression of IL17A (FIG. 33A), IFNg (IFNγ) (FIG. 33B), TNFa (TNFα) (FIG. 33C), and IL-10 (FIG. 33D) in cultured PBMCs stimulated with anti-CD3 (0.25 μg/mL) for 4 days in the presence or absence of anti-IL-27 Ab1 (1 μg/mL).

FIG. 34 is a volcano plot representing the log₂ fold-change in gene expression after IL-27 inhibition compared to control (x-axis) versus the significance (p-value) of gene expression changes after treatment with anti-IL-27 Ab1 compared to control (y-axis).

FIG. 35 is a scatter plot showing TNFSF15 expression in activated PBMCs following culture in anti-IL-27 Ab1 or an isotype control (IgG).

FIGS. 36A-36B are bar graphs showing TNFSF15 expression in activated (FIG. 36A) and resting (FIG. 36B) PBMCs cultured in the presence of two different lots of anti-IL-27 Ab1 (1 μg/mL) or isotype control.

FIG. 37 is a bar graph showing the fold change in TNFSF15 transcript after IL-27 inhibition with anti-IL-27 Ab1 compared to isotype control in various cell types, as indicated.

FIG. 38 is a bar graph showing the fold expression in TNFSF15 transcript following treatment with anti-IL-27 Ab1, an anti-CD39 antibody, and two anti-CD112R antibodies, as indicated.

FIGS. 39A-39B are bar graphs showing TNFSF15 transcript (FIG. 39A) and secreted TNFSF15 protein (FIG. 39B) after blocking IL-27 with anti-IL-27 Ab1 in activated PBMCs with delayed kinetics.

### DETAILED DESCRIPTION

The present disclosure provides, at least in part, antibody molecules that bind to a specific epitope on human IL-27p28 with high affinity and specificity. The terms “IL-27” and “IL27” as used herein refer interchangeably to the heterodimeric cytokine, IL-27 that is composed of two distinct subunits, encoded by two different genes: Epstein-Barr virus-induced gene 3 (EBI3) and IL-27p28. IL-27 has both pro- and anti-inflammatory properties with diverse effects on—hematopoietic and non-hematopoietic cells.

Accordingly, in one aspect, the disclosure provides an isolated antibody that specifically binds to and antagonizes human IL-27, or an antigen binding portion thereof, wherein the antibody or antigen binding portion thereof specifically binds to the epitopes disclosed herein and exhibits at least one or more of the following properties:

(i) binds to human IL-27 with an equilibrium dissociation constant (K_{D}) of 15 nM or less;

(ii) blocks binding of IL-27 to IL-27 receptor;

(iii) inhibits or reduces STAT1 and/or STAT3 phosphorylation in a cell;

(iv) inhibits or reduces IL-27 mediated inhibition of CD161 expression in a cell;

(v) inhibits or reduces IL-27 mediated PD-L1 and/or TIM-3 expression in a cell;

(vi) induces or enhances PD-1 mediated secretion of one or more cytokines from a cell; and

(vii) a combination of (i)-(vi).

Additional aspects of the invention include nucleic acid molecules encoding the antibody molecules, expression vectors, host cells and methods for making the antibody molecules. Immunoconjugates, multi- or bispecific molecules and pharmaceutical compositions comprising the antibody molecules are also provided. The anti-IL-27 antibody molecules disclosed herein can be used to treat, prevent and/or diagnose cancerous or malignant disorders, e.g., solid and liquid tumors (e.g., leukemia, e.g., lymphoma, e.g., AML), lung cancer (e.g., non-small cell lung cancer), pancreatic cancer, breast cancer (e.g., triple-negative breast cancer), melanoma, testicular cancer, sarcoma, head and neck cancer (e.g., squamous head and neck cancer), liver cancer (e.g., hepatocellular carcinoma (HCC)), colorectal cancer, ovarian cancer, brain cancer (e.g., glioblastoma multiforme), or renal cancer (e.g., renal cell carcinoma, e.g. renal clear cell carcinoma).

### Anti-IL-27 Antibodies and Antigen-Binding Fragments Thereof

The present disclosure provides antibodies, and antigen binding portions thereof, that specifically bind to IL-27p28 and antagonize IL-27, in particular human IL-27.

The present disclosure is directed to an isolated antibody that antagonizes human IL-27, or an antigen binding portion thereof, wherein the antibody or antigen binding portion thereof specifically binds to an epitope comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii). In some aspects, an isolated antibody of the disclosure that antagonizes human IL-27, or an antigen binding portion thereof, specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, or Glu164 of SEQ ID NO: 2 (IL-27p28).

In some aspects, an antibody, or antigen binding portion thereof, of the present disclosure specifically binds to an epitope comprising Asp146, Arg149, and/or Phe153 of SEQ ID NO: 2 (IL-27p28). In some aspects, an antibody, or antigen binding portion thereof, of the present disclosure specifically binds to an epitope comprising Asp146, Arg149, and Phe153 of SEQ ID NO: 2 (IL-27p28). In some aspects, the epitope comprises Asp146, Arg149, His150, and Phe153 of SEQ ID NO: 2 (IL-27p28). In some aspects, the epitope comprises Asp146, Arg149, Phe153, and Leu156 of SEQ ID NO: 2 (IL-27p28). In some aspects, the epitope comprises Asp146, Arg149, His150, Phe153, and Leu156 of SEQ ID NO: 2 (IL-27p28).

In some aspects, an antibody, or antigen binding portion thereof, of the present disclosure specifically binds to an epitope comprising Leu142, Asp146, Arg149, His150, Phe153, Leu156, and Glu164 of SEQ ID NO: 2 (IL-27p28). In some aspects, the epitope comprises Gln37, Leu38, Glu42, Asp146, Arg149, His150, Phe153, and Leu156 of SEQ ID NO: 2 (IL-27p28). In some aspects, an antibody, or antigen binding portion thereof, of the present disclosure specifically binds to an epitope comprising Gln37, Leu38, Glu42, Leu142, Asp146, Arg149, His150, Phe153, Leu156, and Glu164 of SEQ ID NO: 2 (IL-27p28).

In some aspects, an antibody, or antigen binding portion thereof, of the present disclosure specifically binds to an epitope comprising Leu142, Asp146, Arg149, His150, Phe153, Leu156, Leu162, and Glu164 of SEQ ID NO: 2 (IL-27p28). In some aspects, an antibody, or antigen binding portion thereof, of the present disclosure specifically binds to an epitope comprising Glu46, Va149, Ser50, Leu142, Asp146, Arg149, His150, Phe153, Leu156, and Glu164 of SEQ ID NO: 2 (IL-27p28). In some aspects, an antibody, or antigen binding portion thereof, of the present disclosure specifically binds to an epitope comprising Gln37, Leu38, Glu42, Glu46, Va149, Ser50, Leu142, Asp146, Arg149, His150, Phe153, Leu156, Leu162, and Glu164 of SEQ ID NO: 2 (IL-27p28).

In some aspects, an antibody, or antigen binding portion thereof, of the present disclosure specifically binds to an epitope consisting of or consisting essentially of Gln37, Leu38, Glu42, Glu46, Va149, Ser50, Leu142, Asp146, Arg149, His150, Phe153, Leu156, Leu162, and Glu164 of SEQ ID NO: 2 (IL-27p28).

In some aspects, an antibody, or antigen binding portion thereof, of the present disclosure specifically binds to an epitope comprising Gln37, Leu38, Glu42, Glu46, Va149, Ser50, Leu142, Asp146, Arg149, His150, Phe153, Leu156, Leu162, and Glu164 of SEQ ID NO: 2 (IL-27p28) and at least one residues selected from the group consisting of: Leu53, Lys56, Asp143, Leu147, Arg152, Ala157, Gly159, Phe160, or Asn161 of SEQ ID NO: 2 (IL-27p28).

In some aspects, an antibody, or antigen binding portion thereof, of the present disclosure specifically binds to an epitope comprising Gln37, Leu38, Glu42, Glu46, Va149, Ser50, Leu142, Asp146, Arg149, His150, Phe153, Leu156, Leu162, and Glu164 of SEQ ID NO: 2 (IL-27p28) and at least one residues selected from the group consisting of: Leu53, Lys56, Asp143, Arg145, Leu147, Arg152, Ala157, Gly159, Phe160, Asn161, or Pro163 of SEQ ID NO: 2 (IL-27p28).

In some aspects, an antibody, or antigen binding portion thereof, of the present disclosure specifically binds to an epitope consisting or consisting essentially of Gln37, Leu38, Glu42, Glu46, Va149, Ser50, Leu53, Lys56, Leu142, Asp143, Asp146, Leu147, Arg149, His150, Arg152, Phe153, Leu156, Ala157, Gly159, Phe160, Asn161, Leu162, and Glu164 of SEQ ID NO: 2 (IL-27p28).

In some aspects, an antibody, or antigen binding portion thereof, of the present disclosure specifically binds to an epitope consisting or consisting essentially of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28).

In some aspects, the disclosure provides an isolated antibody that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) and antagonizes human IL-27, or an antigen binding portion thereof, wherein the antibody or antigen binding portion thereof exhibits at least one or more of the following properties: (i) binds to human IL-27 with an equilibrium dissociation constant (K_{D}) of 15 nM or less; (ii) blocks binding of IL-27 to IL-27 receptor; (iii) inhibits or reduces STAT1 and/or STAT3 phosphorylation in a cell; (iv) inhibits or reduces inhibition of CD161 expression in a cell; (v) inhibits or reduces PD-L1 and/or TIM-3 expression in a cell; (vi) induces or enhances PD-1 mediated secretion of one or more cytokines from a cell; and (vii) a combination of (i)-(vi).

In some aspects, the isolated antibody, or antigen binding portion thereof, binds to an epitope of one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (human IL-27p28) with an equilibrium dissociation constant (K_{D}) of 15 nM or less.

In some aspects, the isolated antibody, or antigen binding portion thereof, binds to recombinant human IL-27p28 or to murine IL-27p28.

In some aspects, the isolated antibody, or antigen binding portion thereof, inhibits or reduces STAT1 and/or STAT3 phosphorylation in a cell. In some aspects, the cell is an immune cell. In some aspects, the cell is a cancer cell.

In some aspects, the isolated antibody, or antigen binding portion thereof, inhibits or reduces inhibition of CD161 expression in a cell (e.g. ameliorates or relieves the inhibition of CD161 expression in a cell). In some aspects, the cell is an immune cell.

In some aspects, the isolated antibody, or antigen binding portion thereof, inhibits or reduces PD-L1 and/or TIM-3 expression in a cell. In some aspects, PD-L1 expression is inhibited or reduced. In some aspects, TIM-3 expression is inhibited or reduced. In some aspects, both PD-L1 expression and TIM-3 expression is reduced. In some aspects, the cell is an immune cell. In some aspects, the antibodies are monoclonal antibodies.

In some aspects, the isolated antibody, or antigen binding portion thereof, induces or enhances the PD-1-mediated secretion of one or more cytokines from a cell. In some aspects, the one or more cytokines is TNFα. In some aspects, the one or more cytokine is IL-6. In some aspects, the one or more cytokine is TNFα and IL-6. In some aspects, the cell is an immune cell.

In some aspects, the isolated antibody, or antigen binding portion thereof, is selected from the group consisting of an IgG1, an IgG2, an IgG3, an IgG4, an IgM, an IgA1 an IgA2, an IgD, and an IgE antibody. In some aspects, the antibody is an IgG1 antibody or an IgG4 antibody. In some aspects, the antibody comprises a wild type IgG1 heavy chain constant region. In some aspects, the antibody comprises a wild type IgG4 heavy chain constant region. In some aspects, the antibody comprises an Fc domain comprising at least one mutation. In some aspects, the antibody comprises a mutant IgG1 heavy chain constant region. In some aspects, the antibody comprises a mutant IgG4 heavy chain constant region. In some aspects, the mutant IgG4 heavy chain constant region comprises any one of the substitutions S228P, L235E, L235A, or a combination thereof, according to EU numbering.

In some aspects, the disclosure provides an isolated antibody, or antigen binding portion thereof, that binds to substantially the same epitope on IL-27 as the antibody, or antigen binding portion thereof, according to any one of the aforementioned aspects.

In some aspects, the disclosure provides an isolated antibody, or antigen binding portion thereof, that binds to at least one of the amino acid residues selected from the group consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28) bound by the antibody, or antigen binding portion thereof, according to any one of the aforementioned aspects.

In some aspects, the disclosure provides an isolated antibody, or antigen binding portion thereof, wherein a mutation of the epitope (Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28)) bound by the antibody or antigen binding portion thereof inhibits, reduces, or blocks binding to both the antibody or antigen binding portion thereof and to the antibody or antigen binding portion thereof according to any one of the aforementioned aspects.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C. In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR3 consists of N-XXXASAXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR2 consists of N-XXSSSXSYXYXXXXXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR3 consists of N-XXXXGRTSYTATXHNXXXX-C, wherein X is any amino acids.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C and light chain CDR3 consists of N-XXXASAXXX-C. In some aspects, the antibody, or antigen binding portion thereof, comprises heavy chain CDR1, heavy chain CDR2, heavy chain CDR3, light chain CDR1, light chain CDR2, and light chain CDR3, wherein heavy chain CDR2 consists of N-XXSSSXSYXYXXXXXXX-C and heavy chain CDR3 consists of N-XXXXGRTSYTATXHNXXXX-C, wherein X is any amino acids.

In some aspects, the antibody, or antigen binding portion thereof, comprises heavy chain CDR1, heavy chain CDR2, heavy chain CDR3, light chain CDR1, light chain CDR2, and light chain CDR3, wherein light chain CDR1 consists of N-XXXXXXLFSSNXKXYXX-C, light chain CDR3 consists of N-XXXASAXXX-C, heavy chain CDR2 consists of N-XXSSSXSYXYXXXXXXX-C, and heavy chain CDR3 consists of N-XXXXGRTSYTATXHNXXXX-C, wherein X is any amino acids.

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;

(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;

(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;

(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;

(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or

(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;

(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;

(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;

(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;

(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or

(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;

(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;

(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;

(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;

(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively; or

(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively.

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;

(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;

(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;

(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 88 and 89, respectively;

(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or

(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;

(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;

(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;

(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 88 and 89, respectively;

(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or

(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise heavy and light chain CDRs selected from the group consisting of:

(i) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;

(ii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;

(iii) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;

(iv) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 88 and 89, respectively;

(v) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; or

(vi) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not consist of N-GFTF[S/A/R][S/R][T/Y][G/S]-C (SEQ ID NO: 144) and/or the heavy chain CDR2 does not consist of N-ISSS[S/G][S/A]YI-C (SEQ ID NO: 146).

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A]YI[L/Y]YADSVKG-C (SEQ ID NO: 149).

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A[YI]L/Y]YADSVKG-C (SEQ ID NO: 149).

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain CDR1, a heavy chain CDR2, a heavy chain CDR3, a light chain CDR1, a light chain CDR2, and a light chain CDR3 and wherein the heavy chain CDR1 does not comprise N-FTF[S/A/R][S/R][T/Y][G/S]MN-C (SEQ ID NO: 148) and/or the heavy chain CDR2 does not comprise N-[G/S]ISSS[S/G][S/A]YI[L/Y]YADSVKG-C (SEQ ID NO: 149).

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise:

(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or

(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof does not comprise:

(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or

(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise:

(i) heavy chain CDR1 consisting of N-GFTFXXXX-C (SEQ ID NO: 145), heavy chain CDR2 consisting of N-ISSSXXYI-C (SEQ ID NO: 147), and heavy chain CDR3 sequence set forth in SEQ ID NO: 121; and light chain CDR1, CDR2 and CDR3 sequences set forth in SEQ ID NOs: 127, 128 and 129, respectively; or

(ii) heavy chain CDR1 consisting of N-FTFXXXXMN-C (SEQ ID NO: 150), heavy chain CDR2 consisting of N-XISSSXXYIXYADSVKG-C (SEQ ID NO: 151), and heavy chain CDR3 sequence set forth in SEQ ID NO: 124; and light chain CDR1, CDR2 and CDR3 sequences set forth in SEQ ID NOs: 130, 131 and 132, respectively.

In some aspects, the present disclosure provides an isolated antibody or antigen binding portion thereof that specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP[X]ₙ₌₀₋₁T-C (SEQ ID NO: 156), respectively.

In some aspects, the present disclosure provides an isolated antibody or antigen binding portion thereof that specifically binds to an epitope comprising or consisting of Gln37, Leu38, Glu42, Glu46, Va149, Ser50, Leu142, Asp146, Arg149, His150, Phe153, Leu156, Leu162, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP[X]ₙ₌₀₋₁T-C (SEQ ID NO: 156), respectively.

In some aspects, the present disclosure provides an isolated antibody or antigen binding portion thereof that specifically binds to an epitope comprising or consisting of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof does not comprise: heavy chain CDR1 consisting of N-GFTFXXXX-C (SEQ ID NO: 145), heavy chain CDR2 consisting of N-IXXXXXXX-C (SEQ ID NO: 152), and heavy chain CDR3 sequence consisting of N-AR[X]ₙ₌₆₋₁₅DX-C (SEQ ID NO: 153); and light chain CDR1 consisting of N-QS[X]ₙ₌₁₋₃SS[X]ₙ₌₀₋₄Y-C (SEQ ID NO: 154), light chain CDR2 consisting of N-XXS-C (SEQ ID NO: 155), and light chain CDR3 sequence consisting of N-QQXXXXP[X]ₙ₌₀₋₁T-C (SEQ ID NO: 156), respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region does not comprise an amino acid sequence selected from the group consisting of SEQ ID NOs: 15, 37, 59, 81, 103, and 125; and wherein the light chain variable region does not comprise an amino acid sequence selected from the group consisting of SEQ ID NOs: 23, 45, 67, 89, 111, and 133.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region and the light chain variable region are not amino acid sequences selected from the group consisting of:

(i) SEQ ID NO: 15 and 65, respectively;

(ii) SEQ ID NO: 37 and 45, respectively;

(iii) SEQ ID NO: 59 and 67, respectively;

(iv) SEQ ID NO: 81 and 89, respectively;

(v) SEQ ID NO: 103 and 111, respectively; and

(vi) SEQ ID NO: 125 and 133, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region does not comprise an amino acid sequence which is at least 90% identical to the amino acid sequence selected from the group consisting of SEQ ID NOs: 15, 37, 59, 81, 103, and 125; and wherein the light chain variable region does not comprise an amino acid sequence which is at least 90% identical to the amino acid sequence selected from the group consisting of SEQ ID NOs: 23, 45, 67, 89, 111, and 133.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises heavy and light chain variable regions, wherein the heavy chain variable region and the light chain variable region do not comprise amino acid sequences at least 90% identical to the amino acid sequences selected from the group consisting of:

(i) SEQ ID NO: 15 and 65, respectively;

(ii) SEQ ID NO: 37 and 45, respectively;

(iii) SEQ ID NO: 59 and 67, respectively;

(iv) SEQ ID NO: 81 and 89, respectively;

(v) SEQ ID NO: 103 and 111, respectively; and

(vi) SEQ ID NO: 125 and 133, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, wherein the heavy chain does not comprise an amino acid sequence selected from the group consisting of SEQ ID NOs: 25, 47, 69, 91, 113, and 135; and wherein the light chain does not comprise an amino acid sequence selected from the group consisting of SEQ ID NOs: 20, 42, 71, 93, and 1115.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain, wherein the heavy chain does not comprise an amino acid sequence which is at least 90% identical to the amino acid sequence selected from the group consisting of SEQ ID NOs: 25, 47, 69, 91, 113, and 135; and wherein the light chain does not comprise an amino acid sequence which is at least 90% identical to the amino acid sequence selected from the group consisting of SEQ ID NOs: 20, 42, 71, 93, and 115.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, wherein the heavy chain does not comprise an amino acid sequence selected from the group consisting of SEQ ID NOs: 29, 51, 73, 95, 117, and 139; and wherein the light chain does not comprise an amino acid sequence selected from the group consisting of SEQ ID NOs: 71, 49, 71, 93, 115, and 137.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain, wherein the heavy chain does not comprise an amino acid sequence which is at least 90% identical to the amino acid sequence selected from the group consisting of SEQ ID NOs: 29, 51, 73, 95, 117, and 139; and wherein the light chain does not comprise an amino acid sequence which is at least 90% identical to the amino acid sequence selected from the group consisting of SEQ ID NOs: 71, 49, 71, 93, 115, and 137.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain, and wherein the heavy chain and the light chain do not comprise amino acid sequences selected from the group consisting of:

(i) SEQ ID NO: 25 and 27, respectively;

(ii) SEQ ID NO: 47 and 49, respectively;

(iii) SEQ ID NO: 69 and 71, respectively;

(iv) SEQ ID NO: 91 and 93, respectively;

(v) SEQ ID NO: 113 and 115, respectively; and

(vi) SEQ ID NO: 135 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy chain and a light chain and wherein the heavy chain and the light chain do not comprise amino acid sequences at least 90% identical to the amino acid sequences selected from the group consisting of:

(i) SEQ ID NO: 25 and 27, respectively;

(ii) SEQ ID NO: 47 and 49, respectively;

(iii) SEQ ID NO: 69 and 71, respectively;

(iv) SEQ ID NO: 91 and 93, respectively;

(v) SEQ ID NO: 113 and 115, respectively; and

(vi) SEQ ID NO: 135 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL-27p28), wherein the antibody or antigen binding portion thereof comprises a heavy and a light chain and wherein the heavy chain and the light chain do not comprise amino acid sequences selected from the group consisting of:

(i) SEQ ID NO: 29 and 27, respectively;

(ii) SEQ ID NO: 51 and 49, respectively;

(iii) SEQ ID NO: 73 and 72, respectively;

(iv) SEQ ID NO: 95 and 93, respectively;

(v) SEQ ID NO: 117 and 115, respectively; and

(vi) SEQ ID NO: 139 and 137, respectively.

In some aspects, the present disclosure provides an isolated antibody or antigen binding portion thereof that antagonizes IL-27 and specifically binds to an epitope comprising one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, and Glu164 of SEQ ID NO: 2 (IL27-p28), wherein the antibody or antigen binding portion thereof comprises a heavy and a light chain and wherein the heavy chain and the light chain do not comprise amino acid sequences at least 90% identical to the amino acid sequences selected from the group consisting of:

(i) SEQ ID NO: 29 and 27, respectively;

(ii) SEQ ID NO: 51 and 49, respectively;

(iii) SEQ ID NO: 73 and 72, respectively;

(iv) SEQ ID NO: 95 and 93, respectively;

(v) SEQ ID NO: 117 and 115, respectively; and

(vi) SEQ ID NO: 139 and 137, respectively.

Methods for Producing the Anti-IL-27 Antibodies and Antigen-binding Fragments Thereof

The disclosure also features methods for producing any of the anti-IL-27 antibodies or antigen-binding fragments thereof described herein. In some aspects, methods for preparing an antibody described herein can include immunizing a subject (e.g., a non-human mammal) with an appropriate immunogen. Suitable immunogens for generating any of the antibodies described herein are set forth herein. For example, to generate an antibody that binds to IL-27p28, a skilled artisan can immunize a suitable subject (e.g., a non-human mammal such as a rat, a mouse, a gerbil, a hamster, a dog, a cat, a pig, a goat, a horse, or a non-human primate) with IL-27. In some aspects, a full-length human IL-27p28 monomer polypeptide comprising the amino acid sequence set forth in SEQ ID NO: 2 is used as the immunogen.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle Bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some aspects, the methods include preparing a hybridoma cell line that secretes a monoclonal antibody that binds to the immunogen. For example, a suitable mammal such as a laboratory mouse is immunized with a IL-27 polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybrid cells are then screened for secretion of the desired antibodies, e.g., an antibody that binds to human IL-27 and In some aspects, a skilled artisan can identify an anti-IL-27 antibody from a non-immune biased library as described in, e.g., U.S. Pat. No. 6,300,064 (to Knappik et al.; Morphosys AG) and Schoonbroodt et al. (2005) Nucleic Acids Res 33(9):e81.

In some aspects, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999)J Immunol Methods 231:119-135; and Hanes et al. (2000) Nat Biotechnol 18:1287-1292).

Methods for identifying antibodies using various phage display methods are known in the art. In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen binding domains are expressed as a recombinantly fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some aspects, the phage display antibody libraries can be generated using mRNA collected from B cells from the immunized mammals. For example, a splenic cell sample comprising B cells can be isolated from mice immunized with IL-27 polypeptide as described above. mRNA can be isolated from the cells and converted to cDNA using standard molecular biology techniques. See, e.g., Sambrook et al. (1989) “Molecular Cloning: A Laboratory Manual, 2^{nd} Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some aspects, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some aspects, require use of a helper phage. In some aspects, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., human IL-27p28) using any immunological or biochemical based method known in the art. For example, specific binding of an antibody to IL-27p28, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immuno-specific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

In aspects where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody.

In some aspects, the anti-IL-27 antibodies described herein comprise an altered heavy chain constant region that has reduced (or no) effector function relative to its corresponding unaltered constant region. Effector functions involving the constant region of the anti-IL-27 antibody may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular aspects, modulation includes situations in which an activity is abolished or completely absent.

In one aspect, the anti-IL-27 antibodies described herein comprise an IgG4 heavy chain constant region. In one aspect, the IgG4 heavy chain constant region is a wild type IgG4 heavy chain constant region. In another aspect, the IgG4 constant region comprises a mutation, e.g., one or both of S228P and L235E or L235A, e.g., according to EU numbering (Kabat, E. A., et al., supra). In one aspect, the anti-IL-27 antibodies described herein comprise an IgG1 constant region. In one aspect, the IgG1 heavy chain constant region is a wild type IgG1 heavy chain constant region. In another aspect, the IgG1 heavy chain constant region comprises a mutation.

An altered constant region with altered FcR binding affinity and/or ADCC activity and/or altered CDC activity is a polypeptide which has either an enhanced or diminished FcR binding activity and/or ADCC activity and/or CDC activity compared to the unaltered form of the constant region. An altered constant region which displays increased binding to an FcR binds at least one FcR with greater affinity than the unaltered polypeptide. An altered constant region which displays decreased binding to an FcR binds at least one FcR with lower affinity than the unaltered form of the constant region. Such variants which display decreased binding to an FcR may possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity may exhibit either increased or reduced ADCC and/or CDC activity compared to the unaltered constant region. For example, in some aspects, the anti-IL-27 antibody comprising an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. An anti-IL-27 antibody described herein comprising an altered constant region displaying reduced ADCC and/or CDC may exhibit reduced or no ADCC and/or CDC activity.

In some aspects, an anti-IL-27 antibody described herein exhibits reduced or no effector function. In some aspects, an anti-IL-27 antibody comprises a hybrid constant region, or a portion thereof, such as a G2/G4 hybrid constant region (see e.g., Burton et al. (1992) Adv Immun 51:1-18; Canfield et al. (1991) J Exp Med 173:1483-1491; and Mueller et al. (1997) Mol Immunol 34(6):441-452). See above.

In some aspects, an anti-IL-27 antibody may contain an altered constant region exhibiting enhanced or reduced complement dependent cytotoxicity (CDC). Modulated CDC activity may be achieved by introducing one or more amino acid substitutions, insertions, or deletions in an Fc region of the antibody. See, e.g., U.S. Pat. No. 6,194,551. Alternatively, or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See, e.g., Caron et al. (1992) J Exp Med 176:1191-1195 and Shopes (1992) Immunol 148:2918-2922; PCT publication nos. WO 99/51642 and WO 94/29351; Duncan and Winter (1988) Nature 322:738-40; and U.S. Pat. Nos. 5,648,260 and 5,624,821.

### Recombinant Antibody Expression and Purification

The antibodies or antigen-binding fragments thereof described herein can be produced using a variety of techniques known in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some aspects, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell and will be easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Modification of the Antibodies or Antigen-Binding Fragments Thereof

The antibodies or antigen-binding fragments thereof can be modified following their expression and purification. The modifications can be covalent or non-covalent modifications. Such modifications can be introduced into the antibodies or fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments.

In some aspects, the antibodies or antigen-binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (FLAG (DYKDDDDK (SEQ ID NO: 141)), polyhistidine (6-His; HHHHHH (SEQ ID NO: 142), hemagglutinin (HA; YPYDVPDYA (SEQ ID NO: 143)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins (e.g., an antibody and a heterologous moiety) can be cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some aspects, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NETS) ester or tetrafluorophenyl (TFP) ester moieties attached to the fluorophores. In some aspects, the fluorophores can be conjugated to a heterobifunctional cross-linker moiety such as sulfo-SMCC. Suitable conjugation methods involve incubating an antibody protein, or fragment thereof, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some aspects, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476 or HESylated (Fresenius Kabi, Germany; see, e.g., Pavisić et al. (2010) Int J Pharm 387(1-2):110-119). The stabilization moiety can improve the stability, or retention of, the antibody (or fragment) by at least 1.5 (e.g., at least 2, 5, 10, 15, 20, 25, 30, 40, or 50 or more) fold.

In some aspects, the antibodies or antigen-binding fragments thereof described herein can be glycosylated. In some aspects, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### Pharmaceutical Compositions and Formulations

In certain aspects, the invention provides for a pharmaceutical composition comprising an anti-IL-27 antibody with a pharmaceutically acceptable diluent, carrier, solubilizer, emulsifier, preservative and/or adjuvant.

In certain aspects, acceptable formulation materials preferably are nontoxic to recipients at the dosages and concentrations employed. In certain aspects, the formulation material(s) are for s.c. and/or I.V. administration. In certain aspects, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain aspects, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain aspects, the formulation comprises PBS; 20 mM NaOAC, pH 5.2, 50 mM NaCl; and/or 10 mM NAOAC, pH 5.2, 9% Sucrose. In certain aspects, the optimal pharmaceutical composition will be determined by one skilled in the art depending upon, for example, the intended route of administration, delivery format and desired dosage. See, for example, Remington's Pharmaceutical Sciences, supra. In certain aspects, such compositions may influence the physical state, stability, rate of in vivo release and/or rate of in vivo clearance of the anti-IL-27 antibody.

In certain aspects, the primary vehicle or carrier in a pharmaceutical composition can be either aqueous or non-aqueous in nature. For example, in certain aspects, a suitable vehicle or carrier can be water for injection, physiological saline solution or artificial cerebrospinal fluid, possibly supplemented with other materials common in compositions for parenteral administration. In certain aspects, the saline comprises isotonic phosphate-buffered saline. In certain aspects, neutral buffered saline or saline mixed with serum albumin are further exemplary vehicles. In certain aspects, pharmaceutical compositions comprise Tris buffer of about pH 7.0-8.5, or acetate buffer of about pH 4.0-5.5, which can further include sorbitol or a suitable substitute therefore. In certain aspects, a composition comprising an anti-IL-27 antibody can be prepared for storage by mixing the selected composition having the desired degree of purity with optional formulation agents (Remington's Pharmaceutical Sciences, supra) in the form of a lyophilized cake or an aqueous solution. Further, in certain aspects, a composition comprising an anti-IL-27 antibody can be formulated as a lyophilizate using appropriate excipients such as sucrose.

In certain aspects, the pharmaceutical composition can be selected for parenteral delivery. In certain aspects, the compositions can be selected for inhalation or for delivery through the digestive tract, such as orally. The preparation of such pharmaceutically acceptable compositions is within the ability of one skilled in the art.

In certain aspects, the formulation components are present in concentrations that are acceptable to the site of administration. In certain aspects, buffers are used to maintain the composition at physiological pH or at a slightly lower pH, typically within a pH range of from about 5 to about 8.

In certain aspects, when parenteral administration is contemplated, a therapeutic composition can be in the form of a pyrogen-free, parenterally acceptable aqueous solution comprising an anti-IL-27 antibody, in a pharmaceutically acceptable vehicle. In certain aspects, a vehicle for parenteral injection is sterile distilled water in which an anti-IL-27 antibody is formulated as a sterile, isotonic solution, and properly preserved. In certain aspects, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain aspects, hyaluronic acid can also be used, and can have the effect of promoting sustained duration in the circulation. In certain aspects, implantable drug delivery devices can be used to introduce the desired molecule.

In certain aspects, a pharmaceutical composition can be formulated for inhalation. In certain aspects, an anti-IL-27 antibody can be formulated as a dry powder for inhalation. In certain aspects, an inhalation solution comprising an anti-IL-27 antibody can be formulated with a propellant for aerosol delivery. In certain aspects, solutions can be nebulized. Pulmonary administration is further described in PCT application No. PCT/US94/001875, which describes pulmonary delivery of chemically modified proteins.

In certain aspects, it is contemplated that formulations can be administered orally. In certain aspects, an anti-IL-27 antibody that is administered in this fashion can be formulated with or without those carriers customarily used in the compounding of solid dosage forms such as tablets and capsules. In certain aspects, a capsule can be designed to release the active portion of the formulation at the point in the gastrointestinal tract when bioavailability is maximized and pre-systemic degradation is minimized. In certain aspects, at least one additional agent can be included to facilitate absorption of an anti-IL-27 antibody. In certain aspects, diluents, flavorings, low melting point waxes, vegetable oils, lubricants, suspending agents, tablet disintegrating agents, and binders can also be employed.

In certain aspects, a pharmaceutical composition can involve an effective quantity of an anti-IL-27 antibody in a mixture with non-toxic excipients which are suitable for the manufacture of tablets. In certain aspects, by dissolving the tablets in sterile water, or another appropriate vehicle, solutions can be prepared in unit-dose form. In certain aspects, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an anti-IL-27 antibody in sustained- or controlled-delivery formulations. In certain aspects, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain aspects, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain aspects, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain aspects, this can be accomplished by filtration through sterile filtration membranes. In certain aspects, where the composition is lyophilized, sterilization using this method can be conducted either prior to or following lyophilization and reconstitution. In certain aspects, the composition for parenteral administration can be stored in lyophilized form or in a solution. In certain aspects, parenteral compositions generally are placed into a container having a sterile access port, for example, an intravenous solution bag or vial having a stopper pierceable by a hypodermic injection needle.

In certain aspects, once the pharmaceutical composition has been formulated, it can be stored in sterile vials as a solution, suspension, gel, emulsion, solid, or as a dehydrated or lyophilized powder. In certain aspects, such formulations can be stored either in a ready-to-use form or in a form (e.g., lyophilized) that is reconstituted prior to administration.

In certain aspects, kits are provided for producing a single-dose administration unit. In certain aspects, the kit can contain both a first container having a dried protein and a second container having an aqueous formulation. In certain aspects, kits containing single and multi-chambered pre-filled syringes (e.g., liquid syringes and lyosyringes) are included.

In certain aspects, the effective amount of a pharmaceutical composition comprising an anti-IL-27 antibody to be employed therapeutically will depend, for example, upon the therapeutic context and objectives. One skilled in the art will appreciate that the appropriate dosage levels for treatment, according to certain aspects, will thus vary depending, in part, upon the molecule delivered, the indication for which an anti-IL-27 antibody is being used, the route of administration, and the size (body weight, body surface or organ size) and/or condition (the age and general health) of the patient. In certain aspects, the clinician can titer the dosage and modify the route of administration to obtain the optimal therapeutic effect.

In certain aspects, the frequency of dosing will take into account the pharmacokinetic parameters of an anti-IL-27 antibody in the formulation used. In certain aspects, a clinician will administer the composition until a dosage is reached that achieves the desired effect. In certain aspects, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain aspects, appropriate dosages can be ascertained through use of appropriate dose-response data.

In certain aspects, the route of administration of the pharmaceutical composition is in accord with known methods, e.g. orally, through injection by intravenous, intraperitoneal, intracerebral (intra-parenchymal), intracerebroventricular, intramuscular, subcutaneously, intraocular, intraarterial, intraportal, or intralesional routes; by sustained release systems or by implantation devices. In certain aspects, the compositions can be administered by bolus injection or continuously by infusion, or by implantation device. In certain aspects, individual elements of the combination therapy may be administered by different routes.

In certain aspects, the composition can be administered locally via implantation of a membrane, sponge or another appropriate material onto which the desired molecule has been absorbed or encapsulated. In certain aspects, where an implantation device is used, the device can be implanted into any suitable tissue or organ, and delivery of the desired molecule can be via diffusion, timed-release bolus, or continuous administration. In certain aspects, it can be desirable to use a pharmaceutical composition comprising an anti-IL-27 antibody in an ex vivo manner. In such instances, cells, tissues and/or organs that have been removed from the patient are exposed to a pharmaceutical composition comprising an anti-IL-27 antibody after which the cells, tissues and/or organs are subsequently implanted back into the patient.

In certain aspects, an anti-IL-27 antibody can be delivered by implanting certain cells that have been genetically engineered, using methods such as those described herein, to express and secrete the polypeptides. In certain aspects, such cells can be animal or human cells, and can be autologous, heterologous, or xenogeneic. In certain aspects, the cells can be immortalized. In certain aspects, in order to decrease the chance of an immunological response, the cells can be encapsulated to avoid infiltration of surrounding tissues. In certain aspects, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Applications

The compositions described herein can be used in a number of diagnostic and therapeutic applications. For example, detectably labeled antigen-binding molecules can be used in assays to detect the presence or amount of the target antigens in a sample (e.g., a biological sample). The compositions can be used in in vitro assays for studying inhibition of target antigen function. In some aspects, e.g., in which the compositions bind to and inhibit a complement protein, the compositions can be used as positive controls in assays designed to identify additional novel compounds that inhibit complement activity or otherwise are useful for treating a complement-associated disorder. For example, a IL-27-inhibiting composition can be used as a positive control in an assay to identify additional compounds (e.g., small molecules, aptamers, or antibodies) that reduce or abrogate IL-27 production. The compositions can also be used in therapeutic methods as elaborated on below.

In some aspects, the disclosure provides a method of detecting IL-27 in a biological sample or in a subject, comprising (i) contacting the sample or the subject (and optionally, a reference sample or subject) with any antibody described herein under conditions that allow interaction of the antibody molecule and IL-27 to occur, and (ii) detecting formation of a complex between the antibody molecule and the sample or the subject (and optionally, the reference sample or subject).

### Kits

A kit can include an anti-IL-27 antibody as disclosed herein, and instructions for use. The kits may comprise, in a suitable container, an anti-IL-27 antibody, one or more controls, and various buffers, reagents, enzymes and other standard ingredients well known in the art. In some aspects, the disclosure provides a kit comprising an anti-IL-27 antibody or antigen-binding portion as disclosed herein, and instructions for use in stimulating an immune response in a subject, or treating cancer in a subject, optionally with instructions for use in combination with one or more additional therapeutic agents or procedure as disclosed herein.

The container can include at least one vial, well, test tube, flask, bottle, syringe, or other container means, into which an anti-IL-27 antibody may be placed, and in some instances, suitably aliquoted. Where an additional component is provided, the kit can contain additional containers into which this component may be placed. The kits can also include a means for containing an anti-IL-27 antibody and any other reagent containers in close confinement for commercial sale. Such containers may include injection or blow-molded plastic containers into which the desired vials are retained. Containers and/or kits can include labeling with instructions for use and/or warnings.

### Methods of Use

The compositions of the present invention have numerous in vitro and in vivo utilities involving the detection and/or quantification of IL-27 and/or the antagonism of IL-27 function.

In some aspects, the disclosure provides a method to inhibit or reduce STAT1 and/or STAT3 phosphorylation in a cell, the method comprising contacting the cell with an isolated antibody, or antigen binding fragment, provided by the disclosure, wherein the antibody, or antigen binding portion thereof, inhibits or reduces STAT1 and/or STAT3 phosphorylation in a cell.

In some aspects, the disclosure provides a method to inhibit or reduce inhibition of CD161 expression in a cell, the method comprising contacting the cell with an isolated antibody, or antigen binding fragment, provided by the disclosure, wherein the antibody, or antigen binding portion thereof, inhibits or reduces inhibition of CD161 expression in a cell.

In some aspects, the disclosure provides a method to inhibit or reduce PD-L1 and/or TIM-3 expression in a cell, the method comprising contacting the cell with an isolated antibody, or antigen binding fragment, provided by the disclosure, wherein the antibody, or antigen binding portion thereof, inhibits or PD-L1 and/or TIM-3 expression in a cell.

In some aspects, the disclosure provides a method to induce or enhance secretion of one or more cytokines from a cell, the method comprising contacting the cell with the isolated antibody, or antigen binding fragment, provided by the disclosure, wherein the antibody, or antigen binding portion thereof, induces or enhances PD-1 mediated secretion of one or more cytokines from a cell.

In some aspects, the disclosure provides a method of stimulating an immune response in a subject, the method comprising administering to the subject an effective amount of an isolated antibody, or antigen binding portion thereof, that specifically binds to and antagonizes IL-27, provided by the disclosure, or a pharmaceutical composition comprising the antibody or antigen binding portion thereof, and a pharmaceutically acceptable carrier.

In some aspects, the disclosure provides a method of treating a cancer in a subject, the method comprising administering to the subject an effective amount an isolated antibody, or antigen binding portion thereof, that specifically binds to and antagonizes IL-27, provided by the disclosure, or a pharmaceutical composition comprising the antibody or antigen binding portion thereof, and a pharmaceutically acceptable carrier.

In some aspects, the disclosure provides a method of stimulating an immune response, or treating a cancer in a subject, the method comprising administering to the subject an effective amount of an isolated antibody, or antigen binding fragment, provided by the disclosure, or a pharmaceutical composition comprising the antibody or antigen binding portion thereof, and a pharmaceutically acceptable carrier, wherein the antibody, or antigen binding portion thereof, or the pharmaceutical composition, inhibits or reduces STAT1 and/or STAT3 phosphorylation in a cell, thereby stimulating the immune response, or treating the cancer.

In some aspects, the disclosure provides a method of stimulating an immune response, or treating a cancer in a subject, the method comprising administering to the subject an effective amount of an isolated antibody, or antigen binding fragment, provided by the disclosure, or a pharmaceutical composition comprising the antibody or antigen binding portion thereof, and a pharmaceutically acceptable carrier, wherein the antibody, or antigen binding portion thereof, or the pharmaceutical composition, inhibits or reduces inhibition of CD161 expression in a cell, thereby stimulating the immune response, or treating the cancer.

In some aspects, the disclosure provides a method of stimulating an immune response, or treating a cancer in a subject, the method comprising administering to the subject an effective amount of an isolated antibody, or antigen binding fragment, provided by the disclosure, or a pharmaceutical composition comprising the antibody or antigen binding portion thereof, and a pharmaceutically acceptable carrier, wherein the antibody, or antigen binding portion thereof, or the pharmaceutical composition, inhibits or reduces PD-L1 and/or TIM-3 expression in a cell, thereby stimulating the immune response, or treating the cancer.

In some aspects, the disclosure provides a method of stimulating an immune response, or treating a cancer in a subject, the method comprising administering to the subject an effective amount of an isolated antibody, or antigen binding fragment, provided by the disclosure, or a pharmaceutical composition comprising the antibody or antigen binding portion thereof, and a pharmaceutically acceptable carrier, wherein the antibody, or antigen binding portion thereof, or the pharmaceutical composition, induces or enhances PD-1-mediated secretion of one or more cytokines from a cell, thereby stimulating the immune response, or treating the cancer.

In some aspects, the cancer is chosen from lung cancer (e.g., non-small cell lung cancer), sarcoma, testicular cancer, ovarian cancer, pancreas cancer, breast cancer (e.g., triple-negative breast cancer), melanoma, head and neck cancer (e.g., squamous head and neck cancer), colorectal cancer, bladder cancer, endometrial cancer, prostate cancer, thyroid cancer, hepatocellular carcinoma, gastric cancer, brain cancer, lymphoma (e.g., DL-BCL), leukemia (e.g., AML) or renal cancer (e.g., renal cell carcinoma, e.g., renal clear cell carcinoma).

The above-described compositions are useful in, inter alia, methods for treating or preventing a variety of cancers in a subject. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as silastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some aspects, an anti-IL-27 antibody or antigen-binding fragment thereof is therapeutically delivered to a subject by way of local administration.

A suitable dose of an antibody or fragment thereof described herein, which dose is capable of treating or preventing cancer in a subject, can depend on a variety of factors including, e.g., the age, sex, and weight of a subject to be treated and the particular inhibitor compound used. For example, a different dose of a whole anti-IL-27 antibody may be required to treat a subject with cancer as compared to the dose of a IL-27-binding Fab′ antibody fragment required to treat the same subject. Other factors affecting the dose administered to the subject include, e.g., the type or severity of the cancer. For example, a subject having metastatic melanoma may require administration of a different dosage of an anti-IL-27 antibody than a subject with glioblastoma. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will also depend upon the judgment of the treating medical practitioner (e.g., doctor or nurse). Suitable dosages are described herein.

A pharmaceutical composition can include a therapeutically effective amount of an anti-IL-27 antibody or antigen-binding fragment thereof described herein. Such effective amounts can be readily determined by one of ordinary skill in the art based, in part, on the effect of the administered antibody, or the combinatorial effect of the antibody and one or more additional active agents, if more than one agent is used. A therapeutically effective amount of an antibody or fragment thereof described herein can also vary according to factors such as the disease state, age, sex, and weight of the individual, and the ability of the antibody (and one or more additional active agents) to elicit a desired response in the individual, e.g., reduction in tumor growth. For example, a therapeutically effective amount of an anti-IL-27 antibody can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the antibodies or fragments thereof described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

In some aspects, the composition contains any of the antibodies or antigen-binding fragments thereof described herein and one or more (e.g., two, three, four, five, six, seven, eight, nine, 10, or 11 or more) additional therapeutic agents such that the composition as a whole is therapeutically effective. For example, a composition can contain an anti-IL-27 antibody described herein and an alkylating agent, wherein the antibody and agent are each at a concentration that when combined are therapeutically effective for treating or preventing a cancer (e.g., melanoma) in a subject.

Toxicity and therapeutic efficacy of such compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. An antibody or antigen-binding fragment thereof that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies or antigen-binding fragments thereof lies generally within a range of circulating concentrations of the antibodies or fragments that include the ED₅₀ with little or no toxicity. The dosage may vary within this range depending upon the dosage form employed and the route of administration utilized. For an anti-IL-27 antibody described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some aspects, e.g., where local administration (e.g., to the eye or a joint) is desired, cell culture or animal modeling can be used to determine a dose required to achieve a therapeutically effective concentration within the local site.

In some aspects, the methods can be performed in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

As described above, the compositions described herein (e.g., anti-IL-27 compositions) can be used to treat a variety of cancers such as but not limited to: Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma (HCC), hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

### Combination Therapy

In some aspects, an anti-IL-27 antibody, or antigen binding portion thereof, provided by the disclosure, can be combined with one or more additional therapeutics or treatments, e.g., another therapeutic or treatment for a cancer. For example, the anti-IL-27 antibody, or antigen binding portion thereof, can be administered to a subject (e.g., a human patient) in combination with one or more additional therapeutics, wherein the combination provides a therapeutic benefit to a subject who has, or is at risk of developing, cancer.

In some aspects, an anti-IL-27 antibody, or antigen binding portion thereof, and the one or more additional therapeutics are administered at the same time (e.g., simultaneously). In other aspects, the anti-IL-27 antibody, or antigen binding portion thereof, is administered first in time and the one or more additional therapeutics are administered second in time (e.g., sequentially). In some aspects, the one or more additional therapeutics are administered first in time and the anti-IL-27 antibody is administered second in time.

An anti-IL-27 antibody or an antigen-binding fragment thereof described herein can replace or augment a previously or currently administered therapy. For example, upon treating with an anti-IL-27 antibody or antigen-binding fragment thereof, administration of the one or more additional therapeutics can cease or diminish, e.g., be administered at lower levels. In some aspects, administration of the previous therapy can be maintained. In some aspects, a previous therapy will be maintained until the level of the anti-IL-27 antibody reaches a level sufficient to provide a therapeutic effect.

In some aspects, the disclosure provides a method of treating cancer in a subject, the method comprising administering to the subject an effective amount of an isolated antibody, or antigen binding portion thereof, that specifically binds to and antagonizes IL-27, provided by the disclosure, in combination with one or more additional therapeutic agents or procedure, wherein the second therapeutic agent or procedure is selected from the group consisting of: a chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cellular immunotherapy, or a combination thereof.

In some aspects, the one or more additional therapeutic agents is a PD-1 antagonist, a TIM-3 inhibitor, a LAG-3 inhibitor, a TIGIT inhibitor, a CD112R inhibitor, a TAM inhibitor, a STING agonist, a 4-1BB agonist, or a combination thereof. In some aspects, the one or more additional therapeutic agents is a CD39 antagonist, a CD73 antagonist, a CCR8 antagonist, or a combination thereof. In some aspects, the anti-CD73 is any anti-CD73 antibody disclosed in, e.g., U.S. Publication No. 2019/0031766 A1, which is incorporated by reference herein in its entirety. In some aspects, the anti-CD39 is any anti-CD39 antibody disclosed in, e.g., Int'l Publication No. WO 2019/178269 A2, which is incorporated by reference herein in its entirety.

In some aspects, the one or more additional therapeutic agents is a PD-1 antagonist. In some aspects, the PD-1 antagonist is selected from the group consisting of: PDR001, nivolumab, pembrolizumab, pidilizumab, MEDI0680, REGN2810, TSR-042, PF-06801591, and AMP-224. In certain aspects, the one or more additional therapeutic agents is a PD-L1 inhibitor. In some aspects, the PD-L1 inhibitor is selected from the group consisting of: FAZ053, Atezolizumab, Avelumab, Durvalumab, and BMS-936559. In some aspects, the disclosure provides a method of enhancing one or more activities of an anti-PD-1 antibody (e.g., enhances PD-1-mediated cytokine secretion; enhances anti-PD-1 mediated TNFα secretion; enhances anti-PD-1 mediated IL-6 secretion from a cell exposed to anti-PD-1 antibodies), the method comprising exposing a cell to an antibody, or antigen binding portion thereof, provided by the disclosure, concurrently with or sequentially to an anti-PD-1 antibody, thereby to enhance one or more activities of the anti-PD1 antibody.

In some aspects, the one or more additional therapeutic agents is Sunitinib (Sutent®), Cabozantinib (CABOMETYX®), Axitinib (INLYTA®), Lenvatinib (LENVIMA®), Everolimus (AFINITOR®) , Bevacizumab (AVASTIN®), epacadostat, NKTR-214 (CD-122-biased agonist), tivozanib (FOTIVDA®), abexinostat, Ipilimumab (YERVOY®), tremelimumab, Pazopanib (VOTRIENT®), Sorafenib (NEXAVAR®), Temsirolimus (TORISEL®), Ramucirumab (CYRAMZA®), niraparib, savolitinib, vorolanib (X-82), Regorafenib (STIVARGO®), Donafenib (multikinase inhibitor), Camrelizumab (SHR-1210), pexastimogene devacirepvec (JX-594), Ramucirumab (CYRAMZA®), apatinib (YN968D1), encapsulated doxorubicin (THERMODOX®), Tivantinib (ARQ197), ADI-PEG 20, binimetinib, apatinib mesylate, nintedanib, lirilumab, Nivolumab (OPDIVO®), Pembrolizumab (KEYTRUDA®), Atezolizumab (TECENTRIQ®), Avelumab (BAVENCIO®), Durvalumab (IMFIMZI®), Cemiplimab-rwlc (LIBTAYO®), tislelizumab, and/or spartalizumab.

In some aspects, the one or more additional therapeutic agents is a TIM-3 inhibitor, optionally wherein the TIM-3 inhibitor is MGB453 or TSR-022.

In some aspects, the one or more additional therapeutic agents is a LAG-3 inhibitor, optionally wherein the LAG-3 inhibitor is selected from the group consisting of LAG525, BMS-986016, and TSR-033.

In some aspects, the one or more additional therapeutic agents is a TIGIT inhibitor. In some aspects, the one or more additional therapeutic agents is a CD112R inhibitor. In some aspects, the one or more additional therapeutic agents is a TAM (Axl, Mer, Tyro) inhibitor. In some aspects, the one or more additional therapeutic agents is a STING agonist. In some aspects, the one or more additional therapeutic agents is a 4-1BB agonist.

In some aspects, the one or more additional therapeutic agents is a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof.

Combination with Chemotherapeutic Agents

Chemotherapeutic agents suitable for combination and/or co-administration with compositions of the present invention include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g., daunorubicin (formerly daunomycin) and doxorubicin), antibiotics (e.g., dactinomcin (formerly actinomycin), bleomycin, mithramycin, and anthramycin (AMC)), and anti-mitotic agents (e.g., vincristine and vinblastine) and temozolomide.

Combination with PD-1/PD-L1 Antagonists

In some aspects, the anti-IL-27 antibodies, or antigen binding portions thereof, provided by the disclosure are combined (e.g., administered in combination) with one or more PD-1 antagonist that specifically binds to human PD-1 or PD-L1 and inhibits PD-1/PD-L1 biological activity and/or downstream pathway(s) and/or cellular processed mediated by human PD-1/PD-L1 signaling or other human PD-1/PD-L1-mediated functions.

Accordingly, provided herein are PD-1 antagonists that directly or allosterically block, antagonize, suppress, inhibit or reduce PD-1/PD-L1 biological activity, including downstream pathways and/or cellular processes mediated by PD-1/PD-L1 signaling, such as receptor binding and/or elicitation of a cellular response to PD-1/PD-L1. Also provided herein are PD-1 antagonists that reduce the quantity or amount of human PD-1 or PD-L1 produced by a cell or subject.

In some aspects, the disclosure provides a PD-1 antagonist that binds human PD-1 and prevents, inhibits or reduces PD-L1 binding to PD-1. In some aspects, the PD-1 antagonist binds to the mRNA encoding PD-1 or PD-L1 and prevents translation. In some aspects, the PD-1 antagonist binds to the mRNA encoding PD-1 or PD-L1 and causes degradation and/or turnover.

In some aspects, the PD-1 antagonist inhibits PD-1 signaling or function. In some aspects, the PD-1 antagonist blocks binding of PD-1 to PD-L1, PD-L2, or to both PD-L1 and PD-L2. In some aspects, the PD-1 antagonist blocks binding of PD-1 to PD-L1. In some aspects, the PD-1 antagonist blocks binding of PD-1 to PD-L2. In some aspects, the PD-1 antagonist blocks the binding of PD-1 to PD-L1 and PD-L2. In some aspects, the PD-1 antagonist specifically binds PD-1. In some aspects, the PD-1 antagonist specifically binds PD-L1. In some aspects, the PD-1 antagonist specifically binds PD-L2.

In some aspects, the PD-1 antagonist inhibits the binding of PD-1 to its cognate ligand. In some aspects, the PD-1 antagonist inhibits the binding of PD-1 to PD-L1, PD-1 to PD-L2, or PD-1 to both PD-L1 and PD-L2. In some aspects, the PD-1 antagonist does not inhibit the binding of PD-1 to its cognate ligand.

In some aspects, the PD-1 antagonist is an isolated antibody (mAb), or antigen binding fragment thereof, which specifically binds to PD-1 or PD-L1. In some aspects, the PD-1 antagonist is an antibody or antigen binding fragment thereof that specifically binds to human PD-1. In some aspects, the PD-1 antagonist is an antibody or antigen binding fragment thereof that specifically binds to human PD-L1. In some aspects, the PD-1 antagonist is an antibody or antigen binding fragment that binds to human PD-L1 and inhibits the binding of PD-L1 to PD-1. In some aspects, the PD-1 antagonist is an antibody or antigen binding fragment that binds to human PD-1 and inhibits the binding of PD-L1 to PD-1.

Several immune checkpoint antagonists that inhibit or disrupt the interaction between PD-1 and either one or both of its ligands PD-L1 and PD-L2 are in clinical development or are currently available to clinicians for treating cancer.

Examples of anti-human PD-1 antibodies, or antigen binding fragments thereof, that may comprise the PD-1 antagonist in any of the compositions, methods, and uses provided by the disclosure include, but are not limited to: KEYTRUDA® (pembrolizumab, MK-3475, h409A11; see U.S. Pat. Nos. 8,952,136, 8,354,509, 8,900,587, and EP2170959, all of which are included herein by reference in their entirety; Merck), OPDIVO® (nivolumab, BMS-936558, MDX-1106, ONO-4538; see U.S. Pat. Nos. 7,595,048, 8,728,474, 9,073,994, 9,067,999, EP1537878, U.S. Pat. Nos. 8,008,449, 8,779,105, and EP2161336, all of which are included herein by reference in their entirety; Bristol Myers Squibb), MEDI0680 (AMP-514), BGB-A317 and BGB-108 (BeiGene), 244C8 and 388D4 (see WO2016106159, which is incorporated herein by reference in its entirety; Enumeral Biomedical), PDR001 (Novartis), and REGN2810 (Regeneron). Accordingly, in some aspects the PD-1 antagonist is pembrolizumab. In some aspects, the PD-1 antagonist is nivolumab.

Examples of anti-human PD-L1 antibodies, or antigen binding fragments thereof, that may comprise the PD-1 antagonist in any of the compositions, methods, and uses provided by the disclosure include, but are not limited to: BAVENCIO® (avelumab, MSB0010718C, see WO2013/79174, which is incorporated herein by reference in its entirety; Merck/Pfizer), IMFINZI® (durvalumab, MEDI4736), TECENTRIQ® (atezolizumab, MPDL3280A, RG7446; see WO2010/077634, which is incorporated herein by reference in its entirety; Roche), MDX-1105 (BMS-936559, 12A4; see U.S. Pat. No. 7,943,743 and WO2013/173223, both of which are incorporated herein by reference in their entirety; Medarex/BMS), and FAZ053 (Novartis). Accordingly, in some aspects the PD-1 antagonist is avelumab. In some aspects, the PD-1 antagonist is durvalumab. In some aspects, the PD-1 antagonist is atezolizumab.

In some aspects, the PD-1 antagonist is an immunoadhesin that specifically bind to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, both of which are incorporated herein by reference in their entirety. In some aspects, the PD-1 antagonist is AMP-224 (also known as B7-DCIg), which is a PD-L2-FC fusion protein that specifically binds to human PD-1.

It will be understood by one of ordinary skill that any PD-1 antagonist which binds to PD-1 or PD-L1 and disrupts the PD-1/PD-L1 signaling pathway, is suitable for compositions, methods, and uses disclosed herein.

In some aspects, the PD-1/PD-L1 antagonist is a small molecule, a nucleic acid, a peptide, a peptide mimetic, a protein, a carbohydrate, a carbohydrate derivative, or a glycopolymer. Exemplary small molecule PD-1 inhibitors are described in Zhan et al., (2016) Drug Discov Today 21(6):1027-1036.

Combinations with TIM-3 Inhibitors

In some aspects, an anti-IL-27 antibody, or antigen binding portion thereof, provided by the disclosure is combined (e.g., administered in combination) with a TIM-3 inhibitor. The TIM-3 inhibitor may be an antibody, an antigen binding fragment thereof, an immunoadhesin, a fusion protein, or an oligopeptide. In some aspects, the TIM-3 inhibitor is chosen from MGB453 (Novartis), TSR-022 (Tesaro), or LY3321367 (Eli Lilly). In some aspects, the anti-IL-27 antibody, or antigen binding portion thereof, is administered in combination with MGB453. In some aspects, the anti-IL-27 antibody, or antigen binding portion thereof, is administered in combination with TSR-022.

Combinations with LAG-3 Inhibitors

In some aspects, an anti-IL-27 antibody, or antigen binding portion thereof, provided by the disclosure is combined (e.g., administered in combination) with a LAG-3 inhibitor. The LAG-3 inhibitor may be an antibody, an antigen binding fragment thereof, an immunoadhesin, a fusion protein, or oligopeptide. In some aspects, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

### Other Combinations

In some aspects, an anti-IL-27 antibody, or antigen binding portion thereof, provided by the disclosure is combined (e.g., administered in combination) with a TIGIT inhibitor, a kinase inhibitor (e.g., a tyrosine kinase inhibitor (TKI)), a CD112R inhibitor, a TAM receptor inhibitor, a STING agonist and/or a 4-1BB agonist, or a combination thereof. In some aspects, an anti-IL-27 antibody, or antigen binding portion thereof, provided by the disclosure is combined (e.g., administered in combination) with a tyrosine kinase inhibitor, an agent targeting the adenosine axis (for example a CD39 antagonist, a CD73 antagonist or a A2AR, A2BR or dual A2AR/A2BR antagonist), a CCR8 antagonist, a CTLA4 antagonist, a VEG-F inhibitor or a combination thereof

### Methods of Detection

In some embodiments, an anti-IL-27 antibody or an antigen-binding fragment thereof described herein can be employed in methods of detection and/or quantification of human IL-27 in a biological sample. Accordingly, an anti-IL-27 antibody, or an antigen-binding fragment thereof, as described herein is useful to diagnose, prognose and/or determine progression of disease (e.g., cancer) in a patient.

Monitoring a subject (e.g., a human patient) for an improvement in a cancer, as defined herein, means evaluating the subject for a change in a disease parameter, e.g., a reduction in tumor growth. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer described herein.

In some embodiments, the disclosure provides a method of detecting IL-27 in a sample from a subject, the method comprising the (a) contacting a sample from the subject with a detection antibody under conditions to permit the detection antibody to form a detection antibody-IL-27 complex, if IL-27 is present in the sample, wherein the detection antibody is an antibody, or antigen binding fragment thereof, provided by the disclosure; and (b) detecting the presence of the complex, if any, produced in step (a).

In some embodiments, the disclosure provides a method of detecting an IL-27-associated cancer in a subject, the method comprising the steps of: (a) contacting a sample from a subject suspected of having an IL-27-associated cancer with a detection antibody under conditions to permit the detection antibody to form a detection antibody-IL-27 complex, if IL-27 is present in the sample, wherein the detection antibody is an antibody, or antigen binding portion thereof, provided by the disclosure; and (b) detecting the presence of the complex, if any, produced in step (a). In some embodiments, the detection antibody is coupled to a detectable label. In some embodiments, the method further comprises contacting the sample with a capture antibody to produce a complex comprising IL-27 and the capture antibody, if IL-27 is present in the sample, wherein the capture antibody is an antibody, or antigen binding portion thereof, provided by the disclosure.

In some embodiments, the capture antibody is immobilized on a solid support. In some embodiments, the sample is contacted with the capture antibody before the detection antibody. In some embodiments, the sample is a body fluid sample. In some embodiments, the fluid sample is blood, serum, plasma, cell lysates or tissue lysates.

In some embodiments, the cancer is selected from renal cell carcinoma (RCC), hepatocellular carcinoma, lung cancer, gastroesophageal cancer, ovarian cancer, endometrial cancer, melanoma, leukemia and lymphoma. In some embodiments, the cancer is renal cell carcinoma (RCC). In other embodiments, the cancer is hepatocellular carcinoma (HCC). In some embodiments, the cancer is selected from leukemia and lymphoma. In some embodiments, the cancer is acute myeloid leukemia (AML).

### EXAMPLES

While the present disclosure has been described with reference to the specific aspects thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Generation of Anti-IL-27 Antibodies in Yeast that Specifically Bind P28 Subunits of Human IL-27

Anti-IL-27 antibodies representing multiple epitope bins were selected from eight naïve human synthetic yeast libraries using methods described below.

### Materials and Methods

Eight naïve human synthetic yeast libraries each of ˜10⁹ diversity was propagated as previously described (see e.g., Xu et al., (2013) Protein Eng Des Sel 26(10):663-670; WO2009036379; WO2010105256; and WO2012009568, all of which are incorporated herein by reference in their entireties). For the first two rounds of selection, a magnetic bead sorting technique utilizing the Miltenyi MACS system was performed, as previously described (see e.g., Siegel et al. (2004) J Immunol Methods 286(1-2):141-153, which is incorporated herein by reference in its entirety).

Briefly, yeast cells (˜10¹⁰ cells/library) were incubated with 3 mL of 100 nM biotinylated antigen (recombinant human IL-27; R&D Systems) for 30 min at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μL) were added to the yeast and incubated for 15 min at 4° C. Next, the yeast cells were pelleted, resuspended in 20 mL wash buffer, and loaded onto a Miltenyi LS column. After the 20 mL was loaded, the column was washed 3 times with 3 mL wash buffer. The column was then removed from the magnetic field, and the yeast cells were eluted with 5 mL of growth media and then grown overnight. The following rounds of selection were performed using flow cytometry. Approximately 2×10⁷ yeast cells were pelleted, washed three times with wash buffer, and incubated at 30° C. with either decreasing concentrations of biotinylated antigen (100 to 1 nM) under equilibrium conditions, 30 nM biotinylated antigens of different species in order to obtain species cross-reactivity, or with a poly-specificity depletion reagent (PSR) to remove non-specific antibodies from the selection. For the PSR depletion, the libraries were incubated with a 1:10 dilution of biotinylated PSR reagent.

Yeast cells were then washed twice with wash buffer and stained with LC-FITC (diluted 1:100) and either SA-633 (diluted 1:500) or EAPE (diluted 1:50) secondary reagents for 15 min at 4° C. After washing twice with wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast cells were plated and individual colonies were picked for characterization.

### Light Chain Diversification

Light chain diversification protocol was used during the primary discovery phase for further discovery and improvement of antibodies.

Light chain batch diversification protocol: Heavy chain plasmids from a naïve selection output were extracted from the yeast via smash and grab, propagated in and subsequently purified from E. coli and transformed into a light chain library with a diversity of 5×10⁶. Selections were performed with one round of MACS and four rounds of FACS employing the same conditions as the naïve discovery.

### Antibody Optimization

Optimization of antibodies was performed by introducing diversities into the heavy chain and light chain variable regions as described below.

CDRH1 and CDRH2 selection: The CDRH3 of a single antibody was recombined into a premade library with CDRH1 and CDRH2 variants of a diversity of 1×10⁸ and selections were performed with one round of MACS and four rounds of FACS as described in the naïve discovery. In the different FACS rounds the libraries were looked at for PSR binding, species cross-reactivity, and affinity pressure by titration or parental Fab pre-complexing, and sorting was performed in order to obtain a population with the desired characteristics.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect (GE Healthcare LifeSciences).

### ForteBio K_{D} Measurements

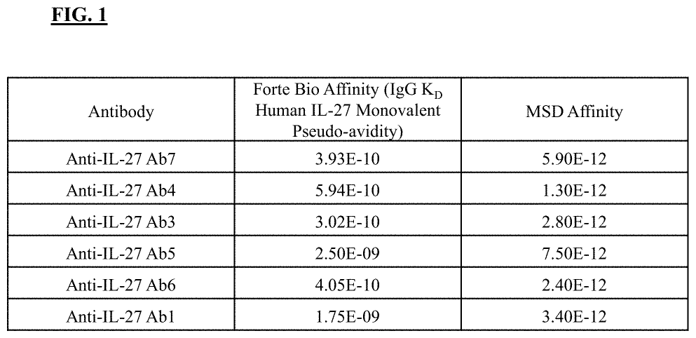

ForteBio affinity measurements were performed on an Octet RED384 generally as previously described (see, e.g., Estep et al, High throughput solution-based measurement of antibody-antigen affinity and epitope binning. Mabs 5(2), 270-278 (2013), herein incorporated by reference in its entirety). Briefly, ForteBio affinity measurements were performed by loading IgGs on-line onto AHQ sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model. Recombinant Human IL-27 Protein (R&D Systems Cat: 2526-IL) was used as an antigen. Affinity measurements for anti-IL-27 antibodies is shown in FIG. 1.

### ForteBio Epitope Binning/Ligand Blocking

Epitope binning/ligand blocking was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM target antigen followed by a second anti-target antibody or ligand. Additional binding by the second antibody or ligand after antigen association indicates an unoccupied epitope (non-competitor), while no binding indicates epitope blocking (competitor or ligand blocking).

### MSD-SET Kinetic Assay

Equilibrium affinity measurements performed as previously described (Estep et al., 2013). Solution equilibrium titrations (SET) were performed in PBS+0.1% IgG-Free BSA (PBSF) with antigen held constant at 10-100 pM and incubated with 3- to 5-fold serial dilutions of antibody starting at 5-100 nM (experimental condition is sample dependent). Antibodies (20 nM in PBS) were coated onto standard bind MSD-ECL plates overnight at 4° C. or at room temperature for 30 min. Plates were then blocked for 30 min with shaking at 700 rpm, followed by three washes with wash buffer (PBSF+0.05% Tween 20). SET samples were applied and incubated on the plates for 150 s with shaking at 700 rpm followed by one wash. Antigen captured on a plate was detected with 250 ng/mL sulfotag-labeled streptavidin in PBSF by incubation on the plate for 3 min. The plates were washed three times with wash buffer and then read on the MSD Sector Imager 2400 instrument using 1× Read Buffer T with surfactant. The percent free antigen was plotted as a function of titrated antibody in Prism and fit to a quadratic equation to extract the K_{D}. To improve throughput, liquid handling robots were used throughout MSD-SET experiments, including SET sample preparation.

### Example 2: Binding of Anti-IL-27 Antibodies to Recombinant Human IL-27

The ability of anti-IL-27 antibodies described in Example 1 to bind to recombinant human IL-27 was assessed by ELISA. Briefly, Nunc MaxiSorp ELISA Plates (Affymetrix #44-2404-21) were coated with 100 μL/well recombinant human IL-27 (R&D Systems #2526-IL/CF) (0.5 μg/mL diluted in PBS), sealed and incubated overnight at 4° C. Plates were washed 3 times with 100 μL/well of wash buffer (PBS+0.01% Tween). Plates were then blocked with 200 μL/well of blocking buffer (PBS+0.1% BSA+0.01% Tween) for 1 hour at room temperature (RT) with shaking. Blocking buffer was decanted and 100 μL per well of diluted control and anti-IL-27 antibodies were added, as indicated. A 10-point serial dilution was created for each antibody by diluting antibodies 1:10 starting from a top concentration of 1 μg/mL. Plates were incubated for 1-2 hours at RT with shaking. Plates were washed 3 times with 100 μL/well of wash buffer. 100 μL/well of anti-human IgG secondary antibody (SouthernBiotech; Cat. #2014-05) was added (1:5000 diluted in blocking buffer). Plates were then incubated for 1 hour at RT with shaking. After the 1 hour incubation, plates were washed 3 times with 100 μL/well of wash buffer. To develop the plates 100 μL/well TMB Buffer (Life Technologies #00-2023) was added. The development of blue color in the wells of the standard curve was observed and as soon as the highest concentration of diluted control antibodies reached a deep blue (5-10 minutes), 50 μL/well STOP Solution (Thermo Fisher #SS04) was added (the color will change to yellow). The developed plates were read at 450 nm (minus 570 nm for wavelength correction) within 30 minutes of stopping the reaction.

For example, biochemical affinity and specificity studies showed that the anti-IL-27 antibody anti-IL-27 Ab1 binds to the p28 subunit (but not the EBI3 subunit) of the heterodimeric cytokine IL-27. Anti-IL-27 Ab1 bound to human, nonhuman primate, and rodent recombinant IL-27, and the extent of the binding differed between species. The binding specificity of anti IL 27 Ab1 to IL-27 was confirmed by testing against a panel of 4500 cell surface and soluble molecules, and no off-target binding was observed. The binding specificity of IL-27 for its receptor IL-27RA (WSX-1) was also confirmed; no other cell surface receptor bound human IL-27. The ability of anti-IL-27 Ab1 to block the interaction between human IL-27 and IL-27RA (WSX-1) was confirmed by Surface Plasmon Resonance.

Binding of the antibodies disclosed herein was assessed in several model systems. Since human IL-27 is biologically active on mouse cells, systemic overexpression of human IL-27 in mice using DNA minicircle delivery was utilized to analyze IL-27-mediated effects in vivo by whole-genome microarray analysis, flow cytometry, and serum cytokine analysis. Many of the markers that were modulated by IL-27 in vivo were consistent with findings in human cell-based assays. Anti-IL-27 Ab3 was also evaluated in a disseminated B16 tumor model. In that setting, treatment with anti-IL-27 Ab3 showed results consistent with phenotypes observed in mice deficient for various components of IL-27 ligand (IL-27p28, EBI3) or receptor (IL-27RA).

Collectively, these studies demonstrate that anti-IL-27 Ab1 (and its sibling anti-IL-27 Ab3) can phenocopy IL 27 deficiency in mice, binds specifically and with high affinity to IL-27 and can inhibit its immunosuppressive effects, either alone or in combination with PD-L1 blocking agents.

### Example 3: Anti-IL27 Antibodies Inhibit Phosphorylation of STAT1 In Vitro

IL-27 signaling through the IL-27 receptor (IL-27R) results in the phosphorylation of the Signal Transducer And Activator Of Transcription 1 (STAT1) polypeptide (pSTAT1). Anti-IL-27 antibodies described in Example 1 were tested for their ability to inhibit IL-27-mediated phosphorylation of STAT1 in human whole blood, human PBMCs, the U937 myeloid cells (histiocytic lymphoma cell line) and HUT-78 T cell lymphoma cells by flow cytometry.

Anti-IL-27 antibodies were tested for their ability to inhibit IL-27-mediated phosphorylation of STAT1 in human whole blood. Briefly, EDTA anticoagulated whole human blood, stored at room temperature, was used in this assay. 45 μL blood was distributed into each well of a deep well, round bottom plate (Phenix #850356) and warmed for 30 minutes at 37° C. on a plate warmer (EchoTherm IC20) or in a 37° C. incubator. Anti-IL-27 antibodies were diluted to a 10× top concentration in endotoxin-free PBS (Teknova #P0300) in a polypropylene V-bottom plate (Corning #3363). Anti-IL-27 antibodies were serially diluted as desired in endotoxin-free PBS. PBS alone was added to wells for unstimulated and stimulated controls. 5 μL of each dilution was added to a well of 45 μL blood and mixed by shaking on plate shaker 15 seconds 1000 RPM (Eppendorf Mix Mate). The plate was incubated for 60 minutes at 37° C. on a plate warmer or in a 37° C. incubator.

A 10 μg vial of recombinant human IL-27 (R&D Systems #2526-IL) was reconstituted to 100 μg/mL by adding 100 μL PBS+0.1% BSA (made from 10% BSA Sigma #A1595). A working stock of the recombinant hIL-27 (rhIL-27) was prepared by dilution to 200 ng/mL in endotoxin-free PBS. After the 60-minute incubation, 5 μL of 200 ng/mL rhIL-27 was added to each well of stimulated blood. 5 μL PBS was added to unstimulated control wells. The plate was shaken on a plate shaker for 15 seconds at 1000 RPM. The plate was incubated for 30 minutes at 37° C.

After the 30-minute incubation, cells were fixed. Lyse/Fix reagent (BD #558049) was diluted 1:5 in sterile water (Hyclone #SH3052902) and warmed to 37° C. in a water bath. 500 μL Lyse/Fix reagent was added to each well of the deep well plate and the plate was mixed on a plate shaker for 15 seconds at 1000 RPM. The plate was incubated for 15 min at 37° C.

After the 15-minute incubation, the plate was centrifuged for 5 minutes at 1500 RPM at room temperature (Eppendorf centrifuge 5810R) and supernatant was discarded by flicking. 1 mL of endotoxin-free PBS was added per well and the plate was shaken on plate shaker for 15 seconds at 1000 RPM. The plate was centrifuged for 5 minutes at 1500 RPM at room temperature (Eppendorf centrifuge 5810R) and supernatant was discarded by flicking. Cell pellets remained in the plate.

Cell pellets were resuspended in 50 μL 1:200 CD14-Pacific Blue (Biolegend #325616) in FACS Buffer (PBS, Gibco #14190-144/2% FBS, Sigma #F8317/1 mM EDTA, Fisher #BP2482) and transferred to U-bottom 96 well plate (Costar #3799). The plate was sealed with plate sealer (VWR #89134-432) and incubated for 30 minutes at room temperature in the dark.

After the 30 minute incubation, 150 μL FACS buffer was added to each well and the plate was centrifuged at 1500 RPM for 5 minutes at room temperature. The cell pellets were then resuspended in 100 μL Perm III (stored at −20° C.) (BD #558050) with pipetting and the plate was sealed with plate sealer and lid. The plate was incubated overnight at −20° C. or 15 minutes at 4° C.

After the incubation, 150 μL PBS was added and the plate was centrifuged at 1500 RPM for 5 minutes at room temperature. The supernatant was discarded from the plate by flicking and the plate was resuspended in 50 μL staining cocktail prepared as described in the Table 6 below:

TABLE 6

| BD Catalog # | Antibody | Color | Dilution |
|---|---|---|---|
| 561807 | CD3 | FITC | 1:10 |
| 562069 | pSTAT1 Y701 | PE | 1:100 |
| 562071 | pSTAT3 Y705 | APC | 1:20 |

The plate was incubated for 1 hour at room temperature in the dark. After the 1-hour incubation, 100 μL of FACS buffer was added and the plate was centrifuged at 1500 RPM for 5 minutes at room temperature. The supernatant was discarded from the plate by flicking and the plate was resuspended in 100 μL FACS buffer for analysis by flow cytometry.

The anti-IL-27 antibodies described in Example 1 were tested for their ability to inhibit IL-27-mediated phosphorylation of STAT1 in pooled human PBMCs by flow cytometry. Briefly, frozen cryovials of human PBMC's (peripheral blood mononuclear cells), obtained from buffy coats, were removed from liquid nitrogen storage and quickly thawed in a 37° C. water bath. The contents of each cryovial was removed with a P1000 pipet and transferred to a 15 mL conical falcon tube. 2-3 mL of complete RPMI-1640 (Gibco, 61870-036) was slowly added to the thawed cells and cells were gently swirled or flicked to suspend. Conical tubes were topped-off up to 10 mL with complete RPMI-1640 and tubes were inverted to mix. Conical tubes were centrifuged tube at 1400 RPM at room temperature for 8 minutes.

PBMC cells were resuspended at a density of 4 million cells per mL in warm, serum-free RPMI-1640 and plated at a density of 200,000 cells per well (50 μL) in a round bottom 96-well plate (Costar, 3799). Anti-IL-27 antibodies were diluted in serum-free RPMI-1640 in the first row of a 96-well polypropylene plate to a top concentration of 40 μg/mL (will be 10 μg/mL final). Serial dilutions as desired (1:2, 1:3, etc) were made in the remainder of the first 10 rows of the plate. Fifty microliters (μL) of the antibody stock (4×) was added to the first 10 rows the plate of PBMC cells in the round bottom plate. In rows 11 and 12, 50 μL of serum-free RPMI-1640 cell media was added. The plate was then incubated at 37° C. for 2 hours.

After the 2-hour incubation, 100μ of 50 ng/ml recombinant human IL-27 (R&D Systems, 2526-IL) diluted in serum-free RPMI-1640 cell media was added to each well (except, the control wells which included serum-free media alone or antibody alone) for a final concentration of 25 ng/ml. 100 μL serum-free RPMI-1640 cell media was added to control wells or wells with antibody alone. The plate was incubated for 20 minutes at 37° C.

After the 20-minute incubation, 50 μL of 4% PFA (Pierce, 28906) in DI water was added directly to each well and the plate was incubated at 37° C. for 5 minutes to fix the cells. The plate was centrifuged at 2000 RPM for 5 minutes. Media was discarded by flicking and plate was washed with 150 μL DPBS. The washing steps were repeated 2 more times. 50μ ice cold 90% methanol (MeOH) (Sigma, 439193) diluted in H₂O was added quickly to each well using a 12-channel pipette. When adding the MeOH special care was taken to mix each well. The plate was incubated at 20° C. for at least 15 minutes. 100 μL of DPBS was added to each well on top of the 90% methanol and the plate was centrifuged at 2000 RPM for 5 minutes. Plate contents were discarded by flicking and the plate was washed 3 times as described previously. After the last wash, cell pellets remained in the wells of the plate.

The pelleted PBMC's were stained with pSTAT1 PE (BD Phosflow, 526069) 1:100 in FACS buffer (2% FBS, 2 mM EDTA in DPBS) for 45 minutes at room temperature in the dark. Special care was taken to mix each well with a 12-channel pipette when adding the stain. After the 45-minute incubation, 100 μL FACS buffer was added into each well and the plate was centrifuged at 2000 RPM for 5 minutes. Supernatant was discarded by flicking and the plate was washed 2 times as described previously. Cells were resuspended in 100 μL FACS buffer and analyzed by flow cytometry.

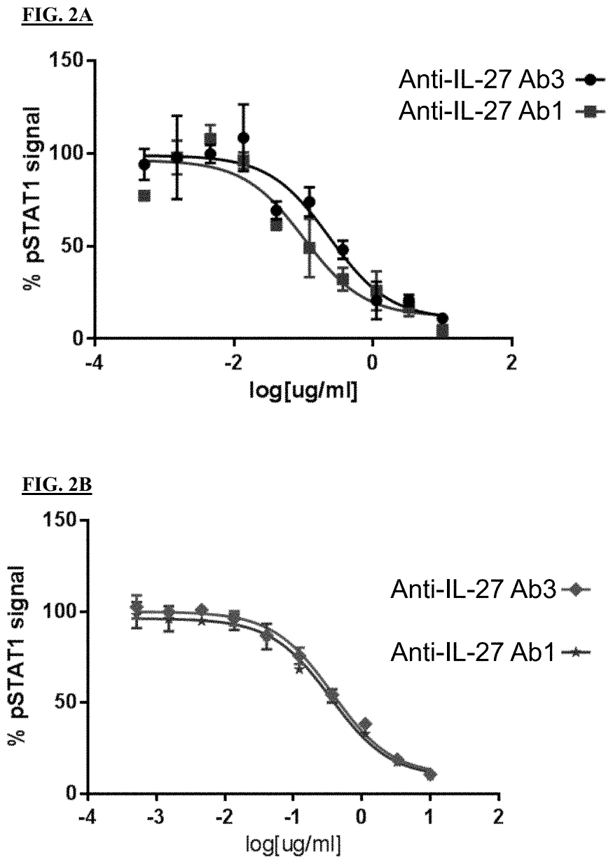

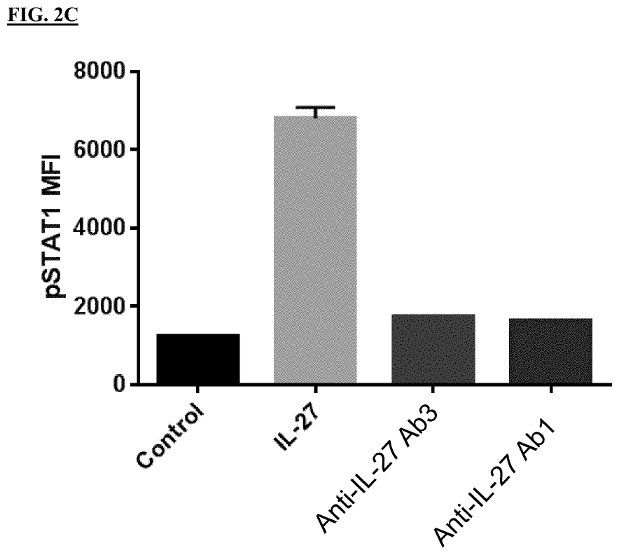

As shown in FIG. 2A, anti-IL-27 antibodies inhibited phosphorylation of STAT1 in human pooled PBMCs. The anti-IL-27 antibody anti-IL-27 Ab3 inhibited phosphorylation of STAT1 at an average IC₅₀ of 140.5 ng/mL (n=2) in pooled human PBMCs. The anti-IL-27 antibody anti-IL-27 Ab1 inhibited phosphorylation of STAT1 at an average IC₅₀ of 58.3 ng/mL (n=3) in pooled human PBMCs.

Anti-IL-27 antibodies were further tested for their ability to inhibit IL-27-mediated phosphorylation of STAT1 in U937 cells, a cell line known to express Fc receptors, by flow cytometry essentially as described for FIG. 2A. As shown in FIG. 2B, anti-IL-27 antibodies inhibit the phosphorylation of STAT1 in U-937 cells, as indicated. Anti-IL-27 Ab3 inhibited the phosphorylation of STAT1 at an average IC₅₀ of 81 ng/mL (n=2) in U937 cells. Antibody anti-IL-27 Ab1 inhibited the phosphorylation of STAT1 at an average IC₅₀ of 96 ng/mL (n=2) in U937 cells.

Anti-IL-27 antibodies were tested for their ability to inhibit IL-27-mediated phosphorylation of STAT1 in the cutaneous T-cell lymphoma line HUT-78, which does not express cell surface Fc receptors, by flow cytometry essentially as described for FIG. 2A. As shown in FIG. 2C, anti-IL-27 antibodies inhibited the phosphorylation of STAT1 in HUT-78 cells. Anti-IL-27 Ab3 inhibited the phosphorylation of STAT1 at an IC50 of 80 ng/mL (n=1) in HUT-78 cells. Antibody anti-IL-27 Ab1 inhibited the phosphorylation of STAT1 at an IC₅₀ of 95 ng/mL (n=1) in HUT-78 cells.

The present disclosure also assessed IL-27 inhibition by anti-IL-27 Ab1 across species in a whole blood assay. To characterize anti-IL-27 Ab1 activity across species, recombinant IL-27 from human, cynomolgus monkey, rat, and mouse was tested to stimulate pSTAT1 signaling in T lymphocytes from whole blood samples obtained from these species (data not shown).

Briefly, whole blood was warmed to 37° C. followed by a 60-minute pre-incubation with anti-IL-27 Ab1, and 20 ng/mL of human IL-27 was added. Samples were incubated for another 30 minutes. White blood cells were fixed, and red blood cells were lysed. After washing, fixed cells were permeabilized and stained with anti-CD3 and anti-phospho-STAT1 (Y701). After a 1-hour incubation, samples were washed and resuspended for flow cytometry. Percent inhibition was calculated using stimulated and unstimulated control wells, and IC50 values were calculated using GraphPad Prism.

Representative data for anti-IL-27 Ab1 signaling inhibition in human T cells are shown in FIG. 3. Consistent with observations made on the affinity of anti-IL-27 Ab1 to different species, the potency of IL-27 signaling inhibition by anti-IL-27 Ab1 was strongest in human, followed by cynomolgus monkey, rat, and mouse (see e.g., Table 7).

TABLE 7
Anti-IL-27 Ab1 IC₅₀ Values in Peripheral Blood T Cells
from Different Species

| | Average IC₅₀, | Standard | |
|---|---|---|---|
| Species | ng/mL | Deviation | Number |
| Human | 78.4 | 35 | 7 |
| Cynomolgus monkey | 118.1 | 36.4 | 4 |
| Rat | 273.2 | 133.5 | 8 |
| Mouse | 1721 | N/A | 1 (pool of 10) |

Abbreviations: IC₅₀ = half maximal inhibitory concentration, N/A = not applicable

### Example 4: Reduction of IL-27-Mediated Inhibition of CD161 by Anti-IL-27 Antibodies

The C-type lectin CD161 is a marker of T cells whose expression is suppressed by IL-27. Anti-IL-27 antibodies described in Example 1 were tested for their ability to reverse the IL-27-mediated inhibition of CD161 in pooled human PBMC cells by flow cytometry. Briefly, frozen cryovials of pooled human PBMC's (peripheral blood mononuclear cells), obtained from buffy coats, were removed from liquid nitrogen storage and quickly thawed in a 37° C. water bath. Contents of each cryovial was removed with a P1000 pipet and transferred to a 15 mL conical falcon tube. 2-3 mL of complete RPMI-1640 (Gibco, 61870-036) was slowly added to the thawed cells and cells were gently swirled or flicked to suspend. Conical tubes were topped-off up to 10 mL with complete RPMI-1640 and tubes were inverted to mix. Conical tubes were centrifuged tube at 1400 RPM, room temperature for 8 minutes.

Use of outer walls was avoided to minimize the effects of evaporation during the 5-day assay. Outer wells should be filled with 200 μL per well of DPBS (Gibco, 14190-144). PBMC cells were resuspended at a density of 2 million cells per mL in warm, complete RPMI-1640. Purified human anti-CD3 antibody (Biolegend, UCTH1, #300402) was added at a concentration of 0.5 μg/mL (this is 2× the final concentration). Plate 100 μL per well of this cell mixture (200,000 cells per well) in a round bottom 96 well plate (Costar, 3799).

Anti-IL-27 antibodies were diluted in complete RPMI-1640 in the first row of a 96 well polypropylene plate to a top concentration of 40 μg/mL (will be 10 ug/mL final). Serial dilutions as desired (1:2, 1:3, etc. . . . ) were made in the remainder of the first 10 rows of the plate. 50 μL of the antibody stock (4×) was added to the first 10 rows the plate of PBMC cells in the round bottom plate. In rows 11 and 12, 50 μL of complete RPMI-1640 was added.

After the addition of the anti-IL-27 antibodies, 50 μL of 100 ng/mL recombinant human IL-27 (R&D Systems, #2526-IL) diluted in complete RPMI-1640 was added to each well (except control wells which included serum free media or antibody alone) for a final concentration of 25 ng/mL. Fifty μL of complete RPMI-1640 was added to control wells. The plate was incubated for 5 days at 37.0 in a tissue culture incubator with minimal interference.

After the 5-day incubation the plate was removed from the incubator and agitated on a plate shaker for 30 seconds at 600 RPM. The plate was centrifuged at 1800 RPM for 5 minutes. Media was removed and set aside for additional assays and the plate was washed with 150 μL DPBS (Gibco, #14190-144). The washing steps were repeated 2 more times. The cell pellets were stained with 50 μL per well of staining cocktail as described in the Table 8 below:

TABLE 8

| Biolegend | Antibody | | |
|---|---|---|---|
| Catalog # | Target | Color | Dilution |
| 300532 | CD4 | BV421 | 1:100 |
| 304204 | CD45RO | FITC | 1:100 |
| 339910 | CD161 | AF647 | 1:100 |
| 353410 | CCR6 | PE | 1:100 |

The plate agitated on a plate shaker for 30 seconds at 600 RPM and the plate was incubated for 30 minutes at room temperature in the dark.

After the 30-minute incubation the plate was centrifuged and supernatant was discarded by flicking. The plate was washed 2 times as described previously. After the last wash, cell pellets were fixed by adding 50 μL 4% PFA (Pierce, 28906) in DI water at room temperature for 10 mins. 100 μL of FASC buffer was added to each well, and the plate was centrifuged at 1800 RPM for 5 minutes. Cells were resuspended in 100 μL FACS buffer and read by flow cytometry.

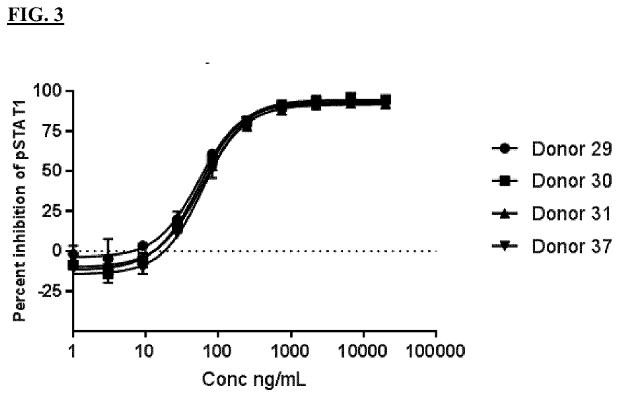

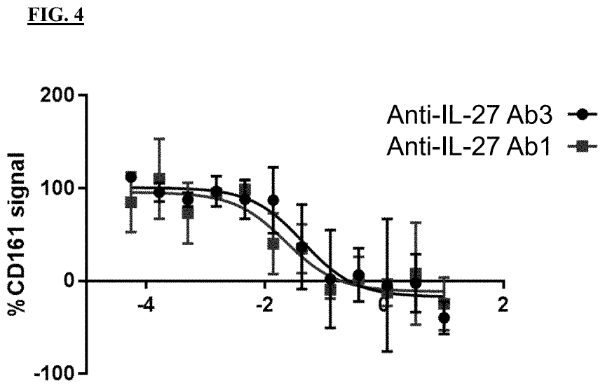

As shown in FIG. 4, anti-IL-27 antibodies, as indicated, reduce the IL-27 mediated inhibition of CD161.

### Example 5: Enhancement of PD-1-Mediated Secretion of TNFα, IL-6 and Other Cytokines by Anti-IL-27 Antibodies, Including Additional In Vitro Characterization of Anti-IL-27 Antibodies

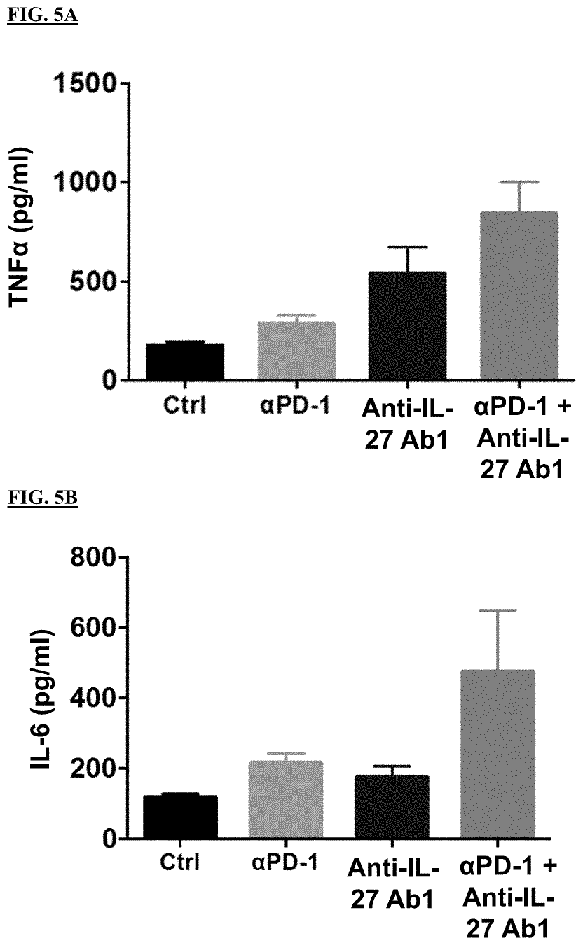

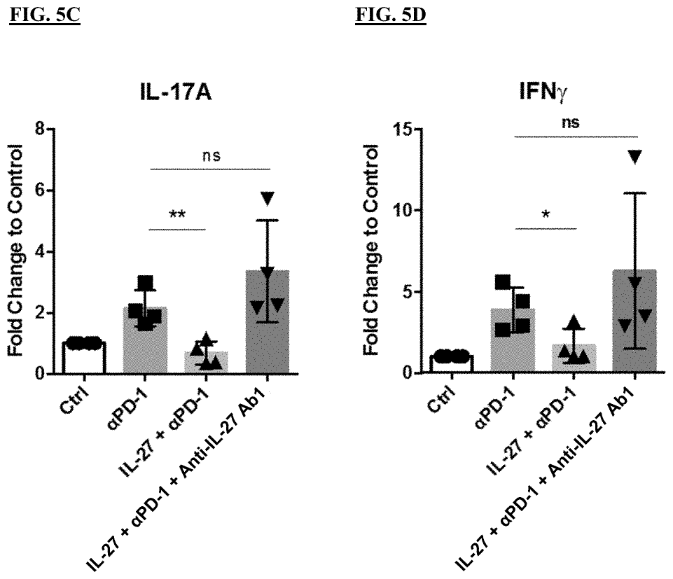

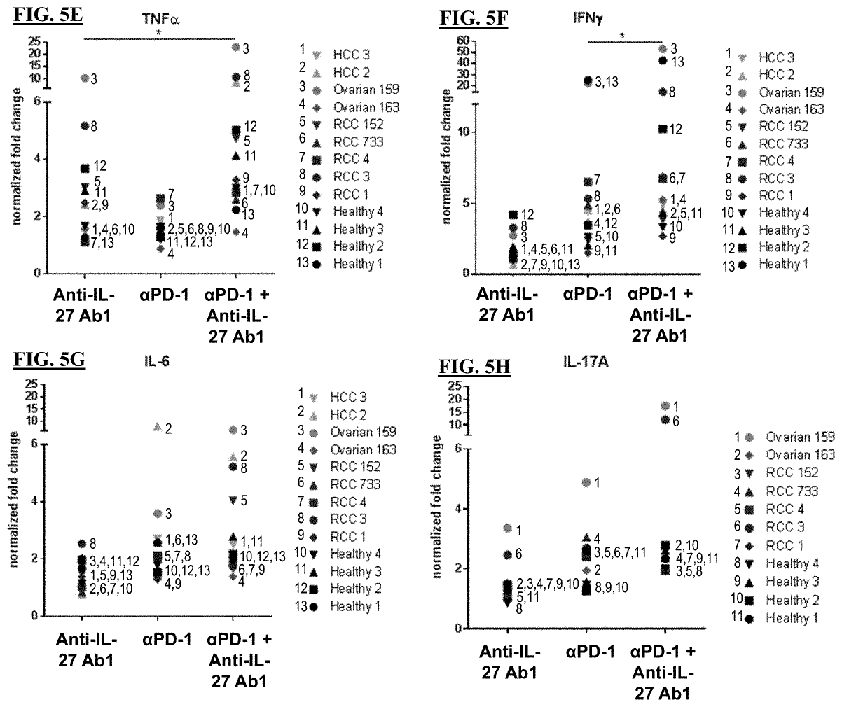

Anti-IL-27 antibodies were tested for their ability to enhance PD-1-mediated secretion of TNFα and IL-6 in human PBMC cells from cancer patients. Human PBMC cells from cancer patients were cultured essentially as described in Example 4 with the addition of wells also receiving anti-PD-1 antibody, as indicated, at 1 μg/mL. Supernatants from the assay were analyzed for TNFα and IL-6 using Human CBA Th1/Th2/Th17 Kit (BD, 560484). As shown in FIGS. 5A and 5B, anti-IL-27 antibodies enhance the PD-1-mediated secretion of TNFα and IL-6 in pooled human PBMC cells.

The techniques herein also show cytokine-inducing activity of anti-IL-27 Ab1 monotherapy and in combination with anti-PD-1 in human PBMCs. IL-27 is known to negatively regulate the expression of several inflammatory cytokines. To determine the effects of IL-27 blockade on cytokine production, human PBMCs from healthy donors, patients with RCC, and patients with ovarian cancer were activated with anti-CD3 in the presence or absence of anti-IL-27 Ab1 for several days and tested for levels of secreted cytokines including IL-17, IFNγ (IFNg), TNFα (yTNFa), and IL-6. Briefly, PBMCs isolated from fresh whole blood from 4 healthy donors, 5 patients with RCC, and 2 patients with ovarian cancer were activated by 0.25 μg/mL anti-CD3 antibody in the absence or presence of anti-IL-27 Ab1 (1 μg/mL), anti PD 1 (pembrolizumab, 1 μg/mL) or both antibodies. After 5 days, supernatants were collected and tested for levels of TNFα (A) or IFNγ (B) by MSD or CBA. Data shown represent the fold-change in cytokine production compared to anti-CD3 stimulation alone. Statistics were calculated by paired t-test (*p<0.005).

Anti-PD-1 antibody was used as a control in these assays and the combination of PD-1 and IL-27 blockade was also explored as shown in FIG. 5C. anti-IL-27 Ab1 treatment led to increased TNFα production in 6 of 11 PBMC samples tested (determined by >2 fold increase) including 2 of 4 healthy donors, 3 of 5 patients with RCC, and 1 of 2 patients with ovarian cancer. When tested in a subset of donors this activity was anti-IL-27 Ab1 dose dependent (data not shown). Anti-PD-1 (pembrolizumab) treatment showed an increase in TNFα in 2 of the 11 donors tested (1 of 5 RCC and 1 of 2 ovarian cancer) while the combination of anti-IL-27 Ab1 and anti-PD-1 led to an increase in 10 of 11 donors. The increased TNFα seen in the combination treatment conditions appeared to be additive in 8 of 10 responders. An additive effect for IFNγ production was observed in these cultures after anti-IL-27 Ab1 and anti-PD-1 treatment (10 of 11 donors); however, responses to anti-PD-1 treatment alone were more frequently seen (10 of 11 donors) compared to anti-IL-27 Ab1 (2 of 11 donors). Together, these data suggest that anti-IL-27 Ab1 increases TNFα levels in activated PBMC cultures from healthy donors and patients with cancer and the combination of anti-IL-27 Ab1 and anti-PD-1 treatment leads to higher levels of TNFα and IFNγ compared to either treatment alone.

To further explore the role of IL-27 and PD-1 blockade, the same activated PBMC culture system was used to determine whether IL-27 could directly counteract the effect of increased cytokine production caused by PD-1 blockade. Briefly, freshly isolated PBMCs from human whole blood were activated by 0.25 μg/mL anti-CD3 antibody. Cells were treated either control IgG1 (1 μg/mL), αPD-1 antibody (pembrolizumab, 1 μg/mL) alone, rhIL-27 (25 ng/mL) plus αPD-1 or rhIL-27 plus αPD-1 with anti-IL-27 Ab1 (1 μg/mL) at 37° C. for 5 days. Supernatants were collected for CBA detection. The example cytokines (IL-17A and IFNγ) from 4 healthy donors were shown as fold change to control. Mean and standard deviation were depicted. Statistics were calculated by paired t-test (* p<0.05, ** p<0.01). Similar results were also seen in PBMCs from patients with RCC.PD-1 blockade increased both IL-17 and IFNγ in these cultures and IL-27 could completely inhibit this activity, a response that was reversed in the presence of anti-IL-27 Ab1 as shown in FIG. 5D. These data show that IL-27 can attenuate the effects of anti PD-1 treatment on cytokine production.

Therefore, IL-27 was shown to inhibit anti-PD-1 mediated pro-inflammatory cytokine production in activated human PBMCs, a property that was blocked by anti-IL-27 Ab1. Moreover, anti-IL-27 Ab1 in combination with PD-1 blockade led to increased cytokine production in activated PBMCs from healthy donors and patients with RCC. Thus, by blocking IL-27, anti-IL-27 Ab1 enhances immune cell activation by altering immunoregulatory receptor expression and increasing inflammatory cytokine production.

In additional characterization of individual anti-IL-27 antibodies in the presence of anti-IL-27 antibody (here, anti-IL-27 Ab1), αPD-1 antibody, or combined anti-IL-27 and αPD-1 antibodies, further characterization of cytokine induction/secretion was performed (FIGS. 5E-511, specifically for TNFα, IFNγ, IL-6 and IL-17A).

### Example 6: Inhibition of IL-27-Mediated Expression of PD-L1 and TIM3 by Anti-IL-27 Antibodies

Anti-IL-27 antibodies described in Example 1 were tested for their ability to inhibit IL-27-mediated expression PD-L1 and TIM-3 in pooled human monocytes by flow cytometry.

Fresh Monocytes were isolated from human buffy coats using ROSETTESEP™ Human Monocyte Enrichment Cocktail (Stemcell #15068).

Use of outer walls was avoided to minimize the effects of evaporation during the 5 day assay. Outer wells should be filled with 200 μL per well of DPBS (Gibco, 14190-144).

Monocytes were resuspended at a density of 2 million cells per mL in warm, complete RPMI-1640. 100 μL per well of this cell mixture was plated (200,000 cells per well) in a round bottom 96-well plate (Costar, 3799).

Anti-IL-27 antibodies were diluted in complete RPMI-1640 in the first row of a 96-well polypropylene plate to a top concentration of 40 μg/ml (10 μg/mL final concentration). Serial dilutions as desired (1:2, 1:3, etc. . . . ) were made in the remainder of the first 10 rows of the plate. 50 μL of the antibody stock (4×) was added to the first 10 rows the plate of PBMC cells in the round bottom plate. In rows 11 and 12, 1250 μL of complete RPMI-1640 was added.

After the addition of the anti-IL-27 antibodies, 50 μL of 80 ng/ml recombinant human IL-27 (R&D Systems, 2526-IL) diluted in complete RPMI-1640 was added to each well (except, control wells which included serum-free media or antibody alone) for a final concentration of 20 ng/mL. 100 μL serum-free RPMI-1640 was added to control wells. The plate was incubated for 3 days at 37° C. with minimal interference.

After the 3-day incubation the plate was removed from the incubator and agitated on a plate shaker for 30 seconds at 600 RPM. The plate was centrifuged at 1800 RPM for 5 minutes. Media was discarded by flicking and plate was washed with 150 μL DPBS (Gibco, 14190-144). The washing steps were repeated twice. The cell pellets were stained with 50 μL per well of staining cocktail as described in the Table 9 below:

TABLE 9

| Biolegend | Antibody | | |
|---|---|---|---|
| Catalog # | Target | Color | Dilution |
| 345006 | TIM3 | PE | 1:100 |
| 301310 | CD11b | APC | 1:100 |
| 329714 | PD-L1 | BV421 | 1:100 |

The plate was agitated on a plate shaker for 30 seconds at 600 RPM and the plate was incubated for 30 minutes 4° C. in the dark.

After the 30-minute incubation, the plate was centrifuged and supernatant was discarded by flicking. The plate was washed 2 times as described previously. After the last wash cell pellets were fixed by adding 50 μL 4% PFA (Pierce, 28906) in deionized (DI) water at room temperature for 10 mins. 100 μL of FACS buffer was added to each well, and the plate was centrifuged at 1800 RPM for 5 minutes. Cells were resuspended in 100 μL FACS buffer and analyzed by flow cytometry.

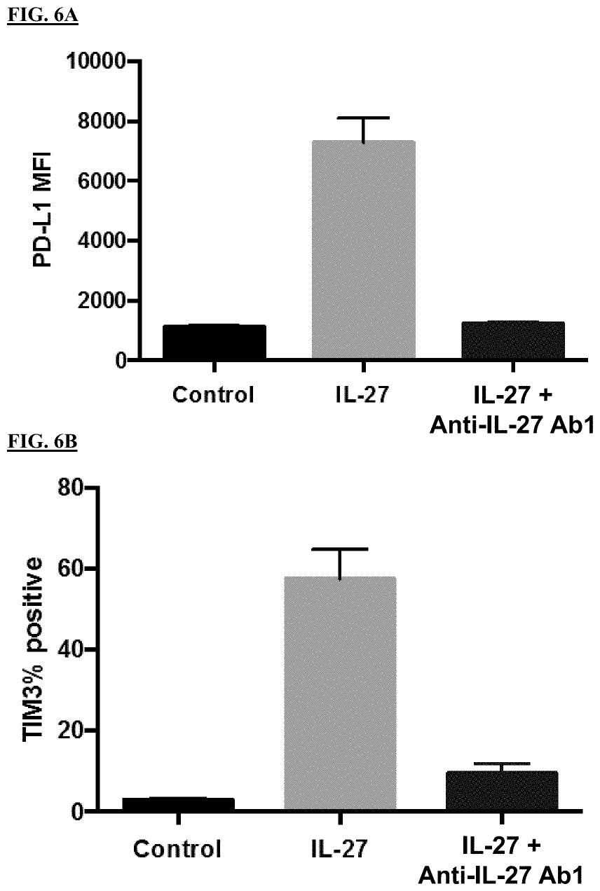

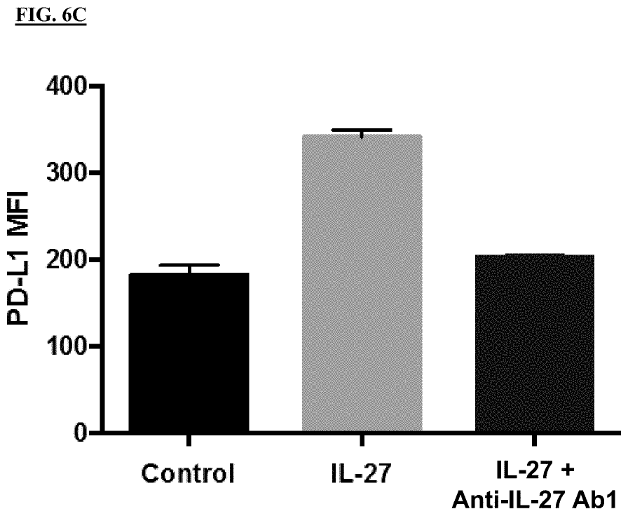

As shown in FIGS. 6A and 6B, anti-IL-27 antibodies potently inhibit the IL-27 mediated expression of PD-L1 and TIM3 in pooled human monocytes.

Anti-IL-27 antibodies were further tested for their ability to inhibit IL-27-mediated expression of PD-L1 in resting T cells (inactivated) essentially as described for FIGS. 6A and 6B. Resting T-cells were isolated from human buffy coats using ROSETTESEP™ Human T cell Enrichment Cocktail (Stemcell #15061).

At the conclusion of the assay, the cell pellets were stained with 50 μL per well of staining cocktail as described in the Table 10 below:

TABLE 10

| Biolegend | Antibody | | |
|---|---|---|---|
| Catalog # | Target | Color | Dilution |
| 345006 | TIM3 | PE | 1:100 |
| 555349 | CD4 | APC | 1:100 |
| 329714 | PD-L1 | BV421 | 1:100 |
| 555366 | CD8 | FITC | 1:100 |

The plate was agitated on a plate shaker for 30 seconds at 600 RPM and the plate was incubated for 30 minutes at 4° C. in the dark.

After the 30-minute incubation, the plate was centrifuged and supernatant was discarded by flicking. The plate was washed 2 times as described previously. After the last wash cell pellets were fixed by adding 50 μL 4% PFA (Pierce, 28906) in DI water at room temperature for 10 min. 100 μL of FACS buffer was added to each well, and the plate was centrifuged at 1800 RPM for 5 minutes. Cells were resuspended in 100 μL FACS buffer and read by flow cytometry. As shown in FIG. 6C, anti-IL-27 antibodies potently inhibit the IL-27-mediated expression of PD-L1 in pooled human resting T cells.

### Example 7: In Vivo Efficacy of an Anti-IL-27 Antibody in a Disseminated B16F10 Model of Melanoma

A model of melanoma lung metastasis was used to assess the antitumor activity of IL-27 blockade using the clinical candidate anti-IL-27 Ab1. The growth of disseminated B16F10 lung metastases is known to be significantly reduced in EBI3 and Il27ra (Wsx-1)-deficient mice (Sauer et al., J. Immunology 181: 6148-6157). Since lung nodule size and growth kinetics are dependent on the number of B16F10 cells transferred and can proceed variably and rapidly, the combination of anti-PD-1 and anti-CTLA-4 was studied as a benchmark for therapeutic activity. Anti-IL-27 Ab1 pre-treatment resulted in a significant reduction in overall tumor burden.

Briefly, six to eight-week-old female C57BL/6 mice (n=10/group) were inoculated intravenously (i.v.) with either 2.5×10⁵ B16F10 cells or 1×10⁵ B16-Luc cells via the tail vein in 200 μL phosphate-buffered saline (PBS). Animals were injected intraperitoneally (i.p.) with anti-IL-27 Ab1 (1 mg dose) (Wuxi; lot 2108SD170316K01X01I01) or polyclonal human IgG isotype control (1 mg dose) (Bioxcell; BE0092; lot 658417D1). Antibodies were dosed once weekly beginning 7 days before tumor injection for a total of four doses (days −7, 0, 7, and 14). For visual enumeration of lung metastases, B16F10 tumor bearing mice were euthanized by CO₂ asphyxiation 18 days-post tumor cell injection and lungs were perfused with PBS via cardiac puncture, removed, and fixed in 10% neutral buffered formalin for 24 hours. Fixed lungs were then transferred to 70% ethanol and surface lung metastases were counted visually. For immunohistochemical analysis, formalin fixed lungs (n=5/group) were paraffin embedded, sectioned and stained with hematoxylin and eosin for quantification of total tumor area as a percentage of total tissue area in each section. For in vivo tumor imaging of lung metastases, B16-Luc tumor-bearing animals were injected i.v. via the tail vein with 3 mg of VivoGlo D-luciferin in 200 μL PBS (Promega) twice weekly. Five minutes after luciferin injection animals were anesthetized and bioluminescent imaging was performed using an IVIS Lumina LT Series III imager. Images were analyzed using Living Image (version 4.5.5) software and represented as total flux measurements in photons/second.

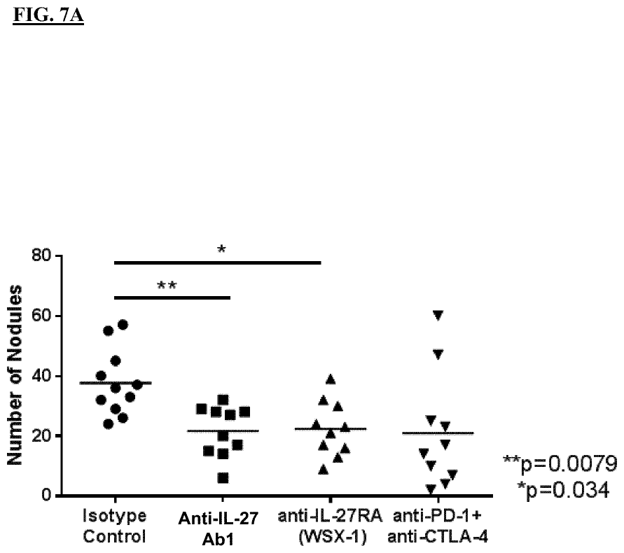

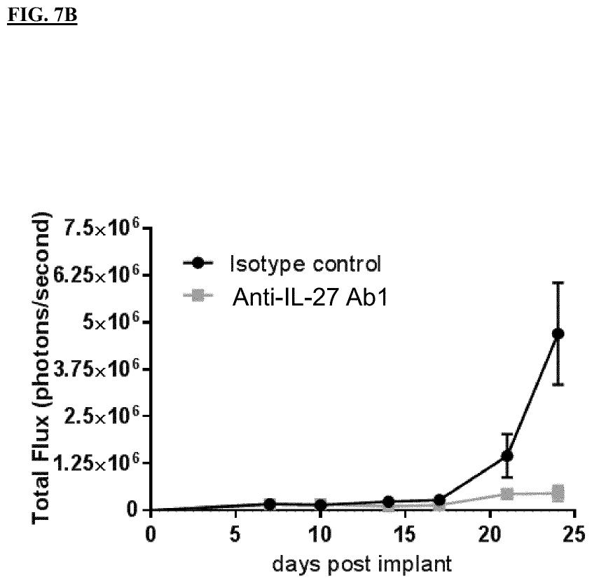

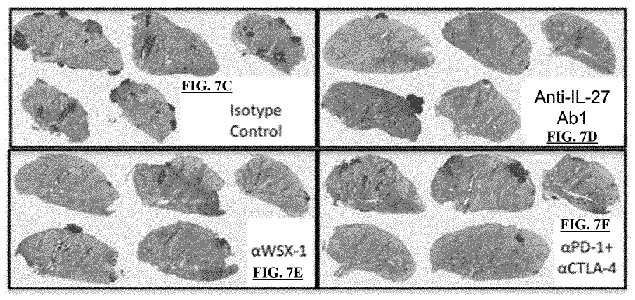

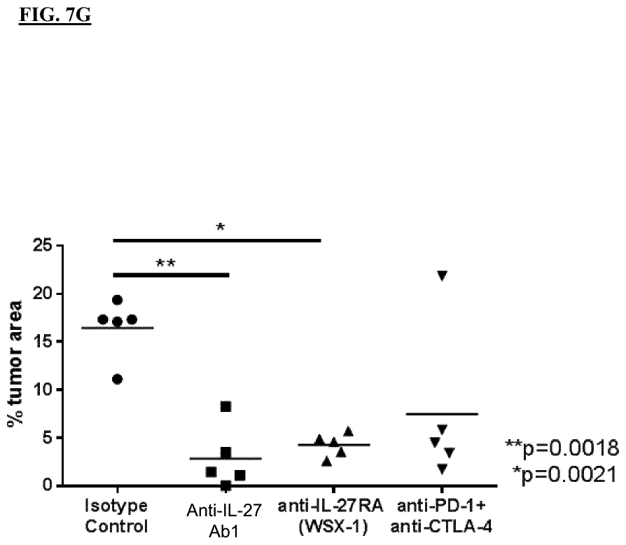

As shown in FIGS. 7A-7G, treatment of B16F10 tumor-bearing mice with the anti-IL-27 antibody anti-IL-27 Ab1 resulted in a significant reduction in overall tumor burden as measured by both total counts of surface lung metastases (#pulmonary nodules, FIG. 7A), and by a reduction of tumor area in lung tissue sections by immunohistochemistry (IHC) analysis (FIGS. 7C-7F and FIG. 7G). Blockade of p28 with anti-IL-27 Ab1 resulted in a 42% reduction in the number of pulmonary B16 nodules compared to isotype control treatment. Anti-IL-27 Ab1 treatment significantly inhibited (p=0.0079) the growth of B16F10 lung metastases compared to isotype control (21.6±8.4 versus 37.6±10.9 lung nodules, respectively). Anti-IL-27 Ab1 treatment resulted in an 83% reduction in overall lung tumor metastasis area as measured by IHC (16.43±1.39% in the isotype control group versus 2.83±1.45% in the anti-IL-27 Ab1 treatment group). Similarly, bioluminescent imaging revealed that anti-IL-27 Ab1 treatment significantly (p=0.0062) delayed the growth of B16-Luc lung metastases (FIG. 7B). A similar reduction in surface lung metastasis number and total tumor area was observed with IL-27RA (WSX-1) mediated antibody blockade and with anti-PD-1+anti-CTLA-4 combination therapy, as shown in FIG. 7G. These data are from 2 independent experiments in which anti-PD-1 and anti-CTLA-4 benchmark combination demonstrated antitumor activity. B16F10 cells (2.5×10⁵) were injected intravenously in C57BL/6 mice (n=10/group). Mice were treated IP with 1 mg of either anti-IL-27 Ab1, anti-IL-27RA (WSX-1), or human IgG isotype control antibody (Days −7, 0, 7, 14). Some animals were treated IP with anti-PD-1 and anti-CTLA-4 (Days 0, 4, 7, and 11). Lungs were collected from animals (n=5/group) bearing B16F10 lung metastases treated as described above were sectioned and stained with H&E. B16F10 tumor tissue was delineated from normal lung tissue in H&E stained lung sections from treated animals (FIG. 7A). Tumor area was calculated as a percentage of total lung area (FIG. 7G). Statistics were calculated by t-test. Collectively, these data indicate that anti-IL-27 Ab1 can phenocopy Il27ra (WSX-1) and EBI3 deficiency in a tumor model and shows similar activity to combined blockade of PD-1 and CTLA-4.

These data demonstrate that treatment with an anti-IL-27 antibody (anti-IL-27 Ab1) results in anti-tumor effects, reducing both tumor growth and metastasis to a greater extent that treatment with an isotype control antibody that does not bind IL-27.

### Example 8: Gene Expression Profiling of Murine Splenocytes from Mice Hydrodynamically Transfected with Human IL-27 Minicircles

To examine the effect of IL-27 on T cell phenotype in vivo, DNA minicircles encoding IL-27 were used to overexpress IL-27 in mice and T cell responses were assessed by RNA-Seq and flow cytometry. Human IL-27 is known to be species cross-reactive and can induce pSTAT1 signaling and PD L1 in murine splenocytes in vitro. This species cross-reactivity was used to study the effects of human IL-27 overexpression in mice and its inhibition by anti-IL-27 Ab1. To do this, DNA plasmid minicircles encoding human IL-27 (p28 tethered to EBI3 by a glycine serine linker) were administered to mice by hydrodynamic transfection, as described below, which resulted in high systemic levels of IL-27.

### Hydrodynamic Transfection of Human IL-27 Minicircles

Six-week-old female BALB/c mice were injected with 20 μg of either empty vector or linked human IL-27 minicircle DNA (System Biosciences, Palo Alto, Calif.) in 2 mL 0.9% normal saline via the tail vein over the course of 5 seconds. Injected animals were transferred to an empty cage with a heating pad to recover for 5 minutes. Whole blood was collected into K2-EDTA tubes for plasma separation 24 hours after minicircle injection and plasma IL-27 levels were confirmed by ELISA. PBMCs and total splenocytes were collected 5 days after transfection and cells were stained and analyzed by flow cytometry. Expression of the indicated markers were analyzed on CD4+ T cells and CD8+ T cells. Analysis was performed using FlowJo software.

### Gene Expression Profiling

Mouse splenocytes were prepared by mechanical dissociation of whole spleens, followed by ACK lysis of red blood cells. Total RNA was extracted from splenocytes with the RNEASY® Mini Kit (Qiagen, Cat. No: 74104) and adjusted to 20 ng/uL in nuclease free water (Qiagen, Cat. No: 19101). Gene expression profiling on was performed on Affymetrix GENECHIP™ Mouse Gene 2.0 ST Arrays (Applied Biosystems, Cat. No: 902118). Processing of RNA samples, hybridization and array scanning were carried out using standard Affymetrix GENECHIP™ protocols at the Boston University Microarray and Sequencing Resource (BUMSR). All CEL files were normalized by Robust Multi-array Average (RMA) (Irizarry et al., 2003) and gene expression data were preprocessed by removing unexpressed probes and discarding transcripts with high inter-replicate coefficient of variance. Subsequent analyses (mean expression, fold change, t test) were performed in R (version R 3.6.2).

### Flow Cytometric Analysis

Whole blood and spleens were collected from mice five days after minicircle injection. Splenocytes were collected from IL 27-expressing mice 5 days after transfection. Single cell splenocyte suspensions were prepared by mechanical dissociation through a 40 μm nylon cell strainer followed by red blood cell lysis in ACK buffer. Whole blood cells were stained directly followed by red blood cell lysis and fixation in BD Phosflow Lyse/Fix Buffer according to the manufacturer's instructions (BD Biosciences, San Jose, Calif.). FcγRIII/II was blocked by preincubating cells with rat anti-mouse CD16/CD32 mAb (1 μg per million cells; Biolegend, San Diego, Calif.) in PBS with 2% FBS and 2 mM EDTA. Cells were stained with APC-, PE-, Brilliant Violet 510-, and Brilliant Violet 711-conjugated mAbs against murine CD4 (clone GK1.5), CD8 (53-6.7), PD-L1 (10F.9G2), TIM3 (RMT3-23), LAG3 (C9B7W), and TIGIT (1G9) (Biolegend). Cell-associated fluorescence was measured using an LSRFortessa X-20 flow cytometer (BD Biosciences), and analysis was performed using FlowJo software (Tree Star, Ashland, Oreg.).

### Statistical Analysis

Statistical significance was determined using GraphPad Prism software, using a paired, unpaired, or ratio Student's t test, as indicated. When the ratio t test was used, 0.1 was added to zero values to make them non-zero. P values less than 0.05 were considered significant.

### IL-27 Promotes Expression of Inhibitory Receptors by T Cells In Vivo

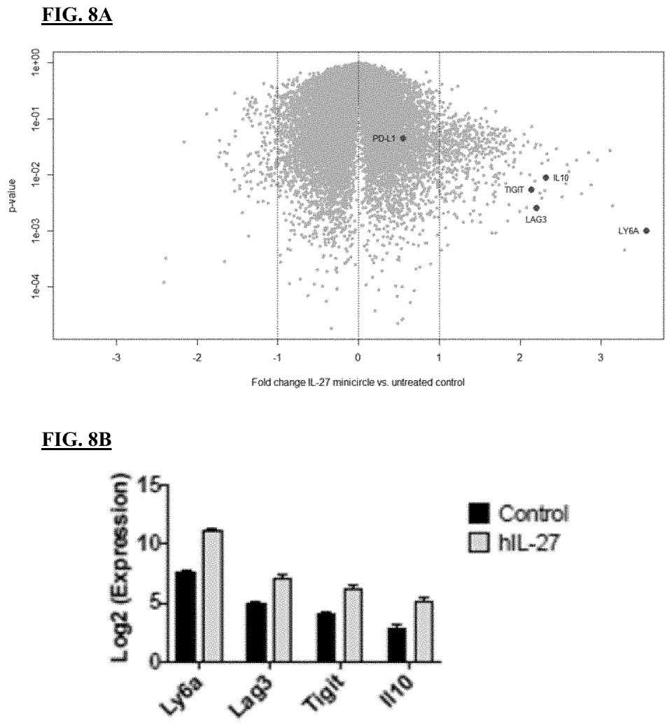

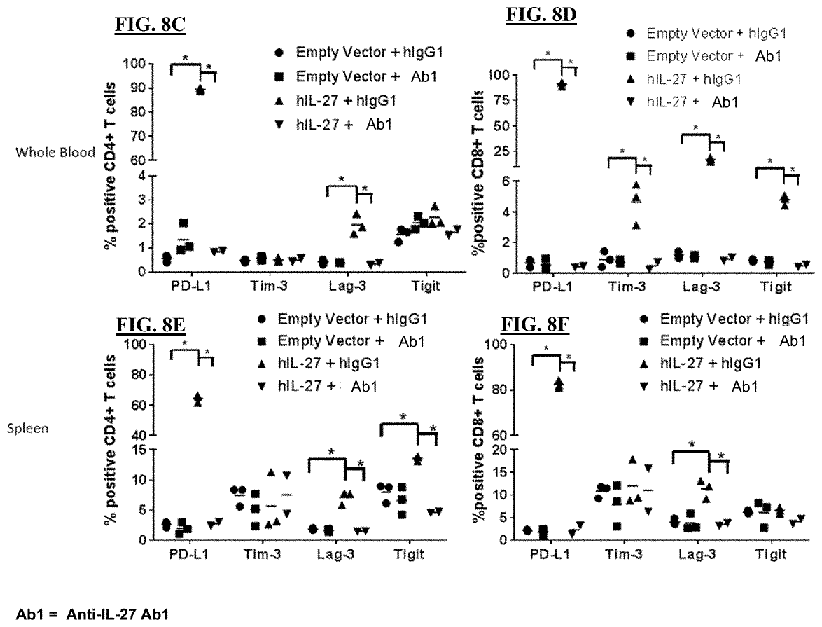

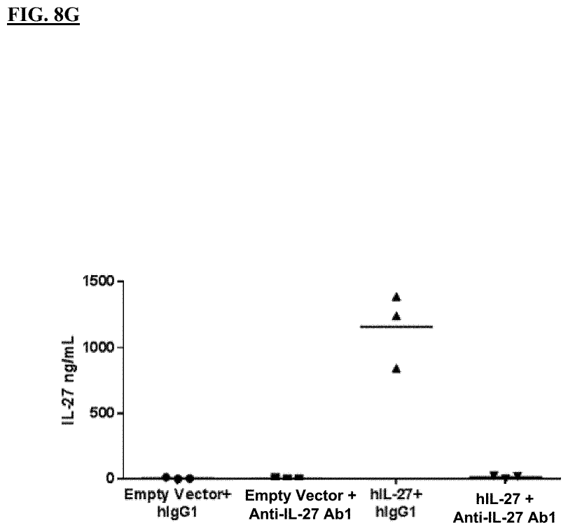

Over 400 genes were changed by ≥Log 2 fold in response to administration of IL-27, as shown in FIG. 8A. A subset of these genes is shown in Tables 11A-11B. Among these genes were those that encode immune inhibitory receptors that play key roles in the immune response. As shown in FIG. 8B, Ly6a (encodes Sca-1), Lag3, Tigit and 1110 were upregulated on splenocytes in response to IL-27. There was also a trend toward IL-27-mediated upregulation of Ctla4 and Cd274 (encodes PD-L1) that was less than 1-fold induction (data not shown). To validate the expression data, flow cytometry was utilized to assess the protein expression of PD-L1, LAG-3, TIGIT and TIM-3 on T cells from these mice. Administration of IL-27 minicircles led to upregulation of PD-L1, LAG-3 and TIGIT in splenic (Spleen) and peripheral blood (PBMC) CD4⁺ T cells. In CD8⁺ T cells, IL-27 minicircles upregulated PD-L1, LAG-3, TIGIT, and TIM-3. As shown in FIGS. 8C-8F, administration of IL-27 minicircles led to upregulation of PD-L1, Lag-3, and Tigit in splenic and peripheral blood CD4⁺ T cells. In CD8⁺ T cells, IL-27 minicircles upregulated PD-L1, Lag-3, Tigit, and Tim-3. These data suggest that IL-27 can play a key role in driving immunoregulatory receptor expression in vivo.

To investigate the ability of anti-IL-27 Ab1 to block minicircle-derived human IL-27 in vivo, both target engagement by enzyme-linked immunosorbent assay (ELISA) and immunoregulatory receptor expression in splenocytes were studied. Five days after IL-27 transfection and treatment with anti-IL-27 Ab1 (50 mg/kg), plasma was collected from mice to analyze IL-27 heterodimer and EBI3 levels by Meso Scale Discovery (MSD). The IL-27 heterodimer assay utilizes a p28 capture antibody that cross blocks anti-IL-27 Ab1 and a human specific EBI3 detection antibody; therefore, if anti-IL-27 Ab1 is bound to IL-27 then its detection will be masked. The EBI3 assay utilizes both capture and detection antibodies specific for 2 distinct epitopes of human EBI3 and since the minicircle derived IL-27 is a tethered heterodimer this assay allows for detection of total IL 27 irrespective of anti-IL-27 Ab1 binding.

Briefly, Six-week-old female Balb/c mice were injected with empty vector (control) or human IL-27. Mice were treated with 1 mg of either anti-IL-27 Ab1 or anti-DNP IgG1 isotype control antibody 7 days before and on the day of minicircle transfection (Day −7 and 0). Whole blood was collected, and plasma was analyzed for IL-27 (FIG. 8G) by Meso Scale Discovery. FIG. 8G shows that anti-IL-27 Ab1 treatment completely inhibits IL-27 detection in plasma by MSD. Similar data were seen when a dose of 25 mg/kg of anti-IL-27 Ab1 was tested. These data suggest that anti-IL-27 Ab1 at a dose of 25 mg/kg or higher can completely saturate minicircle derived IL-27 in vivo. This complete target engagement was also confirmed in a pSTAT1 functional assay.

To assess the ability of anti-IL-27 Ab1 to block the activity of IL-27 in vivo, the expression of PD L1, Tim-3, Lag-3, and Tigit were analyzed in murine PBMCs and splenocytes by flow cytometry. anti-IL-27 Ab1 significantly blocked IL 27-induced PD-L1 and Lag 3 expression in CD4+ PBMCs and PD-L1, Tim-3, Lag-3, and TIGIT expression in CD8+ PBMCs. anti-IL-27 Ab1 treatment also blocked IL 27 induced PD-L1, Lag-3, and TIGIT expression in CD4+ splenocytes and PD-L1 and Lag 3 expression in CD8+ splenocytes. These data suggest that anti-IL-27 Ab1 can both engage and block the activity of human IL-27 in vivo.

These results demonstrate that ectopic expression of IL-27 in vivo leads to upregulation of multiple inhibitory receptors by T cells, and several other molecules with immunomodulatory activity in splenocytes. These data suggest that IL-27 antagonism (e.g., by treatment with an anti-IL-27 antibody) would decrease the expression of inhibitor receptors on T cells, thereby increasing immune responses.

TABLE 11A
Genes Upregulated in Response to Administration of IL-27

| Gene Symbol | Fold Change | p value |
|---|---|---|
| GM4841 | 3.824228667 | 0.012331653 |
| LY6A | 3.568709 | 0.000991642 |
| IIGP1 | 3.294783 | 0.000455248 |
| TUBB1 | 3.145617 | 0.002782618 |
| MPO | 3.112051333 | 0.026667968 |
| CTSG | 2.954178333 | 0.018177934 |
| PPBP | 2.878417333 | 0.006623845 |
| ELANE | 2.845762333 | 0.024770656 |
| MT2 | 2.757525667 | 0.004548988 |
| MUC13 | 2.696042667 | 0.018195326 |
| F830016B08RIK | 2.603319667 | 0.011330985 |
| GM4951 | 2.583066667 | 0.01529863 |
| APOL11B | 2.565688667 | 0.006885943 |
| GZMB | 2.515642667 | 0.004012308 |
| PRTN3 | 2.450033 | 0.028785527 |
| CLCA3A1 | 2.345778333 | 0.018175529 |
| GM11505 | 2.331628667 | 0.023451392 |
| IL10 | 2.324278 | 0.008857442 |
| GBP11 | 2.307386 | 0.005204674 |
| PF4 | 2.270045 | 0.016210658 |
| IRG1 | 2.269440667 | 0.003861367 |
| CES2G | 2.252403333 | 0.033921335 |
| OASL2 | 2.243393 | 0.00470682 |
| LAG3 | 2.203067 | 0.002539095 |
| HIST1H2AG | 2.197938 | 0.03499933 |
| OAS1G | 2.184084667 | 0.007645573 |
| MFSD2B | 2.179382333 | 0.030664254 |
| RHAG | 2.146212667 | 0.072130267 |
| TIGIT | 2.145155 | 0.005421349 |
| SLC6A4 | 2.121439 | 0.009945773 |
| SHCBP1 | 2.092208667 | 0.038685767 |
| BC023105 | 2.079652 | 0.002582842 |
| PKLR | 2.045597333 | 0.053459171 |
| TFR2 | 2.037887333 | 0.033394313 |
| F13A1 | 2.010803333 | 0.010612157 |
| HIST1H2AB | 2.002687333 | 0.025241 |
| SERPINA3F | 1.991916 | 0.002220854 |
| ERMAP | 1.940590333 | 0.042680673 |
| MCPT8 | 1.930212667 | 0.018921256 |
| SLC26A1 | 1.926420667 | 0.025126602 |
| PRKAR2B | 1.924056667 | 0.016881189 |
| BIRC5 | 1.914826333 | 0.032959509 |
| FADS2 | 1.91223 | 0.013830993 |
| TOP2A | 1.88339 | 0.034852729 |
| NCAPG | 1.881082333 | 0.036612626 |
| A730089K16RIK | 1.870258667 | 0.061491704 |
| MNS1 | 1.860366 | 0.014834618 |
| GP9 | 1.859234333 | 0.002956835 |
| GFI1B | 1.852962 | 0.0291761 |
| NUF2 | 1.850953 | 0.032872517 |
| CHIL3 | 1.848470667 | 0.001494932 |
| KIF11 | 1.83252 | 0.040535377 |
| ALOX12 | 1.823174 | 0.006931665 |
| ADGRG7 | 1.822065 | 0.017542834 |
| KLF1 | 1.820262333 | 0.060689421 |
| E2F8 | 1.817455333 | 0.069143254 |
| ATP1B2 | 1.811274333 | 0.024016123 |
| KIF2C | 1.811223333 | 0.061246458 |
| FADS3 | 1.803859667 | 0.050197663 |
| MS4A6D | 1.801871667 | 0.01036878 |
| SLC25A21 | 1.801518667 | 0.048282826 |
| HIST1H1B | 1.800434 | 0.037242995 |
| CKAP2L | 1.783850333 | 0.061646634 |
| SAMD14 | 1.782388333 | 0.02384128 |
| CAR1 | 1.770098667 | 0.025511071 |
| DEPDC1A | 1.765839667 | 0.03875749 |
| CENPE | 1.765095667 | 0.039288425 |
| ASPM | 1.753558 | 0.054145246 |
| CCNB2 | 1.751291667 | 0.036336189 |
| RYK | 1.749673 | 0.035413093 |
| MMP14 | 1.747348667 | 0.010510984 |
| BUB1 | 1.738322667 | 0.02139336 |
| MYO1D | 1.734508 | 0.006655486 |
| PARVB | 1.733820333 | 0.010799396 |
| GM5593 | 1.728317 | 0.008578526 |
| CCNA2 | 1.724975667 | 0.026630845 |
| PRR11 | 1.724352667 | 0.04875805 |
| AQP1 | 1.719081667 | 0.064562051 |
| CASP3 | 1.709594333 | 0.009087444 |
| KIF15 | 1.708689667 | 0.026782535 |
| ASNS | 1.708074333 | 0.037928738 |
| CPOX | 1.706113 | 0.030298001 |
| MT1 | 1.699002667 | 0.010159345 |
| CDC6 | 1.694586667 | 0.049970924 |
| GBP2B | 1.694018 | 0.000929009 |
| GBP2 | 1.689558667 | 0.004961011 |
| HMMR | 1.687253333 | 0.063769534 |
| KIF20A | 1.686718333 | 0.020639142 |
| GSTM5 | 1.681807333 | 0.04316743 |
| REEP6 | 1.677204667 | 0.056071877 |
| GM12250 | 1.675485 | 0.003251181 |
| GBP10 | 1.673458 | 0.009333312 |
| ATP7B | 1.671614 | 0.029798535 |
| GM22973 | 1.663495667 | 0.004472758 |
| CASC5 | 1.659578 | 0.044333895 |
| ADD2 | 1.659553667 | 0.053938067 |
| CAMP | 1.659111667 | 0.066745225 |
| CLEC5A | 1.654882667 | 0.00665055 |
| MUP-PS12 | 6.332 | 0.0021 |
| GM26184 | 5.617 | 0.0850 |
| GKN2 | 4.822 | 0.0378 |
| MYH8 | 3.854 | 0.3190 |
| MIR101B | 3.695 | 0.0111 |
| GKN1 | 3.485 | 0.0451 |
| SLN | 3.473 | 0.1822 |
| MUC5AC | 3.341 | 0.0628 |
| 2310057J18RIK | 3.041 | 0.4100 |
| PSCA | 3.027 | 0.1219 |
| GM25623 | 2.948 | 0.0133 |
| FAM83B | 2.905 | 0.0202 |
| GM23852 | 2.882 | 0.0763 |
| SPTSSB | 2.873 | 0.0952 |
| MIRLET7F-1 | 2.794 | 0.0447 |
| MUP-PS16 | 2.778 | 0.0114 |
| ANXA10 | 2.689 | 0.1047 |
| LGALS2 | 2.685 | 0.1337 |
| CHIA1 | 2.672 | 0.4078 |
| AKP3 | 2.660 | 0.3665 |
| SLC13A1 | 2.622 | 0.2936 |
| GM766 | 2.621 | 0.4035 |
| GM24537 | 2.617 | 0.1649 |
| GM24138 | 2.606 | 0.0374 |
| SIS | 2.585 | 0.3777 |
| 2210407C18RIK | 2.584 | 0.0600 |
| S100G | 2.580 | 0.3877 |
| GM26354 | 2.540 | 0.1097 |
| CKMT2 | 2.538 | 0.2680 |
| GM26055 | 2.525 | 0.2157 |
| GM5885 | 2.479 | 0.0305 |
| AGR2 | 2.458 | 0.1845 |
| VMN1R167 | 2.454 | 0.0932 |
| GM25167 | 2.444 | 0.0264 |
| ATP5C1-PS | 2.437 | 0.0477 |
| GM24470 | 2.434 | 0.1676 |
| MYH2 | 2.403 | 0.1632 |
| MIR326 | 2.375 | 0.0059 |
| MIR122 | 2.375 | 0.0242 |
| MCPT9 | 2.375 | 0.0623 |
| PSG18 | 2.359 | 0.0115 |
| MUC6 | 2.358 | 0.3108 |
| VMN2R115 | 2.352 | 0.1111 |
| GM25498 | 2.351 | 0.0086 |
| 2010106E10RIK | 2.339 | 0.4607 |
| ERBB4 | 2.328 | 0.0350 |
| LYPD8 | 2.320 | 0.1374 |
| CYP2A5 | 2.305 | 0.2330 |
| BTNL5-PS | 2.301 | 0.1684 |
| GM24549 | 2.289 | 0.1138 |
| GM24465 | 2.287 | 0.2356 |
| SLC26A3 | 2.286 | 0.3487 |
| DPCR1 | 2.285 | 0.0653 |
| GM11844 | 2.279 | 0.1054 |
| CYP4A32 | 2.269 | 0.2133 |
| MIR1928 | 2.267 | 0.0134 |
| GKN3 | 2.266 | 0.3840 |
| KRT4 | 2.264 | 0.1539 |
| PGC | 2.262 | 0.2583 |
| HIST1H2BA | 2.260 | 0.0390 |
| SPRR2A3 | 2.254 | 0.1974 |
| IGKV12-41 | 2.235 | 0.3548 |
| PARD3BOS3 | 2.227 | 0.1149 |
| CLCA4B | 2.225 | 0.4177 |
| GM23744 | 2.208 | 0.1217 |
| VMN2R-PS129 | 2.200 | 0.0021 |
| GM23241 | 2.194 | 0.1644 |
| MYOZ2 | 2.191 | 0.2091 |
| MIR192 | 2.191 | 0.0553 |
| MYH1 | 2.188 | 0.1653 |
| 5033403H07RIK | 2.187 | 0.1231 |
| MIRLET7F-2 | 2.186 | 0.1617 |
| GM25009 | 2.185 | 0.1243 |
| MUC13 | 2.182 | 0.4412 |
| PPP1R3A | 2.178 | 0.1968 |
| OLFR798 | 2.172 | 0.0036 |
| SERPINA12 | 2.154 | 0.0309 |
| GM25864 | 2.152 | 0.0404 |
| N-R5S155 | 2.151 | 0.0691 |
| CYP2C65 | 2.142 | 0.1438 |
| GM14750 | 2.141 | 0.0525 |
| GM23026 | 2.134 | 0.0863 |
| HAMP2 | 2.125 | 0.2675 |
| GM24527 | 2.125 | 0.0777 |
| OLFR891 | 2.125 | 0.0731 |
| OLFR68 | 2.125 | 0.1746 |
| TRAV6-2 | 2.123 | 0.1684 |
| CYP4F40 | 2.119 | 0.1848 |
| TRDN | 2.119 | 0.2396 |
| CFTR | 2.117 | 0.3208 |
| XIRP2 | 2.109 | 0.2031 |
| CYP8B1 | 2.107 | 0.0912 |
| GM23629 | 2.105 | 0.0590 |
| GM25076 | 2.104 | 0.0980 |
| 1700080G11RIK | 2.103 | 0.1621 |
| N-R5S96 | 2.100 | 0.0493 |
| GM11027 | 2.097 | 0.3009 |
| SLC22A29 | 2.089 | 0.2663 |
| GSTT2 | 2.082 | 0.2372 |
| GM23911 | 2.081 | 0.1903 |
| OLFR724 | 2.079 | 0.0925 |
| GM23277 | 2.078 | 0.0912 |
| A4GNT | 2.076 | 0.2431 |
| MUP21 | 2.074 | 0.1149 |
| GM24147 | 2.074 | 0.2669 |
| MYBPC1 | 2.067 | 0.1392 |
| CYP3A59 | 2.063 | 0.2628 |
| ACTN2 | 2.063 | 0.1471 |
| MIR29A | 2.062 | 0.0340 |
| TFF1 | 2.061 | 0.0803 |
| PLA2G1B | 2.060 | 0.4477 |
| GM23021 | 2.060 | 0.1111 |
| GM22607 | 2.045 | 0.1383 |
| RPL10L | 2.044 | 0.0692 |
| ACE2 | 2.038 | 0.3505 |
| PRAMEL3 | 2.038 | 0.1490 |
| SULT1B1 | 2.032 | 0.2495 |
| ARL14 | 2.026 | 0.0952 |
| GM11337 | 2.024 | 0.0270 |
| CNN1 | 2.020 | 0.0217 |
| LRIT2 | 2.019 | 0.0533 |
| ACOT4 | 2.015 | 0.2120 |
| LOC100125594 | 2.014 | 0.0208 |
| GLB1L2 | 2.012 | 0.0396 |
| GM25605 | 2.008 | 0.1329 |
| GM15384 | 2.002 | 0.0255 |
| GM94 | 1.997 | 0.1095 |
| OLFR1113 | 1.997 | 0.1277 |
| G630018N14RIK | 1.997 | 0.3017 |
| GM23232 | 1.994 | 0.0672 |
| GM16378 | 1.994 | 0.0357 |
| GM22838 | 1.994 | 0.0875 |
| HSD3B5 | 1.992 | 0.2019 |
| OLFR746 | 1.989 | 0.0331 |
| GM24254 | 1.988 | 0.1301 |
| GM24232 | 1.986 | 0.2212 |
| PRAP1 | 1.986 | 0.3720 |
| ALPI | 1.986 | 0.4192 |
| MIR3964 | 1.983 | 0.0301 |
| GM22787 | 1.983 | 0.0183 |
| GM26081 | 1.982 | 0.1771 |
| GM22242 | 1.980 | 0.3169 |
| IGHV1-77 | 1.980 | 0.4742 |
| OLFR740 | 1.980 | 0.2191 |
| OLFR1201 | 1.979 | 0.0339 |
| GM22654 | 1.976 | 0.5101 |
| OLFR747 | 1.975 | 0.0719 |
| GM22521 | 1.973 | 0.1073 |
| VMN1R172 | 1.972 | 0.0731 |
| GM13773 | 1.971 | 0.2179 |
| CLEC2H | 1.970 | 0.2041 |
| OLFR714 | 1.967 | 0.0816 |
| GM22284 | 1.965 | 0.1376 |
| GM26342 | 1.962 | 0.0242 |
| OLFR190 | 1.961 | 0.0184 |
| CCDC152 | 1.960 | 0.0375 |
| GM26048 | 1.959 | 0.1807 |
| GM24410 | 1.959 | 0.1844 |
| LGALS4 | 1.957 | 0.0215 |
| OLFR969 | 1.956 | 0.0946 |
| GM6222 | 1.956 | 0.0950 |
| OLFR913 | 1.953 | 0.3111 |
| 4930557A04RIK | 1.953 | 0.0645 |
| GM24750 | 1.950 | 0.2385 |
| GM23043 | 1.948 | 0.0490 |
| STFA1 | 1.946 | 0.0803 |
| SUCNR1 | 1.942 | 0.1078 |
| MYOM1 | 1.941 | 0.1753 |
| KLK1B22 | 1.938 | 0.1263 |
| GM23917 | 1.934 | 0.1949 |
| GM15949 | 1.932 | 0.0450 |
| OLFR709-PS1 | 1.930 | 0.0722 |
| OBP1A | 1.928 | 0.3421 |
| SULT2A1 | 1.927 | 0.3528 |
| GM24786 | 1.923 | 0.2103 |
| SGK2 | 1.916 | 0.1567 |
| MIRLET7C-1 | 1.911 | 0.0725 |
| VMN1R90 | 1.910 | 0.1018 |
| GM23342 | 1.909 | 0.0298 |
| REG3G | 1.908 | 0.1599 |
| GM24839 | 1.906 | 0.2631 |
| N-R5S157 | 1.905 | 0.1167 |
| GM25023 | 1.905 | 0.0253 |
| CES2B | 1.902 | 0.1386 |
| IGHV8-11 | 1.901 | 0.1555 |
| GM25982 | 1.899 | 0.0542 |
| ATP4A | 1.899 | 0.3968 |
| GM26162 | 1.898 | 0.0147 |
| PNPLA3 | 1.898 | 0.0031 |
| AU015336 | 1.893 | 0.0794 |
| GM24621 | 1.891 | 0.0840 |
| CYP2C40 | 1.891 | 0.4000 |
| GM25602 | 1.891 | 0.1527 |
| ADGRG7 | 1.889 | 0.3205 |
| SLC30A10 | 1.888 | 0.1288 |
| GM25836 | 1.888 | 0.1934 |
| GM25629 | 1.887 | 0.1192 |
| GM24861 | 1.886 | 0.0396 |
| GM21057 | 1.884 | 0.0850 |

TABLE 11B
Genes Downregulated in Response to Administration of IL-27

| Gene Symbol | Fold Change | p value |
|---|---|---|
| DEFB44-PS | 0.187 | 0.1696 |
| C1S2 | 0.187 | 0.0076 |
| HIST1H1B | 0.204 | 0.1347 |
| GM15114 | 0.205 | 0.1127 |
| GM2005 | 0.205 | 0.0169 |
| KHDC1C | 0.211 | 0.0820 |
| GM2005 | 0.224 | 0.0023 |
| SERPINB7 | 0.224 | 0.1389 |
| SERPINB11 | 0.225 | 0.0594 |
| CXCL3 | 0.230 | 0.1516 |
| CDH10 | 0.230 | 0.0466 |
| MIR1949 | 0.235 | 0.1307 |
| SLC7A11 | 0.236 | 0.0195 |
| LUZP4 | 0.249 | 0.0952 |
| IGHV1-81 | 0.252 | 0.5116 |
| PTPRTOS | 0.252 | 0.0338 |
| RP1 | 0.254 | 0.1538 |
| GM15127 | 0.254 | 0.0500 |
| CRISP1 | 0.255 | 0.0865 |
| GM15107 | 0.255 | 0.0591 |
| SERPINB5 | 0.256 | 0.0402 |
| GM7665 | 0.256 | 0.0034 |
| TMEM252 | 0.259 | 0.0173 |
| PNMA5 | 0.262 | 0.0922 |
| CXCL15 | 0.263 | 0.1037 |
| CLEC2G | 0.265 | 0.0388 |
| MMP13 | 0.267 | 0.1687 |
| GM15093 | 0.269 | 0.0741 |
| VMN1R53 | 0.270 | 0.0648 |
| GM14409 | 0.276 | 0.1558 |
| GM11884 | 0.278 | 0.1682 |
| SLC16A4 | 0.278 | 0.1159 |
| MMP12 | 0.280 | 0.0786 |
| KIF5C | 0.281 | 0.1003 |
| APELA | 0.283 | 0.0601 |
| AFP | 0.283 | 0.0424 |
| IGKV1-122 | 0.286 | 0.0090 |
| A630095E13RIK | 0.292 | 0.0905 |
| GM15109 | 0.297 | 0.0058 |
| OLFR111 | 0.299 | 0.2107 |
| TNS4 | 0.301 | 0.0381 |
| PLEKHS1 | 0.301 | 0.0784 |
| LNCENC1 | 0.305 | 0.0939 |
| THBS1 | 0.309 | 0.0243 |
| PLATR14 | 0.313 | 0.0812 |
| RASSF9 | 0.314 | 0.1125 |
| ITGA2 | 0.319 | 0.0699 |
| LOC102634388 | 0.320 | 0.0213 |
| LOC102634388 | 0.320 | 0.0213 |
| GM15093 | 0.320 | 0.0380 |
| CEP55 | 0.321 | 0.1007 |
| A630038E17RIK | 0.321 | 0.1334 |
| GM25552 | 0.323 | 0.1261 |
| RPS26 | 0.327 | 0.1840 |
| STRA6 | 0.330 | 0.0498 |
| GM15093 | 0.331 | 0.0403 |
| PSAT1 | 0.332 | 0.0926 |
| AKR1C18 | 0.332 | 0.0983 |
| DEPDC1A | 0.333 | 0.1814 |
| GM10439 | 0.335 | 0.0694 |
| GM24916 | 0.335 | 0.2031 |
| STC1 | 0.337 | 0.0829 |
| DPPA2 | 0.337 | 0.0887 |
| E030011O05RIK | 0.337 | 0.0486 |
| GM20756 | 0.337 | 0.0218 |
| GM22771 | 0.339 | 0.2897 |
| IGKV6-32 | 0.340 | 0.4435 |
| AIM2 | 0.340 | 0.0091 |
| C920009B18RIK | 0.341 | 0.0220 |
| BUB1 | 0.349 | 0.0755 |
| TICRR | 0.350 | 0.1772 |
| MIS18BP1 | 0.353 | 0.0909 |
| MAGEA6 | 0.353 | 0.2067 |
| CHIL3 | 0.354 | 0.0651 |
| IGHV1-78 | 0.357 | 0.2850 |
| STIL | 0.358 | 0.1413 |
| GM26735 | 0.360 | 0.0654 |
| REG2 | 0.360 | 0.4969 |
| SPRR1A | 0.361 | 0.0056 |
| PARPBP | 0.362 | 0.1471 |
| PADI4 | 0.366 | 0.0975 |
| GM14139 | 0.367 | 0.0423 |
| GPC3 | 0.368 | 0.2139 |
| MS4A6D | 0.370 | 0.0176 |
| ATP10A | 0.371 | 0.0977 |
| KIF23 | 0.371 | 0.0432 |
| GM20757 | 0.371 | 0.2248 |
| GM2318 | 0.372 | 0.0136 |
| GM2318 | 0.372 | 0.0136 |
| GM2318 | 0.372 | 0.0136 |
| GM2318 | 0.372 | 0.0136 |
| IGHV1-9 | 0.372 | 0.3585 |
| CPB1 | 0.374 | 0.5445 |
| TNFSF4 | 0.374 | 0.1420 |
| RNASE1 | 0.375 | 0.2935 |
| TIMP1 | 0.376 | 0.0765 |
| GM6020 | 0.379 | 0.4836 |
| 1700049E17RIK2 | 0.380 | 0.0045 |
| IGH-VJ558 | 0.382 | 0.4348 |
| PRRG4 | 0.382 | 0.1000 |
| GM7391 | 0.383 | 0.0206 |
| CD109 | 0.384 | 0.0892 |
| TMEM173 | 0.385 | 0.0912 |
| CDH17 | 0.385 | 0.0338 |
| KITL | 0.385 | 0.0287 |
| SKA3 | 0.388 | 0.1991 |
| PRC1 | 0.390 | 0.0796 |
| MBOAT1 | 0.390 | 0.0339 |
| TOP2A | 0.394 | 0.0659 |
| 1700049E17RIK2 | 0.395 | 0.0084 |
| SPEER4D | 0.396 | 0.0119 |
| HIST1H3I | 0.396 | 0.3818 |
| TUBA1A | 0.396 | 0.0139 |
| GM22265 | 0.396 | 0.0909 |
| TRBJ1-7 | 0.397 | 0.1567 |
| KIF20B | 0.398 | 0.0922 |
| SERPINB2 | 0.398 | 0.0259 |
| GM14402 | 0.398 | 0.2496 |
| PLK4 | 0.399 | 0.0728 |
| C330027C09RIK | 0.400 | 0.1046 |
| GM15091 | 0.401 | 0.0763 |
| NDC80 | 0.401 | 0.0773 |
| MYBL1 | 0.401 | 0.1659 |
| KIF2C | 0.401 | 0.0703 |
| GM12603 | 0.401 | 0.0856 |
| CYP11A1 | 0.403 | 0.2635 |
| FANCI | 0.403 | 0.1782 |
| CCNA2 | 0.404 | 0.0598 |
| RACGAP1 | 0.406 | 0.0734 |
| NCAPH | 0.406 | 0.1125 |
| GM16094 | 0.407 | 0.0317 |
| CH25H | 0.407 | 0.3075 |
| GM15398 | 0.408 | 0.1043 |
| KNTC1 | 0.409 | 0.1335 |
| BST1 | 0.409 | 0.0928 |
| KIF11 | 0.412 | 0.0752 |
| GM23576 | 0.412 | 0.2618 |
| SHCBP1 | 0.412 | 0.1361 |
| IGHV1-42 | 0.413 | 0.4474 |
| GPRC5A | 0.415 | 0.0333 |
| TNFRSF10B | 0.416 | 0.1047 |
| IL23A | 0.418 | 0.1414 |
| ERICH2 | 0.419 | 0.1444 |
| ANLN | 0.420 | 0.0409 |
| CASC5 | 0.420 | 0.1178 |
| GM17689 | 0.421 | 0.2773 |
| SOX4 | 0.422 | 0.0795 |
| GM22069 | 0.422 | 0.0494 |
| PRR11 | 0.423 | 0.0750 |
| SEMA3C | 0.423 | 0.0208 |
| RETNLA | 0.423 | 0.0429 |
| FRMD7 | 0.424 | 0.0211 |
| TNFRSF11B | 0.425 | 0.0592 |
| RAD54L | 0.426 | 0.1604 |
| GM10488 | 0.426 | 0.0074 |
| PLAT | 0.427 | 0.0647 |
| GM13790 | 0.427 | 0.1161 |
| DTL | 0.427 | 0.1700 |
| SERPINB9B | 0.429 | 0.1064 |
| ASNS | 0.430 | 0.0471 |
| GM15097 | 0.430 | 0.1722 |
| TMED6 | 0.432 | 0.2648 |
| SERPINE1 | 0.433 | 0.0345 |
| TPX2 | 0.433 | 0.0588 |
| CENPK | 0.433 | 0.1772 |
| 2810429I04RIK | 0.434 | 0.2171 |
| 4930461G14RIK | 0.434 | 0.1799 |
| HIST1H2AG | 0.435 | 0.1874 |
| IER3 | 0.435 | 0.1414 |
| CHRNB1 | 0.436 | 0.0500 |
| GM5431 | 0.437 | 0.1300 |
| GM13247 | 0.438 | 0.2035 |
| AI506816 | 0.438 | 0.0773 |
| GM7942 | 0.438 | 0.0432 |
| CCNB1 | 0.440 | 0.0362 |
| ZFP345 | 0.440 | 0.2490 |
| IGHV8-8 | 0.440 | 0.4507 |
| ATAD5 | 0.442 | 0.1143 |
| KDELR3 | 0.444 | 0.0873 |
| CDC25C | 0.445 | 0.0353 |
| IGKV4-72 | 0.445 | 0.1803 |
| OLFR99 | 0.447 | 0.1622 |
| GM25544 | 0.448 | 0.1385 |
| MIR101C | 0.448 | 0.0818 |
| HYDIN | 0.448 | 0.1499 |
| IGHV5-16 | 0.449 | 0.3210 |
| NOP58 | 0.449 | 0.1379 |
| CEL | 0.449 | 0.5062 |
| MS4A4A | 0.449 | 0.1126 |
| BCL2A1B | 0.450 | 0.0035 |
| IGKV4-59 | 0.450 | 0.4975 |
| CCL3 | 0.450 | 0.1070 |
| GM2933 | 0.451 | 0.1650 |
| FAM102B | 0.451 | 0.1330 |

TABLE 12
SEQUENCE LISTING

| SEQ | | |
|---|---|---|
| ID NO | Description | Sequence |
| | | anti-IL-27 Ab2-A |
| 9 | HCDR1 | GFTFSSYS |
| | (IMGT) | |
| 10 | HCDR2 | ISSSSSYI |
| | (IMGT) | |
| 11 | HCDR3 | ARDGGRTSYTATAHNWFDP |
| | (IMGT) | |
| 12 | HCDR1 (NT) | FTFSSYSMN |
| 13 | HCDR2 (NT) | SISSSSSYIYYADSVKG |
| 14 | HCDR3 (NT) | ARDGGRTSYTATAHNWFDP |
| 15 | VH | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 16 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 17 | LCDR1 | QSVLFSSNNKNY |
| | (IMGT) | |
| 18 | LCDR2 | WAS |
| | (IMGT) | |
| 19 | LCDR3 | QQHASAPPT |
| | (IMGT) | |
| 20 | LCDR1 (NT) | KSSQSVLFSSNNKNYLA |
| 21 | LCDR2 (NT) | WASTRES |
| 22 | LCDR3 (NT) | QQHASAPPT |
| 23 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 24 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 25 | Heavy Chain | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 26 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 27 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 28 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab2-B |
| 29 | Heavy Chain | EVQLVESGGGLVKPGGSLRLSCAASGFTFSSYSMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 30 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGCTATAGCATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| | | anti-IL-27 Ab3-A |
| 31 | HCDR1 | GFTFRSYG |
| | (IMGT) | |
| 32 | HCDR2 | ISSSSSYI |
| | (IMGT) | |
| 33 | HCDR3 | ARDGGRTSYTATAHNWFDP |
| | (IMGT) | |
| 34 | HCDR1 (NT) | FTFRSYGMN |
| 35 | HCDR2 (NT) | SISSSSSYIYYADSVKG |
| 36 | HCDR3 (NT) | ARDGGRTSYTATAHNWFDP |
| 37 | VH | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 38 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 39 | LCDR1 | QSVLFSSNNKNY |
| | (IMGT) | |
| 40 | LCDR2 | WAS |
| | (IMGT) | |
| 41 | LCDR3 | QQHASAPPT |
| | (IMGT) | |
| 42 | LCDR1 (NT) | KSSQSVLFSSNNKNYLA |
| 43 | LCDR2 (NT) | WASTRES |
| 44 | LCDR3 (NT) | QQHASAPPT |
| 45 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 46 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 47 | Heavy Chain | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 48 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 49 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 50 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab3-B |
| 51 | Heavy Chain | EVQLVESGGGLVKPGGSLRLSCAASGFTFRSYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 52 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GGAGCTATGGGATGAACTGGGTCCGCCAGGCTCCAGGGAAGGGG |
| | | CTGGAGTGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| | | anti-IL-27 Ab4-A |
| 53 | HCDR1 | GFTFSRTG |
| | (IMGT) | |
| 54 | HCDR2 | ISSSSSYI |
| | (IMGT) | |
| 55 | HCDR3 | ARDGGRTSYTATAHNWFDP |
| | (IMGT) | |
| 56 | HCDR1 (NT) | FTFSRTGMN |
| 57 | HCDR2 (NT) | SISSSSSYIYYADSVKG |
| 58 | HCDR3 (NT) | ARDGGRTSYTATAHNWFDP |
| 59 | VH | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 60 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 61 | LCDR1 | QSVLFSSNNKNY |
| | (IMGT) | |
| 62 | LCDR2 | WAS |
| | (IMGT) | |
| 63 | LCDR3 | QQHASAPPT |
| | (IMGT) | |
| 64 | LCDR1 (NT) | KSSQSVLFSSNNKNYLA |
| 65 | LCDR2 (NT) | WASTRES |
| 66 | LCDR3 (NT) | QQHASAPPT |
| 67 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 68 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 69 | Heavy Chain | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 70 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 71 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 72 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab4-B |
| 73 | Heavy Chain | EVQLVESGGGLVKPGGSLRLSCAASGFTFSRTGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 74 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGACTGGGATGAACTGGGTCCGCCAGGCTCCAGGGAAGGGG |
| | | CTGGAATGGGTCTCATCCATTAGTAGTAGTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| | | anti-IL-27 Ab5-A |
| 75 | HCDR1 | GFTFSRYG |
| | (IMGT) | |
| 76 | HCDR2 | ISSSSAYI |
| | (IMGT) | |
| 77 | HCDR3 | ARDGGRTSYTATAHNWFDP |
| | (IMGT) | |
| 78 | HCDR1 (NT) | FTFSRYGMN |
| 79 | HCDR2 (NT) | SISSSSAYILYADSVKG |
| 80 | HCDR3 (NT) | ARDGGRTSYTATAHNWFDP |
| 81 | VH | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSS |
| 82 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 83 | LCDR1 | QSVLFSSNNKNY |
| | (IMGT) | |
| 84 | LCDR2 | WAS |
| | (IMGT) | |
| 85 | LCDR3 | QQHASAPPT |
| | (IMGT) | |
| 86 | LCDR1 (NT) | KSSQSVLFSSNNKNYLA |
| 87 | LCDR2 (NT) | WASTRES |
| 88 | LCDR3 (NT) | QQHASAPPT |
| 89 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 90 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 91 | Heavy Chain | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 92 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 93 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 94 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab5-B |
| 95 | Heavy Chain | EVQLVESGGGLVKPGGSLRLSCAASGFTFSRYGMNWVRQAPGKG |
| | | LEWVSSISSSSAYILYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 96 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCA |
| | | GTAGGTATGGGATGAACTGGGTCCGCCAGGCTCCAGGGAAGGGG |
| | | CTGGAGTGGGTCTCATCCATTAGTAGTAGTAGTGCTTACATACT |
| | | GTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| | | anti-IL-27 Ab6-A |
| 97 | HCDR1 | GFTFASYG |
| | (IMGT) | |
| 98 | HCDR2 | ISSSSSYI |
| | (IMGT) | |
| 99 | HCDR3 | ARDGGRTSYTATAHNWFDP |
| | (IMGT) | |
| 100 | HCDR1 (NT) | FTFASYGMN |
| 101 | HCDR2 (NT) | SISSSSSYIYYADSVKG |
| 102 | HCDR3 (NT) | ARDGGRTSYTATAHNWFDP |
| 103 | VH | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSS |
| 104 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 105 | LCDR1 | QSVLFSSNNKNY |
| | (IMGT) | |
| 106 | LCDR2 | WAS |
| | (IMGT) | |
| 107 | LCDR3 | QQHASAPPT |
| | (IMGT) | |
| 108 | LCDR1 (NT) | KSSQSVLFSSNNKNYLA |
| 109 | LCDR2 (NT) | WASTRES |
| 110 | LCDR3 (NT) | QQHASAPPT |
| 111 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 112 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 113 | Heavy Chain | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 114 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 115 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 116 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab6-B |
| 117 | Heavy Chain | EVQLVESGGGLVKPGGSLRLSCAASGFTFASYGMNWVRQAPGKG |
| | | LEWVSSISSSSSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 118 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCG |
| | | CTAGCTATGGGATGAACTGGGTCCGCCAGGCTCCAGGGAAGGGG |
| | | CTGGAGTGGGTCTCATCCATTAGTAGTTCTAGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| | | anti-IL-27 Ab1-A |
| 119 | HCDR1 | GFTFRSYG |
| | (IMGT) | |
| 120 | HCDR2 | ISSSGSYI |
| | (IMGT) | |
| 121 | HCDR3 | ARDGGRTSYTATAHNWFDP |
| | (IMGT) | |
| 122 | HCDR1 (NT) | FTFRSYGMN |
| 123 | HCDR2 (NT) | GISSSGSYIYYADSVKG |
| 124 | HCDR3 (NT) | ARDGGRTSYTATAHNWFDP |
| 125 | VH | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWFDPWGQGTLVTVSS |
| 126 | DNA VH | GAGGTGCAGCTGGTGGAGTCTGGGGGAGGCCTGGTCAAGCCTGG |
| | | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCA |
| 127 | LCDR1 | QSVLFSSNNKNY |
| | (IMGT) | |
| 128 | LCDR2 | WAS |
| | (IMGT) | |
| 129 | LCDR3 | QQHASAPPT |
| | (IMGT) | |
| 130 | LCDR1 (NT) | KSSQSVLFSSNNKNYLA |
| 131 | LCDR2 (NT) | WASTRES |
| 132 | LCDR3 (NT) | QQHASAPPT |
| 133 | VL | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIK |
| 134 | DNA VL | GACATCGTGATGACCCAGTCTCCAGACTCCCTGGCTGTGTCTCT |
| | | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAA |
| 135 | Heavy Chain | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWFDPWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALPAPIEKTISKAKGQP |
| | | REPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 136 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCGAGCACCAAAGGCCCG |
| | | AGCGTGTTTCCGCTGGCGCCGAGCAGCAAAAGCACCAGCGGCGG |
| | | CACCGCGGCGCTGGGCTGCCTGGTGAAAGATTATTTTCCGGAAC |
| | | CGGTGACCGTGAGCTGGAACAGCGGCGCGCTGACCAGCGGCGTG |
| | | CATACCTTTCCGGCGGTGCTGCAGAGCAGCGGCCTGTATAGCCT |
| | | GAGCAGCGTGGTGACCGTGCCGAGCAGCAGCCTGGGCACCCAGA |
| | | CCTATATTTGCAACGTGAACCATAAACCGAGCAACACCAAAGTG |
| | | GATAAAAAAGTGGAACCGAAAAGCTGCGATAAAACCCATACCTG |
| | | CCCGCCGTGCCCGGCGCCGGAACTGCTGGGCGGCCCGAGCGTGT |
| | | TTCTGTTTCCGCCGAAACCGAAAGATACCCTGATGATTAGCCGC |
| | | ACCCCGGAAGTGACCTGCGTGGTGGTGGATGTGAGCCATGAAGA |
| | | TCCGGAAGTGAAATTTAACTGGTATGTGGATGGCGTGGAAGTGC |
| | | ATAACGCGAAAACCAAACCGCGCGAAGAACAGTATAACAGCACC |
| | | TATCGCGTGGTGAGCGTGCTGACCGTGCTGCATCAGGATTGGCT |
| | | GAACGGCAAAGAATATAAATGCAAAGTGAGCAACAAAGCGCTGC |
| | | CGGCGCCGATTGAAAAAACCATTAGCAAAGCGAAAGGCCAGCCG |
| | | CGCGAACCGCAGGTGTATACCCTGCCGCCGAGCCGCGATGAACT |
| | | GACCAAAAACCAGGTGAGCCTGACCTGCCTGGTGAAAGGCTTTT |
| | | ATCCGAGCGATATTGCGGTGGAATGGGAAAGCAACGGCCAGCCG |
| | | GAAAACAACTATAAAACCACCCCGCCGGTGCTGGATAGCGATGG |
| | | CAGCTTTTTTCTGTATAGCAAACTGACCGTGGATAAAAGCCGCT |
| | | GGCAGCAGGGCAACGTGTTTAGCTGCAGCGTGATGCATGAAGCG |
| | | CTGCATAACCATTATACCCAGAAAAGCCTGAGCCTGAGCCCGGG |
| | | CAAA |
| 137 | Light Chain | DIVMTQSPDSLAVSLGERATINCKSSQSVLFSSNNKNYLAWYQQ |
| | | KPGQPPKLLIYWASTRESGVPDRFSGSGSGTDFTLTISSLQAED |
| | | VAVYYCQQHASAPPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| 138 | DNA Light | GACATCGTGATGACCCAGTCTCCAGACTCCCTGGCTGTGTCTCT |
| | Chain | GGGCGAGAGGGCCACCATCAACTGCAAGTCCAGCCAGAGTGTTT |
| | | TATTCAGCTCCAACAATAAGAACTACTTAGCTTGGTACCAGCAG |
| | | AAACCAGGACAGCCTCCTAAGCTGCTCATTTACTGGGCATCTAC |
| | | CCGGGAATCCGGGGTCCCTGACCGATTCAGTGGCAGCGGGTCTG |
| | | GGACAGATTTCACTCTCACCATCAGCAGCCTGCAGGCTGAAGAT |
| | | GTGGCAGTTTATTACTGTCAGCAGCACGCCAGTGCCCCTCCTAC |
| | | TTTTGGCGGAGGGACCAAGGTTGAGATCAAACGTACGGTGGCCG |
| | | CTCCCTCCGTGTTCATCTTCCCACCCTCCGACGAGCAGCTGAAG |
| | | TCCGGCACCGCCTCCGTCGTGTGCCTGCTGAACAACTTCTACCC |
| | | TCGCGAGGCCAAAGTGCAGTGGAAAGTGGACAACGCCCTGCAGT |
| | | CCGGCAACTCCCAGGAATCCGTCACCGAGCAGGACTCCAAGGAC |
| | | AGCACCTACTCCCTGTCCTCCACCCTGACCCTGTCCAAGGCCGA |
| | | CTACGAGAAGCACAAAGTGTACGCCTGCGAAGTGACCCACCAGG |
| | | GCCTGTCCAGCCCCGTGACCAAGTCCTTCAACCGGGGCGAGTGC |
| | | anti-IL-27 Ab1-B |
| 139 | Heavy Chain | EVQLVESGGGLVKPGGSLRLSCAASGFTFRSYGMNWVRQAPGKG |
| | | LEWVSGISSSGSYIYYADSVKGRFTISRDNAKNSLYLQMNSLRA |
| | | EDTAVYYCARDGGRTSYTATAHNWFDPWGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRVESKYGPPCPSCPAPEFLGGPSVFLFPPKPKDTLMISRTPE |
| | | VTCVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLG |
| 140 | DNA Heavy | GAGGTGCAGCTGGTGGAGTCTGGGGGAGGCCTGGTCAAGCCTGG |
| | Chain | GGGGTCCCTGAGACTCTCCTGTGCAGCCTCTGGATTCACCTTCC |
| | | GTAGCTATGGGATGAACTGGGTCCGCCAGGCTCCAGGGAAGGGG |
| | | CTGGAGTGGGTCTCAGGTATTAGTAGTAGTGGTAGTTACATATA |
| | | CTACGCAGACTCAGTGAAGGGCCGATTCACCATCTCCAGAGACA |
| | | ACGCCAAGAACTCACTGTATCTGCAAATGAACAGCCTGAGAGCC |
| | | GAGGACACGGCGGTGTACTACTGCGCCAGAGATGGTGGAAGAAC |
| | | GTCCTACACCGCCACAGCCCACAATTGGTTCGACCCCTGGGGAC |
| | | AGGGTACATTGGTCACCGTCTCCTCAGCTTCCACCAAGGGCCCC |
| | | TCCGTGTTCCCTCTGGCCCCTTGCTCCCGGTCCACCTCCGAGTC |
| | | TACCGCCGCTCTGGGCTGCCTCGTGAAGGACTACTTCCCCGAGC |
| | | CCGTGACCGTGTCCTGGAACTCTGGCGCCCTGACCTCCGGCGTG |
| | | CACACCTTCCCTGCCGTGCTGCAGTCCTCCGGCCTGTACTCCCT |
| | | GTCCAGCGTCGTGACCGTGCCCTCCTCCAGCCTGGGCACCAAGA |
| | | CCTACACCTGTAACGTGGACCACAAGCCCTCCAACACCAAAGTG |
| | | GACAAGCGGGTGGAATCTAAGTACGGCCCTCCCTGCCCTTCCTG |
| | | CCCTGCCCCTGAGTTCCTGGGCGGACCTTCCGTGTTCCTGTTCC |
| | | CTCCAAAGCCCAAGGACACCCTGATGATCTCCCGGACCCCTGAA |
| | | GTGACCTGCGTGGTGGTGGACGTGTCCCAGGAAGATCCCGAAGT |
| | | CCAGTTCAATTGGTACGTGGACGGCGTGGAAGTGCACAACGCCA |
| | | AGACCAAGCCCAGAGAGGAACAGTTCAACTCCACCTACCGGGTG |
| | | GTGTCCGTGCTGACCGTGCTGCACCAGGACTGGCTGAACGGCAA |
| | | AGAGTACAAGTGCAAAGTGTCCAACAAGGGCCTGCCCTCCAGCA |
| | | TCGAAAAGACCATCTCCAAGGCCAAGGGCCAGCCCCGCGAGCCC |
| | | CAAGTGTACACCCTGCCTCCCAGCCAGGAAGAGATGACCAAGAA |
| | | TCAAGTGTCCCTGACTTGTCTGGTCAAGGGCTTCTACCCCTCCG |
| | | ATATCGCCGTGGAGTGGGAGTCCAACGGCCAGCCCGAGAACAAC |
| | | TACAAGACCACCCCTCCCGTGCTGGACTCCGACGGCTCCTTCTT |
| | | CCTGTACTCTCGGCTGACCGTGGACAAGTCCCGGTGGCAGGAAG |
| | | GCAACGTCTTCTCCTGCTCCGTGATGCACGAGGCCCTGCACAAC |
| | | CACTACACCCAGAAGTCCCTGTCCCTGTCTCTGGGC |
| 141 | FLAG | DYKDDDDK |
| 142 | 6-HIS | HHHHHH |
| 143 | HA | YPYDVPDYA |

TABLE 13
Fc Sequences (=CH2 + CH3)

| Name | Alias | Amino Acid Sequence |
|---|---|---|
| Human | 1.0 | EPKSCDKTHTCPPCPAPELLGGPSVFLFPPKP |
| IgG1 | | KDTLMISRTPEVTCVVVDVSHEDPEVKFNWYV |
| | | DGVEVHNAKTKPREEQYNSTYRVVSVLTVLHQ |
| | | DWLNGKEYKCKVSNKALPAPIEKTISKAKGQP |
| | | REPQVYTLPPSRDELTKNQVSLTCLVKGFYPS |
| | | DIAVEWESNGQPENNYKTTPPVLDSDGSFFLY |
| | | SKLTVDKSRWQQGNVFSCSVMHEALHNHYTQK |
| | | SLSLSPGK (SEQ ID NO: 5) |
| Human | 4.0 | ESKYGPPCPSCPAPEFLGGPSVFLFPPKPKDT |
| IgG4 | | LMISRTPEVTCVVVDVSQEDPEVQFNWYVDGV |
| | | EVHNAKTKPREEQFNSTYRVVSVLTVLHQDWL |
| | | NGKEYKCKVSNKGLPSSIEKTISKAKGQPREP |
| | | QVYTLPPSQEEMTKNQVSLTCLVKGFYPSDIA |
| | | VEWESNGQPENNYKTTPPVLDSDGSFFLYSRL |
| | | TVDKSRWQEGNVFSCSVMHEALHNHYTQKSLS |
| | | LSLGK (SEQ ID NO: 6) |
| Human | 4.1 | ESKYGPPCPPCPAPEFLGGPSVFLFPPKPKDT |
| IgG4 | | LMISRTPEVTCVVVDVSQEDPEVQFNWYVDGV |
| (S228P) | | EVHNAKTKPREEQFNSTYRVVSVLTVLHQDWL |
| | | NGKEYKCKVSNKGLPSSIEKTISKAKGQPREP |
| | | QVYTLPPSQEEMTKNQVSLTCLVKGFYPSDIA |
| | | VEWESNGQPENNYKTTPPVLDSDGSFFLYSRL |
| | | TVDKSRWQEGNVFSCSVMHEALHNHYTQKSLS |
| | | LSLGK (SEQ ID NO: 7) |
| Human | 4.2 | ESKYGPPCPPCPAPEFEGGPSVFLFPPKPKDT |
| IgG4 | | LMISRTPEVTCVVVDVSQEDPEVQFNWYVDGV |
| (S228P/ | | EVHNAKTKPREEQFNSTYRVVSVLTVLHQDWL |
| L235E) | | NGKEYKCKVSNKGLPSSIEKTISKAKGQPREP |
| | | QVYTLPPSQEEMTKNQVSLTCLVKGFYPSDIA |
| | | VEWESNGQPENNYKTTPPVLDSDGSFFLYSRL |
| | | TVDKSRWQEGNVFSCSVMHEALHNHYTQKSLS |
| | | LSLGK (SEQ ID NO: 8) |

### Example 9: Anti-IL-27 Ab1 Binding Properties and IL-27 Receptor Blockade

The association and dissociation of recombinant human IL-27 at concentrations ranging from 0 to 5.0 μg/mL with an anti-IL-27 Ab1 concentration of 1 μg/mL were determined. Final binding kinetic parameters are shown in Table 14 along with binding model fit parameters (R2 and χ2) that demonstrate goodness of the model fitting to the data.

Human IL-27 displayed the strongest binding affinity for anti-IL-27 Ab1 of all species tested in this study (3.86 pM). Recombinant rat and cynomolgus monkey IL-27 also showed strong affinities for anti-IL-27 Ab1 with values of 80.9 and 37.4 pM, respectively, although somewhat weaker than the human protein. Recombinant mouse IL-27 had the weakest affinity for anti-IL-27 Ab1 by comparison with the human protein, with a value in the nM range (4.43 nM) as indicated by its slower association and faster dissociation rates.

TABLE 14
Data Summary for IL-27 Binding to Anti-IL-27 Ab1 and
Species Cross-Reactivity

| Analyte | K_{D} (M) | kₐ (1/Ms) | k_{d} (1/s) | Full χ² | Full R² |
|---|---|---|---|---|---|
| Human IL-27 | 3.86E−12 | 5.10E+05 | 1.97E−06 | 0.4055 | 0.9991 |
| Mouse IL-27 | 4.43E−09 | 5.50E+04 | 2.44E−04 | 0.6732 | 0.9963 |
| Rat IL-27 | 8.09E−11 | 2.34E+06 | 1.89E−04 | 0.4685 | 0.9945 |
| Cynomolgus | 3.74E−11 | 3.18E+05 | 1.19E−05 | 1.3431 | 0.9979 |
| monkey IL-27 | | | | | |

Abbreviations: IL-27 = interleukin 27, kₐ = association constant, k_{d} = dissociation constant, K_{D} = binding affinity
Note:
R² values > 0.95 and χ² values < 3.0 are demonstrative of a good fit of the model to the data.

### Example 10: CDR Sequence Alignments

A number of sub-selections of anti-IL-27 antibodies of the instant disclosure share sequence homology across their CDR regions, providing a diversity of variant CDR sequences that have been validated as retaining functionality. It is expressly contemplated herein that the following consensus CDR sequences are fully supported by—and are therefore within the scope of—the instant disclosure.

For anti-IL-27 Ab1, anti-IL-27 Ab3, anti-IL-27 Ab4, anti-IL-27 Ab5, anti-IL-27 Ab6, and anti-IL-27 Ab7 antibodies, alignments of the CDR sequences of each of these anti-IL-27 antibodies revealed extensive homology, punctuated by variable residues. In particular, heavy chain CDR1 alignments revealed the following variable residues:

HCDR1 (IMGT)
CLUSTAL O(1.2.4) multiple sequence alignment

| | | | |
|---|---|---|---|
| 1 | GFTFRSYG | 8 | (SEQ ID NO: 119) |
| 5 | GFTFRSYG | 8 | (SEQ ID NO: 31) |
| 4 | GFTFASYG | 8 | (SEQ ID NO: 97) |
| 2 | GFTFSRTG | 8 | (SEQ ID NO: 53) |
| 3 | GFTFSRYG | 8 | (SEQ ID NO: 75) |
| 6 | GFTFSSYS | 8 | (SEQ ID NO: 9) |
| | **** | | |

A consensus heavy chain CDR1 (IMGT) sequence for these homologous antibodies is therefore N-GFTF[S/A/R][S/R][T/Y][G/S]-C (SEQ ID NO: 144) and, accordingly, more generally contemplated herein as a consensus heavy chain CDR1 (IMGT) sequence is N-GFTFXXXX-C(SEQ ID NO: 145), where X is any amino acid residue.

Alignment of the anti-IL-27 Ab1, anti-IL-27 Ab3, anti-IL-27 Ab4, anti-IL-27 Ab5, anti-IL-27 Ab6, and anti-IL-27 Ab7 antibody heavy chain CDR2 (IMGT) sequences revealed the following:

HCDR2 (IMGT)
CLUSTAL O(1.2.4) multiple sequence alignment

| | | | |
|---|---|---|---|
| 10 | ISSSGSYI | 8 | (SEQ ID NO: 120) |
| 11 | ISSSSSYI | 8 | (SEQ ID NO: 98) |
| 7 | ISSSSSYI | 8 | (SEQ ID NO: 32) |
| 9 | ISSSSSYI | 8 | (SEQ ID NO: 54) |
| 8 | ISSSSAYI | 8 | (SEQ ID NO: 76) |
| 12 | ISSSSSYI | 8 | (SEQ ID NO: 10) |
| | ****.:** | | |

A consensus heavy chain CDR2 (IMGT) sequence for these homologous antibodies is therefore N-ISSS[S/G][S/A]YI-C (SEQ ID NO: 146) and, accordingly, more generally contemplated herein as a consensus heavy chain CDR2 (IMGT) sequence is N-ISSSXXYI-C (SEQ ID NO: 147), where X is any amino acid residue.

Alignments of the human CDR1 (NT) and human CDR2 (NT) sequences also revealed the following:

HCDR1 (NT)
CLUSTAL O(1.2.4) multiple sequence alignment

| | | | |
|---|---|---|---|
| 13 | FTFRSYGMN | 9 | (SEQ ID NO: 34) |
| 16 | FTFRSYGMN | 9 | (SEQ ID NO: 122) |
| 17 | FTFASYGMN | 9 | (SEQ ID NO: 100) |
| 14 | FTFSRTGMN | 9 | (SEQ ID NO: 56) |
| 15 | FTFSRYGMN | 9 | (SEQ ID NO: 78) |
| 18 | FTFSSYSMN | 9 | (SEQ ID NO: 12) |
| | *** *** | | |

HCDR2 (NT)
CLUSTAL O(1.2.4) multiple sequence alignment

| | | | |
|---|---|---|---|
| 23 | GISSSGSYIYYADSVKG | 17 | (SEQ ID NO: 123) |
| 19 | SISSSSSYIYYADSVKG | 17 | (SEQ ID NO: 35) |
| 20 | SISSSSSYIYYADSVKG | 17 | (SEQ ID NO: 57) |
| 22 | SISSSSSYIYYADSVKG | 17 | (SEQ ID NO: 101) |
| 21 | SISSSSAYILYADSVKG | 17 | (SEQ ID NO: 79) |
| 24 | SISSSSSYIYYADSVKG | 17 | (SEQ ID NO: 13) |
| | .****.:** ******* | | |

Consensus heavy chain CDR1 (NT) and CDR2 (NT) sequences for these homologous antibodies are therefore N-FTF[S/A/R][S/R][T/Y][G/S]MN-C (SEQ ID NO: 148) and N-[G/S]ISSS[S/G][S/A]YI[L/Y]YADSVKG-C (SEQ ID NO: 149), respectively. In view of these consensus sequences, more generally contemplated herein are consensus heavy chain CDR1 (NT) and CDR2 (NT) sequences N-FTFXXXXMN-C (SEQ ID NO: 150) and N-XISSSXXYIXYADSVKG-C (SEQ ID NO: 151), respectively, where X is any amino acid residue.

Heavy chain CDR3 (IMGT or NT) and light chain CDRs CDR1 (IMGT or NT), CDR2 (IMGT or NT) and CDR3 (IMGT or NT) were fully conserved between anti-IL-27 Ab1, anti-IL-27 Ab3, anti-IL-27 Ab4, anti-IL-27 Ab5, anti-IL-27 Ab6, and anti-IL-27 Ab7.

### Example 11: Crystallization and Epitope Determination of IL-27-Anti-IL-27 Ab1 Fab Complex

Initial crystallization trials were set up with Human IL-27-anti-IL-27 Ab1 Fab complex at 10.1 mg/ml in 25 mM Tris pH 7.5, approximately 30 mM sodium chloride and 5% glycerol. Preliminary crystallization conditions were identified using a PACT screen (Newman et al., (2005) Acta Cryst. D 61: 1426).

Crystals were obtained under multiple conditions, including PACT A2 (0.1 M SPG (succinic acid, sodium dihydrogen phosphate, and glycine) pH 5 and 25% PEG 1500). These crystals were used to make a new seed stock and set up a micro matrix seeding (MMS) experiment using a JCSG+ screen (D'Arcy et al., (2007) Acta Cryst. D Biol Cryst. 63: 550-54).

Data sets was collected at 100K at station 104, Diamond Light Source, Didcot, England (λ=0.9795 Å) equipped with an Eiger2 XE 16M detector. The data were processed using autoPROC (Kabash, (2010) Acta. Cryst. D. Biol. Cyrst. 66: 125-32; Vonrhein et al., (2011) Acta Cryst. Biol. Cryst. 67: 292-302) and anisotropically truncated using the STARANISO software (Tickle et al., STRANISO. Cambridge, United Kingdon: Global Phasing Ltd. (2018)) also including the Aimless program (Evans et al., (2013) Acta Cryst. Biol. Cryst. 69: 1204-14).

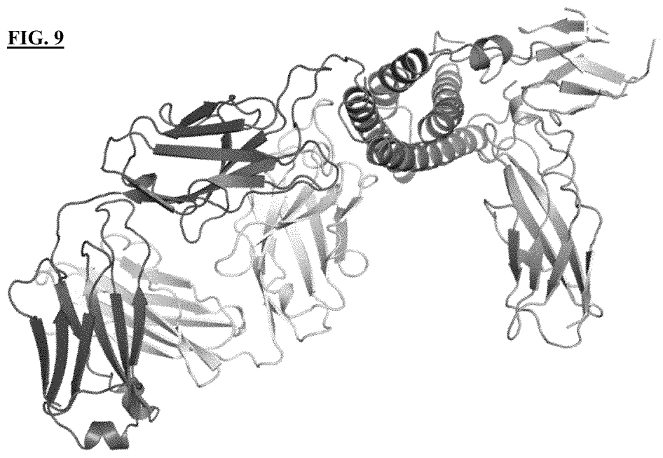

The structure was determined using the molecular replacement software Phaser (McCoy et al., (2007) J. Appl. Cyrst. 40: 658-74) and Molrep (Vagin et al., (1997) J. Appl. Cyrst. 30: 1022-25) (FIG. 9). As shown in FIG. 9, the anti-IL-27 Ab1 Fab is bound to the p28 molecule of IL-27. Electron density for the whole epitope-paratope region is well defined. The interactions between anti-IL-27 Ab1 and p28 are shown in Table 15.

TABLE 15
Interaction Map for Anti-IL-27 Ab1 with p28 (≤4.0 Å)

| | | Anti-IL-27 | | | | |
|---|---|---|---|---|---|---|
| IL27 | | Ab1 Fab | | | | |
| (p28) | | | | CDR | Distance | |
| Residue | Atom | Residue | Atom | loop | (Å) | Type |
| Gln37 | O^{e1} | Ser32 | N | L1 | 3.0 | H-bond |
| | C^{b} | Phe31 | C^{d1} | L1 | 3.6 | Hydrophobic |
| Leu38 | C^{g} | Phe31 | C^{z} | L1 | 4.1 | Hydrophobic |
| Glu42 | O^{e1} | Ser99 | C^{g} | L3 | 2.7 | H-bond |
| Glu46 | O^{e2} | Tyr59 | O^{h} | H2 | 2.7 | H-bond |
| Val49 | C^{g1} | Tyr57 | C^{d1} | H2 | 3.5 | Hydrophobic |
| Ser50 | O^{g} | Tyr57 | O^{h} | H2 | 2.7 | H-bond |
| Leu142 | C^{d2} | Ser54 | C^{a} | H2 | 3.8 | Hydrophobic |
| Asp146 | O^{d1, 2} | Ser53 | O^{g} | H2 | 3.7 | H-bond |
| | O^{d2} | Ser54 | O^{g} | H2 | 2.7 | H-bond |
| | O^{d2} | Ser56 | O^{g} | H2 | 4.0 | H-bond |
| Arg149 | N^{e} | Thr103 | O | H3 | 3.1 | H-bond |
| | N^{e} | Thr106 | O | H3 | 2.9 | H-bond |
| | NH² | Gly101 | O | H3 | 3.0 | H-bond |
| His150 | N^{e2} | Thr108 | O^{g1} | H3 | 2.7 | H-bond |
| | N^{d1}, C^{e1} | Tyr57 | C^{e2}, C^{d2} | H2 | 3.5-3.7 | p-Stacking |
| Phe153 | C^{z} | His110 | N^{d1} | H3 | 3.6 | Hydrophobic |
| | C^{d2} | Ala107 | C^{b} | H3 | 3.7 | Hydrophobic |
| | C^{e1} | Tyr38 | C^{e2} | L1 | 3.8 | Hydrophobic |
| Leu156 | O | Ser33 | O^{g} | L1 | 2.6 | H-bond |
| | C^{d2} | Tyr105 | C^{d1} | H3 | 3.9 | Hydrophobic |
| | C^{d1} | Ser33 | C^{b} | L1 | 3.9 | Hydrophobic |
| Leu162 | O | Tyr105 | O^{h} | H3 | 2.8 | H-bond |
| Glu164 | O^{e1} | Thr103 | O^{g} | H3 | 2.6 | H-bond |
| | O^{e2} | Lys36 | N^{z} | L1 | 3.9 | Ionic |

### Example 12: Additional Epitope Mapping Studies

Further epitope mapping studies were carried out using the IL-27-anti-IL-27 Ab1 Fab complex crystal structure. Interaction areas between the anti-IL-27 Ab1 Fab molecule and IL-27 were investigated using the Qt-PISA and NCONT programs in the CCP4 package (Winn, et al., (2011) Acta Cryst. D Biol. Cryst. 67: 235-42). Coot (Emsley, et al., (2010) Acta Cryst. D Biol. Cyrst. 66: 486-501) was used for data analysis.

Epitope residues were defined as IL-27 amino acids having atoms within 4 Å of Fab atoms, evaluated using NCONT in CCP4. As well as the residues previously identified and listed in Table 15, these studies found additional epitope residues. All interactions between IL-27p28 and anti-IL-27 Ab1 Fab within 4 Å is shown below in Table 16A.

TABLE 16A

| | Anti-IL-27 | |
|---|---|---|
| CDR | Ab1 Fab | IL-27 (p28) Residue |
| LC-CDR1 | Leu 30 | Gln 37 |
| | Phe 31 | Gln 37, Leu 38, Phe 153, Ala 157 |
| | Ser 32 | Gln 37 |
| | Ser 33 | Gln 37, Leu 156, Gly 159, Phe 160, Asn 161 |
| | Asn 34 | Leu 156 |
| | Lys 36 | Glu 164 |
| | Tyr 38 | Phe 153 |
| LC-CDR3 | Ala 98 | Leu 38 |
| | Ser 99 | Leu 38, Glu 42 |
| | Ala 100 | Glu 42 |
| HC-CDR1 | Ser 31 | Arg 145* |
| HC-CDR1 | Ser 52 | Asp 146, His 150 |
| | Ser 53 | Leu 142, Asp 146 |
| | Ser 54 | Leu 142, Asp 143, Asp 146 |
| | Ser 56 | Leu 53, Lys 56, Asp 146 |
| | Tyr 57 | Val 49, Ser 50, Leu 53, Asp 146, Leu 147, |
| | | His 150 |
| | Tyr 59 | Glu 46 |
| | Asn 74 | Leu 142** |
| HC-CDR3 | Gly 101 | Arg 149 |
| | Arg 102 | Arg 149 |
| | Thr 103 | Arg 149, Glu 164 |
| | Ser 104 | Arg 149, Arg 152, Phe 153, Leu 156 |
| | Tyr 105 | Phe 153, Leu 156, Leu 162, Pro 163*, Glu 164 |
| | Thr 106 | Arg 149, Phe 153 |
| | Ala 107 | Arg 149, His 150, Phe 153 |
| | Thr 108 | His 150 |
| | His 110 | Glu 42, Glu 46, Phe 153 |
| | Asn 111 | Arg 149 |

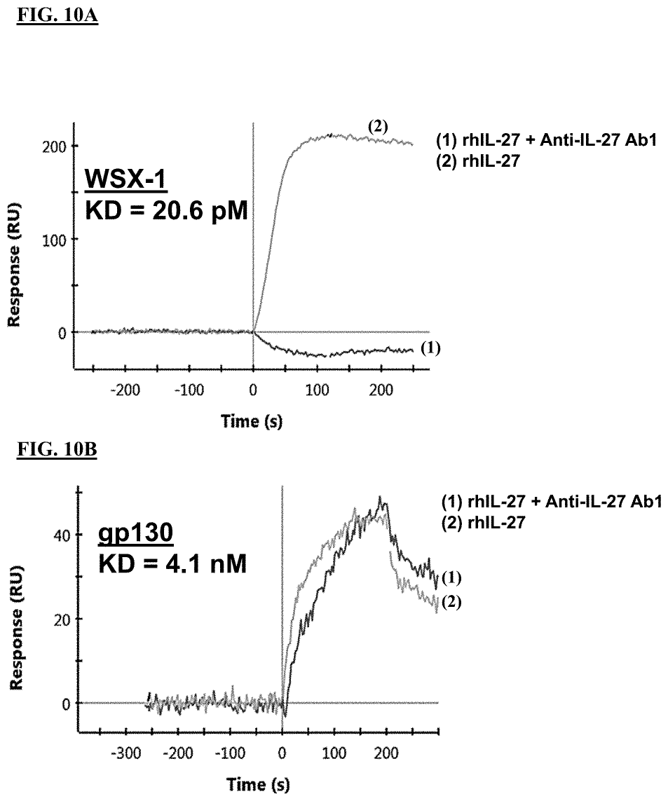

Binding and blocking studies were performed by SPR for both WSX-1 and gp130 for the human IL-27 heterodimer. Human IL-27 bound with high affinity to WSX-1 and anti-IL-27 Ab1 was able to completely inhibit binding (FIG. 10A). Human IL-27 bound with lower affinity to gp130, and anti-IL-27 Ab1 did not inhibit binding of IL-27 to gp130 (FIG. 10B).

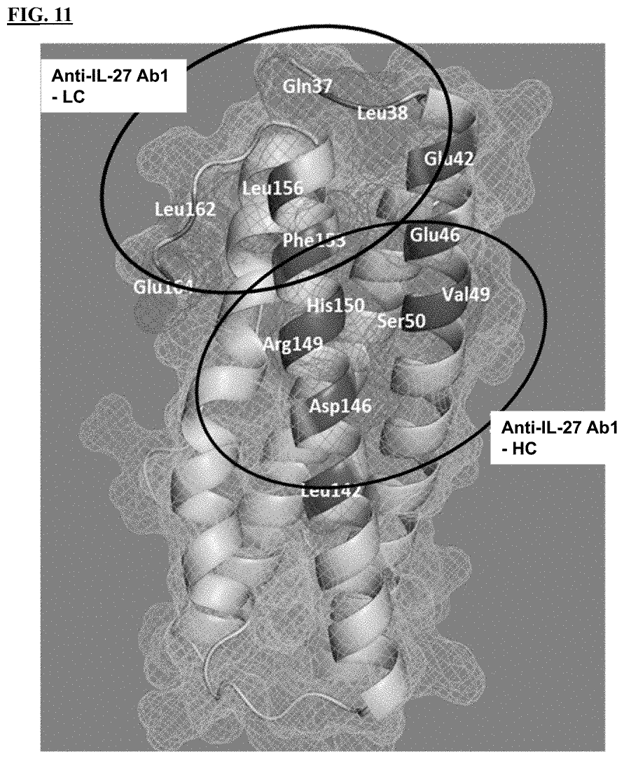

Anti-IL-27 Ab1 Interacts with the αA and αC helices and the initial portion of the poly-Glu sequence (FIG. 11). Heavy-chain CDR's 2 and 3 have the most extensive contacts with p28 (Table 16B).

TABLE 16B
Contacts between IL27-p28 and Anti-IL-27 Ab1.

| | | Anti-IL-27 | | | | |
|---|---|---|---|---|---|---|
| IL27 | | Ab1 Fab | | | | |
| (p28) | | | | CDR | Distance | |
| Residue | Atom | Residue | Atom | loop | (Å) | Type |
| Gln37 | O^{e1} | Ser32 | O^{g} | L1 | 3.0 | H-bond |
| | C^{b} | Phe31 | C^{d1} | L1 | 3.6 | Hydrophobic |
| Leu38 | C^{g} | Phe31 | C^{z} | L1 | 3.8 | Hydrophobic |
| Glu42 | O^{e1} | Ser99 | O^{g} | L3 | 2.8 | H-bond |
| Glu46 | O^{e1} | Tyr59 | O^{h} | H2 | 2.8 | H-bond |
| Val49 | C^{g1} | Tyr57 | C^{d2} | H2 | 3.6 | Hydrophobic |
| Ser50 | O^{g} | Tyr57 | O^{h} | H2 | 2.7 | H-bond |
| Leu142 | C^{d2} | Ser54 | C^{a} | H2 | 3.9 | Hydrophobic |
| Asp146 | O^{d1} | Ser53 | O^{g} | H2 | 2.7 | H-bond |
| | O^{d2} | Ser54 | O^{g} | H2 | 2.7 | H-bond |
| | O^{d2} | Ser56 | O^{g} | H2 | 2.9 | H-bond |
| Arg149 | N^{e} | Thr103 | O | H3 | 2.8 | H-bond |
| | N^{e} | Thr106 | O | H3 | 2.8 | H-bond |
| | NH² | Gly101 | O | H3 | 3.0 | H-bond |
| His150 | N^{e2} | Thr108 | O^{g1} | H3 | 2.8 | H-bond |
| | N^{d1}, C^{e1} | Tyr57 | C^{e1}, C^{d1} | H2 | 3.5-3.6 | p-Stacking |
| Phe153 | C^{z} | His110 | N^{d1} | H3 | 3.8 | Hydrophobic |
| | C^{d2} | Ala107 | C^{b} | H3 | 3.6 | Hydrophobic |
| | C^{e1} | Tyr38 | C^{e2} | L1 | 3.7 | Hydrophobic |
| Leu156 | O | Ser33 | O^{g} | L1 | 2.7 | H-bond |
| | C^{d1} | Tyr105 | C^{d1} | H3 | 3.7 | Hydrophobic |
| | C^{d2} | Ser33 | C^{b} | L1 | 3.8 | Hydrophobic |
| Leu162 | O | Tyr105 | O^{h} | H3 | 3.1 | H-bond |
| Glu164 | O^{e1} | Thr103 | O^{g} | H3 | 2.7 | H-bond |
| | O^{e2} | Lys36 | N^{z} | L1 | 3.9 | Ionic |

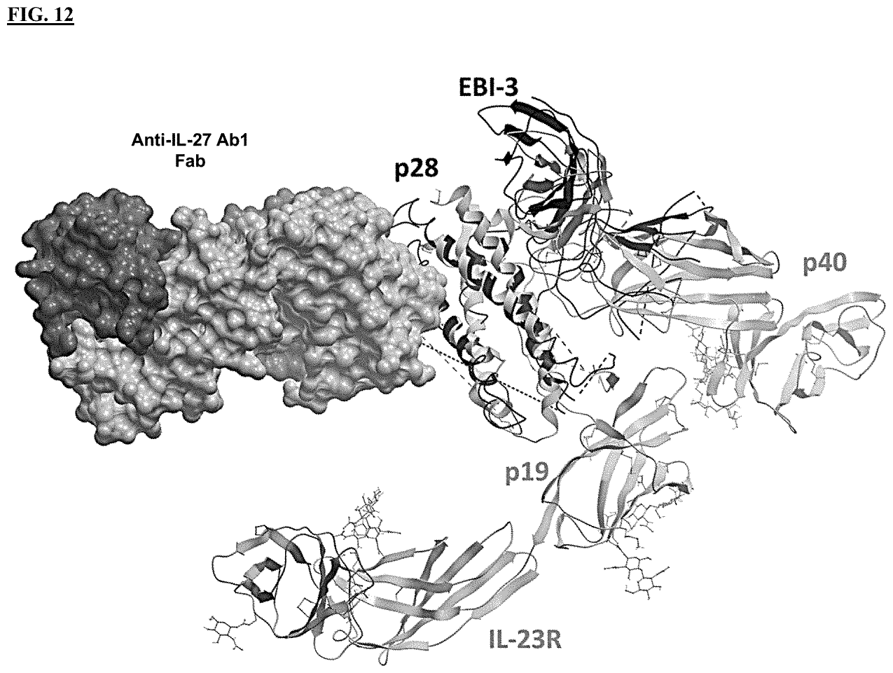

FIG. 12 shows superimposition of complexes of IL27/anti-IL-27 Ab1 with IL23/IL23R using p28 and IL6 for the alignment in 3-dimensional space. The gp130 binding site on IL6 overlaps with the anti-IL-27 Ab1 binding site on p28. However, the IL23R binding site on p19 does not overlap with the anti-IL-27 Ab1 binding site on p28.

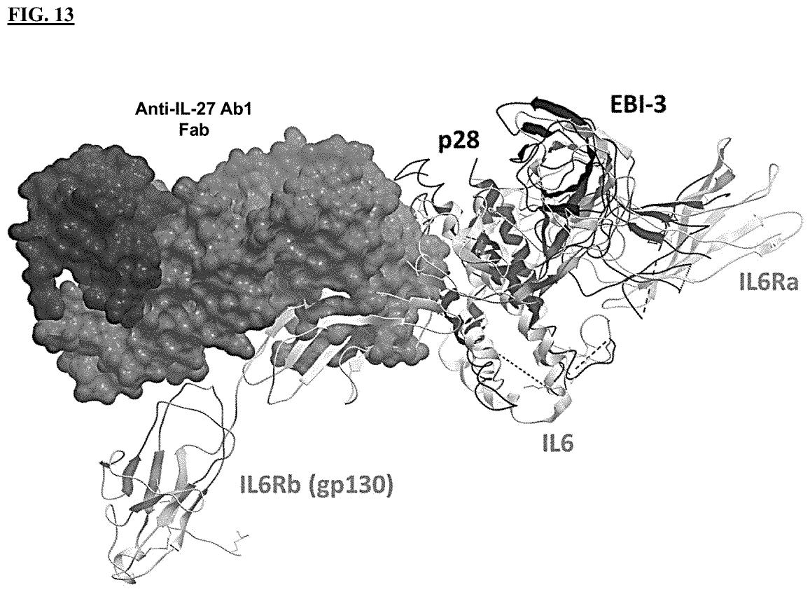

FIG. 13 shows superimposition of complexes of IL27/anti-IL-27 Ab1 with IL6/IL6Ra/gp130 using p28 and IL6 for the alignment in 3-dimensional space. Here, the gp130 binding site on IL6 again overlaps with the anti-IL-27 Ab1 binding site on p28. In addition, IL6Ra aligns with EBI-3.

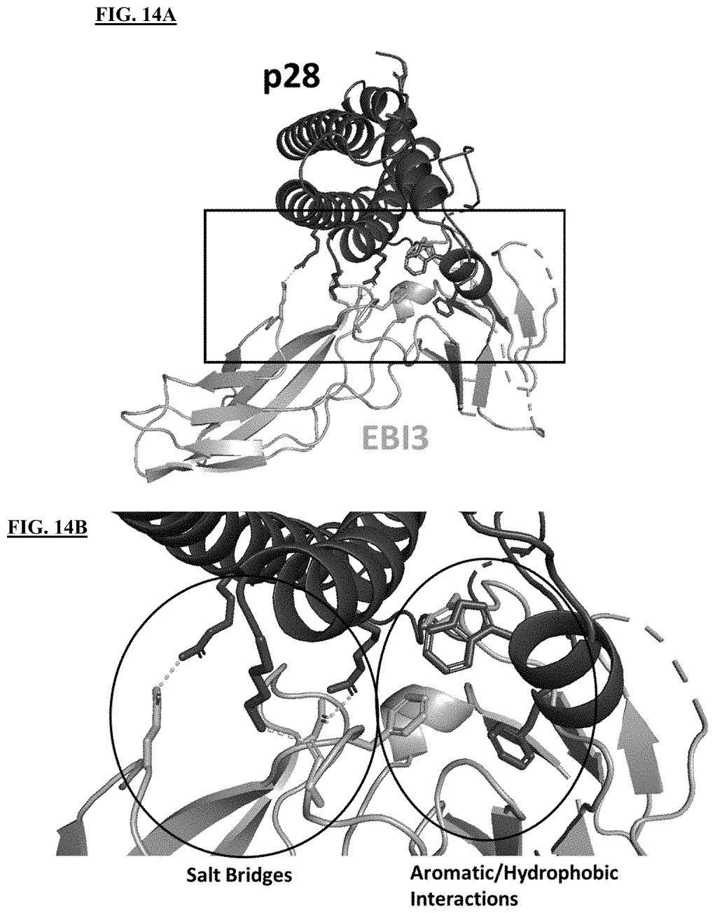

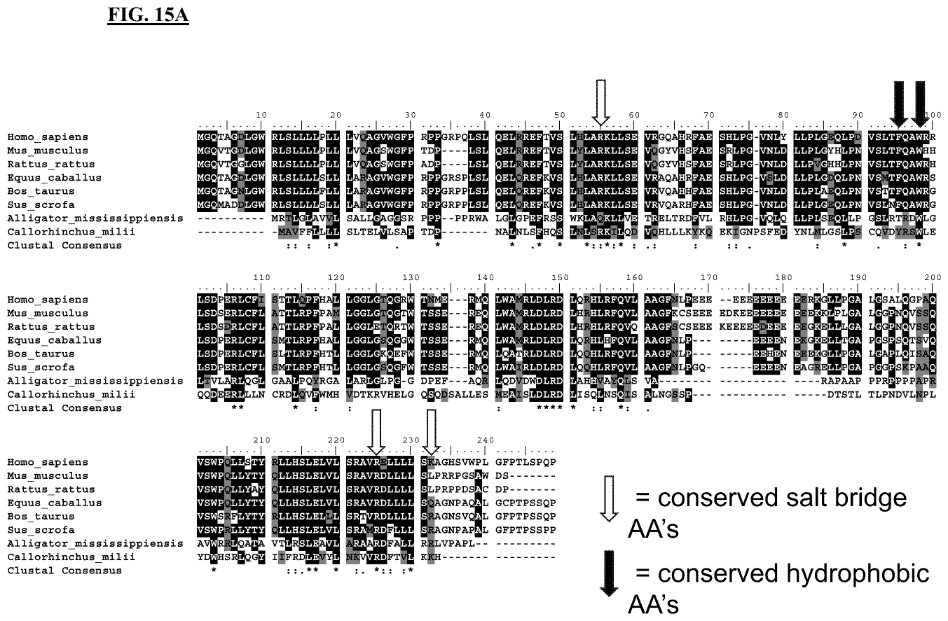

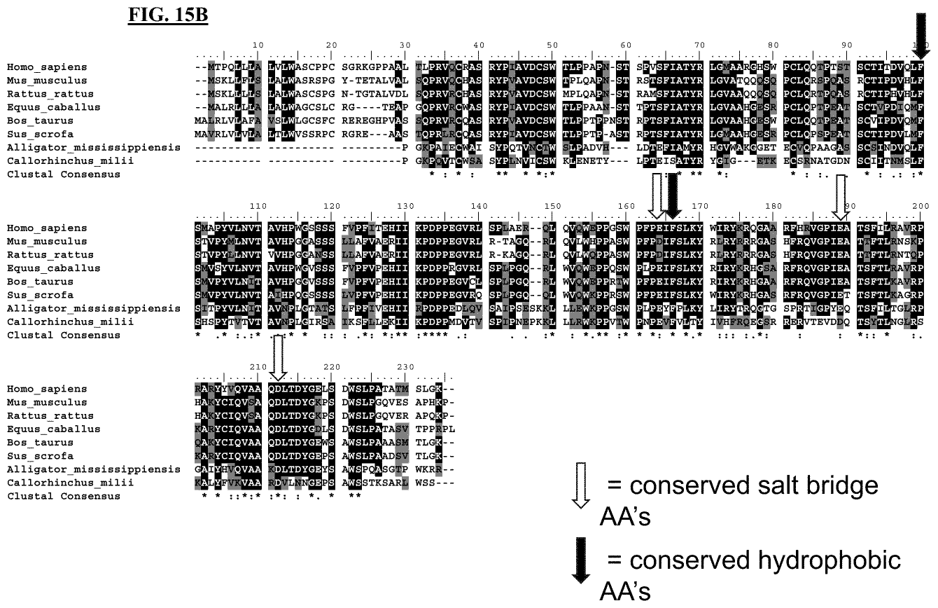

A sequence alignment across various animals reveals that the p28 residues that are involved in specific interactions with EBI3 are well conserved, and the same is true for the EBI3 residues involved in specific interactions with p28 (FIGS. 14A-15B). This includes several conserved salt bridge amino acid residues and several conserved hydrophobic amino acid residues, marked by arrows.

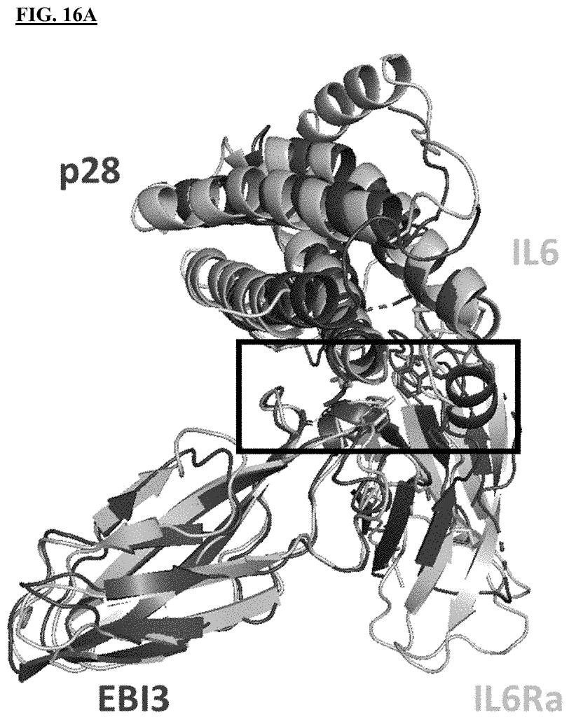

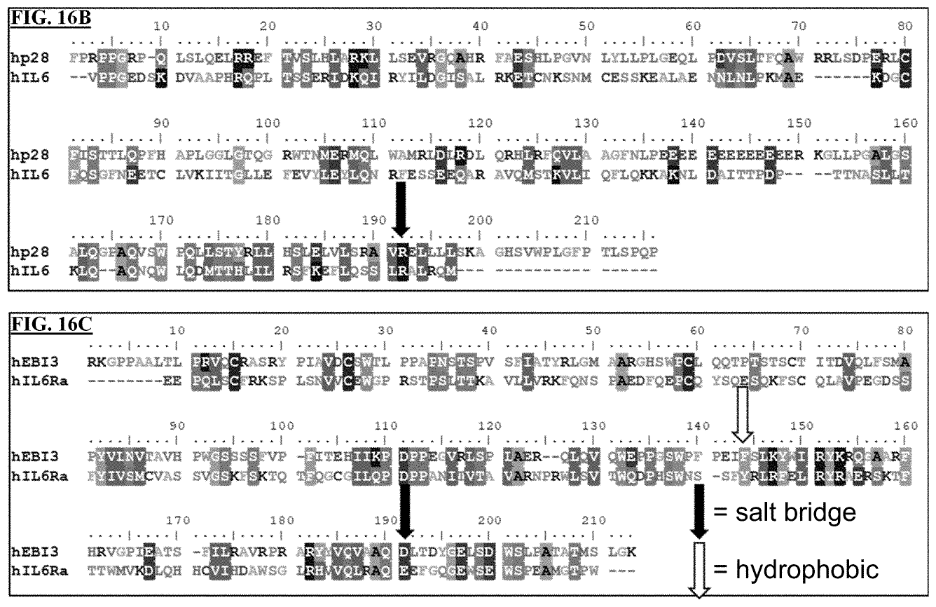

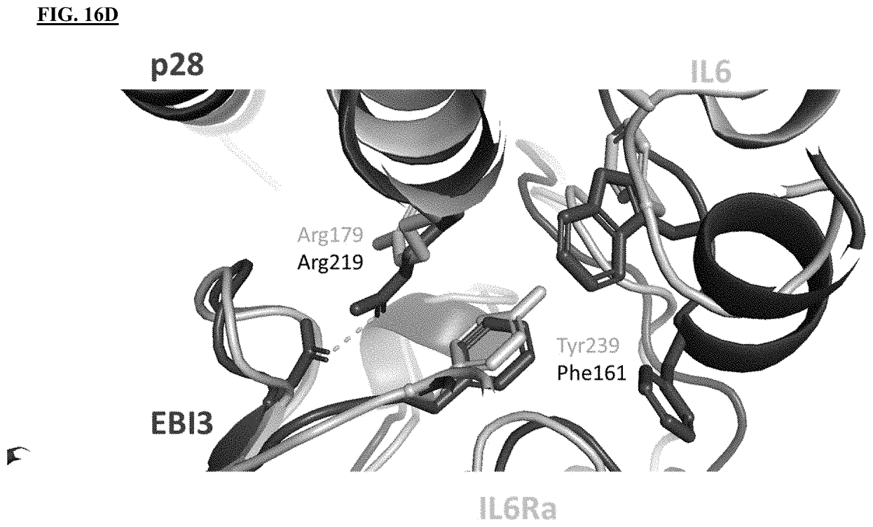

Structural alignment of IL-27 heterodimer with IL6/IL6RA shows that the secondary structure, domains, and alpha carbon backbone align well for both heterodimers (FIGS. 16A-16D), and several p28 interactions with EBI3 are potentially conserved with IL6RA (FIG. 16D).

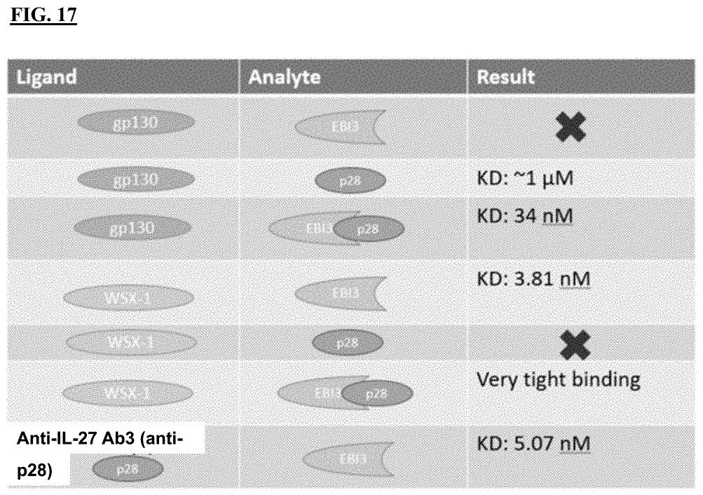

Binding affinity data for human IL-27 indicate that p28 had weak or no binding to either gp130 or WSX-1 alone (FIG. 17). EBI3 had no binding for gp130 alone, but moderate binding affinity for WSX-1. High affinity binding for Human IL27 was only observed when the heterodimer was assembled. The affinity of EBI3 for p28 was 5 nM.

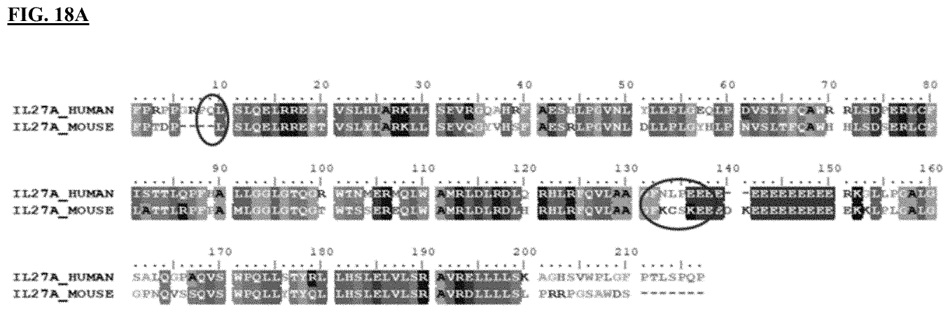

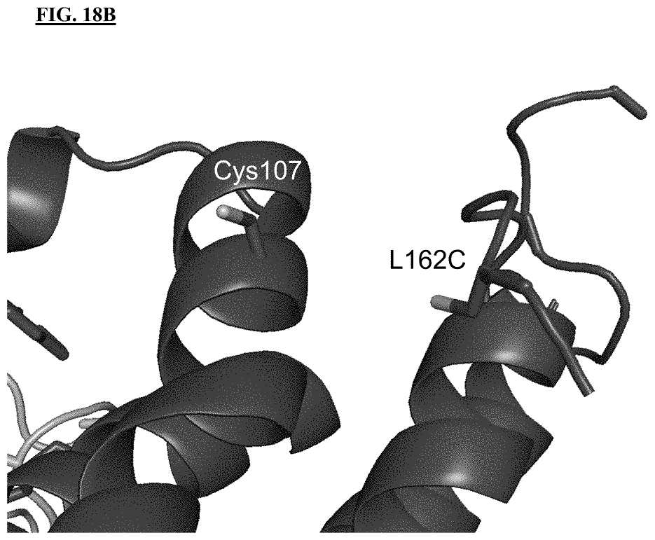

Amino acids in human p28 with anti-IL-27 Ab1 binding interactions are mostly conserved in the mouse sequence (FIG. 18A); however, anti-IL-27 Ab1 interacts with both Gln37 and Leu162 for human p28, and Gln37 is not present in the mouse sequence, and Leu162 corresponds to a Cys in the mouse sequence (FIG. 18B). Additional disulfide bonds in mouse p28 may also perturb localized structural epitope where anti-IL-27 Ab1 LC binds.

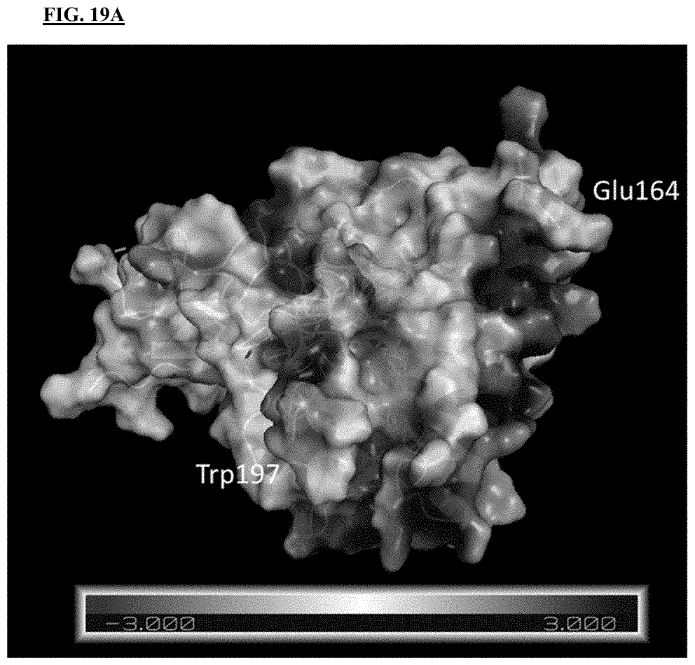

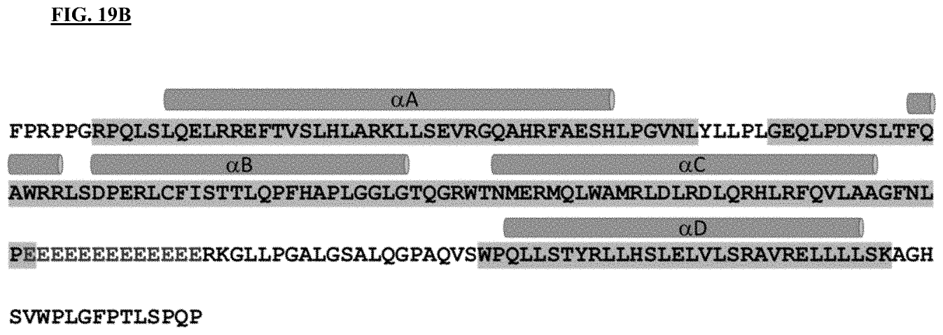

There was also an unresolved CD loop with poly-Glu sequence, including a large region of positive charges from Arg residues in αC helix (FIGS. 19A-19B).

### Example 13: Targeting of IL-27 Expression in Renal Cell Carcinoma

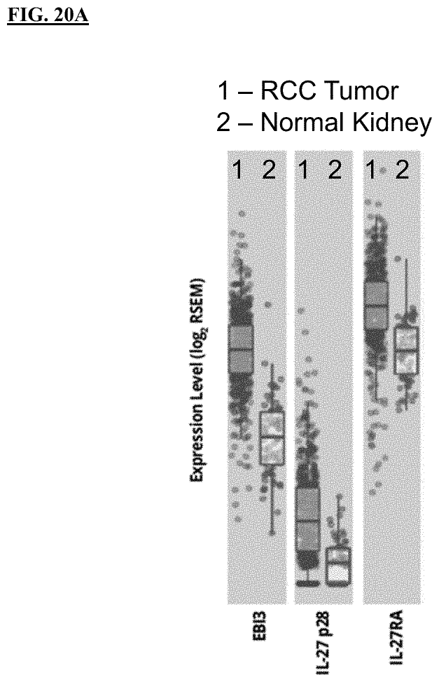

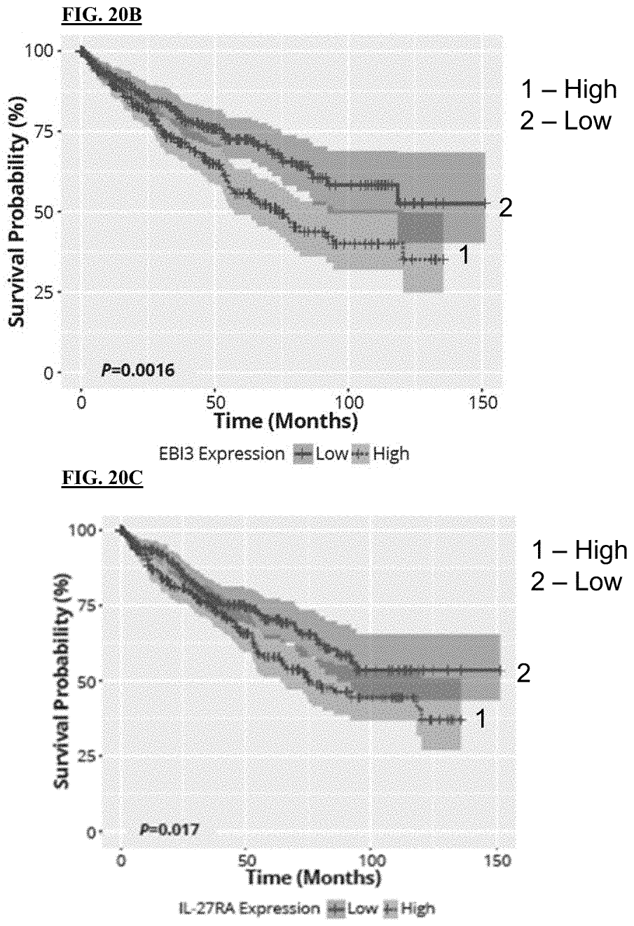

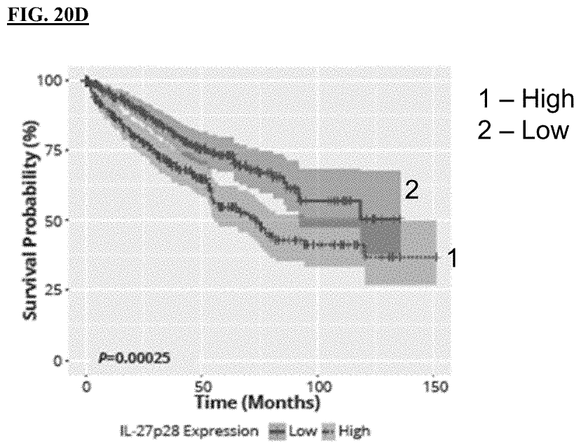

The expression of IL-27 is increased in renal cell carcinoma (RCC), with increased levels of EBI3, IL-27p28, and IL-27RA in RCC tumor tissue, relative to the expression of each in normal kidney tissue (FIG. 20A). High expression of each of EBI3 (FIG. 20B), IL-27RA (FIG. 20C), and IL-27p28 (FIG. 20D) correlates with decreased survival probability in human subjects, as compared to low expression of each of these transcripts.

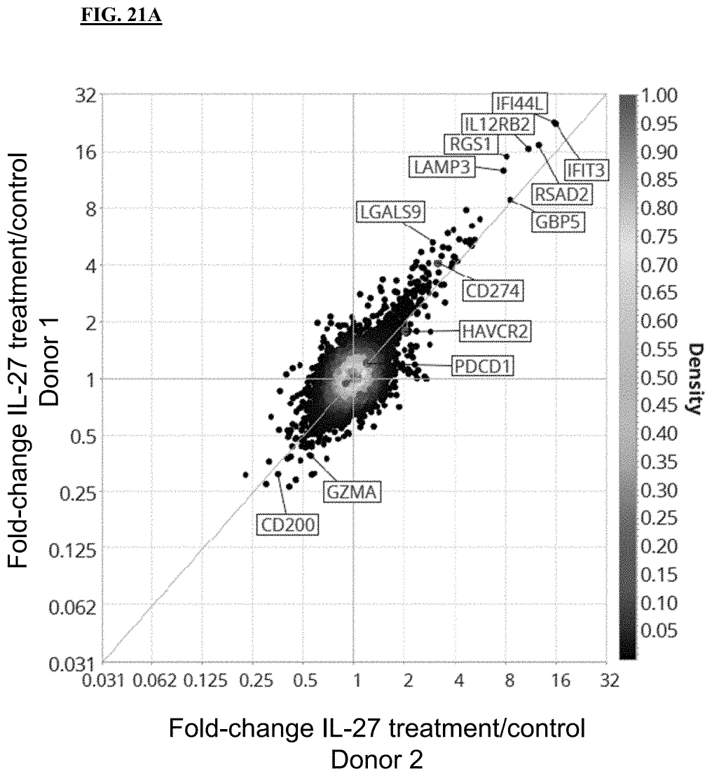

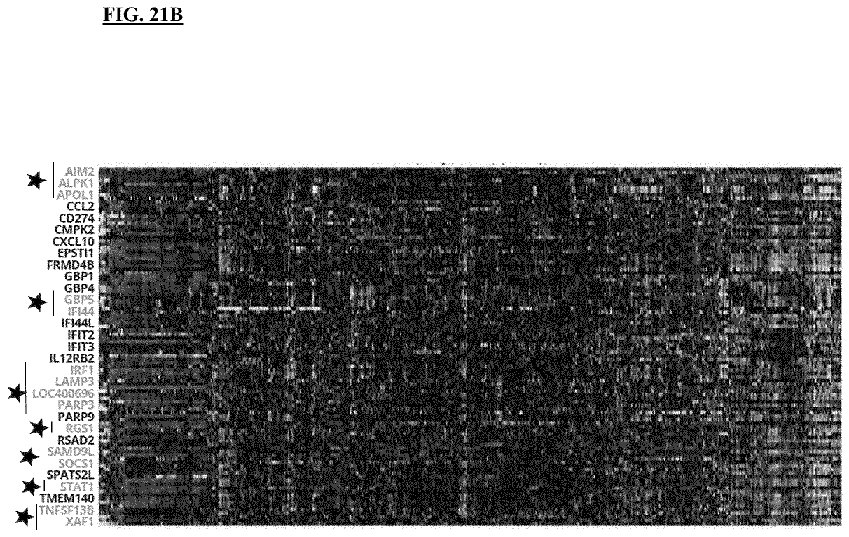

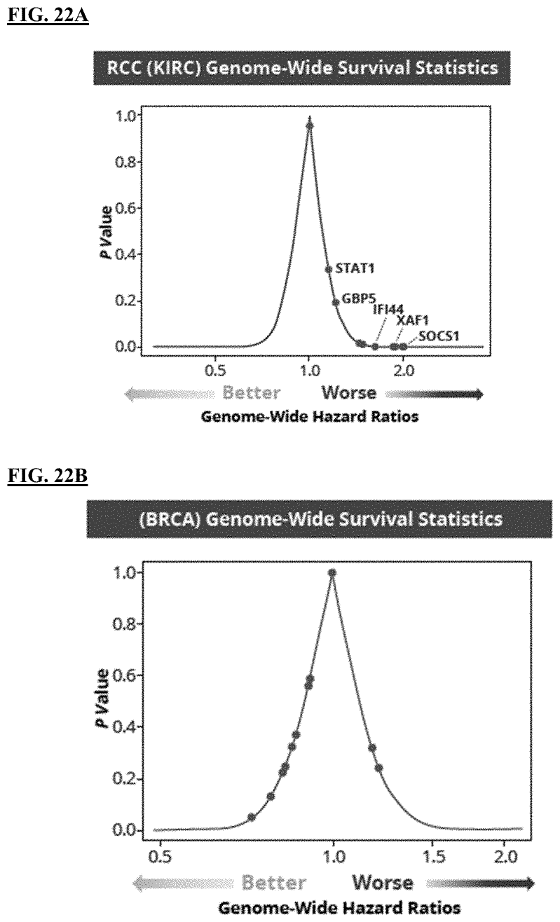

IL-27 induces a reproducible gene expression signature in activated human CD4⁺ T cells. Peripheral blood mononuclear cells (PBMCs) from individual donors were activated with anti-CD3 ±recombinant human IL-27 (rhIL-27) for 3 days. CD4⁺ T cells were FACS sorted and gene expression was analyzed by microarray. Numerous genes were observed to be upregulated or downregulated in T cells activated with CD3 and rhIL-27, including increased expression of PDCD1, HAVCR2, CD274, LGALS9, GBP5, LAMP3, RGS1, IL12RB2, RSAD2, IFIT3, and IFI44L, which are upregulated, and GZMA and CD200, which are downregulated (FIG. 21A). The top 31 genes in the IL-27 signature in CD4⁺ T cells are listed in FIG. 21B. Notably, 15 of the 31 genes were associated with poor outcome, namely AIM2, ALPK1, APOL1, GBP5, IFI44, IRF1, LAMP3, LOC400696, PARP3, RGS1, SAMD9L, SOCS1, STAT1, TNFSF13B, and XAF1. Twelve of the top signature genes (including STAT1, GBP5, IFI44, XAF1, and SOCS1) were associated with poor outcomes in RCC (FIG. 22A) but these same genes were not associated with poor outcome in breast cancer (BRCA) (FIG. 22B).

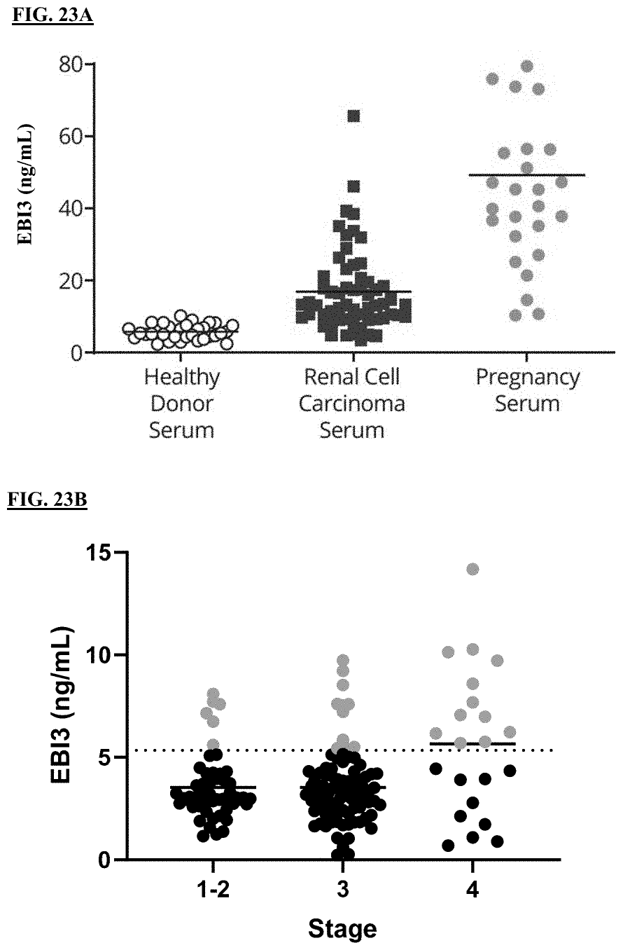

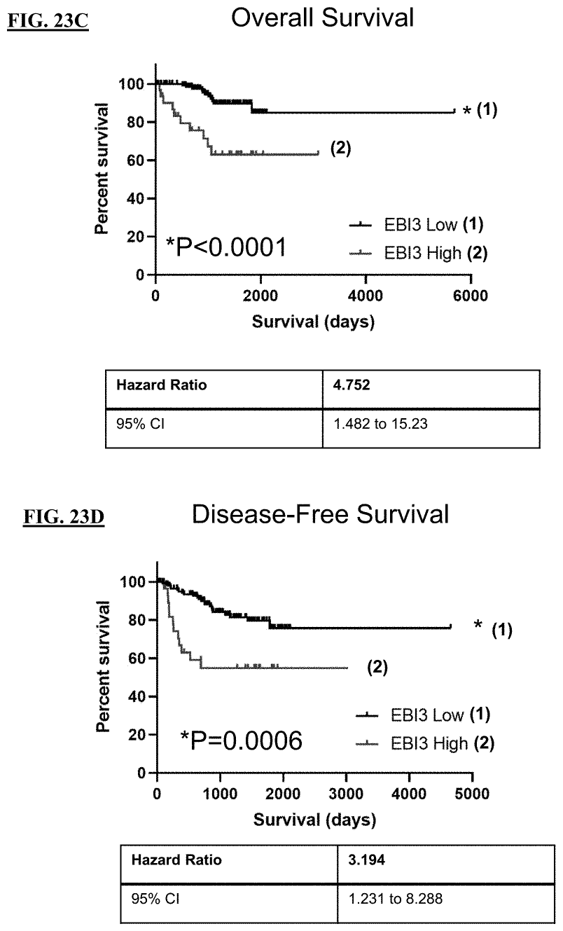

Further, plasma levels of EBI3 in RCC patients can be predictraive of outcome. Serum samples were collected from RCC patients at the time of nephrectomy surgery, and EBI3 levels were measured using an EBI3-specific antibody pair. Average EBI3 levels were elevated in serum from patients with RCC as compared with serum from healthy donors (FIG. 23A). Serum from pregnant donors was included as a positive control. EBI3 levels were highest in subjects stage 4 RCC relative to stage 2 or stage 3 (FIG. 23B); and overall survival (FIG. 23C) and disease-free survival (FIG. 23D) were higher in RCC patients with low serum EBI3 levels.

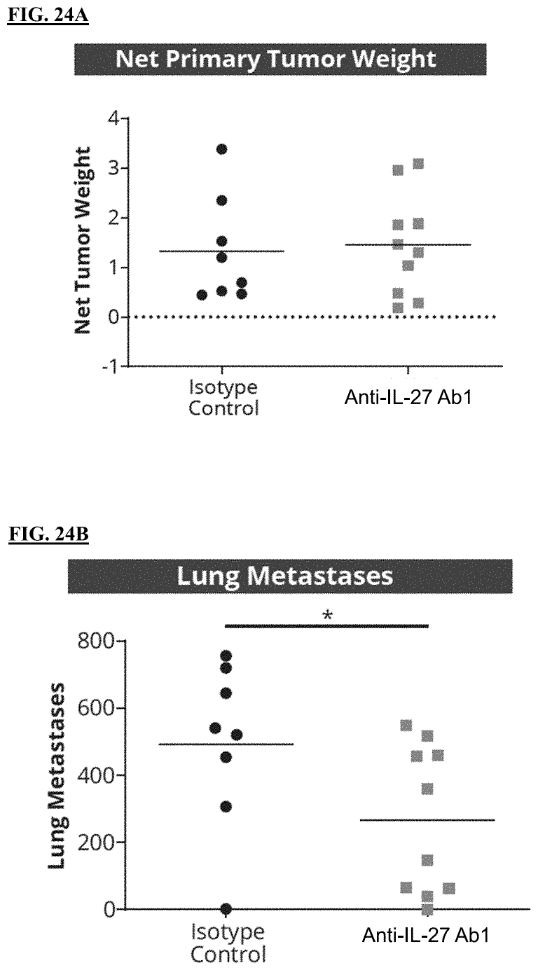

To test the in vivo efficacy of anti-IL-27 Ab1 treatment in a murine model of RCC, Renca cells were orthotopically implanted into the left kidney. Three days postimplant mice were treated intraperitoneally with anti-IL-27 Ab1 or human IgG1 isotype control (50 mg/kg twice weekly) for 2 weeks. After 21 days tissues were harvested, both kidneys were weighed to calculate net tumor weight, and lung metastases were counted visually. Though average tumor weight remained largely constant (FIG. 24A), lung metastasis was significantly reduced in anti-IL-27 Ab1-treated mice as compared to isotype control-treated mice (FIG. 24B).

These data show that increased IL-27p28, EBI3, and IL-27RA transcript levels in tumors from patients with RCC are associated with a poor prognosis. Anti-IL-27 Ab1 demonstrates single-agent activity in an orthotopic model of RCC in vivo, and blockade of IL-27 with anti-IL-27 Ab1 represents a promising strategy for patients with RCC who have high levels of circulating EBI3.

### Example 14: In Vivo Targeting of IL-27 Using Anti-IL-27 Ab1 in an Orthotopic Hepa1-6 HCC Mouse Model

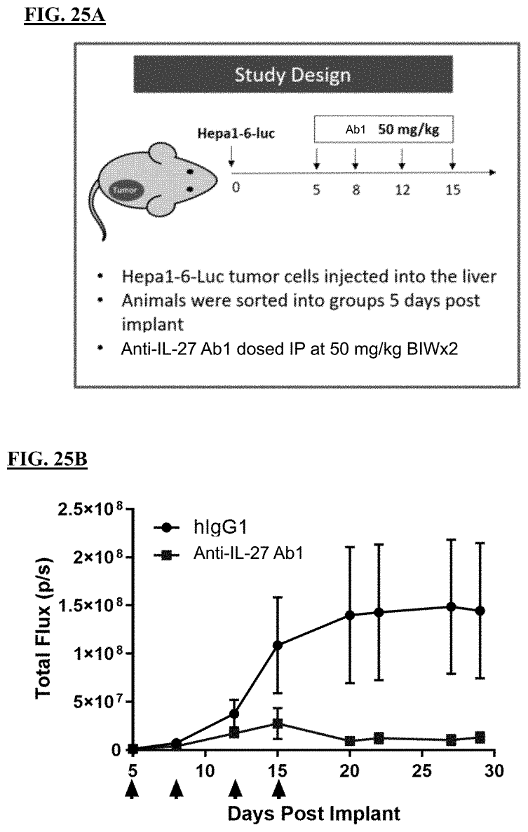

To study the in vivo efficacy of anti-IL-27 Ab1 antibody in a liver cancer model, Hepa1-6-Luc tumor cells injected into the liver of a mouse, and animals were dosed at days 5, 8, 12, and 15 post-implant with 50 mg/kg anti-IL-27 Ab1 by IP injection (FIG. 25A). Total flux was reduced to near baseline in anti-IL-27 Ab1 treated mice as compared to hIgG1 isotype control-treated mice (FIG. 25B).

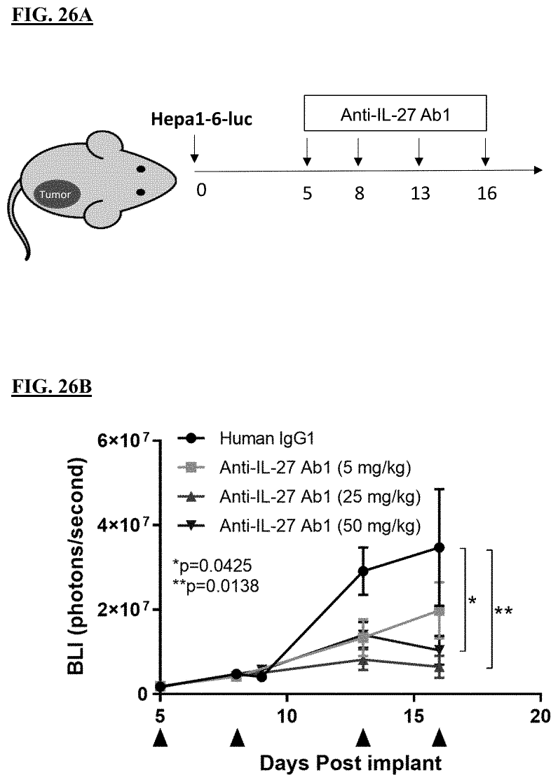

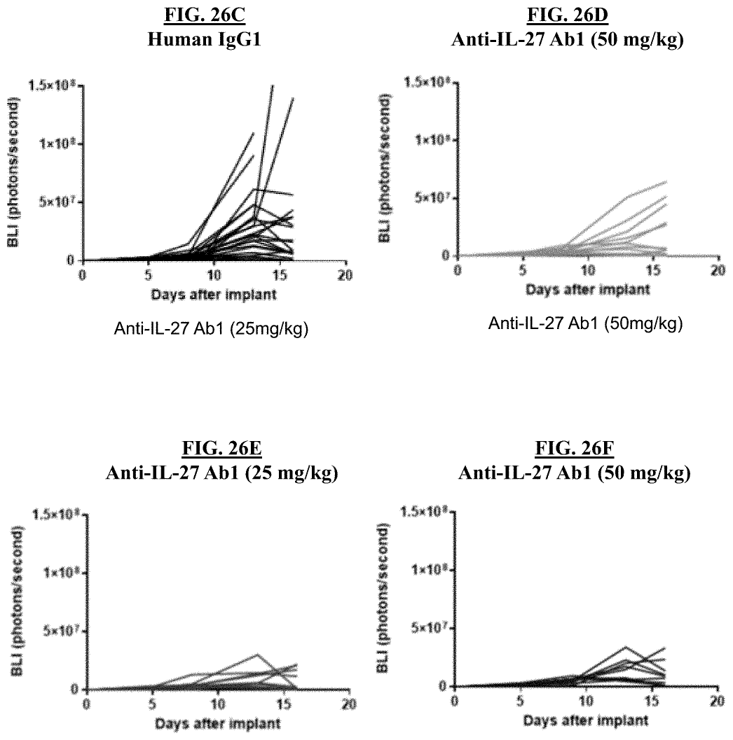

Responsiveness to anti-IL-27 Ab1 in the mouse model was dose dependent. Mice were treated with an isotype control, 5 mg/kg anti-IL-27 Ab1, 25 mg/kg anti-IL-27 Ab1, or 50 mg/kg anti-IL-27 Ab1 at days 5, 8, 12, and 15 post-implant (FIG. 26A). Tumor growth was lowest, near baseline, in mice treated with 25 or 50 mg/kg anti-IL-27 Ab1 (FIGS. 26B-26F).

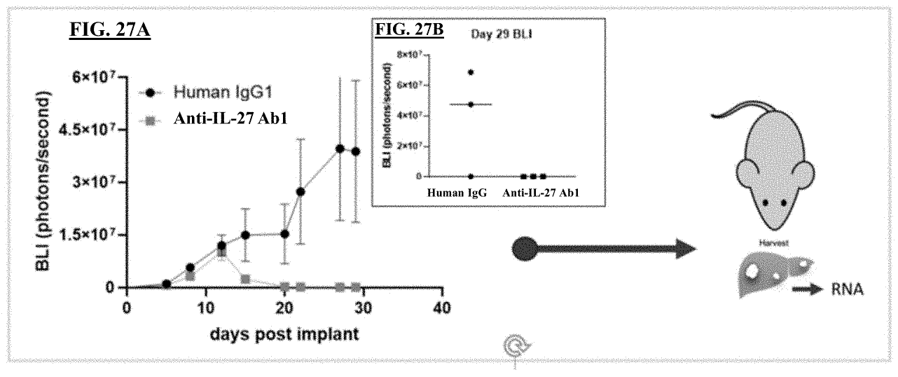

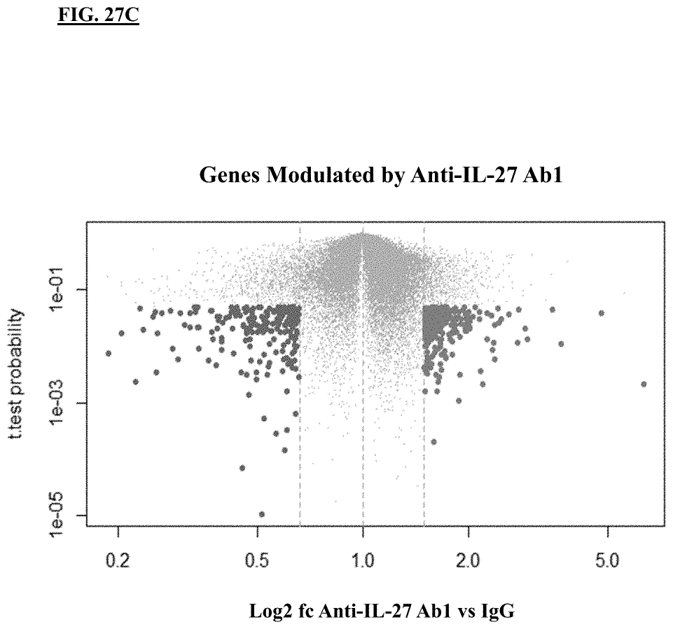

To determine whether previously defined preclinical changes in gene expression induced by anti-IL-27 Ab1 are relevant in this model, the expression of an array of biomarker genes were assays following administration of anti-IL-27 Ab1 (FIGS. 27A-27C). The top 200 repressed genes are shown in Table 17A, and the top 200 induced genes are shown in Table 17B. The full lists of upregulated and downregulated genes are presented above in Tables 11A-11B.

TABLE 17A
Top 200 repressed genes in Hepa1-6 mouse livers following
anti-IL-27 Ab1 administration.

| | Fold | |
|---|---|---|
| Gene | Change | p Value |
| DEFB44-PS | 0.18731 | 0.16963 |
| C1S2 | 0.18735 | 0.00760 |
| HIST1H1B | 0.20429 | 0.13468 |
| GM15114 | 0.20467 | 0.11272 |
| GM2005 | 0.20501 | 0.01692 |
| KHDC1C | 0.21082 | 0.08196 |
| GM2005 | 0.22434 | 0.00235 |
| SERPINB7 | 0.22443 | 0.13886 |
| SERPINB11 | 0.22512 | 0.05938 |
| CXCL3 | 0.22982 | 0.15165 |
| CDH10 | 0.23008 | 0.04656 |
| M1R1949 | 0.23543 | 0.13071 |
| SLC7A11 | 0.23650 | 0.01951 |
| LUZP4 | 0.24896 | 0.09520 |
| IGHV1-81 | 0.25160 | 0.51163 |
| PTPRTOS | 0.25229 | 0.03379 |
| RP1 | 0.25355 | 0.15378 |
| GM15127 | 0.25399 | 0.05002 |
| CRISP1 | 0.25472 | 0.08655 |
| GM15107 | 0.25489 | 0.05910 |
| SERPINB5 | 0.25593 | 0.04019 |
| GM7665 | 0.25600 | 0.00338 |
| TMEM252 | 0.25889 | 0.01729 |
| PNMA5 | 0.26178 | 0.09220 |
| CXCL15 | 0.26260 | 0.10373 |
| CLEC2G | 0.26543 | 0.03883 |
| MMP13 | 0.26736 | 0.16866 |
| GM15093 | 0.26907 | 0.07413 |
| VMN1R53 | 0.27031 | 0.06485 |
| GM14409 | 0.27619 | 0.15580 |
| GM11884 | 0.27767 | 0.16819 |
| SLC16A4 | 0.27777 | 0.11590 |
| MMP12 | 0.27973 | 0.07864 |
| KIF5C | 0.28118 | 0.10032 |
| APELA | 0.28255 | 0.06005 |
| AFP | 0.28256 | 0.04236 |
| IGKV1-122 | 0.28592 | 0.00900 |
| A630095E13RIK | 0.29162 | 0.09055 |
| GM15109 | 0.29701 | 0.00582 |
| OLFR111 | 0.29889 | 0.21068 |
| TNS4 | 0.30067 | 0.03812 |
| PLEKHS1 | 0.30148 | 0.07842 |
| LNCENC1 | 0.30470 | 0.09388 |
| THBS1 | 0.30908 | 0.02429 |
| PLATR14 | 0.31317 | 0.08123 |
| RASSF9 | 0.31383 | 0.11251 |
| ITGA2 | 0.31895 | 0.06993 |
| LOC102634388 | 0.32002 | 0.02127 |
| LOC102634388 | 0.32002 | 0.02127 |
| GM15093 | 0.32036 | 0.03800 |
| CEP55 | 0.32092 | 0.10073 |
| A630038E17RIK | 0.32118 | 0.13340 |
| GM25552 | 0.32317 | 0.12614 |
| RPS26 | 0.32662 | 0.18403 |
| STRA6 | 0.33040 | 0.04984 |
| GM15093 | 0.33144 | 0.04030 |
| PSAT1 | 0.33163 | 0.09265 |
| AKR1C18 | 0.33224 | 0.09827 |
| DEPDC1A | 0.33251 | 0.18136 |
| GM10439 | 0.33495 | 0.06941 |
| GM24916 | 0.33509 | 0.20312 |
| STC1 | 0.33666 | 0.08291 |
| DPPA2 | 0.33711 | 0.08874 |
| E030011O05RIK | 0.33714 | 0.04863 |
| GM20756 | 0.33749 | 0.02179 |
| GM22771 | 0.33862 | 0.28971 |
| IGKV6-32 | 0.33990 | 0.44350 |
| AIM2 | 0.34040 | 0.00912 |
| C920009B18RIK | 0.34064 | 0.02201 |
| BUB1 | 0.34878 | 0.07548 |
| TICRR | 0.34960 | 0.17718 |
| MIS18BP1 | 0.35250 | 0.09094 |
| MAGEA6 | 0.35303 | 0.20666 |
| CHIL3 | 0.35389 | 0.06510 |
| IGHV1-78 | 0.35720 | 0.28499 |
| STIL | 0.35848 | 0.14130 |
| GM26735 | 0.36001 | 0.06537 |
| REG2 | 0.36041 | 0.49690 |
| SPRR1A | 0.36134 | 0.00562 |
| PARPBP | 0.36204 | 0.14708 |
| PADI4 | 0.36592 | 0.09746 |
| GM14139 | 0.36657 | 0.04225 |
| GPC3 | 0.36787 | 0.21392 |
| MS4A6D | 0.37021 | 0.01764 |
| ATP10A | 0.37100 | 0.09774 |
| KIF23 | 0.37107 | 0.04316 |
| GM20757 | 0.37125 | 0.22483 |
| GM2318 | 0.37176 | 0.01361 |
| GM2318 | 0.37176 | 0.01361 |
| GM2318 | 0.37176 | 0.01361 |
| GM2318 | 0.37176 | 0.01361 |
| IGHV1-9 | 0.37183 | 0.35845 |
| CPB1 | 0.37389 | 0.54454 |
| TNFSF4 | 0.37410 | 0.14201 |
| RNASE1 | 0.37521 | 0.29355 |
| TIMP1 | 0.37633 | 0.07652 |
| GM6020 | 0.37895 | 0.48362 |
| 1700049E17RIK2 | 0.37958 | 0.00452 |
| IGH-VJ558 | 0.38192 | 0.43479 |
| PRRG4 | 0.38250 | 0.10001 |
| GM7391 | 0.38252 | 0.02063 |
| GM7391 | 0.38252 | 0.02063 |
| GM7391 | 0.38252 | 0.02063 |
| GM7391 | 0.38252 | 0.02063 |
| GM7391 | 0.38252 | 0.02063 |
| GM7391 | 0.38252 | 0.02063 |
| CD109 | 0.38422 | 0.08923 |
| TMEM173 | 0.38467 | 0.09117 |
| CDH17 | 0.38479 | 0.03381 |
| KITL | 0.38506 | 0.02871 |
| SKA3 | 0.38794 | 0.19907 |
| PRC1 | 0.38977 | 0.07960 |
| MBOAT1 | 0.39032 | 0.03388 |
| TOP2A | 0.39357 | 0.06590 |
| 1700049E17RIK2 | 0.39548 | 0.00842 |
| SPEER4D | 0.39557 | 0.01189 |
| HIST1H3I | 0.39572 | 0.38176 |
| TUBA1A | 0.39616 | 0.01385 |
| GM22265 | 0.39624 | 0.09085 |
| TRBJ1-7 | 0.39684 | 0.15669 |
| KIF20B | 0.39753 | 0.09225 |
| SERPINB2 | 0.39766 | 0.02587 |
| GM14402 | 0.39768 | 0.24963 |
| PLK4 | 0.39883 | 0.07277 |
| C330027C09RIK | 0.40017 | 0.10461 |
| GM15091 | 0.40058 | 0.07631 |
| NDC80 | 0.40091 | 0.07729 |
| MYBL1 | 0.40096 | 0.16592 |
| KIF2C | 0.40131 | 0.07030 |
| GM12603 | 0.40134 | 0.08558 |
| CYP11A1 | 0.40271 | 0.26346 |
| FANCI | 0.40316 | 0.17817 |
| CCNA2 | 0.40376 | 0.05984 |
| RACGAP1 | 0.40619 | 0.07336 |
| NCAPH | 0.40648 | 0.11248 |
| GM16094 | 0.40657 | 0.03168 |
| CH25H | 0.40668 | 0.30753 |
| GM15398 | 0.40844 | 0.10428 |
| KNTC1 | 0.40858 | 0.13345 |
| BST1 | 0.40915 | 0.09282 |
| KIF11 | 0.41151 | 0.07520 |
| GM23576 | 0.41226 | 0.26181 |
| SHCBP1 | 0.41247 | 0.13607 |
| IGHV1-42 | 0.41274 | 0.44744 |
| GPRC5A | 0.41537 | 0.03331 |
| TNERSF10B | 0.41617 | 0.10473 |
| IL23A | 0.41843 | 0.14139 |
| ERICH2 | 0.41910 | 0.14436 |
| ANLN | 0.41967 | 0.04088 |
| CASC5 | 0.41996 | 0.11775 |
| GM17689 | 0.42053 | 0.27734 |
| SOX4 | 0.42211 | 0.07946 |
| GM22069 | 0.42215 | 0.04943 |
| PRR11 | 0.42277 | 0.07500 |
| SEMA3C | 0.42285 | 0.02076 |
| RETNLA | 0.42307 | 0.04289 |
| FRMD7 | 0.42356 | 0.02106 |
| TNERSF11B | 0.42500 | 0.05920 |
| RAD54L | 0.42591 | 0.16039 |
| GM10488 | 0.42650 | 0.00744 |
| PLAT | 0.42677 | 0.06470 |
| GM13790 | 0.42685 | 0.11605 |
| DTL | 0.42697 | 0.16996 |
| SERPINB9B | 0.42886 | 0.10643 |
| ASNS | 0.42974 | 0.04714 |
| GM15097 | 0.42977 | 0.17217 |
| TMED6 | 0.43183 | 0.26485 |
| SERPINE1 | 0.43312 | 0.03450 |
| TPX2 | 0.43325 | 0.05879 |
| CENPK | 0.43347 | 0.17724 |
| 2810429I04RIK | 0.43385 | 0.21706 |
| 4930461G14RIK | 0.43432 | 0.17990 |
| HIST1H2AG | 0.43516 | 0.18735 |
| IER3 | 0.43524 | 0.14140 |
| CHRNB1 | 0.43554 | 0.04999 |
| GM5431 | 0.43690 | 0.13001 |
| GM13247 | 0.43774 | 0.20349 |
| AI506816 | 0.43795 | 0.07730 |
| GM7942 | 0.43806 | 0.04319 |
| CCNB1 | 0.44021 | 0.03617 |
| ZFP345 | 0.44040 | 0.24896 |
| IGHV8-8 | 0.44047 | 0.45075 |
| ATAD5 | 0.44240 | 0.11428 |
| KDELR3 | 0.44372 | 0.08725 |
| CDC25C | 0.44462 | 0.03533 |
| IGKV4-72 | 0.44464 | 0.18026 |
| OLFR99 | 0.44744 | 0.16218 |
| GM25544 | 0.44759 | 0.13854 |
| MIR101C | 0.44785 | 0.08179 |
| HYDIN | 0.44832 | 0.14992 |
| IGHV5-16 | 0.44859 | 0.32098 |
| NOP58 | 0.44866 | 0.13787 |
| CEL | 0.44913 | 0.50615 |
| MS4A4A | 0.44926 | 0.11256 |
| BCL2A1B | 0.44968 | 0.00346 |
| IGKV4-59 | 0.44969 | 0.49749 |
| CCL3 | 0.45007 | 0.10701 |
| GM2933 | 0.45077 | 0.16505 |
| FAM102B | 0.45149 | 0.13304 |

TABLE 17B
Top 200 induced genes in Hepa1-6 mouse livers following
anti-IL-27 Ab1 administration.

| | Fold | |
|---|---|---|
| Gene | Change | p Value |
| MUP-PS12 | 6.33152 | 0.00215 |
| GM26184 | 5.61658 | 0.08504 |
| GKN2 | 4.82174 | 0.03776 |
| MYH8 | 3.85386 | 0.31903 |
| MIR101B | 3.69523 | 0.01108 |
| GKN1 | 3.48462 | 0.04511 |
| SLN | 3.47316 | 0.18221 |
| MUC5AC | 3.34061 | 0.06276 |
| 2310057J18RIK | 3.04114 | 0.41002 |
| PSCA | 3.02691 | 0.12186 |
| GM25623 | 2.94838 | 0.01329 |
| FAM83B | 2.90452 | 0.02016 |
| GM23852 | 2.88192 | 0.07627 |
| SPTSSB | 2.87259 | 0.09524 |
| MIRLET7F-1 | 2.79416 | 0.04469 |
| MUP-PS16 | 2.77768 | 0.01138 |
| ANXA10 | 2.68901 | 0.10472 |
| LGALS2 | 2.68519 | 0.13369 |
| CHIA1 | 2.67246 | 0.40779 |
| AKP3 | 2.66042 | 0.36655 |
| SLC13A1 | 2.62180 | 0.29356 |
| GM766 | 2.62086 | 0.40345 |
| GM24537 | 2.61729 | 0.16494 |
| GM24138 | 2.60556 | 0.03739 |
| SIS | 2.58535 | 0.37774 |
| 2210407C18RIK | 2.58374 | 0.06005 |
| S100G | 2.58040 | 0.38773 |
| GM26354 | 2.54047 | 0.10968 |
| CKMT2 | 2.53798 | 0.26801 |
| GM26055 | 2.52487 | 0.21572 |
| GM5885 | 2.47858 | 0.03053 |
| AGR2 | 2.45816 | 0.18450 |
| VMN1R167 | 2.45428 | 0.09317 |
| GM25167 | 2.44373 | 0.02641 |
| ATP5C1-PS | 2.43743 | 0.04767 |
| GM24470 | 2.43383 | 0.16763 |
| MYH2 | 2.40309 | 0.16324 |
| MIR326 | 2.37544 | 0.00590 |
| MIR122 | 2.37543 | 0.02420 |
| MCPT9 | 2.37520 | 0.06227 |
| PSG18 | 2.35852 | 0.01151 |
| MUC6 | 2.35850 | 0.31084 |
| VMN2R115 | 2.35187 | 0.11114 |
| GM25498 | 2.35108 | 0.00863 |
| 2010106E10RIK | 2.33919 | 0.46065 |
| ERBB4 | 2.32759 | 0.03502 |
| LYPD8 | 2.31999 | 0.13737 |
| CYP2A5 | 2.30476 | 0.23296 |
| BTNL5-PS | 2.30131 | 0.16835 |
| GM24549 | 2.28941 | 0.11379 |
| GM24465 | 2.28686 | 0.23556 |
| SLC26A3 | 2.28613 | 0.34866 |
| DPCR1 | 2.28459 | 0.06528 |
| GM11844 | 2.27856 | 0.10541 |
| CYP4A32 | 2.26879 | 0.21331 |
| MIR1928 | 2.26702 | 0.01338 |
| GKN3 | 2.26609 | 0.38398 |
| KRT4 | 2.26351 | 0.15392 |
| PGC | 2.26227 | 0.25834 |
| HIST1H2BA | 2.26012 | 0.03903 |
| SPRR2A3 | 2.25406 | 0.19740 |
| IGKV12-41 | 2.23462 | 0.35478 |
| PARD3BOS3 | 2.22662 | 0.11494 |
| CLCA4B | 2.22470 | 0.41766 |
| GM23744 | 2.20755 | 0.12172 |
| VMN2R-PS129 | 2.20013 | 0.00212 |
| GM23241 | 2.19443 | 0.16439 |
| MYOZ2 | 2.19124 | 0.20907 |
| MIR192 | 2.19083 | 0.05530 |
| MYH1 | 2.18765 | 0.16532 |
| 5033403H07RIK | 2.18709 | 0.12308 |
| MIRLET7F-2 | 2.18612 | 0.16167 |
| GM25009 | 2.18474 | 0.12429 |
| MUC13 | 2.18238 | 0.44121 |
| PPP1R3A | 2.17804 | 0.19680 |
| OLFR798 | 2.17172 | 0.00360 |
| SERPINA12 | 2.15429 | 0.03094 |
| GM25864 | 2.15155 | 0.04041 |
| N-R5S155 | 2.15090 | 0.06906 |
| CYP2C65 | 2.14174 | 0.14376 |
| GM14750 | 2.14105 | 0.05245 |
| GM23026 | 2.13427 | 0.08632 |
| HAMP2 | 2.12517 | 0.26750 |
| GM24527 | 2.12509 | 0.07768 |
| OLFR891 | 2.12488 | 0.07314 |
| OLFR68 | 2.12477 | 0.17461 |
| TRAV6-2 | 2.12335 | 0.16838 |
| CYP4F40 | 2.11940 | 0.18482 |
| TRDN | 2.11902 | 0.23956 |
| CFTR | 2.11685 | 0.32080 |
| XIRP2 | 2.10948 | 0.20311 |
| CYP8B1 | 2.10691 | 0.09121 |
| GM23629 | 2.10532 | 0.05899 |
| GM25076 | 2.10385 | 0.09801 |
| 1700080G11RIK | 2.10309 | 0.16206 |
| N-R5S96 | 2.09968 | 0.04925 |
| GM11027 | 2.09669 | 0.30087 |
| SLC22A29 | 2.08861 | 0.26634 |
| GSTT2 | 2.08187 | 0.23716 |
| GM23911 | 2.08111 | 0.19027 |
| OLFR724 | 2.07902 | 0.09250 |
| GM23277 | 2.07764 | 0.09120 |
| A4GNT | 2.07584 | 0.24306 |
| MUP21 | 2.07390 | 0.11490 |
| GM24147 | 2.07383 | 0.26694 |
| MYBPC1 | 2.06732 | 0.13917 |
| CYP3A59 | 2.06303 | 0.26277 |
| ACTN2 | 2.06253 | 0.14714 |
| MIR29A | 2.06200 | 0.03405 |
| TFF1 | 2.06146 | 0.08032 |
| PLA2G1B | 2.05956 | 0.44772 |
| GM23021 | 2.05954 | 0.11112 |
| GM22607 | 2.04477 | 0.13834 |
| RPL10L | 2.04430 | 0.06920 |
| ACE2 | 2.03841 | 0.35049 |
| PRAMEL3 | 2.03798 | 0.14902 |
| SULT1B1 | 2.03237 | 0.24951 |
| ARL14 | 2.02633 | 0.09522 |
| GM11337 | 2.02394 | 0.02702 |
| CNN1 | 2.02048 | 0.02173 |
| LRIT2 | 2.01905 | 0.05334 |
| ACOT4 | 2.01479 | 0.21202 |
| LOC100125594 | 2.01433 | 0.02079 |
| GLB1L2 | 2.01156 | 0.03963 |
| GM25605 | 2.00818 | 0.13287 |
| GM15384 | 2.00171 | 0.02548 |
| GM94 | 1.99674 | 0.10946 |
| OLFR1113 | 1.99669 | 0.12774 |
| G630018N14RIK | 1.99666 | 0.30168 |
| GM23232 | 1.99430 | 0.06721 |
| GM16378 | 1.99415 | 0.03571 |
| GM22838 | 1.99362 | 0.08752 |
| HSD3B5 | 1.99243 | 0.20188 |
| OLFR746 | 1.98892 | 0.03312 |
| GM24254 | 1.98755 | 0.13006 |
| GM24232 | 1.98629 | 0.22120 |
| PRAP1 | 1.98616 | 0.37196 |
| ALPI | 1.98615 | 0.41922 |
| MIR3964 | 1.98298 | 0.03007 |
| GM22787 | 1.98276 | 0.01829 |
| GM26081 | 1.98214 | 0.17707 |
| GM22242 | 1.98012 | 0.31686 |
| IGHV1-77 | 1.97991 | 0.47423 |
| OLFR740 | 1.97966 | 0.21907 |
| OLFR1201 | 1.97856 | 0.03392 |
| GM22654 | 1.97558 | 0.51008 |
| OLFR747 | 1.97523 | 0.07186 |
| GM22521 | 1.97342 | 0.10732 |
| VMN1R172 | 1.97211 | 0.07313 |
| GM13773 | 1.97144 | 0.21792 |
| CLEC2H | 1.97034 | 0.20412 |
| OLFR714 | 1.96697 | 0.08163 |
| GM22284 | 1.96490 | 0.13765 |
| GM26342 | 1.96171 | 0.02416 |
| OLFR190 | 1.96140 | 0.01845 |
| CCDC152 | 1.96048 | 0.03753 |
| GM26048 | 1.95935 | 0.18068 |
| GM24410 | 1.95853 | 0.18441 |
| LGALS4 | 1.95650 | 0.02146 |
| OLFR969 | 1.95620 | 0.09460 |
| GM6222 | 1.95616 | 0.09501 |
| OLFR913 | 1.95340 | 0.31106 |
| 4930557A04RIK | 1.95314 | 0.06455 |
| GM24750 | 1.95027 | 0.23848 |
| GM23043 | 1.94750 | 0.04897 |
| STFA1 | 1.94574 | 0.08031 |
| SUCNR1 | 1.94177 | 0.10776 |
| MYOM1 | 1.94136 | 0.17534 |
| KLK1B22 | 1.93841 | 0.12634 |
| GM23917 | 1.93358 | 0.19490 |
| GM15949 | 1.93187 | 0.04498 |
| OLFR709-PS1 | 1.93037 | 0.07218 |
| OBP1A | 1.92773 | 0.34212 |
| SULT2A1 | 1.92697 | 0.35277 |
| GM24786 | 1.92300 | 0.21032 |
| SGK2 | 1.91561 | 0.15674 |
| MIRLET7C-1 | 1.91086 | 0.07251 |
| VMN1R90 | 1.90960 | 0.10180 |
| GM23342 | 1.90947 | 0.02984 |
| REG3G | 1.90771 | 0.15987 |
| GM24839 | 1.90586 | 0.26313 |
| N-R5S157 | 1.90509 | 0.11667 |
| GM25023 | 1.90492 | 0.02529 |
| CES2B | 1.90228 | 0.13857 |
| IGHV8-11 | 1.90067 | 0.15551 |
| GM25982 | 1.89862 | 0.05418 |
| ATP4A | 1.89851 | 0.39680 |
| GM26162 | 1.89809 | 0.01472 |
| PNPLA3 | 1.89778 | 0.00313 |
| AU015336 | 1.89289 | 0.07944 |
| GM24621 | 1.89139 | 0.08404 |
| CYP2C40 | 1.89106 | 0.40000 |
| GM25602 | 1.89074 | 0.15275 |
| ADGRG7 | 1.88894 | 0.32051 |
| SLC30A10 | 1.88809 | 0.12880 |
| GM25836 | 1.88784 | 0.19345 |
| GM25629 | 1.88723 | 0.11919 |
| GM24861 | 1.88583 | 0.03961 |
| GM21057 | 1.88436 | 0.08496 |

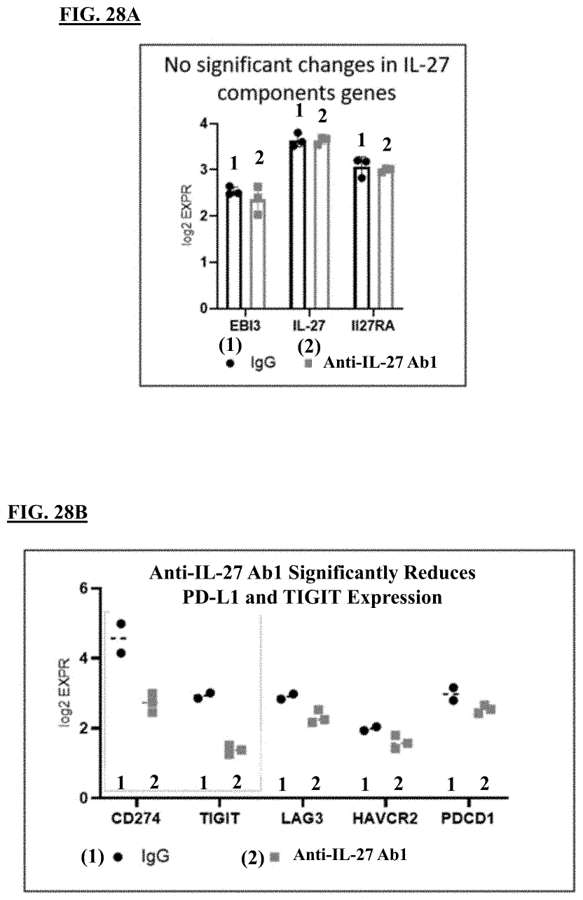

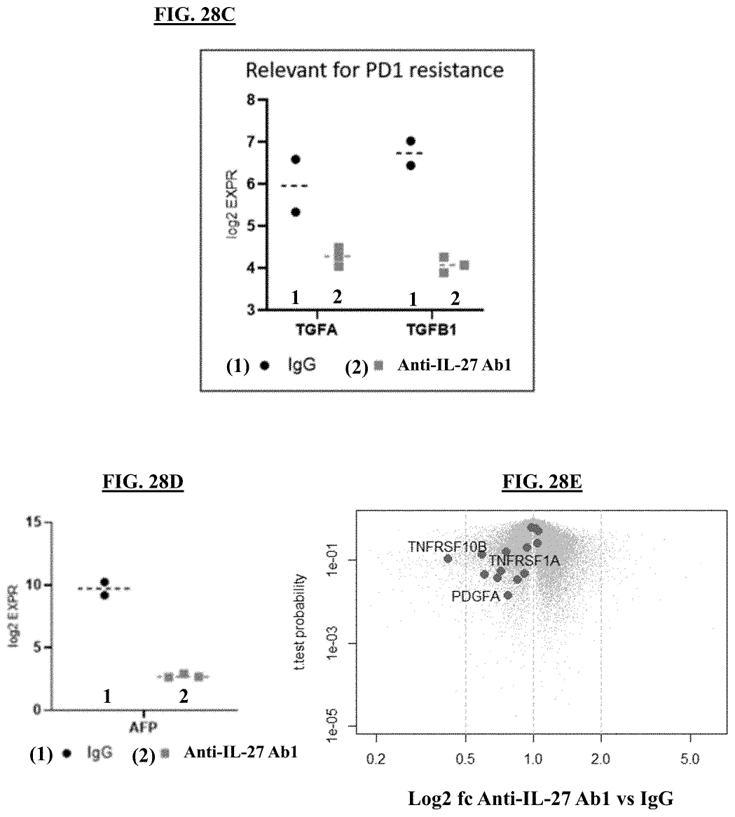

Notably, anti-IL-27 Ab1 was found to downregulate several key inhibitory genes, including PD-L1, TIGIT, and AFP expression (FIGS. 28B-28D), with no significant changes in the expression of EBI3, IL-27, and IL27RA (FIG. 28A). The TGFβ pathway was also found to be suppressed following anti-IL-27 Ab1 administration, with decreased expression of TNFRSF10B, TNFRSF1a, and PDGFA (FIG. 28C and FIG. 28E).

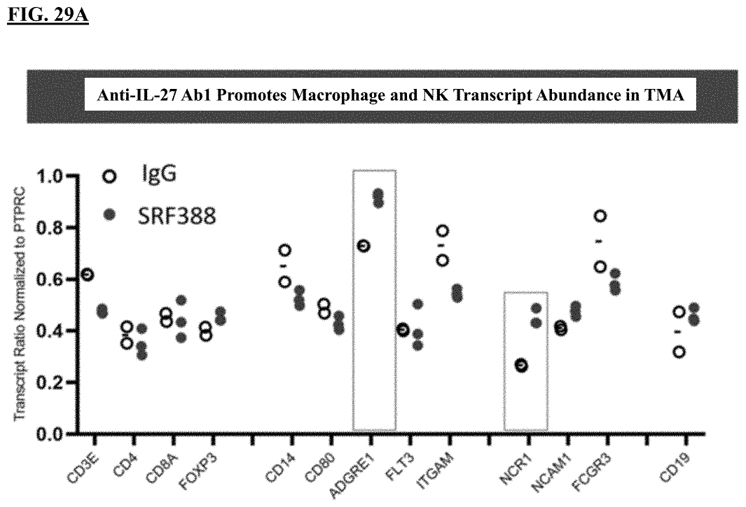

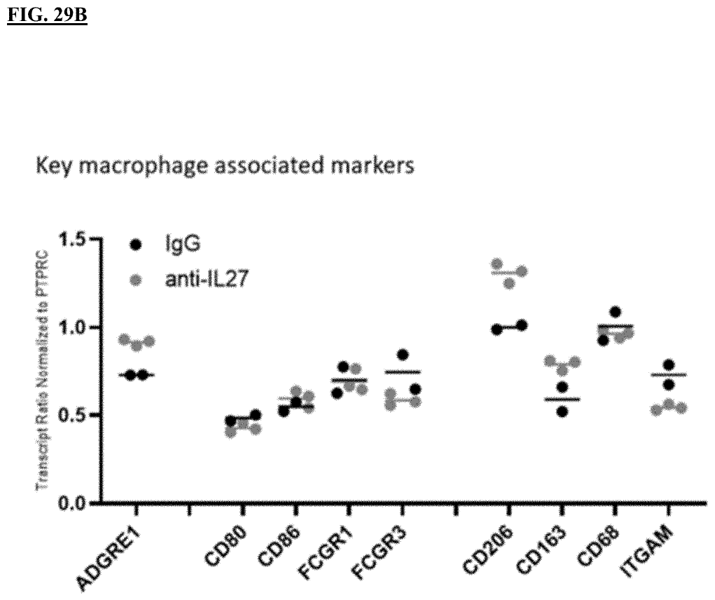

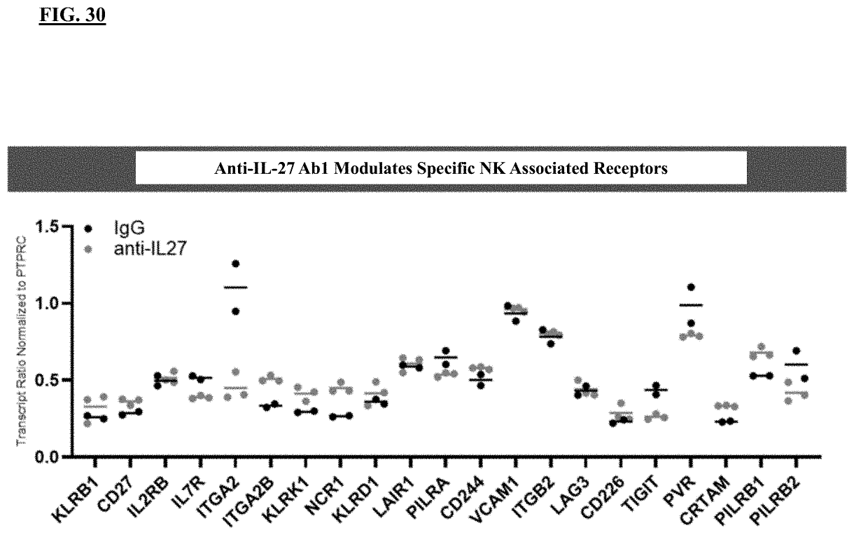

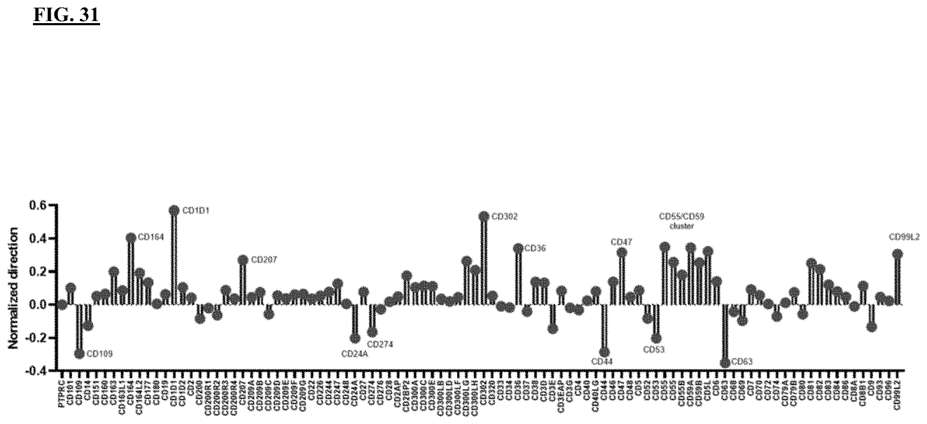

Further, treatment using anti-IL-27 Ab1 altered tumor immune cell infiltration. anti-IL-27 Ab1 promotes macrophage and NK transcript abundance in the tumor microenvironment (TME; FIG. 29A). In particular, anti-IL-27 Ab1 enriches for CD206 and CD163 (FIG. 29B), which are key macrophage associated markers; and anti-IL-27 Ab1 was found to modulate specific NK-associated receptors (FIG. 30). Heterogeneity in NK marker modulation suggests that anti-IL-27 Ab1 influences NK function and not only infiltration in HEPA1-6 tumors. In addition, various other cell surface markers showed modulated expression following treatment with anti-IL-27 Ab1 (FIG. 31).

### Example 15: TNFSF15 as a Biomarker of IL-27 Inhibition

TNFSF15 (tumor necrosis factor soluble factor 15), also known as TL1A (TNF like ligand 1A) or VEGFI (vascular endothelial growth factor inhibitor), is a cytokine within the tumor necrosis factor family known to play a role in inhibiting angiogenesis (Reference: VEGI, a novel cytokine of the tumor necrosis factor family, is an angiogenesis inhibitor that suppresses the growth of colon carcinomas in vivo. FASEB J. 1999 Human Genome Sciences, Inc.) and can function as a T cell co-stimulator by inducing the production of inflammatory cytokines (See Migone et al., Immunity 16(3):479-92 (March 2002); Jin et al., Mucosal Immunology 6:886-99 (Dec. 19, 2012)). TNFSF15 was shown to be upregulated after blocking IL-27 subsequent to treatment with an IL-27 inhibitor, establishing its utility as a biomarker in assessing the effectiveness IL-27 inhibition following the administration of an therapeutic intended to inhibit IL-27.

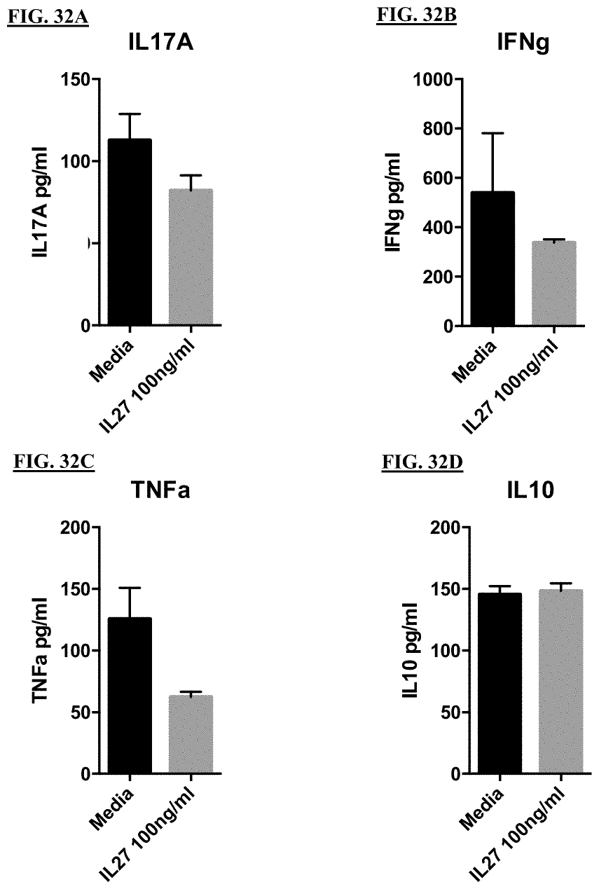

IL-27 inhibited the production of cytokines IL-17A, IFNγ (or IFNg) and TNFα (or TNFa) in activated PBMCs. Pooled human PBMCs from 3 donors were stimulated with anti-CD3 (0.25 μg/mL) for 3 days in the presence or absence of IL-27 (100 ng/mL). The supernatants were harvested and tested for the effect on IL-17A, IFNγ, TNFα, and IL-10 by cytometric bead array; IL-27 resulted in decreased production of IL-17A, IFNγ, and TNFα and had no effect on the production of IL-10 (FIGS. 32A-32D).

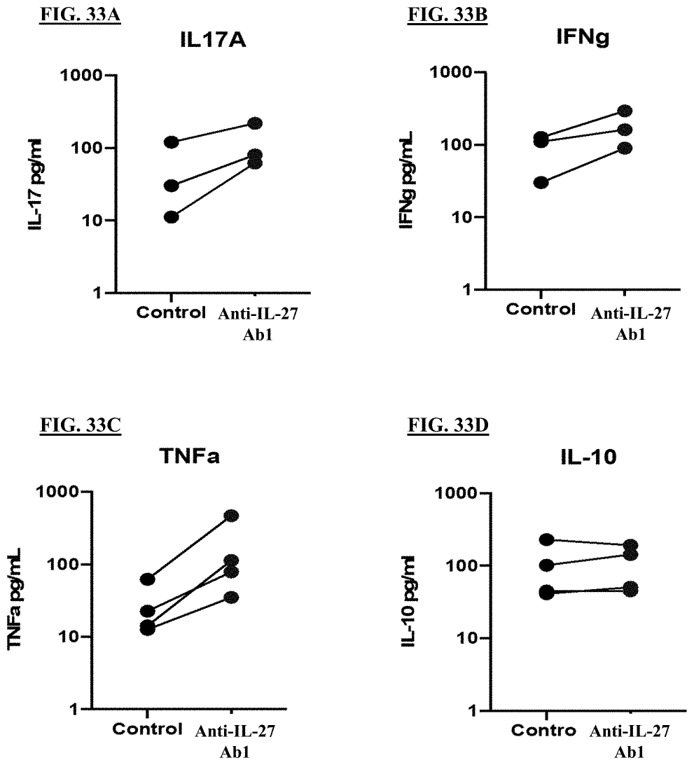

Conversely, blocking IL-27 with anti-IL-27 Ab1 in activated PBMCs lead to increased cytokine production. PBMCs from 3-4 individual donors were stimulated with anti-CD3 (0.25 μg/mL) for 4 days in the presence or absence of anti-IL-27 Ab1 (1 μg/mL). The supernatants were harvested and tested for the effect on IL-17A, IFNγ, TNFα, and IL-10 by cytometric bead array; anti-IL-27 Ab1 resulted in the increased production of IL-17A, IFNγ, and TNFα (FIGS. 33A-33D).

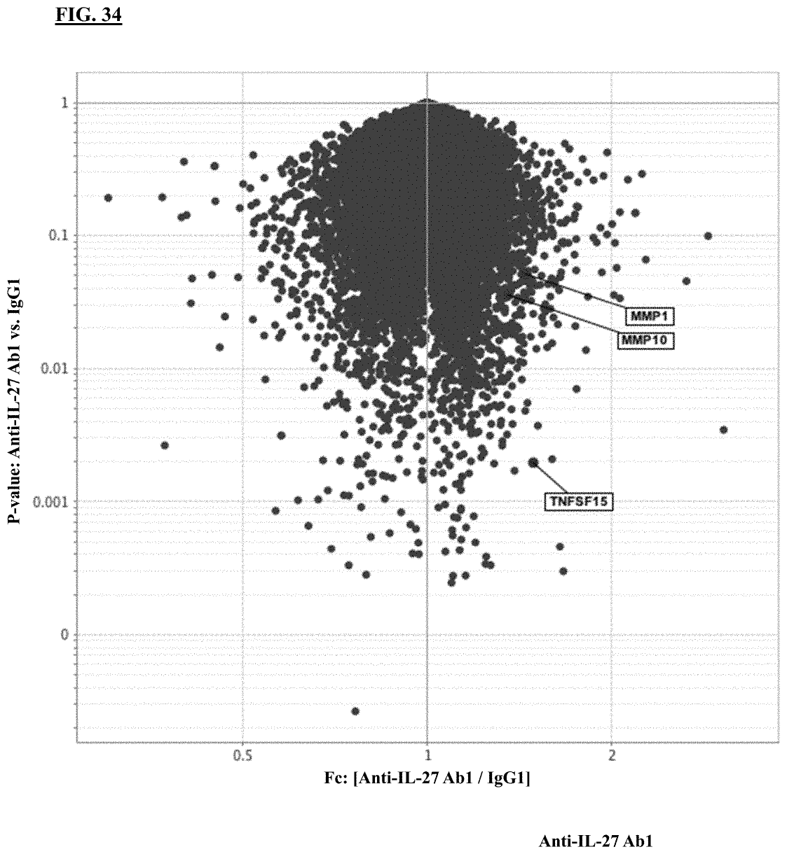

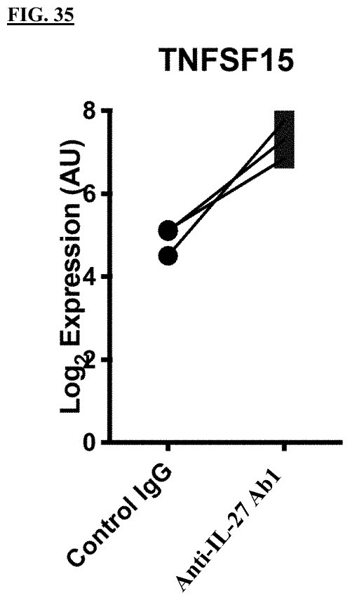

In order to determine other changes in gene expression that might be tracked as a pharmacodynamic or biomarker of anti-IL-27 Ab1 activity, gene expression profiling was performed by microarray analysis in activated PBMCs. PBMCs from three individual donors were stimulated with anti-CD3 (0.25 μg/mL) with or without anti-IL-27 Ab1 (1 μg/mL) for 24 hours. RNA from each sample was isolated and processed for gene expression profiling by microarray to determine the effect of IL-27 inhibition. Several genes were differentially expressed by volcano plot analysis subsequent to IL-27 inhibition with anti-IL-27 Ab1 as compared to isotype control, including MMP1, MMP10 and TNFSF15, as shown in Table 18. FIG. 34 shows a volcano plot representing the log₂ fold-change in gene expression after IL-27 inhibition compared to control (x-axis) versus the significance (p-value) of gene expression changes after treatment with anti-IL-27 Ab1 compared to control (y-axis). The TNFSF15 transcript is significantly increased after IL-27 blockade. TNFSF15 showed a reproducible increase in gene expression levels after treatment with anti-IL-27 Ab1 compared to treatment with isotype control in all 3 individual donors as shown in FIG. 35.

TABLE 18
Transcriptional Profiles from PBMCs in vitro Following IL-27 Inhibition

| Top 200 genes induced by IL-27 Inhibition | | | Top 200 genes represssed by IL-27 Inhibition | | |
|---|---|---|---|---|---|
| | Fold Change: | P-value: | | Fold Change: | P-value: |
| | [Anti-IL-27 | Anti-IL-27 | | [Anti-IL-27 | Anti-IL-27 |
| Gene Symbol | Ab1/IgG1] | Ab1 vs. IgG1 | Gene Symbol | Ab1/IgG1] | Ab1 vs. IgG1 |
| LOC105375002 | 1.645118 | 0.000457 | MIR519B | 0.542762 | 0.017631 |
| OR2AG1 | 1.633194 | 0.02143 | MIR4272 | 0.633358 | 0.045813 |
| SNORD115-10 | 1.61366 | 0.042825 | MIR4712 | 0.662079 | 0.013235 |
| MIR548AK | 1.594121 | 0.047753 | MIR4738 | 0.666032 | 0.008113 |
| VTRNA2-1 | 1.539071 | 0.018135 | MIR4668 | 0.676758 | 0.002044 |
| LOC105375837 | 1.528625 | 0.042765 | MIR634 | 0.67895 | 0.010185 |
| MIR4540 | 1.506249 | 0.014019 | LINC01233 | 0.695199 | 0.03774 |
| TNFSF15 | 1.48874 | 0.00196 | MIR19B2 | 0.706896 | 0.045961 |
| LOC105377950 | 1.474449 | 0.017305 | LOC105378073 | 0.712528 | 0.010495 |
| LOC105375291 | 1.455875 | 0.037183 | LOC105370770 | 0.716513 | 0.010176 |
| LOC392232 | 1.442977 | 0.034733 | KCCAT198 | 0.72041 | 0.040506 |
| IFNA6 | 1.435639 | 0.032471 | LOC101927552 | 0.721552 | 0.002026 |
| MIG7 | 1.433659 | 0.011024 | MIR1302-1 | 0.72292 | 0.031298 |
| LOC105369473 | 1.428921 | 0.045657 | APOH | 0.729982 | 0.018512 |
| GAGE1 | 1.427557 | 0.025117 | OR56A3 | 0.731084 | 0.014148 |
| LOC100506098 | 1.426037 | 0.008256 | SDIM1 | 0.73208 | 0.001115 |
| LOC105377643 | 1.416114 | 0.011246 | LOC101928395 | 0.736856 | 0.03984 |
| LOC105370479 | 1.414996 | 0.016319 | LCE1F | 0.739268 | 0.025556 |
| LOC101927342 | 1.387702 | 0.001705 | LOC100129476 | 0.740716 | 0.005034 |
| SPO11 | 1.384138 | 0.019734 | C6orf10 | 0.741035 | 0.045023 |
| LOC105374146 | 1.372296 | 0.042536 | DDX46 | 0.744975 | 0.031001 |
| MMP10 | 1.35563 | 0.035631 | MIR544B | 0.745497 | 0.000331 |
| MIR513A1 | 1.349552 | 0.003188 | LOC105371152 | 0.745893 | 0.001107 |
| LOC105373187 | 1.33789 | 0.011411 | HBZ | 0.746633 | 0.032099 |
| MIR302E | 1.329896 | 0.049451 | LINC00173 | 0.75377 | 0.046112 |
| LOC105374715 | 1.328902 | 0.039526 | MIR95 | 0.754058 | 0.029894 |
| CRYGC | 1.326275 | 0.002843 | LOC105369893 | 0.758578 | 0.042461 |
| NR0B1 | 1.322219 | 0.03641 | RNU6-58P | 0.761545 | 0.022808 |
| LOC105372823 | 1.322197 | 0.008592 | EPYC | 0.763574 | 0.015155 |
| KRT74 | 1.321165 | 0.032883 | WWC2-AS1 | 0.764054 | 2.67E−05 |
| LOC105375538 | 1.319595 | 0.019808 | LOC105370359 | 0.766521 | 0.01068 |
| LOC105378220 | 1.319239 | 0.004044 | MIR519A2 | 0.766808 | 0.045329 |
| SNORD116-23 | 1.314349 | 0.035597 | NSAP11 | 0.768519 | 0.020859 |
| ROCK1P1 | 1.311881 | 0.009781 | TDGF1 | 0.771131 | 0.010419 |
| LOC105375734 | 1.310484 | 0.01621 | LOC646588 | 0.771593 | 0.022006 |
| MIR874 | 1.30894 | 0.00796 | LOC101927281 | 0.773448 | 0.028332 |
| LOC102467216 | 1.306665 | 0.029327 | LOC105374670 | 0.777386 | 0.028457 |
| MIR18B | 1.305409 | 0.02179 | A2MP1 | 0.778456 | 0.001304 |
| LOC105378688 | 1.30501 | 0.005119 | SLC7A11 | 0.779049 | 0.048778 |
| PIPSL | 1.303637 | 0.013713 | PRH2 | 0.780025 | 0.029219 |
| LOC105374484 | 1.302454 | 0.01914 | SNORD114-19 | 0.781894 | 0.040583 |
| LRRC46 | 1.299851 | 0.040688 | LOC105373550 | 0.78246 | 0.003341 |
| SMG7-AS1 | 1.298713 | 0.039351 | LOC101927413 | 0.785604 | 0.025946 |
| LOC105378749 | 1.295692 | 0.02812 | MIR765 | 0.785901 | 0.027588 |
| CT55 | 1.295094 | 0.029095 | ALDH2 | 0.787067 | 0.03251 |
| MPDZ | 1.288722 | 0.005299 | LOC105370607 | 0.787785 | 0.029023 |
| HIST1H4B | 1.288471 | 0.02363 | ZNF717 | 0.788458 | 0.019536 |
| LOC105379223 | 1.287376 | 0.007854 | MIR7-2 | 0.78937 | 0.024631 |
| POM121L4P | 1.28655 | 0.006754 | OR2Y1 | 0.791148 | 0.005362 |
| HIST1H2AH | 1.285763 | 0.045671 | FCGR1C | 0.791398 | 0.036053 |
| OR6C74 | 1.284028 | 0.021357 | LOC102723879 | 0.792285 | 0.021851 |
| KRT33A | 1.28161 | 0.041615 | LOC153910 | 0.793028 | 0.026202 |
| PLCE1-AS1 | 1.280197 | 0.023448 | MIR410 | 0.793302 | 0.007555 |
| ALX1 | 1.279203 | 0.042523 | LOC102725105 | 0.794407 | 0.019386 |
| KCNN3 | 1.274928 | 0.01882 | LOC105373461 | 0.798351 | 0.021561 |
| LOC105377124 | 1.274321 | 0.01119 | LOC105373109 | 0.799694 | 0.035357 |
| BMP15 | 1.272919 | 0.013839 | LOC105376899 | 0.8002 | 0.011314 |
| RPLP0P2 | 1.271495 | 0.003828 | LOC105375857 | 0.800606 | 0.049069 |
| LOC105374024 | 1.271237 | 0.040873 | LOC105371883 | 0.802577 | 0.001618 |
| OR4C5 | 1.270368 | 0.008923 | MIR3121 | 0.804293 | 0.01562 |
| LOC105376234 | 1.269085 | 0.000332 | KRTAP19-2 | 0.805488 | 0.007923 |
| LINC01591 | 1.268954 | 0.010567 | LOC105370057 | 0.805791 | 0.036764 |
| LOC101928663 | 1.265242 | 0.003128 | LOC105375969 | 0.806373 | 0.002891 |
| LOC105375890 | 1.264046 | 0.005132 | TMED3 | 0.807779 | 0.049844 |
| IGHV3-16 | 1.2619 | 0.010831 | LOC105371856 | 0.809355 | 0.000543 |
| TRIM55 | 1.260372 | 0.014182 | EYA1 | 0.809515 | 0.008405 |
| LOC105371018 | 1.259721 | 0.026344 | LOC105372147 | 0.810403 | 0.018231 |
| LOC105371976 | 1.256944 | 0.030406 | SNORD115-20 | 0.81087 | 0.01113 |
| LOC100129345 | 1.256654 | 0.011358 | LOC105378424 | 0.812013 | 0.025289 |
| LOC105375875 | 1.254562 | 0.047657 | LOC105379104 | 0.812998 | 0.026458 |
| TM4SF1 | 1.253654 | 0.008913 | LOC105378078 | 0.815137 | 0.043079 |
| LOC105375273 | 1.25346 | 0.035173 | PCAT18 | 0.815152 | 0.012051 |
| LOC101928279 | 1.252743 | 0.015819 | CRYBA1 | 0.815488 | 0.008079 |
| MIR4493 | 1.251366 | 0.046562 | DOCK4-AS1 | 0.816945 | 0.003652 |
| LOC105376425 | 1.249945 | 0.029786 | PFN4 | 0.817389 | 0.017892 |
| LOC105376243 | 1.248505 | 0.004647 | LOC105373456 | 0.817412 | 0.047871 |
| OR4K17 | 1.24847 | 0.035361 | LOC105376207 | 0.817593 | 0.03227 |
| LINC00865 | 1.248322 | 0.007719 | RUFY4 | 0.817735 | 0.047204 |
| CAV3 | 1.246213 | 0.024171 | PGM5P2 | 0.820132 | 0.047774 |
| LOC105369830 | 1.244553 | 0.000338 | FLJ20712 | 0.822198 | 0.042585 |
| RS1 | 1.244259 | 0.009886 | LOC105378741 | 0.822688 | 0.023776 |
| LOC101929284 | 1.243628 | 0.009664 | KIR3DL2 | 0.822791 | 0.017144 |
| SPATA17-AS1 | 1.243442 | 0.036463 | IRS4 | 0.824149 | 0.020883 |
| C9orf129 | 1.241344 | 0.008005 | RNF175 | 0.824277 | 0.040132 |
| TEX12 | 1.239195 | 0.039282 | MIR4473 | 0.824601 | 0.01576 |
| OLIG2 | 1.235907 | 0.016016 | C21orf62-AS1 | 0.825875 | 0.029134 |
| SRGAP1 | 1.232365 | 0.0168 | TPI1P3 | 0.826368 | 0.005535 |
| LOC105374323 | 1.23136 | 0.006316 | OR6K2 | 0.82656 | 0.027951 |
| MIR1283-1 | 1.230642 | 0.005018 | LOC105375971 | 0.827067 | 0.028154 |
| GJD4 | 1.229305 | 0.016043 | LOC105377841 | 0.827522 | 0.046396 |
| LOC105374770 | 1.229204 | 0.023076 | LOC105370772 | 0.828585 | 0.019601 |
| IGFL2 | 1.226273 | 0.0209 | OR5H6 | 0.828925 | 0.033792 |
| LOC105376944 | 1.225754 | 0.017134 | LOC400958 | 0.829718 | 0.027201 |
| ALG1L | 1.224177 | 0.002154 | KCNAB1-AS2 | 0.830199 | 0.046563 |
| PCDHB18P | 1.223959 | 0.009905 | ANXA8L1 | 0.830719 | 0.017418 |
| RIMBP2 | 1.223519 | 0.039249 | LOC102724520 | 0.831113 | 0.019589 |
| OR52B4 | 1.22324 | 0.024734 | ALS2CR11 | 0.831379 | 0.005442 |
| ADGRL2 | 1.223099 | 0.02921 | LINC01052 | 0.831661 | 0.038213 |
| SCG5 | 1.222368 | 0.013456 | MIR492 | 0.8348 | 0.041657 |
| LOC105376570 | 1.221999 | 0.007463 | LOC101928119 | 0.835502 | 0.004564 |
| LOC654841 | 1.221214 | 0.016472 | SYT4 | 0.836312 | 0.025009 |
| HAS2-AS1 | 1.221198 | 0.047307 | LOC101928421 | 0.836388 | 0.04031 |
| SPATA24 | 1.220361 | 0.009933 | CASC23 | 0.838526 | 0.026392 |
| LINC01538 | 1.218128 | 0.007969 | LOC105375855 | 0.838859 | 0.027901 |
| CCDC85C | 1.217577 | 0.040344 | LINC00366 | 0.839834 | 0.009281 |
| LOC105370571 | 1.216124 | 0.013425 | DEPDC7 | 0.841512 | 0.035067 |
| LOC101927499 | 1.214266 | 0.029007 | LOC102724330 | 0.842675 | 0.033285 |
| TFPI2 | 1.213888 | 0.020451 | PLA2G2D | 0.843078 | 0.032033 |
| TSSK3 | 1.212747 | 0.003353 | LINC01166 | 0.844555 | 0.044378 |
| LOC105371818 | 1.21189 | 0.040895 | RPE65 | 0.845494 | 0.005261 |
| NPY1R | 1.211641 | 0.033039 | GLIS3 | 0.845755 | 0.047774 |
| LOC105376530 | 1.211121 | 0.008511 | LINC01523 | 0.84612 | 0.021712 |
| EVX1-AS | 1.208195 | 0.026851 | LOC105374787 | 0.84724 | 0.001573 |
| NPAS3 | 1.207602 | 0.019453 | IGLV3-32 | 0.84882 | 0.010713 |
| OR51Q1 | 1.206806 | 0.037877 | LOC105373595 | 0.849597 | 0.020159 |
| LOC101927418 | 1.206024 | 0.018913 | LOC728040 | 0.849791 | 0.01671 |
| SSTR2 | 1.20578 | 0.043155 | LINC00597 | 0.849976 | 0.037685 |
| LOC653653 | 1.205761 | 0.036703 | MIR3591 | 0.850417 | 0.045077 |
| LOC105372249 | 1.203904 | 0.010203 | KRTAP10-3 | 0.851393 | 0.03041 |
| LOC102723639 | 1.203543 | 0.007388 | ZC3H12B | 0.853207 | 0.026583 |
| LOC105375476 | 1.203038 | 0.015508 | LOC105370690 | 0.853241 | 0.001048 |
| FKBP7 | 1.201988 | 0.042042 | ZIC3 | 0.853712 | 0.039829 |
| HRAT56 | 1.201942 | 0.020195 | FGF10-AS1 | 0.853729 | 0.035366 |
| MIR4778 | 1.201939 | 0.017579 | PMS2P5 | 0.854195 | 0.048446 |
| LOC105378180 | 1.201664 | 0.020417 | SUGT1P1 | 0.854792 | 0.034214 |
| SCARA3 | 1.200146 | 0.008278 | LOC105369293 | 0.855263 | 0.044491 |
| LOC101930294 | 1.199177 | 0.022741 | DEFB119 | 0.85586 | 0.008256 |
| GUCY1A2 | 1.197934 | 0.000492 | LOC105369488 | 0.856419 | 0.018871 |
| MGAM2 | 1.197312 | 0.042246 | NRXN1 | 0.856769 | 0.047832 |
| TEX14 | 1.197177 | 0.037082 | LVCAT8 | 0.856829 | 0.028297 |
| OR2L13 | 1.19687 | 0.022817 | KIZ-AS1 | 0.857975 | 0.033864 |
| IGSF10 | 1.195837 | 0.02574 | LOC105378283 | 0.858492 | 0.036091 |
| SFRP2 | 1.194806 | 0.008784 | MIR1290 | 0.858598 | 0.031938 |
| GALNT16 | 1.194743 | 0.022064 | GKAP1 | 0.858622 | 0.042287 |
| TM4SF19 | 1.194237 | 0.042563 | LGR5 | 0.858793 | 0.01244 |
| LOC101927055 | 1.194215 | 0.029334 | NUDT10 | 0.85898 | 0.015489 |
| XIRP2-AS1 | 1.194173 | 0.045563 | DSCAM-AS1 | 0.85983 | 0.003654 |
| KCNJ16 | 1.194028 | 0.018938 | LOC105376640 | 0.86136 | 0.004118 |
| LOC105370561 | 1.194006 | 0.046886 | LOC105377693 | 0.862866 | 0.038576 |
| ASIC2 | 1.193784 | 0.020987 | SFTA3 | 0.863502 | 0.038347 |
| LOC401191 | 1.19219 | 0.013408 | CYYR1-AS1 | 0.864454 | 0.033992 |
| INCA1 | 1.189251 | 0.007965 | C9orf3 | 0.864736 | 0.006764 |
| LOC105376161 | 1.188411 | 0.016143 | LOC105373886 | 0.864772 | 0.012476 |
| ADAMTS3 | 1.188383 | 0.048302 | LOC105370060 | 0.865766 | 0.0341 |
| LOC101928231 | 1.187861 | 0.031467 | CTBP2 | 0.866251 | 0.009633 |
| CMYA5 | 1.187321 | 0.024627 | LOC101927328 | 0.86679 | 0.033119 |
| IGSF5 | 1.187268 | 0.046046 | ZNF449 | 0.866794 | 0.036778 |
| MIR3177 | 1.187017 | 0.041887 | OGN | 0.86879 | 0.02991 |
| GPC4 | 1.186929 | 0.023815 | MAP1LC3B2 | 0.869085 | 0.00058 |
| OC90 | 1.186462 | 0.046711 | ASPN | 0.87022 | 0.049977 |
| ST8SIA6-AS1 | 1.185617 | 0.034159 | LOC105370018 | 0.873226 | 0.033161 |
| SPDYE4 | 1.185393 | 0.007728 | LOC105376347 | 0.87418 | 0.033977 |
| CHODL-AS1 | 1.185235 | 0.029759 | OR52D1 | 0.874677 | 0.037917 |
| USP17L17 | 1.184052 | 0.037856 | SFTA1P | 0.87492 | 0.012883 |
| LOC105376975 | 1.182382 | 0.011412 | LOC105376658 | 0.877883 | 0.038524 |
| LOC101927109 | 1.180499 | 0.008781 | LINC00092 | 0.878605 | 0.03258 |
| CLCNKB | 1.179758 | 0.033508 | ERVK3-2 | 0.878835 | 0.001504 |
| NR1I2 | 1.179102 | 0.020533 | LOC105379385 | 0.878896 | 0.02955 |
| CASC17 | 1.179066 | 0.018346 | ATP8B3 | 0.879183 | 0.00631 |
| FAM19A3 | 1.177994 | 0.002009 | CTRB2 | 0.880249 | 0.025135 |
| LOC105375586 | 1.177235 | 0.025561 | LINC01527 | 0.880518 | 0.040258 |
| SUMO1P3 | 1.176228 | 0.027566 | KRTAP19-7 | 0.880746 | 0.030687 |
| FLJ44874 | 1.175661 | 0.037981 | LOC105376244 | 0.881151 | 0.036515 |
| NACAP1 | 1.175123 | 0.02547 | LOC105376554 | 0.881259 | 0.022378 |
| PTPN3 | 1.175009 | 0.028207 | ZNF30 | 0.8823 | 0.024368 |
| SSX2B | 1.174369 | 0.003748 | LINC00894 | 0.88431 | 0.020883 |
| SSX2B | 1.174369 | 0.003748 | MIR592 | 0.884366 | 0.023417 |
| LOXHD1 | 1.173847 | 0.008937 | LOH12CR2 | 0.886427 | 0.036694 |
| LINC01104 | 1.172936 | 0.038443 | ZNF846 | 0.887147 | 0.019874 |
| SERPINI1 | 1.172725 | 0.00357 | LINC01202 | 0.887201 | 0.036865 |
| LOC101928387 | 1.172215 | 0.007858 | CACNG4 | 0.8877 | 0.031884 |
| CDH12 | 1.171839 | 0.00615 | LOC642648 | 0.888364 | 0.019056 |
| LPIN3 | 1.171832 | 0.027473 | LOC105370526 | 0.888491 | 0.002669 |
| LOC105369575 | 1.171058 | 0.022101 | TRIM45 | 0.889074 | 0.046329 |
| SHC3 | 1.170409 | 0.014611 | AQP11 | 0.889428 | 0.005461 |
| AIFM3 | 1.170074 | 0.001628 | ANKEF1 | 0.889878 | 0.019532 |
| LOC105370564 | 1.16947 | 0.031193 | PTGDR2 | 0.890525 | 0.042594 |
| KCNJ14 | 1.169446 | 0.03716 | LOC100129869 | 0.891023 | 0.036875 |
| LOC102723323 | 1.16941 | 0.005804 | RBM12B-AS1 | 0.892387 | 0.00632 |
| LOC101929608 | 1.169147 | 0.005278 | LOC105378081 | 0.894442 | 0.029207 |
| LINC01039 | 1.168287 | 0.028847 | MIR365A | 0.895999 | 0.04973 |
| LOC105374980 | 1.168286 | 0.039283 | LINC00581 | 0.896004 | 0.048047 |
| GAL | 1.167357 | 0.013476 | LYPD8 | 0.896025 | 0.042973 |
| MIR107 | 1.1655 | 0.014498 | OR1411 | 0.896295 | 0.007798 |
| SCGB1A1 | 1.165175 | 0.007183 | FAM217A | 0.89866 | 0.019317 |
| LOC101927277 | 1.164914 | 0.022585 | TMEM254-AS1 | 0.898681 | 0.049183 |
| LMOD3 | 1.164303 | 0.013973 | LARS2-AS1 | 0.898922 | 0.014049 |
| MIR520G | 1.163434 | 0.033228 | CCDC150 | 0.899017 | 0.002703 |
| LINC01618 | 1.162641 | 0.049271 | GLI1 | 0.899604 | 0.00209 |
| LOC101929528 | 1.16255 | 0.024828 | CD163L1 | 0.90077 | 0.019563 |
| LOC105375710 | 1.162468 | 0.036178 | MAP2K2 | 0.901533 | 0.028712 |
| LOC102724732 | 1.162206 | 0.015044 | DIRC3 | 0.901772 | 0.047553 |
| GEM | 1.161606 | 0.005818 | IQCC | 0.901863 | 0.009081 |
| HLX-AS1 | 1.161435 | 0.03007 | IRX6 | 0.901966 | 0.041501 |
| LOC441239 | 1.160728 | 0.018365 | TRIM63 | 0.903387 | 0.048605 |
| LOC105373823 | 1.157242 | 0.000638 | SPATA18 | 0.90354 | 0.004953 |
| TINAG | 1.157194 | 0.039893 | LPEQ6126 | 0.903553 | 0.030286 |
| MFAP2 | 1.155841 | 0.026956 | RIPPLY3 | 0.905238 | 0.027728 |
| TUB-AS1 | 1.154582 | 0.031326 | ZNF192P1 | 0.905716 | 0.011822 |

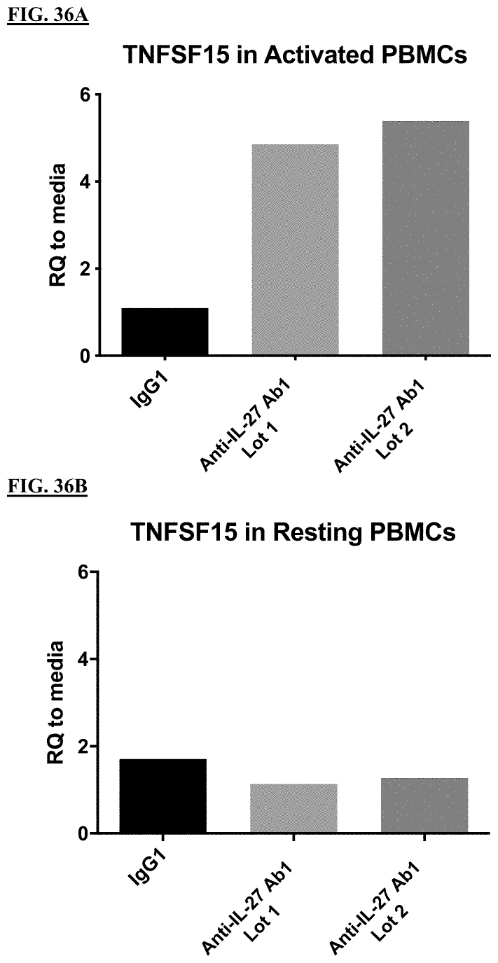

In a subsequent experiment, pooled human PBMCs were either stimulated with anti-CD3 (0.25 μg/ml) for 24 hours or left unstimulated in the presence of two different lots of anti-IL-27 Ab1 (1 μg/mL) or isotype control. RNA was harvested and TNFSF15 relative quantity (RQ) was determined by qPCR, measuring TNFSF15 transcripts using gene specific Taqman probes. The results confirmed an increase in TNFSF15 expression after IL-27 inhibition and was found to be dependent of immune cell activation, as TNFSF15 expression did not increase after treatment with anti-IL-27 Ab1 in resting PBMCs, as shown in FIGS. 36A-36B.

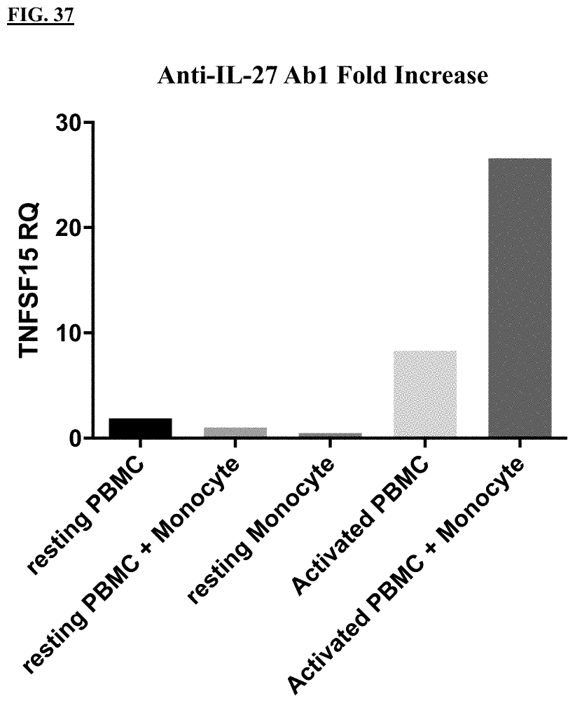

The relative increase in TNFSF15 transcript was further enhanced when monocytes were supplemented into the PBMC cultures. Pooled human PBMCs were either left resting (resting PBMC), PBMCs supplemented with a 1:2 ratio of monocytes enriched from PBMC (resting PBMC+ Monocyte), monocytes alone left resting (resting Monocyte), PBMCs that were activated with anti-CD3 (0.25 μg/mL) (Activated PMBC), or PBMCs supplemented with a 1:2 ratio of monocytes and activated with anti-CD3 (0.25 μg/mL) (Activated PMBC+Monocytes). Cells were cultured for 24 hours then RNA was isolated for determining TNFSF15 levels by qPCR. The values in FIG. 37 represent the fold change in TNFSF15 transcript after IL-27 inhibition with anti-IL-27 Ab1 compared to isotype control.

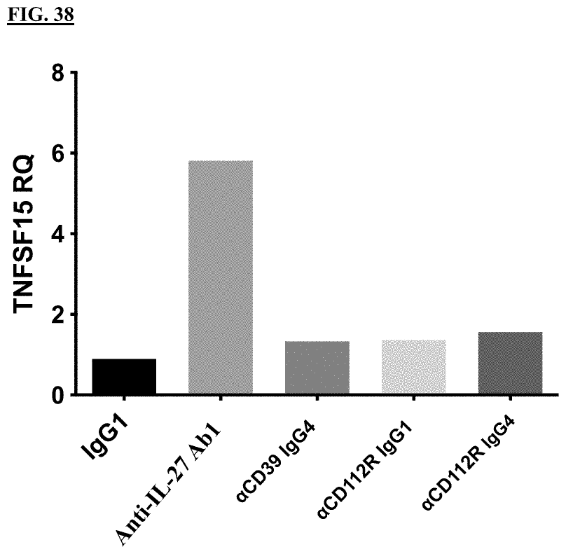

The effect of enhancing TNFSF15 transcript was specific to IL-27 blockade and was not observed with other antibodies targeting CD39 or CD112R, as shown in FIG. 38. Here, pooled human PBMCs were supplemented with monocyte derived macrophages and stimulated with anti-CD3 (0.25 μg/mL) for 24 hours in the presence of IgG1 control antibody, anti-IL-27 antibody (anti-IL-27 Ab1, 1 μg/mL), an anti-CD39 IgG4 antibody (1 μg/mL), an anti-CD112R IgG1 antibody (1 μg/mL), or an anti-CD112R IgG4 antibody (1 μg/mL). The RNA was harvested at 24 hours; the TNFSF15 transcript was measured in cell culture supernatants using human TL1A/TNFSF15 DuoSet ELISA (R&D Systems).

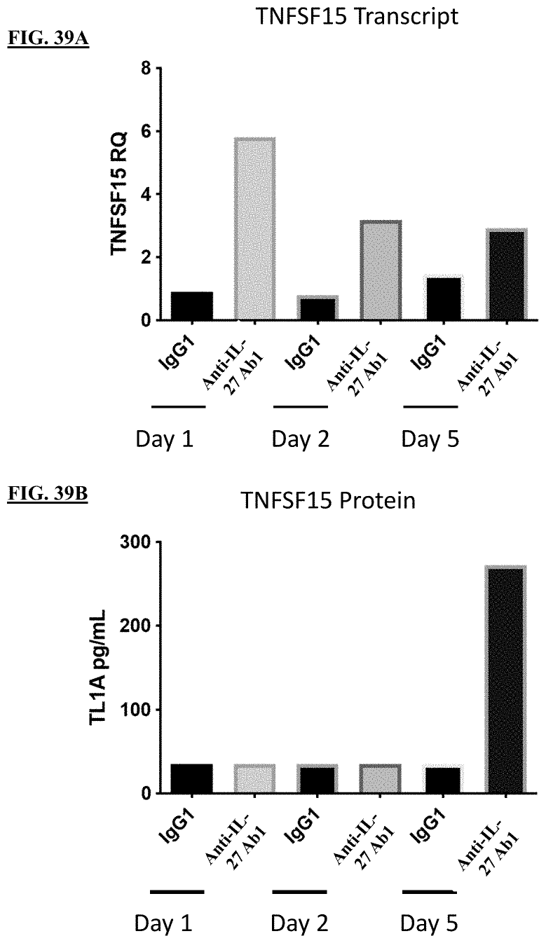

Further, as shown in FIG. 39A and FIG. 39B, an increase in secreted TNFSF15 protein was also seen after blocking IL-27 with anti-IL-27 Ab1 in activated PBMCs with delayed kinetics compared to the transcript (FIGS. 39A-39B). Pooled human PBMCs were supplemented with monocyte derived macrophages and stimulated with anti-CD3 (0.25 μg/mL) in the presence of IgG1 control or anti-IL-27 antibody (anti-IL-27 Ab1, 1 μg/mL). RNA was harvested at days 1, 2 and 5 and TNFSF15 transcript relative quantity (RQ) was determined by qPCR for each timepoint. Culture supernatants were harvested at different times; TNFSF15 protein was measured using a sandwich ELISA.

1. An isolated antibody that antagonizes human IL-27, or an antigen binding portion thereof, wherein the antibody or antigen binding portion thereof specifically binds to an epitope comprising one or more amino acids of (i) amino acids 37 to 56 corresponding to SEQ ID NO: 2 (IL-27p28), (ii) amino acids 142 to 164 corresponding to SEQ ID NO: 2 (IL-27p28), or (iii) both (i) and (ii).

2. The antibody, or antigen binding portion thereof, of claim 1, wherein the epitope comprises one or more amino acids of Gln37, Leu38, Glu42, Glu46, Va149, Ser50, Leu53, Lys56, Leu142, Asp143, Arg145, Asp146, Leu147, Arg149, His150, Arg152, Phe153, Leu156, Ala157, Gly159, Phe160, Asn161, Leu162, Pro163, or Glu164 of SEQ ID NO: 2 (IL-27p28).

3-11. (canceled)

12. The antibody, or antigen binding portion thereof, of claim 1, which comprise a heavy chain CDR1, a heavy chain CDR2, a heavy chain CDR3, a light chain CDR1, a light chain CDR2, and a light chain CDR3, wherein (i) the light chain CDR1 consists of N-XXXXXXLFSSNXKXYXX-C, the light chain CDR3 consists of N-XXXASAXXX-C, the heavy chain CDR2 consists of N-XXSSSXSYXYXXXXXXX-C, and the heavy chain CDR3 consists of N-XXXXGRTSYTATXHNXXXX-C, wherein X is any amino acid.

13. The antibody, or antigen binding portion thereof, of claim 1, wherein the antibody or antigen binding portion thereof does not comprise heavy and light chain CDRs selected from the group consisting of:
(i) heavy chain CDR1, CDR2 and CDR3 sequences set forth in SEQ ID NOs: 9, 10 and 11, respectively, and light chain CDR1, CDR2 and CDR3 sequences set forth in SEQ ID NOs: 17, 18 and 19, respectively;
(ii) heavy chain CDR1, CDR2 and CDR3 sequences set forth in SEQ ID NOs: 31, 32 and 33, respectively, and light chain CDR1, CDR2 and CDR3 sequences set forth in SEQ ID NOs: 39, 40 and 41, respectively;
(iii) heavy chain CDR1, CDR2 and CDR3 sequences set forth in SEQ ID NOs: 53, 54 and 55, respectively, and light chain CDR1, CDR2 and CDR3 sequences set forth in SEQ ID NOs: 61, 62 and 63, respectively;
(iv) heavy chain CDR1, CDR2 and CDR3 sequences set forth in SEQ ID NOs: 75, 76 and 77, respectively, and light chain CDR1, CDR2 and CDR3 sequences set forth in SEQ ID NOs: 83, 84 and 85, respectively;
(v) heavy chain CDR1, CDR2 and CDR3 sequences set forth in SEQ ID NOs: 97, 98 and 99, respectively, and light chain CDR1, CDR2 and CDR3 sequences set forth in SEQ ID NOs: 105, 106 and 107, respectively;
(vi) heavy chain CDR1, CDR2 and CDR3 sequences set forth in SEQ ID NOs: 119, 120 and 121, respectively, and light chain CDR1, CDR2 and CDR3 sequences set forth in SEQ ID NOs: 127, 128 and 129, respectively;
(vii) heavy chain CDR1, CDR2 and CDR3 sequences set forth in SEQ ID NOs: 12, 13 and 14, respectively, and light chain CDR1, CDR2 and CDR3 sequences set forth in SEQ ID NOs: 20, 21 and 22, respectively;
(viii) heavy chain CDR1, CDR2 and CDR3 sequences set forth in SEQ ID NOs: 34, 35 and 36, respectively, and light chain CDR1, CDR2 and CDR3 sequences set forth in SEQ ID NOs: 42, 43 and 44, respectively;
(ix) heavy chain CDR1, CDR2 and CDR3 sequences set forth in SEQ ID NOs: 56, 57 and 58, respectively, and light chain CDR1, CDR2 and CDR3 sequences set forth in SEQ ID NOs: 64, 65 and 66, respectively;
(x) heavy chain CDR1, CDR2 and CDR3 sequences set forth in SEQ ID NOs: 78, 79 and 80, respectively, and light chain CDR1, CDR2 and CDR3 sequences set forth in SEQ ID NOs: 86, 87 and 88, respectively;
(xi) heavy chain CDR1, CDR2 and CDR3 sequences set forth in SEQ ID NOs: 100, 101 and 102, respectively, and light chain CDR1, CDR2 and CDR3 sequences set forth in SEQ ID NOs: 108, 109 and 110, respectively; and
(xii) heavy chain CDR1, CDR2 and CDR3 sequences set forth in SEQ ID NOs: 122, 123 and 124, respectively, and light chain CDR1, CDR2 and CDR3 sequences set forth in SEQ ID NOs: 130, 131 and 132, respectively.

14-18. (canceled)

19. The antibody, or antigen binding portion thereof, of claim 1, wherein the antibody or antigen binding portion thereof exhibits at least one or more of the following properties:
(i) binds to human IL-27 with an equilibrium dissociation constant (K_{D}) of 15 nM or less;
(ii) blocks binding of IL-27 to IL-27 receptor;
(iii) inhibits or reduces STAT1 and/or STAT3 phosphorylation in a cell;
(iv) inhibits or reduces IL-27-mediated inhibition of CD161 expression in a cell;
(v) inhibits or reduces IL-27-mediated PD-L1 and/or TIM-3 expression in a cell; and
(vi) induces or enhances PD-1-mediated secretion of one or more cytokines from a cell.

20-30. (canceled)

31. The isolated antibody, or antigen binding portion thereof, of any one of claims 1 to 30, wherein the antibody is selected from the group consisting of an IgG1, an IgG2, an IgG3, an IgG4, an IgM, an IgA1 an IgA2, an IgD, and an IgE antibody.

32-33. (canceled)

34. A pharmaceutical composition comprising the isolated antibody or antigen binding portion thereof of claim 1, and a pharmaceutically acceptable carrier.

35. A nucleic acid molecule or a set of nucleic acid molecules encoding the isolated antibody, or antigen binding portion thereof, of claim 1.

36. An expression vector comprising the nucleic acid of claim 35.

37. A cell comprising the expression vector of claim 36.

38. A method for producing an antibody that specifically binds human IL-27, or an antigen binding portion thereof, the method comprising maintaining a cell according to claim 37 under conditions permitting expression of the antibody or antigen binding portion thereof.

39. (canceled)

40. A method to inhibit or reduce STAT1 and/or STAT3 phosphorylation in a cell, the method comprising contacting the cell with the isolated antibody, or antigen binding fragment, of claim 1, wherein the antibody, or antigen binding portion thereof, inhibits or reduces STAT1 and/or STAT3 phosphorylation in a cell.

41. A method to inhibit or reduce inhibition of CD161 expression in a cell, the method comprising contacting the cell with the isolated antibody, or antigen binding fragment, of claim 1, wherein the antibody, or antigen binding portion thereof, inhibits or reduces inhibition of CD161 expression in a cell.

42. A method to inhibit or reduce PD-L1 and/or TIM-3 expression in a cell, the method comprising contacting the cell with the isolated antibody, or antigen binding fragment, of claim 1, wherein the antibody, or antigen binding portion thereof, inhibits or PD-L1 and/or TIM-3 expression in a cell.

43. A method to induce or enhance secretion of one or more cytokines from a cell, the method comprising contacting the cell with the isolated antibody, or antigen binding fragment, of claim 1, wherein the antibody, or antigen binding portion thereof, induces or enhances PD-1 mediated secretion of one or more cytokines from a cell.

44. A method of stimulating an immune response in a subject, the method comprising administering to the subject an effective amount of the isolated antibody, or antigen binding fragment, of claim 1.

45. A method of treating a cancer in a subject, the method comprising administering to the subject an effective amount of the isolated antibody, or antigen binding fragment, of claim 1.

46-50. (canceled)

51. A method of enhancing one or more activities of an anti-PD-1 antibody (e.g., enhances PD-1-mediated cytokine secretion; enhances anti-PD-1 mediated TNFα secretion; enhances anti-PD-1 mediated IL-6 secretion from a cell exposed to anti-PD-1 antibodies), the method comprising exposing a cell to an antibody, or antigen binding portion thereof, of claim 1, concurrently with or sequentially to an anti-PD-1 antibody, thereby to enhance one or more activities of anti-PD1 antibodies.

52-67. (canceled)

68. A kit comprising the isolated monoclonal antibody, or antigen binding portion thereof, of claim 1 and instructions for use in stimulating an immune response in a subject, or treating cancer in a subject.

69-76. (canceled)

77. A method of determining the efficacy of an agent that antagonizes human IL-27, comprising measuring the expression of TNFSF15 in a sample obtained from a subject, administering the agent that antagonizes human IL-27 to the subject, and measuring the expression of TNFSF15 in a sample obtained from the subject after the administering.

78-81. (canceled)


SEQUENCE LISTING (156 sequences)

SEQ ID NO: 1 - protein, 229 aa, Artificial Sequence
  human EBI3 protein
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 2 - protein, 243 aa, Artificial Sequence
  human p28 protein
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - protein, 636 aa, Artificial Sequence
  human WSX1
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 4 - protein, 918 aa, Artificial Sequence
  human gp130 protein
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 5 - protein, 232 aa, Artificial Sequence
  Human IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 229 aa, Artificial Sequence
  Human IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 229 aa, Artificial Sequence
  Human IgG4 (S228P)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 229 aa, Artificial Sequence
  Human IgG4 (S228P / L235E)
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 8 aa, Artificial Sequence
  Heavy chain CDR1
GFTFSSYS

SEQ ID NO: 10 - protein, 8 aa, Artificial Sequence
  Heavy chain CDR2
ISSSSSYI

SEQ ID NO: 11 - protein, 19 aa, Artificial Sequence
  Heavy chain CDR3
ARDGGRTSYTATAHNWFDP

SEQ ID NO: 12 - protein, 9 aa, Artificial Sequence
  Heavy chain CDR1
FTFSSYSMN

SEQ ID NO: 13 - protein, 17 aa, Artificial Sequence
  Heavy chain CDR2
SISSSSSYIYYADSVKG

SEQ ID NO: 14 - protein, 19 aa, Artificial Sequence
  Heavy Chain CDR3
ARDGGRTSYTATAHNWFDP

SEQ ID NO: 15 - protein, 126 aa, Artificial Sequence
  Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 16 - DNA, 378 bp, Artificial Sequence
  DNA Heavy Chain
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 17 - protein, 12 aa, Artificial Sequence
  Light Chain CDR1
QSVLFSSNNKNY

SEQ ID NO: 18 - protein, 3 aa, Artificial Sequence
  Light Chain CDR2
WAS

SEQ ID NO: 19 - protein, 9 aa, Artificial Sequence
  Light Chain CDR3
QQHASAPPT

SEQ ID NO: 20 - protein, 17 aa, Artificial Sequence
  Light Chain CDR1
KSSQSVLFSSNNKNYLA

SEQ ID NO: 21 - protein, 7 aa, Artificial Sequence
  Light Chain CDR2
WASTRES

SEQ ID NO: 22 - protein, 9 aa, Artificial Sequence
  Light Chain CDR3
QQHASAPPT

SEQ ID NO: 23 - protein, 113 aa, Artificial Sequence
  Light Chain
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIK

SEQ ID NO: 24 - DNA, 339 bp, Artificial Sequence
  DNA Light Chain
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaa

SEQ ID NO: 25 - protein, 456 aa, Artificial Sequence
  Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1368 bp, Artificial Sequence
  DNA Heavy Chain
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 27 - protein, 220 aa, Artificial Sequence
  Light Chain
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQHASAPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 660 bp, Artificial Sequence
  DNA Light Chain
gacatcgtgatgacccagtctccagactccctggctgtgtctctgggcgagagggccaccatcaactgcaagtccagccagagtgttttattcagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagcacgccagtgcccctcctacttttggcggagggaccaaggttgagatcaaacgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 29 - protein, 452 aa, Artificial Sequence
  Heavy Chain
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - DNA, 1356 bp, Artificial Sequence
  DNA Heavy Chain
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 31 - protein, 8 aa, Artificial Sequence
  Heavy Chain CDR1
GFTFRSYG

SEQ ID NO: 32 - protein, 8 aa, Artificial Sequence
  Heavy chain CDR2
ISSSSSYI

SEQ ID NO: 33 - protein, 19 aa, Artificial Sequence
  Heavy Chain CDR3
ARDGGRTSYTATAHNWFDP

SEQ ID NO: 34 - protein, 9 aa, Artificial Sequence
  Heavy Chain CDR1
FTFRSYGMN

SEQ ID NO: 35 - protein, 17 aa, Artificial Sequence
  Heavy Chain CDR2
SISSSSSYIYYADSVKG

SEQ ID NO: 36 - protein, 19 aa, Artificial Sequence
  Heavy Chain CDR3
ARDGGRTSYTATAHNWFDP

SEQ ID NO: 37 - protein, 126 aa, Artificial Sequence
  Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 38 - DNA, 378 bp, Artificial Sequence
  DNA Heavy Chain
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 39 - protein, 12 aa, Artificial Sequence
  Light Chain CDR1
QSVLFSSNNKNY

SEQ ID NO: 40 - protein, 3 aa, Artificial Sequence
  Light Chain CDR2
WAS

SEQ ID NO: 41 - protein, 9 aa, Artificial Sequence
  Light Chain CDR3
QQHASAPPT

SEQ ID NO: 42 - protein, 17 aa, Artificial Sequence
  Light Chain CDR1
KSSQSVLFSSNNKNYLA

SEQ ID NO: 43 - protein, 7 aa, Artificial Sequence
  Light Chain CDR2
WASTRES

SEQ ID NO: 44 - protein, 9 aa, Artificial Sequence
  Light Chain CDR3
QQHASAPPT

SEQ ID NO: 45 - protein, 113 aa, Artificial Sequence; identical to SEQ ID NO: 23
  Light Chain

SEQ ID NO: 46 - DNA, 339 bp, Artificial Sequence; identical to SEQ ID NO: 24
  DNA Light Chain

SEQ ID NO: 47 - protein, 456 aa, Artificial Sequence
  Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1368 bp, Artificial Sequence
  DNA Heavy Chain
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 49 - protein, 220 aa, Artificial Sequence; identical to SEQ ID NO: 27
  Light Chain

SEQ ID NO: 50 - DNA, 660 bp, Artificial Sequence; identical to SEQ ID NO: 28
  DNA Light Chain

SEQ ID NO: 51 - protein, 452 aa, Artificial Sequence
  Heavy Chain
EVQLVESGGGLVKPGGSLRLSCAASGFTFRSYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 52 - DNA, 1356 bp, Artificial Sequence
  DNA Heavy Chain
gaggtgcagctggtggagtctgggggaggcctggtcaagcctggggggtccctgagactctcctgtgcagcctctggattcaccttccggagctatgggatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 53 - protein, 8 aa, Artificial Sequence
  Heavy chain CDR1
GFTFSRTG

SEQ ID NO: 54 - protein, 8 aa, Artificial Sequence
  Heavy chain CDR2
ISSSSSYI

SEQ ID NO: 55 - protein, 19 aa, Artificial Sequence
  Heavy chain CDR3
ARDGGRTSYTATAHNWFDP

SEQ ID NO: 56 - protein, 9 aa, Artificial Sequence
  Heavy chain CDR1
FTFSRTGMN

SEQ ID NO: 57 - protein, 17 aa, Artificial Sequence
  Heavy chain CDR2
SISSSSSYIYYADSVKG

SEQ ID NO: 58 - protein, 19 aa, Artificial Sequence
  Heavy chain CDR3
ARDGGRTSYTATAHNWFDP

SEQ ID NO: 59 - protein, 126 aa, Artificial Sequence
  Heavy chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 60 - DNA, 378 bp, Artificial Sequence
  DNA Heavy chain
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 61 - protein, 12 aa, Artificial Sequence
  Light chain CDR1
QSVLFSSNNKNY

SEQ ID NO: 62 - protein, 3 aa, Artificial Sequence
  Light chain CDR2
WAS

SEQ ID NO: 63 - protein, 9 aa, Artificial Sequence
  Light chain CDR3
QQHASAPPT

SEQ ID NO: 64 - protein, 17 aa, Artificial Sequence
  Light chain CDR1
KSSQSVLFSSNNKNYLA

SEQ ID NO: 65 - protein, 7 aa, Artificial Sequence
  Light chain CDR2
WASTRES

SEQ ID NO: 66 - protein, 9 aa, Artificial Sequence
  Light chain CDR3
QQHASAPPT

SEQ ID NO: 67 - protein, 113 aa, Artificial Sequence; identical to SEQ ID NO: 23
  Light chain

SEQ ID NO: 68 - DNA, 339 bp, Artificial Sequence; identical to SEQ ID NO: 24
  DNA Light chain

SEQ ID NO: 69 - protein, 456 aa, Artificial Sequence
  Heavy Chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1368 bp, Artificial Sequence
  DNA Heavy Chain
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 71 - protein, 220 aa, Artificial Sequence; identical to SEQ ID NO: 27
  Light Chain

SEQ ID NO: 72 - DNA, 660 bp, Artificial Sequence; identical to SEQ ID NO: 28
  DNA Light Chain

SEQ ID NO: 73 - protein, 452 aa, Artificial Sequence
  Heavy Chain
EVQLVESGGGLVKPGGSLRLSCAASGFTFSRTGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - DNA, 1356 bp, Artificial Sequence
  DNA Heavy Chain
gaggtgcagctggtggagtctgggggaggcctggtcaagcctggggggtccctgagactctcctgtgcagcctctggattcaccttcagtaggactgggatgaactgggtccgccaggctccagggaaggggctggaatgggtctcatccattagtagtagtagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 75 - protein, 8 aa, Artificial Sequence
  Heavy chain CDR1
GFTFSRYG

SEQ ID NO: 76 - protein, 8 aa, Artificial Sequence
  Heavy chain CDR2
ISSSSAYI

SEQ ID NO: 77 - protein, 19 aa, Artificial Sequence
  Heavy chain CDR3
ARDGGRTSYTATAHNWFDP

SEQ ID NO: 78 - protein, 9 aa, Artificial Sequence
  Heavy chain CDR1
FTFSRYGMN

SEQ ID NO: 79 - protein, 17 aa, Artificial Sequence
  Heavy chain CDR2
SISSSSAYILYADSVKG

SEQ ID NO: 80 - protein, 19 aa, Artificial Sequence
  Heavy chain CDR3
ARDGGRTSYTATAHNWFDP

SEQ ID NO: 81 - protein, 126 aa, Artificial Sequence
  Heavy chain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSS

SEQ ID NO: 82 - DNA, 378 bp, Artificial Sequence
  DNA Heavy chain
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 83 - protein, 12 aa, Artificial Sequence
  Light chain CDR1
QSVLFSSNNKNY

SEQ ID NO: 84 - protein, 3 aa, Artificial Sequence
  Light chain CDR2
WAS

SEQ ID NO: 85 - protein, 9 aa, Artificial Sequence
  Light chain CDR3
QQHASAPPT

SEQ ID NO: 86 - protein, 17 aa, Artificial Sequence
  Light chain CDR1
KSSQSVLFSSNNKNYLA

SEQ ID NO: 87 - protein, 7 aa, Artificial Sequence
  Light chain CDR2
WASTRES

SEQ ID NO: 88 - protein, 9 aa, Artificial Sequence
  Light chain CDR3
QQHASAPPT

SEQ ID NO: 89 - protein, 113 aa, Artificial Sequence; identical to SEQ ID NO: 23
  Light Chain

SEQ ID NO: 90 - DNA, 339 bp, Artificial Sequence; identical to SEQ ID NO: 24
  DNA Light Chain

SEQ ID NO: 91 - protein, 456 aa, Artificial Sequence
  Heavy Chain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 1368 bp, Artificial Sequence
  DNA Heavy Chain
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 93 - protein, 220 aa, Artificial Sequence; identical to SEQ ID NO: 27
  Light Chain

SEQ ID NO: 94 - DNA, 660 bp, Artificial Sequence; identical to SEQ ID NO: 28
  DNA Light Chain

SEQ ID NO: 95 - protein, 452 aa, Artificial Sequence
  Heavy CHain
EVQLVESGGGLVKPGGSLRLSCAASGFTFSRYGMNWVRQAPGKGLEWVSSISSSSAYILYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - DNA, 1356 bp, Artificial Sequence
  DNA Heavy Chain
gaggtgcagctggtggagtctgggggaggcctggtcaagcctggggggtccctgagactctcctgtgcagcctctggattcaccttcagtaggtatgggatgaactgggtccgccaggctccagggaaggggctggagtgggtctcatccattagtagtagtagtgcttacatactgtacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 97 - protein, 8 aa, Artificial Sequence
  Heavy chain CDR1
GFTFASYG

SEQ ID NO: 98 - protein, 8 aa, Artificial Sequence
  Heavy chain CDR2
ISSSSSYI

SEQ ID NO: 99 - protein, 19 aa, Artificial Sequence
  Heavy chain CDR3
ARDGGRTSYTATAHNWFDP

SEQ ID NO: 100 - protein, 9 aa, Artificial Sequence
  Heavy chain CDR1
FTFASYGMN

SEQ ID NO: 101 - protein, 17 aa, Artificial Sequence
  Heavy chain CDR2
SISSSSSYIYYADSVKG

SEQ ID NO: 102 - protein, 19 aa, Artificial Sequence
  Heavy chain CDR3
ARDGGRTSYTATAHNWFDP

SEQ ID NO: 103 - protein, 126 aa, Artificial Sequence
  Heavy chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSS

SEQ ID NO: 104 - DNA, 378 bp, Artificial Sequence
  DNA Heavy Chain
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 105 - protein, 12 aa, Artificial Sequence
  Light Chain CDR1
QSVLFSSNNKNY

SEQ ID NO: 106 - protein, 3 aa, Artificial Sequence
  Light Chain CDR2
WAS

SEQ ID NO: 107 - protein, 9 aa, Artificial Sequence
  Light Chain CDR3
QQHASAPPT

SEQ ID NO: 108 - protein, 17 aa, Artificial Sequence
  Light Chain CDR1
KSSQSVLFSSNNKNYLA

SEQ ID NO: 109 - protein, 7 aa, Artificial Sequence
  Light Chain CDR2
WASTRES

SEQ ID NO: 110 - protein, 9 aa, Artificial Sequence
  Light Chain CDR3
QQHASAPPT

SEQ ID NO: 111 - protein, 113 aa, Artificial Sequence; identical to SEQ ID NO: 23
  Light Chain

SEQ ID NO: 112 - DNA, 339 bp, Artificial Sequence; identical to SEQ ID NO: 24
  DNA Light Chain

SEQ ID NO: 113 - protein, 456 aa, Artificial Sequence
  Heavy Chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - DNA, 1368 bp, Artificial Sequence
  DNA Heavy Chain
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 115 - protein, 220 aa, Artificial Sequence; identical to SEQ ID NO: 27
  Light Chain

SEQ ID NO: 116 - DNA, 660 bp, Artificial Sequence; identical to SEQ ID NO: 28
  DNA Light Chain

SEQ ID NO: 117 - protein, 452 aa, Artificial Sequence
  Heavy Chain
EVQLVESGGGLVKPGGSLRLSCAASGFTFASYGMNWVRQAPGKGLEWVSSISSSSSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 118 - DNA, 1356 bp, Artificial Sequence
  DNA Heavy Chain
gaggtgcagctggtggagtctgggggaggcctggtcaagcctggggggtccctgagactctcctgtgcagcctctggattcaccttcgctagctatgggatgaactgggtccgccaggctccagggaaggggctggagtgggtctcatccattagtagttctagtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 119 - protein, 8 aa, Artificial Sequence
  Heavy chain CDR1
GFTFRSYG

SEQ ID NO: 120 - protein, 8 aa, Artificial Sequence
  Heavy chain CDR2
ISSSGSYI

SEQ ID NO: 121 - protein, 19 aa, Artificial Sequence
  Heavy chain CDR3
ARDGGRTSYTATAHNWFDP

SEQ ID NO: 122 - protein, 9 aa, Artificial Sequence
  Heavy chain CDR1
FTFRSYGMN

SEQ ID NO: 123 - protein, 17 aa, Artificial Sequence
  Heavy chain CDR2
GISSSGSYIYYADSVKG

SEQ ID NO: 124 - protein, 19 aa, Artificial Sequence
  Heavy chain CDR3
ARDGGRTSYTATAHNWFDP

SEQ ID NO: 125 - protein, 126 aa, Artificial Sequence
  Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSS

SEQ ID NO: 126 - DNA, 378 bp, Artificial Sequence
  DNA Heavy Chain
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctca

SEQ ID NO: 127 - protein, 12 aa, Artificial Sequence
  Light Chain CDR1
QSVLFSSNNKNY

SEQ ID NO: 128 - protein, 3 aa, Artificial Sequence
  Light Chain CDR2
WAS

SEQ ID NO: 129 - protein, 9 aa, Artificial Sequence
  Light Chain CDR3
QQHASAPPT

SEQ ID NO: 130 - protein, 17 aa, Artificial Sequence
  Light Chain CDR1
KSSQSVLFSSNNKNYLA

SEQ ID NO: 131 - protein, 7 aa, Artificial Sequence
  Light Chain CDR2
WASTRES

SEQ ID NO: 132 - protein, 9 aa, Artificial Sequence
  Light Chain CDR3
QQHASAPPT

SEQ ID NO: 133 - protein, 113 aa, Artificial Sequence; identical to SEQ ID NO: 23
  Light Chain

SEQ ID NO: 134 - DNA, 339 bp, Artificial Sequence; identical to SEQ ID NO: 24
  DNA Light Chain

SEQ ID NO: 135 - protein, 456 aa, Artificial Sequence
  Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - DNA, 1368 bp, Artificial Sequence
  DNA Heavy Chain
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 137 - protein, 220 aa, Artificial Sequence; identical to SEQ ID NO: 27
  Light Chain

SEQ ID NO: 138 - DNA, 660 bp, Artificial Sequence; identical to SEQ ID NO: 28
  DNA Light CHain

SEQ ID NO: 139 - protein, 452 aa, Artificial Sequence
  Heavy Chain
EVQLVESGGGLVKPGGSLRLSCAASGFTFRSYGMNWVRQAPGKGLEWVSGISSSGSYIYYADSVKGRFTISRDNAKNSLYLQMNSLRAEDTAVYYCARDGGRTSYTATAHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 140 - DNA, 1356 bp, Artificial Sequence
  DNA Heavy Chain
gaggtgcagctggtggagtctgggggaggcctggtcaagcctggggggtccctgagactctcctgtgcagcctctggattcaccttccgtagctatgggatgaactgggtccgccaggctccagggaaggggctggagtgggtctcaggtattagtagtagtggtagttacatatactacgcagactcagtgaagggccgattcaccatctccagagacaacgccaagaactcactgtatctgcaaatgaacagcctgagagccgaggacacggcggtgtactactgcgccagagatggtggaagaacgtcctacaccgccacagcccacaattggttcgacccctggggacagggtacattggtcaccgtctcctcagcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgcccttcctgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggc

SEQ ID NO: 141 - protein, 8 aa, Artificial Sequence
  FLAG
DYKDDDDK

SEQ ID NO: 142 - protein, 6 aa, Artificial Sequence
  6-HIS
HHHHHH

SEQ ID NO: 143 - protein, 9 aa, Artificial Sequence
  Synthetic HA
YPYDVPDYA

SEQ ID NO: 144 - protein, 9 aa, Artificial Sequence
  consensus heavy chain CDR1
  misc_feature (5)..(5): Wherein Xaa can be Ser, Ala, or Arg
  misc_feature (6)..(6): Wherein Xaa can be Ser or Arg
  misc_feature (7)..(7): Wherein Xaa can be Thr or Tyr
  misc_feature (8)..(8): Wherein Xaa can be Gly or Ser
  misc_feature (9)..(9): Xaa can be any naturally occurring amino acid
NGFTFXXXX

SEQ ID NO: 145 - protein, 8 aa, Artificial Sequence
  consensus heavy chain CDR1
  misc_feature (5)..(8): Xaa can be any naturally occurring amino acid
GFTFXXXX

SEQ ID NO: 146 - protein, 8 aa, Artificial Sequence
  consensus heavy chain CDR2
  misc_feature (5)..(5): Wherein Xaa can be Ser or Gly
  misc_feature (6)..(6): Wherein Xaa can be Ser or ALa
ISSSXXYI

SEQ ID NO: 147 - protein, 8 aa, Artificial Sequence
  consensus heavy chain CDR2
  misc_feature (5)..(6): Xaa can be any naturally occurring amino acid
ISSSXXYI

SEQ ID NO: 148 - protein, 9 aa, Artificial Sequence
  consensus heavy chain CDR1
  misc_feature (4)..(4): Wherein Xaa can be Ser, Ala, or Arg
  misc_feature (5)..(5): Wherein Xaa can be Ser, or Arg
  misc_feature (6)..(6): Wherein Xaa can be Thr or Tyr
  misc_feature (6)..(6): Wherein Xaa can be Gly or Ser
  misc_feature (7)..(7): Xaa can be any naturally occurring amino acid
FTFXXXXMN

SEQ ID NO: 149 - protein, 17 aa, Artificial Sequence
  consensus heavy chain CDR2
  misc_feature (1)..(1): Wherein Xaa can be Gly or Ser
  misc_feature (6)..(6): Wherein Xaa can be Gly or Ser
  misc_feature (7)..(7): Wherein Xaa can be Ser or Ala
  misc_feature (10)..(10): Wherein Xaa can be Leu or Tyr
XISSSXXYIXYADSVKG

SEQ ID NO: 150 - protein, 9 aa, Artificial Sequence
  consensus heavy chain CDR1
  misc_feature (4)..(7): Xaa can be any naturally occurring amino acid
FTFXXXXMN

SEQ ID NO: 151 - protein, 17 aa, Artificial Sequence
  consensus heavy chain CDR2
  misc_feature (1)..(1): Xaa can be any naturally occurring amino acid
  misc_feature (6)..(7): Xaa can be any naturally occurring amino acid
  misc_feature (10)..(10): Xaa can be any naturally occurring amino acid
XISSSXXYIXYADSVKG

SEQ ID NO: 152 - protein, 8 aa, Artificial Sequence
  heavy chain CDR2
  misc_feature (2)..(8): Xaa can be any naturally occurring amino acid
IXXXXXXX

SEQ ID NO: 153 - protein, 6 aa, Artificial Sequence
  Heavy chain CDR3
  misc_Feature (3)..(3): Wherein Xaa can be any amino acid and can repeat between 6 and 15 times
  misc_feature (5)..(5): Xaa can be any naturally occurring amino acid
ARXDXC

SEQ ID NO: 154 - protein, 7 aa, Artificial Sequence
  Light chain CDR1
  Misc_feature (3)..(3): Wherein Xaa can be any amino acid and repeat itself between 1 and 3 times
  Misc_feature (6)..(6): Wherein Xaa can be any amino acid and repeat itself between 0 and 4 times
QSXSSXY

SEQ ID NO: 155 - protein, 3 aa, Artificial Sequence
  light chain CDR2
  misc_feature (1)..(2): Xaa can be any naturally occurring amino acid
XXS

SEQ ID NO: 156 - protein, 9 aa, Artificial Sequence
  Light chain CDR3
  misc_feature (3)..(6): Xaa can be any naturally occurring amino acid
  misc_feature (8)..(8): Wherein Xaa can be any amino acid and repeat itself 0 to 1 times
QQXXXXPXT